SEQUENCE LISTING (110 sequences)

SEQ ID NO: 1 - DNA, 696 bp, Mus musculus
atggagagctctggcaagatggagagtggagccggccagcagccgcagcccccgcagcccttcctgcctcccgcagcctgcttctttgcgaccgcggcggcggcggcagcggcggcggccgcggcagctcagagcgcgcagcagcaacagccgcaggcgccgccgcagcaggcgccgcagctgagcccggtggccgacagccagccctcagggggcggtcacaagtcagcggccaagcaggtcaagcgccagcgctcgtcctctccggaactgatgcgctgcaaacgccggctcaacttcagcggcttcggctacagcctgccacagcagcagccggccgccgtggcgcgccgcaacgagcgcgagcgcaaccgggtcaagttggtcaacctgggttttgccaccctccgggagcatgtccccaacggcgcggccaacaagaagatgagcaaggtggagacgctgcgctcggcggtcgagtacatccgcgcgctgcagcagctgctggacgagcacgacgcggtgagcgctgcctttcaggcgggcgtcctgtcgcccaccatctcccccaactactccaacgacttgaactctatggcgggttctccggtctcgtcctactcctccgacgagggatcctacgaccctcttagcccagaggaacaagagctgctggactttaccaactggttctga

SEQ ID NO: 2 - protein, 231 aa, Mus musculus
MESSGKMESGAGQQPQPPQPFLPPAACFFATAAAAAAAAAAAAQSAQQQQPQAPPQQAPQLSPVADSQPSGGGHKSAAKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 3 - DNA, 2490 bp, Homo sapiens
agcactctctcacttctggccagggaacgtggaaggcgcaccgacagggatccggccagggagggcgagtgaaagaaggaaatcagaaaggaagggagttaacaaaataataaaaacagcctgagccacggctggagagaccgagacccggcgcaagagagcgcagccttagtaggagaggaacgcgagacgcggcagagcgcgttcagcactgacttttgctgctgcttctgcttttttttttcttagaaacaagaaggcgccagcggcagcctcacacgcgagcgccacgcgaggctcccgaagccaacccgcgaagggaggaggggagggaggaggaggcggcgtgcagggaggagaaaaagcattttcactttttttgctcccactctaagaagtctcccggggattttgtatatattttttaacttccgtcagggctcccgcttcatatttccttttctttccctctctgttcctgcacccaagttctctctgtgtccccctcgcgggccccgcacctcgcgtcccggatcgctctgattccgcgactccttggccgccgctgcgcatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcagggtgatcgcacaacctgcatctttagtgctttcttgtcagtggcgttgggagggggagaaaaggaaaagaaaaaaaaaagaagaagaagaagaaaagagaagaagaaaaaaacgaaaacagtcaaccaaccccatcgccaactaagcgaggcatgcctgagagacatggctttcagaaaacgggaagcgctcagaacagtatctttgcactccaatcattcacggagatatgaagagcaactgggacctgagtcaatgcgcaaaatgcagcttgtgtgcaaaagcagtgggctcctggcagaagggagcagcacacgcgttatagtaactcccatcacctctaacacgcacagctgaaagttcttgctcgggtcccttcacctcctcgccctttcttaaagtgcagttcttagccctctagaaacgagttggtgtctttcgtctcagtagcccccaccccaataagctgtagacattggtttacagtgaaactatgctattctcagccctttgaaactctgcttctcctccagggcccgattcccaaaccccatggcttccctcacactgtcttttctaccattttcattatagaatgcttccaatcttttgtgaattttttattataaaaaatctatttgtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttgccttttggagatttctattactgcctttttttttcttactgttttattacaaacttacaaaaatatgtataaccctgttttatacaaactagtttcgtaataaaactttttcctttttttaaaatgaaaataaaaaaaa

SEQ ID NO: 4 - protein, 236 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 5 - DNA, 1797 bp, Mus musculus
atgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcgggtcggtttactacaagccctcttcgcccccgacacccagcaccccgagcttccaggtgcagcatagcccgatgtgggacgatccgggctcccttcacaacttccaccagaactacgtggccactacgcatatgatcgagcagaggaagacacctgtctcccgcctgtcactcttctcctttaagcagtcgcccccgggcactcctgtgtctagctgccagatgcgcttcgacgggcctctgcacgtccccatgaacccggagcccgcgggcagccaccacgtagtggatgggcagaccttcgccgtgcccaaccccattcgcaagccggcatccatgggcttcccgggcctgcagatcggccacgcatcgcagttgcttgacacgcaggtgccctcgccgccgtcccggggctctccctccaatgagggtctgtgcgctgtttgcggtgacaacgcggcctgtcagcactacggtgttcgcacttgtgagggctgcaaaggtttctttaagcgcacggtgcaaaaaaacgcgaaatatgtgtgtttagcaaataaaaactgcccagtggacaagcgccgccgaaatcgttgtcagtactgtcggtttcagaagtgcctagctgttgggatggttaaagaagtggttcgcacggacagtttaaaaggccggagaggtcgtttaccctcgaagccgaagagcccacaggatccctctcccccctcacctccggtgagtctgatcagtgccctcgtcagagcccacgtcgattccaatccggcaatgaccagcctggactattccaggttccaggcaaaccctgactatcagatgagtggagatgatacccaacatatccagcagttctacgatctcctgaccggctctatggagatcatcagagggtgggcagagaagatccctggctttgctgacctgcccaaagccgaccaggacctgctttttgaatcagctttcttagaattatttgttctgcgcttagcatacaggtccaacccagtggagggtaaactcatcttttgcaatggggtggtcttgcacaggttgcaatgcgtgcgtggctttggggaatggattgattccattgttgaattctcctccaacttgcagaatatgaacatcgacatttctgccttctcctgcattgctgccctggctatggtcacagagagacacgggctcaaggaacccaagagagtggaagagctacaaaacaaaattgtaaattgtcttaaagaccatgtgactttcaataatgggggtttgaaccgacccaactacctgtctaaactgttggggaagctgccagaactccgcaccctttgcacacagggcctccagcgcattttctacctgaaattggaagacttggtaccaccaccagcaataattgacaaacttttcctggacaccttacctttctaa

SEQ ID NO: 6 - protein, 598 aa, Mus musculus
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPSTPSFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQDPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 7 - DNA, 3546 bp, Homo sapiens
gctgacgcgcgctgacgcgcggagactttaggtgcatgttggcagcggcagcgcaagccacataaacaaaggcacattggcggccagggccagtccgcccggcggctcgcgcacggctccgcggtcccttttgcctgtccagccggccgcctgtccctgctccctccctccgtgaggtgtccgggttcccttcgcccagctctcccacccctacccgaccccggcgcccgggctcccagagggaactgcacttcggcagagttgaatgaatgaagagagacgcggagaactcctaaggaggagattggacaggctggactccccattgcttttctaaaaatcttggaaactttgtccttcattgaattacgacactgtccacctttaatttcctcgaaaacgcctgtaactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataatgtataatgggaaatgtttggccatgcttggttgttgcagttcagacaaatgtaacacacacacacatacacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgaacagaaaaaaaaaaaaaaaaa

SEQ ID NO: 8 - protein, 598 aa, Homo sapiens
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 9 - DNA, 1149 bp, Mus musculus
atgttggacggcctgaagatggaggagaactttcaaagtgcgattgagacctcggcatctttctcctctttgctgggcagagcggtgagccccaagtctgtctgcgagggctgtcagcgggtcatctcggacaggtttctgctgcggctcaacgacagcttctggcacgagcaatgcgtgcagtgtgcctcctgcaaagagcccctggagaccacctgcttctaccgggacaagaagctctactgcaagtaccactacgagaaactgtttgctgtcaaatgtgggggctgcttcgaggccattgcgcccaatgagtttgtcatgcgtgcccagaagagcgtataccacctgagctgcttctgctgctgcgtctgtgagcgacagctgcagaagggtgacgagtttgtcctgaaggagggccagctgctctgcaaaggggactatgagaaagaacgggagctgctgagcctggtgagccctgcggcctcagactcaggcaaaagcgatgatgaggagagcctttgcaagtcagcccatggggcaggaaaaggagcatcagaggacggcaaggaccataagcgacccaaacgtcccagaaccatcctgaccactcagcagaggagagcattcaaggcctcgtttgaagtatcctccaagccctgcagaaaggtgagggagactctggctgcggagacagggctgagtgtccgtgtggttcaggtgtggttccagaaccagcgagccaagatgaagaagctggcccggcgacagcagcaacagcaacaggaccaacagaacacccagaggctgacttctgctcagacaaatggtagtgggaatgcgggcatggaagggatcatgaacccctatacaacgttgcccaccccacagcagctgctggccattgaacagagcgtctacaactctgatcccttccgacagggtctcaccccaccccagatgcctggagatcacatgcacccctatggtgctgaacctcttttccatgacttggatagtgatgacacatctctcagtaacctgggagactgcttcctggcaacctcagaagctgggcccctgcagtccagagtgggaaaccccattgaccatctgtactccatgcagaattcctatttcacctcttga

SEQ ID NO: 10 - protein, 381 aa, Mus musculus
MLDGLKMEENFQSAIETSASFSSLLGRAVSPKSVCEGCQRVISDRFLLRLNDSFWHEQCVQCASCKEPLETTCFYRDKKLYCKYHYEKLFAVKCGGCFEAIAPNEFVMRAQKSVYHLSCFCCCVCERQLQKGDEFVLKEGQLLCKGDYEKERELLSLVSPAASDSGKSDDEESLCKSAHGAGKGASEDGKDHKRPKRPRTILTTQQRRAFKASFEVSSKPCRKVRETLAAETGLSVRVVQVWFQNQRAKMKKLARRQQQQQQDQQNTQRLTSAQTNGSGNAGMEGIMNPYTTLPTPQQLLAIEQSVYNSDPFRQGLTPPQMPGDHMHPYGAEPLFHDLDSDDTSLSNLGDCFLATSEAGPLQSRVGNPIDHLYSMQNSYFT

SEQ ID NO: 11 - DNA, 3555 bp, Homo sapiens
agaagctgcaggatccgccccggcgaagcagggccgactcgcacccaggaccctgggcctctgccttccctcctagccttggagaagcaactggccctctcctcccgctgaggagcgacgcgggctggtaggacgtcccgggaaggccggcagctcgcgaccacgtcccggcccagcctgggcgcgccgaggagcagagccagcggccggcgttcgctccggctccctccccggcgctccgaagccgagggcggctcctccggctgcagtctcgggggcgacgccttcccgggcagaagcttccagcagcgctccgcaacttctctctgctccagtcactgggagagagctcgcctaccaggtcctcccggcccggcccgaacatgctggacggcctaaagatggaggagaacttccaaagcgcgatcgacacctcggcctccttctcctcgctgctgggcagagcggtgagccccaagtctgtctgcgagggctgtcagcgggtcatcttggacaggtttctgctgcggctcaacgacagcttctggcatgagcagtgcgtgcagtgcgcctcctgcaaagagcccctggagaccacctgcttctaccgggacaagaagctgtactgcaagtatgactacgagaagctgtttgctgttaaatgtgggggctgcttcgaggccatcgctcccaatgagtttgttatgcgggcccagaagagtgtataccacctgagctgcttctgctgctgtgtctgcgagcgacagcttcagaagggtgatgagtttgtcctgaaggaggggcagctgctctgcaaaggggactatgagaaggagcgggagctgctcagcctggtgagcccagcagcctcagactcaggtaaaagtgatgatgaagaaagtctctgcaagtcagcccatggggcagggaaaggaactgctgaggaaggcaaggaccataagcgccccaaacgtccgagaaccatcttgacaactcaacagaggcgagcattcaaggcctcatttgaagtatcctccaagccctgcaggaaggtgagagagactctggctgcagagacagggctgagtgtccgtgtcgtccaggtgtggttccaaaaccagagagcgaagatgaagaagctggccaggcgacagcagcagcagcagcaagatcagcagaacacccagaggctgagctctgctcagacaaacggtggtgggagtgctgggatggaaggaatcatgaacccctacacggctctgcccaccccacagcagctcctggccatcgagcagagtgtctacagctcagatcccttccgacagggtctcaccccaccccagatgcctggagaccacatgcacccttatggtgccgagccccttttccatgacctggatagcgacgacacctccctcagtaacctgggtgattgtttcctagcaacctcagaagctgggcctctgcagtccagagtgggaaaccccattgaccatctgtactccatgcagaattcttacttcacatcttgagtcttcccctagagttctgtgactaggctcccatatggaacaaccatattctttgaggggtcactggctttaggacagggaggccagggaagaggtgggttggggagggagttttgttggggatgctgttgtataatgatatggtgtagctcagcatttccaaagactgaatacattatggattgcatagtttaatgtttctaataagagtcttagcattagatatgaagacgtgtttatcattaaggacagagacttttaatatagacattctcatgcaaactagatacttagggactcctaacaacttcccaccatgtcggggaagctcttgtcaagaggtgcatatgtctatccatctacacaccaatagacagaaggacagatagatagatgtgtgtgtgtgagtgtgtaacctttcgtattttaccctcaaagtttattcctaattataacagacaccaactgtacagcaaaagtaactttattttcagtgtgaactatatttaaggaaatgcttgatgcacttaagttataaaatgagataatttacttttataaactttatttttagcttgacaagacttgtcagcagggcagagagggctgctccacctagccccatagctttgagtgctggggttcattctgttttcagagtgtctttcagatctggaaagaaattctgtgtggctgatggtgttctctcttgcattcttgctctctttggggttgaatcactgggcaggggtgggacagaataatctctgatcatgttctgagaaaatgtaaagcccagactcctgggctttcttttaaattctgacaagtggttgttgggcagtgctaggatgattggttcagctcttgagcttcagcatctgcaaatgtggatgaggctaatagtatgtacctacctcactgggaaacaccaaggcttaattcattcccaggacacatgagcagggctgagactaatatctgatatttgtttaagatacaaccaggccactcacttggcaaaggagggtacatagggttgcagagcaggagggctcctgaactccagagggcagttctgcctgctgaagtccctctgcaaagcctgtgctgaaggagacaccagctcagagcagttcagagggatcccagagtcccagagtggggaggaggtgaaggctgaggggatagaggagggcctggtggtgttctagagcagggttgggcaaactcctgcttgcgggcctgctttctatggcttgccagcaaagaatggtttttactttttttttgaggtcattaaaaaaaaggagaagaagaatatataacaggctgtctgtggcctggaaagcctgaaatatttgctatctgtattgtctggcccttacagaaaaagtttggggccccttgttttagagggtctgtttctaaagaacctcatggcgctcatagaggcagaaggttccagtggaaacccttggctcttccttccaactcactcctctgatcctcggcacagaagacccagcagccattgtacatggggacagttccacaccctggtctccagttgcggtgctaggatggtattgttctgtgctaggaagtctcctgggaacccagaatgagttggtggggaagacagcgggtcactgtggacccatccaggaggggccaggataggcttggcctcatttctggggacatcattggagacttgaacacagagacacgtccctatcactctggcaaggccagagggaacatgtccccttatggtagagtctatgttgtgtgatttttgtgctcttgtttataatttatgcaaaccaccaagaaacccaaaccagtctgatgagtgaaaattatgcagatgctgtatggccccacaggtttctgtggtaaagaccagttggagaatgtaggagatactatgtgagtgaaaatgaatagagatccttattccactccttaatggcataccaagatgaaattaaaatctcttacaaatgaaaaaaaaaaa

SEQ ID NO: 12 - protein, 382 aa, Homo sapiens
MLDGLKMEENFQSAIDTSASFSSLLGRAVSPKSVCEGCQRVILDRFLLRLNDSFWHEQCVQCASCKEPLETTCFYRDKKLYCKYDYEKLFAVKCGGCFEAIAPNEFVMRAQKSVYHLSCFCCCVCERQLQKGDEFVLKEGQLLCKGDYEKERELLSLVSPAASDSGKSDDEESLCKSAHGAGKGTAEEGKDHKRPKRPRTILTTQQRRAFKASFEVSSKPCRKVRETLAAETGLSVRVVQVWFQNQRAKMKKLARRQQQQQQDQQNTQRLSSAQTNGGGSAGMEGIMNPYTALPTPQQLLAIEQSVYSSDPFRQGLTPPQMPGDHMHPYGAEPLFHDLDSDDTSLSNLGDCFLATSEAGPLQSRVGNPIDHLYSMQNSYFTS

SEQ ID NO: 13 - DNA, 1219 bp, Mus musculus
atgttggacggcatcaagatggaggagcacgcccttcgccccgggcccgccaccctgggggtgctgctgggctccgactgcccgcatcccgccgtctgcgagggctgccagcggcccatctccgaccgcttcctgatgcgagtcaacgagtcgtcctggcacgaggagtgtttgcagtgcgcggcatgtcagcaagccctcaccaccagctgctacttccgggatcggaaactgtactgcaaacaagactaccaacagctcttcgcggcaaagtgcagcggctgcatggagaagatcgcccctaccgagttcgtcatgcgggcgctggagtgtgtgtaccacttgggctgtttctgctgctgtgtgtgcgagaggcaactgcgcaagggggacgagttcgtgctcaaggagggccagctgctgtgcaagggtgactatgagaaggagaaagacctgctcagctccgtgagcccggacgagtctgactctgtgaagagtgaggatgaagatggagacatgaagccggccaaggggcagggcagccagagtaaaggcagtggagatgacgggaaagacccgagaaggcccaaacggccccgaaccatcctcaccacacagcagcgaagagctttcaaggcatcctttgaggtctcctccaagccctgtcggaaggtccgagagacattggcagcagagacaggcctcagcgtgcgtgtggtccaggtctggtttcagaaccaaagagcaaagatgaagaagctggcccggagacaccagcaacagcaggagcagcagaactcccagcggctgggccaagaggttctgtcaagccgcatggagggcatgatggcctcctacacgccgctggcccctccgcagcagcagatcgtggccatggagcagagcccctacggaagtagcgaccccttccaacagggcctcacgccgccccaaatgccagggaacgactccatcttccacgatatcgatagtgatacctccctcaccagcctcagcgactgcttcctcggctcttccgacgtgggctccctgcaggcccgcgtggggaaccccattgaccggctctactccatgcagagctcctactttgcctcctgagagccagccgggccgcatggacgcttgggcctgggcctagggtggagccacaggcctctgcagccagccggccccccagcccaccacccgctcagactct

SEQ ID NO: 14 - protein, 372 aa, Mus musculus
MLDGIKMEEHALRPGPATLGVLLGSDCPHPAVCEGCQRPISDRFLMRVNESSWHEECLQCAACQQALTTSCYFRDRKLYCKQDYQQLFAAKCSGCMEKIAPTEFVMRALECVYHLGCFCCCVCERQLRKGDEFVLKEGQLLCKGDYEKEKDLLSSVSPDESDSVKSEDEDGDMKPAKGQGSQSKGSGDDGKDPRRPKRPRTILTTQQRRAFKASFEVSSKPCRKVRETLAAETGLSVRVVQVWFQNQRAKMKKLARRHQQQQEQQNSQRLGQEVLSSRMEGMMASYTPLAPPQQQIVAMEQSPYGSSDPFQQGLTPPQMPGNDSIFHDIDSDTSLTSLSDCFLGSSDVGSLQARVGNPIDRLYSMQSSYFAS

SEQ ID NO: 15 - DNA, 5783 bp, Homo sapiens
gcgtcccatggatatagcaacaggtcccgagtcgctggagaggtgcttccctcgcgggcagacggactgcgccaagatgttggacggcatcaagatggaggagcacgccctgcgccccgggcccgccactctgggggtgctgctgggctccgactgcccgcatcccgccgtctgcgagggctgccagcggcccatctccgaccgcttcctgatgcgagtcaacgagtcgtcctggcacgaggagtgtttgcagtgcgcggcgtgtcagcaagccctcaccaccagctgctacttccgggatcggaaactgtactgcaaacaagactaccaacagctcttcgcggccaagtgcagcggctgcatggagaagatcgcccccaccgagttcgtgatgcgggcgctggagtgcgtgtaccacctgggctgcttctgctgctgcgtgtgtgaacggcagctacgcaagggcgacgaattcgtgctcaaggagggccagctgctgtgcaagggtgactacgagaaggagaaggacctgctcagctccgtgagccccgacgagtccgactccgtgaagagcgaggatgaagatggggacatgaagccggccaaggggcagggcagtcagagcaagggcagcggggatgacgggaaggacccgcggaggcccaagcgaccccggaccatcctcaccacgcagcagcgaagagccttcaaggcctccttcgaggtctcgtcgaagccttgccgaaaggtccgagagacactggcagctgagacgggcctcagtgtgcgcgtggtccaggtctggtttcagaaccaaagagcaaagatgaagaagctggcgcggcggcaccagcagcagcaggagcagcagaactcccagcggctgggccaggaggtcctgtccagccgcatggagggcatgatggcttcctacacgccgctggccccaccacagcagcagatcgtggccatggaacagagcccctacggcagcagcgaccccttccagcagggcctcacgccgccccaaatgccagggaacgactccatcttccatgacatcgacagcgatacctccttaaccagcctcagcgactgcttcctcggctcctcagacgtgggctccctgcaggcccgcgtggggaaccccatcgaccggctctactccatgcagagttcctacttcgcctcctgagagccagccaggcgcacggacgcttgggcaggggcctgggggggactgccagcctctgcggccagcctggccacccccgccctgctctccgcacagactacagacagccatacggtgccctcccctcggccagctgggcctgaccactgtgcccgttgggtacagccagaccggtagatgggcacagcctgggcaggggctgtgtcctgcccacagagaccttgtcatccccagggacccagagctctcggacggccactcgcctcccagccccacctcggcctccatcgcctcctccccatctcttttttgggaagcttaaattctctctatttttttaaatgtcctctctgtgtccatggccctccatgcaagccccaggacaatggtgtcatgaggcggtgacctgagaagcgtgtgtacctgtgccccagcaagggcaggggtggcctctgggggcaggcccactgcctggaaccgcacacccctcagcctgagtctggagcagcagtggagaggggcctgaggggaggcactgtcaggaggcgggctcggagcctgagcctgggcaggcgcaaagggacagagaggcacgtgcagacacatgcacacttgcagacaaacccacgcaaacacacacacagctgtatggggacaccagaagggacagggatgctcagcgggtctgtcctgccttgtcagaaagagaaaaggaggccaggcaggggaccccccagttcttaagagcgattggaaagggaggaaggggagaggaagaggcgaacttgaagcatcggacccagttgtatcccagcctgggcccaaatgggggcagcctgggcagggagggcagccccaggccccaccaactctagaggcagatggagcccccagaaccaggtagcatcagaccagacaacagagcctccaggggtcagggacttcagaagcacctgctgggcaccccatctgcaatgtggtcctctccccagccacctctgcctcccctcacatacctccagtgacaaggagctcactaggtcagcgagcccacagcagctgtgctgtcctgcatcccagagccaggcttccccagctctccctcttaacactgtcccccagcaggcctccggctgtccctctaaaggtgtggggcaggtatcacttcaccttcccactgatgtcagccggccagaagtgagcaggcacatcacctctcctgctgtggcacccttcctctgttaatttggcccaaaagacaatgatttggccacatgaccttagagattcaccctgccctgctgtagctaaatccctgggccccacacgcaagtgacagctaagccacatctgttttctgtgtatatgcaggatgggggcacctactgttttgttttgttttgttttgttttgttttgttttgttttgttttgttttgtttgagacggagtttcgctcttgttgcccaggctggagtgcaatggcgcgatctcggctcaccacaacctccgcctcccaggttcaagtgattctgatgcctcagcctccctagtagctgagattacaggcatgcgccaccacacccagctaattttgtatttttagtagcaacggggtttctccatgttggtcaggctggtctccaacccccgacctcaggtgatccgcctgcctcggcctcccaaagtgctgggattacaggcgtgagccaccgcacccagtctgcacttactgtttagactgaatgagggaccgtgacctctttccttttccattccttcttactcgattcattccagcctgtggaatttctctgcaccctgattcagtgaccactgctctcctctctcccagcacatctgcccagtgaggagttggccctgggtctcacctgaggtgtgtggaccgggctggcctctccctgtttgacattggcccattaatgcatcctctttgggggacacattccaattgcatttcctgcccccttctcccagggcaattgcagaagattgtgtcaggcgccctgctggaagtcaggtgcactagatccatccccagccccagtctgctcaactctatccctgtcagagcaaggaggctgggctgctggggcctgactggtgagcccaccctgtcccctggtgatcactgtgtccccttgttcaggtgctcacaaccctacctttaactctgaggtcaagccctaggccaccaccctaaagtctgcctggtccaacctttgagcaagtaaggataatgaatgtcccttttccacctttggggccctctgcctggatctctggaatcctctaagttcaacctgttctgtggttttgctcccgtttgctgggaaattcagtccccccagaatgtcctgggccaacctccttgcctgacatgtggcctcgtgtcacccattgggccccagcagccagctagcccttctgcagctcttcttacaaacagagcctctccaaggacctcagttgatgttctggtccttctgccgcctcagcccaccagggtccgtgccaccatgggtctcttgagcagcagctgcactggcttctggagagacacccctctttctccttttgcacatgcaccatctgaatcgtgccagggacatcctgggcagattcaggggcagatgccctatcccccaggagacctggcccttctctctcagacccaataagttggaagggacgtcagaagcggtcatctcatctgccccttattttatagttggaaaccctgaggcaagagagggaaagaggcctgtccaaggtccgggttagtgacagagctgagctgagaacagggacgttgtgccccactgtcccctgtggtttgtgaatgacctccaggtcagggggtcacaacttgttcttagtaaacttgccagctgttggggtcacatattcccattctggggcctcacaaacccccgaatccagccgggaccccatgccaggagctggtctagggacagcatgcttgtgacccacagactgttaaagccagaagggacctcagagagtcccttatgctggaggcgccctgtcagccgtggctaggggccccttgctctatgctgtgccttgctgcccacaggctcccagacaccagtgcccactctgcccagccccggactgggtgtggctcgcagatgaacaagatgcagggcctgccttgaggggtgtctcctagaaggaaagccagactctccggcccagccagagagtccagacatggcagggacccgtttctcagatgaggagcctgaggctcagagaagggaggcgatgtgttcagggccacccagcagaagcctgtggggctgggcaaccttctcccactttatgggaggagctgcagccttggctgggagctgggcggggagtagccaggaccaccccttgcccgtgccgtgacatggaaccttcatcactaagggggctggagtgggaagagggagataactgtgtggtctccagagcaaaagagaatgagaggtgggcagggggagtcttggcaaaagaccaagttccacttccctgctggggaagtcaaggctcagaaagaggaaataattgccccaggtaacacagggcagaggagggacaaaaagctgggcatggccccagccagagcctcatctgcctactccgtgaagcctcccaggtactctgctatcctgggaaacgcacagggaggccacacagagacactgctcacaagagtcagaccaaggtgccagcacagcctggaaagagctcagaaagggggttggtgcacgtggctgggcatcttaggaggcttcctgagggtgggtaaaggtgggaaggccctggcgctgcatcagatgagcagggcctggcagggacaagcctcttctcctttgggaagccctgcagcctcctagcaagaggctgattccccactctgcccccatctgaatgtccttttcatgttgcacgcagggaacctcaggaaggaggattgcctgatgcctgcctggctccatccttgagctctgggcaccacctagggtgagggagagcctgcagctctggggctaagtctgccctggggggaaagggctccacgctcacacgcacgcgctcgcacacacacactcacacctggacgcacacggaggcttgcggacccatactcacaggcacatgtggcctggggactgggggagcaggaaagacccctccaacatttggcccttggaaggcaccattgccaatgagcctctttgctggttcccccgaccccacctgggggtcccatgggagcccagcccagccaggtgtggggatgggccaccggccattcctgttttccttgtacagacagattctcactacccacccgccatccccagacacattttatttaataacttgtcattgttaaattatttattagcgtttaccacaccaccacccccaccctgccctccactctcaccttccacctcttcccacaacagcagaaaatggaaacaacaacaaaaaaagatgagacatcagtatatttgtaaataaaccgacctgtacactcaaaaaaa

SEQ ID NO: 16 - protein, 395 aa, Homo sapiens
MDIATGPESLERCFPRGQTDCAKMLDGIKMEEHALRPGPATLGVLLGSDCPHPAVCEGCQRPISDRFLMRVNESSWHEECLQCAACQQALTTSCYFRDRKLYCKQDYQQLFAAKCSGCMEKIAPTEFVMRALECVYHLGCFCCCVCERQLRKGDEFVLKEGQLLCKGDYEKEKDLLSSVSPDESDSVKSEDEDGDMKPAKGQGSQSKGSGDDGKDPRRPKRPRTILTTQQRRAFKASFEVSSKPCRKVRETLAAETGLSVRVVQVWFQNQRAKMKKLARRHQQQQEQQNSQRLGQEVLSSRMEGMMASYTPLAPPQQQIVAMEQSPYGSSDPFQQGLTPPQMPGNDSIFHDIDSDTSLTSLSDCFLGSSDVGSLQARVGNPIDRLYSMQSSYFAS

SEQ ID NO: 17 - DNA, 1737 bp, Mus musculus
caggtttatctggtctcactccatcccctctagttttggagctgctggggggtgggggggacggcgggggtgggggacgcatctgcaactcctttaaaagcctgtgcccagcgtctcccgggttctttttagttagtgctggaacgtggaggaagctgctccctccgaagcagtaaaccagcatttctgtttgtttgtttgctttgcccttagttccgtcactccaaatctacccaccaaggaccctgaccctgtccactccaggcgaatcgagaccgtccggctgggtccccccaatttgggccgactttgcgcctccaaacaaccttagcatgatgtcttatctaaagcaaccgccttacgcagtcaatgggctgagtctgaccacttcgggtatggacttgctgcatccctccgtgggctaccccgccaccccccggaaacagcgaagggagaggacgacatttactagggcacagctcgacgttctggaagctctgtttgccaagacccggtacccagacatcttcatgagggaagaggtggcactgaaaatcaacttgccagaatccagggtgcaggtatggtttaagaatcgaagagctaagtgccgccaacagcagcagcagcagcagaatggaggtcagaacaaagtgaggcctgccaagaagaagagctctccagctcgggaagtgagttcagagagtggaacaagtggccagttcagtcccccctctagtacctcagtcccaaccattgccagcagcagtgctccagtgtctatctggagcccagcgtccatctccccactgtctgaccccttgtccacttcctcctcctgcatgcagaggtcctatcccatgacctatactcaggcttcaggttatagtcaaggctatgctggctcaacttcctactttgggggcatggactgtggatcttatttgacccctatgcatcaccagcttcctggaccaggggccacactcagtcccatgggtaccaatgctgttaccagccatctcaatcagtccccagcttctctttccacccagggatatggagcttcaagcttgggttttaactcaaccactgattgcttggattataaggaccaaactgcctcttggaagcttaacttcaatgctgactgcttggattataaagatcagacgtcctcatggaaattccaggttttgtgaagacctgtagaagctatttttgtgggtgatttttaaatatgctgggctgaacattccagttttagccaggcattggttaaaaaagttagatggaacgatgctctcagactcctgatcaaagttaccgagaggcatagaaggaaaaaggaaggggccttagaagggtccatcaaccagcaacctgaaatggacaaaccaatctacttaagattctgttatagttctagatcattggtttcctgatttgcaaatgattgatcaaatatattctagcgacatgcaaccaaataccactcaaaacaaaaatccagcaaaactgagttgtgagggaagggagggaaggtcatggccttcaaagcagaggtgatccggtgttttagccaatctttggttgaatcttaggaatggacaatgtcccaggctcattcacgtttcatgaccaacaggtagttggcactgaaaaacttttcagggctgtgtggattgtgcgactgattgtcctagatgcactactttatttaaaaaaaaaaaaaaa

SEQ ID NO: 18 - protein, 289 aa, Mus musculus
MMSYLKQPPYAVNGLSLTTSGMDLLHPSVGYPATPRKQRRERTTFTRAQLDVLEALFAKTRYPDIFMREEVALKINLPESRVQVWFKNRRAKCRQQQQQQQNGGQNKVRPAKKKSSPAREVSSESGTSGQFSPPSSTSVPTIASSSAPVSIWSPASISPLSDPLSTSSSCMQRSYPMTYTQASGYSQGYAGSTSYFGGMDCGSYLTPMHHQLPGPGATLSPMGTNAVTSHLNQSPASLSTQGYGASSLGFNSTTDCLDYKDQTASWKLNFNADCLDYKDQTSSWKFQVL

SEQ ID NO: 19 - DNA, 2209 bp, Homo sapiens
gagagcgggaccggcctcagctccaacacagcctccactgtgattaaaaataaaaattgctagagcagccctcactcgccacatctactttgatagctggctatttggaatttaaaggatatttgactttttctaacctcccatgaggctgtaagttccactgctccaaacccacccaccaaggactctgaacctgtccaccccgggcgcatcaagatcttccagctgggtacccccgatttgggccgactttgcacctccaaacaaccttagcatgatgtcttatcttaagcaaccgccttacgcagtcaatgggctgagtctgaccacttcgggtatggacttgctgcacccctccgtgggctacccggggccctgggcttcttgtcccgcagccaccccccggaaacagcgccgggagaggacgacgttcactcgggcgcagctagatgtgctggaagcactgtttgccaagacccggtacccagacatcttcatgcgagaggaggtggcactgaaaatcaacttgcccgagtcgagggtgcaggtatggtttaagaatcgaagagctaagtgccgccaacaacagcaacaacagcagaatggaggtcaaaacaaagtgagacctgccaaaaagaagacatctccagctcgggaagtgagttcagagagtggaacaagtggccaattcactcccccctctagcacctcagtcccgaccattgccagcagcagtgctcctgtgtctatctggagcccagcttccatctccccactgtcagatcccttgtccacctcctcttcctgcatgcagaggtcctatcccatgacctatactcaggcttcaggttatagtcaaggatatgctggctcaacttcctactttgggggcatggactgtggatcatatttgacccctatgcatcaccagcttcccggaccaggggccacactcagtcccatgggtaccaatgcagtcaccagccatctcaatcagtccccagcttctctttccacccagggatatggagcttcaagcttgggttttaactcaaccactgattgcttggattataaggaccaaactgcctcctggaagcttaacttcaatgctgactgcttggattataaagatcagacatcctcgtggaaattccaggttttgtgaagacctgtagaacctctttttgtgggtgatttttaaatatactgggctggacattccagttttagccaggcattggttaaaagagttagatgggatgatgctcagactcatctgatcaaagttccgagaggcatagaaggaaaaacgaagggccttagaggggcctacaaaccagcaacatgaaatggacaaaccaatctgcttaagatcctgtcatagttttagatcattggttatcctgatttgcaaagtgatcaaaagcattctagccatgtgcaaccaaacaccaccaaaaataaaatcaaacaaaactaagttgtgaaggaagggagggaaggtcatagccttcttaagcagaggtgttccattgttttagccaatccttggttgaatcttaggaatgaacagtgtctcaagctcattcacgtttcatgaccaactggtagttggcactgaaaaaacttttcagggctgtgtgaattgtgtgactgattgtcctagatgcactactttatttaaaaaataatgttcataaggagtcaatatgtagtttaagagacaatcagtgtgtgtcttataaatggtacatctgtggtttttaatctgtgctagacttcaaaactgtgatctcctgttattgtatgcaaccttgaactccacctctgcaggggttcttctgtgattaaataggttataattataagcaaaattcagagcaactgagtactgatctaaaaagattacctttggctggaggtgagctgcactgaaactttacgacaaaatgtctctggacaaagagagtcagagaagagaagcaaaaggacactaattcatctgtaatttactgttggtaagcctagcagtaaagagacattggtcaattgctctgaccctgatgaattattaaactgagatcattgtcgtttatgcttgcagatgttaaatggaaaagttatatatgcataaaccttttcttcctggatttggcagatatgtataattatattaaaatggttctagcacaaaaaaaaaaaaaaa

SEQ ID NO: 20 - protein, 297 aa, Homo sapiens
MMSYLKQPPYAVNGLSLTTSGMDLLHPSVGYPGPWASCPAATPRKQRRERTTFTRAQLDVLEALFAKTRYPDIFMREEVALKINLPESRVQVWFKNRRAKCRQQQQQQQNGGQNKVRPAKKKTSPAREVSSESGTSGQFTPPSSTSVPTIASSSAPVSIWSPASISPLSDPLSTSSSCMQRSYPMTYTQASGYSQGYAGSTSYFGGMDCGSYLTPMHHQLPGPGATLSPMGTNAVTSHLNQSPASLSTQGYGASSLGFNSTTDCLDYKDQTASWKLNFNADCLDYKDQTSSWKFQVL

SEQ ID NO: 21 - DNA, 1379 bp, Mus musculus
gcggccgccccagagcaggggggcggcccccaccccgcagggtgcctggcccctggcccctgcctgcgctccagaacgccgccgccacagccaccacccggagtctgcctgctgcgggacgcactagacctccctccatggagtttgggctgcttggtgaggcagaggcgcgaagccctgcgctgtcgttatcggacgcaggcactccacaccctccgcttccagaacatggctgcaaggggcaggagcacagtgactcggagaaggcctcggcctcactgccggggggctcccccgaggacggctctctgaagaagaagcagcggcggcagcgcacgcacttcaccagccagcagctgcaggagctggaggccaccttccagaggaatcgctaccctgacatgagcacccgcgaagagatcgcggtgtggaccaacctcactgaggcccgcgtgcgggtgtggttcaagaaccggcgcgccaagtggcggaagcgggagcgcagccagcaggcggagctgtgcaaaggtggcttcgcagccccgctcgggggcctggtgccaccctacgaggaggtgtacccgggctactcgtacggcaactggccgcccaaggctctcgccccgccgctcgccgccaagaccttcccgttcgccttcaactcggtcaacgtggggcctctggcttcacagcctgtattctcaccgcccagctccatcgccgcttctatggtgccctcggccgccgctgccccgggcaccgtaccaggtcccggagccttgcagggcctgggcggggcaccccccgggctggctccagccgccgtgtcctccggggcagtgtcctgcccttacgcctcggccgccgcagccgccgctgcagccgcctcctccccctatgtataccgggacccgtgtaactcgagcctggctagcctgcggctcaaagccaagcagcacgcctctttcagctatcccgccgtgcccgggccgccgccggccgctaaccttagcccctgccagtacgccgtggaacggccggtgtgagccgcaggtctgtggatccatccccgagggcggggcagtaattcacagcctctccggacaggggtcgcctagactggcttgccctcgtcccagggtctgaaaggggtgccagagcacccgggaagaggccgcgggcttcgaagagggccttttccctcgcagcccccgagcggtggtctgacccctatgcggagaccgcgcccctaggactaaggccaggaacagggaccagctcccccagggccaattcacccttggctcaccccgccttctccagactccccctatcccattttcaaagatcaatgaaataaacgtgcgcggactgtcaaa

SEQ ID NO: 22 - protein, 302 aa, Mus musculus
MEFGLLGEAEARSPALSLSDAGTPHPPLPEHGCKGQEHSDSEKASASLPGGSPEDGSLKKKQRRQRTHFTSQQLQELEATFQRNRYPDMSTREEIAVWTNLTEARVRVWFKNRRAKWRKRERSQQAELCKGGFAAPLGGLVPPYEEVYPGYSYGNWPPKALAPPLAAKTFPFAFNSVNVGPLASQPVFSPPSSIAASMVPSAAAAPGTVPGPGALQGLGGAPPGLAPAAVSSGAVSCPYASAAAAAAAAASSPYVYRDPCNSSLASLRLKAKQHASFSYPAVPGPPPAANLSPCQYAVERPV

SEQ ID NO: 23 - DNA, 1407 bp, Homo sapiens
ggagcgcccgagcggagaggcggcccgggagcaggggggcggcccccactccggccgggtgcccggcccctggcccctgcctgccctctagatcgccgccgcagccgccgctactgggagtctgcctgttgcaggacgcactagccctccctccatggagttcggcctgctcagcgaggcagaggcccggagccctgccctgtcgctgtcagacgctggcactccgcacccccagctcccagagcacggctgcaagggccaggagcacagcgactcagaaaaggcctcggcttcgctgcccggcggctccccagaggacggttcgctgaaaaagaagcagcggcggcagcgcacgcacttcaccagccagcagctacaggagctagaggcgaccttccagaggaaccgctaccccgacatgagcacgcgcgaggagatcgccgtgtggaccaacctcaccgaggcccgcgtgcgggtgtggttcaagaaccggcgcgccaaatggcggaagcgcgagcgcagccagcaggccgagctatgcaaaggcagcttcgcggcgccgctcggggggctggtgccgccctacgaggaggtgtaccccggctactcgtacggcaactggccgcccaaggctcttgccccgccgctcgccgccaagacctttccattcgccttcaactcggtcaacgtggggcctctggcttcgcagcccgtcttctcgccacccagctccatcgccgcctccatggtgccctccgccgcggctgccccgggcaccgtgccagggcctggggccctgcagggcctgggcgggggcccccccgggctggctccggccgccgtgtcctccggggccgtgtcctgcccttatgcctcggccgccgccgccgccgcggctgccgcctcttccccctacgtctatcgggacccgtgtaactcgagcctggccagcctgcggctcaaagccaaacagcacgcctccttcagctaccccgctgtgcacgggccgcccccggcagccaaccttagtccgtgccagtacgccgtggaaaggcccgtatgagcggccccgcccgtagatcatccccgagggcgggggcaacgattcacagcctccgcggactggggtcattttgactggcttgctcccgccccagggtctgaaaggggtgtttgggcagctggggggcaccggctcaggagagggccttcccctcccagccctgaggggtggactaggccctacacacagaccgcgcccctgggactaaagccaggaacagggaccagctccccgggggccaactcacccttggcccatcccgccttctccaggcttcccctccctcgttttcaaagataaatgaaataaacgtgcgcggactgtcaaaaaaaaaaaaaaaaaaa

SEQ ID NO: 24 - protein, 302 aa, Homo sapiens
MEFGLLSEAEARSPALSLSDAGTPHPQLPEHGCKGQEHSDSEKASASLPGGSPEDGSLKKKQRRQRTHFTSQQLQELEATFQRNRYPDMSTREEIAVWTNLTEARVRVWFKNRRAKWRKRERSQQAELCKGSFAAPLGGLVPPYEEVYPGYSYGNWPPKALAPPLAAKTFPFAFNSVNVGPLASQPVFSPPSSIAASMVPSAAAAPGTVPGPGALQGLGGGPPGLAPAAVSSGAVSCPYASAAAAAAAAASSPYVYRDPCNSSLASLRLKAKQHASFSYPAVHGPPPAANLSPCQYAVERPV

SEQ ID NO: 25 - DNA, 2244 bp, Mus musculus
gcagccactgaaccacaagcagctcggctttaactggagtgccttggagtcgcgtgccagcagccacacggccagggactgactgacagacaaccacgcacgagaacgacaacacacgagactcgggcgagctgccgcggtcgtccgggctcttggcaaagtcgcccagccgagaggcccccccgcggaggtgcgcctaggaagcgccaagcccgcggcgcggaggacaccgtgctcggttccgggctgcggggacattcccggacacacaccggagcagcagctgcgccgcgacacatctggagccgcgtaggatgttcgtcaaatctgagactctggagttgaaggaggaagaggaggtactgatgctgctgggctcggcttccccggcctcggcgaccctgaccccgatgtcctccagcgcggacgaggaggaggacgaggagctgcgccggccgggctccgcgcgtgggcagcgtggagcggaagccgggcagggggtgcagggcagtccggcgtcgggtgccgggggttgccggccagggcggctgctgggcctgatgcacgagtgcaagcgtcgcccgtcgcgctcacgggccgtctcccgaggtgccaagacggcggagacggtgcagcgcatcaagaagacccgcaggctcaaggccaacaaccgcgagcgcaaccgcatgcacaacctaaacgccgcgctggacgcgctgcgcgaggtgctgcccaccttccccgaggatgccaagctcacgaagatcgagacgctgcgcttcgcccacaattacatctgggcgctcaccgagactctgcgcctggcggaccactgcgccggcgccggtggcctccagggggcgctcttcacggaggcggtgctcctgagcccgggagctgcgctcggcgccagcggggacagcccttctccaccttcctcctggagctgcaccaacagcccggcgtcatcctccaactccacgtccccatacagctgcactttatcgcccgctagccccgggtcagacgtggactactggcagcccccacctccggagaagcatcgttatgcgcctcacctgcccctcgccagggactgtatctagagctgcgggtctccctctctcgtcctctacccggccctcttcccatccttctcccgcccctcaccctccacgccccggactccacttcacagagcagaggtggcccttgcaatcccctcggcggctggtgcattcgggggtggagaccagctctggtttattgaagatgtgaggatttatggtcaaagaggactatggcgtgtgggagtgggggctggcgtggggaacctcgtaagactgtaaaagacactgagaaaaagtaccataactaacgagtgtgcagagcagactgacgctcctcccctctctcagagctgctggaggagaactccgggcaggcagttcgtgtgaatctctcagagggaatgcaactggtccctgtgatcttttcaccttcgtttctacatagagatgttaatgtcagtcgaaagaaatgtattttagcatctgaatgaatttactggtaataatattatccacacatttgcaatggctggcatctgctctattcccattgctgtctgcaggctgtgggaatttcacctgtcaaaccaaactttccctctctgatgtgcactttgtttttttcccagattcgtcacaatgcctattgtcccgcccttctttttgctttttttctccattttgccatctgtctcttatgatttataagggggaaaaacttgttttgttagagggccaggttagaagtcattgtataatttgtaggcttttgtaagggttgaatgcaagcgtggaaatttaggctgaattctctatcaaaagaaaaaatgtgaaggaaaaaggaaaaatcaggagggaggattgcttcatgcattatttatctcgaccttttaggggagaaggaactcccccatcctttcaagagattaaaaataaatcaacagtctgaaaacctaagcagacacggggcattgccaggatcagccacacacgtgtttccttctatttattttgaagaaaaatttcatgggaaagtatgtatttttttgtatattctacagagtttattctagtatgtatttacatcccgaagaataagaaaattgttttgtgattaagctataaataaagtatctaattttcataaaaaaaaaaaaaaaaaaaaaaaaaaaaa

SEQ ID NO: 26 - protein, 263 aa, Mus musculus
MFVKSETLELKEEEEVLMLLGSASPASATLTPMSSSADEEEDEELRRPGSARGQRGAEAGQGVQGSPASGAGGCRPGRLLGLMHECKRRPSRSRAVSRGAKTAETVQRIKKTRRLKANNRERNRMHNLNAALDALREVLPTFPEDAKLTKIETLRFAHNYIWALTETLRLADHCAGAGGLQGALFTEAVLLSPGAALGASGDSPSPPSSWSCTNSPASSSNSTSPYSCTLSPASPGSDVDYWQPPPPEKHRYAPHLPLARDCI

SEQ ID NO: 27 - DNA, 2370 bp, Homo sapiens
cgcagccactgaaccacaagcagcttcgcgttaactggagtgcctgggagtcgcgtgccaggagccgcacggccagggactgactgacagacagacacgcaccaccaccacaacacacgagacccgggcgggccgccgccgccgccgccggggctcttggcaaactcgccggtcgcagaggtcccccgcggagctgcgccacagtagcgccgggcttgcagctttcacgccgggcgaaggacccggcgctgcgctcgcagctgcgcggagattcccggcacaggccaaagtcacagcaacgctgaggcacagttagagccaactaagatgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggcgccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatctagagctgccatttctgctacccacgccaggccttagtgggttccctttcctgtccccagtcgagccctcctcccttcccctgcccctcctttccacgccctggaaaccatctcacttcacagggcaggtgtagcctttctgattcctcggttgtttcttgcatttcttggctttgggtatccttcattcagacgggctctgatttactgaaggtgtgatggagcttattgtcaaagccaagggtggcgttttgggggcgcttcttgagacgaaaaagaccctgggaagagatgatggtggcatatctaaagagtttgcagagcggactgacgctcctcccctttctctttaacgccgaaggacttggtgcagttcgtgtgaatctcacagggggaatgcaactggttcctgtgatctcttcacctttgcttctacatagagatgttaatgtcgagtagaaagaaatgtatcttagcatctgaatgattttgctggtaataatattatccacagatttgcaatggctggcatctgctttattcccattgctgtctgcaggctgtgggaatttcacctgtcaaaccaaacttccctctctgatgtgcactttgttctgtttcccagattcgtcacaatgcctattgtcctgtccttctctttcctttttcttccccattttgccatctgtctcttatgatttataaggggaaaaaaacttgttttgttagaggggcaggttagaagtcattgtataatttgtaggctttgtaatgattgaatgcaagcgtggaaatttaggctgaactctctatcaaaaggaaaaatgtggaggaaaagggaaaaatcaggagggaggattgcctcatgtattatttatttcgaccttttaggggagaaggaactcccccattctttcaagagattaaaaataaatcaacagtctgaaaacctaagcagacacggagcattatccggatcagccacacacgtgttcccttctatttattataaagaaatttttcatgggaaaatatgtattttttgtatattctacagagtttattctagtatgtatttacatcttgaagaacaagaaagttgttcttgtgattaaactataaataaactatctaattttcataaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - protein, 272 aa, Homo sapiens
MFVKSETLELKEEEDVLVLLGSASPALAALTPLSSSADEEEEEEPGASGGARRQRGAEAGQGARGGVAAGAEGCRPARLLGLVHDCKRRPSRARAVSRGAKTAETVQRIKKTRRLKANNRERNRMHNLNAALDALREVLPTFPEDAKLTKIETLRFAHNYIWALTETLRLADHCGGGGGGLPGALFSEAVLLSPGGASAALSSSGDSPSPASTWSCTNSPAPSSSVSSNSTSPYSCTLSPASPAGSDMDYWQPPPPDKHRYAPHLPIARDCI

SEQ ID NO: 29 - DNA, 2554 bp, Mus musculus
cgattaaaggcgctgccagcctcgctctctgggcacagctgagcgtgacactggggaagtcaaacccctctactgcctaggaagatggctagactttaaagattatttttttccctttaaggaaaaagtctcggagctttaaaaaaaattcctttttctctttttttttctcccctctttttttttttttctgagccgtggcttatccccccattaagaccaatcactgaaatcttgttgctgaaagaaaaaaaagaaagaaagaaaaagaaaagaaaataatagccaagtgtcttcactgtatctggatgtctacaaattagagagagggagagagcgagatttgctccaccagagcgggcgagagccaggccagacgctcgcctttcttttttccgcctgcatccgccctgtgccttcgctgaggcttcgctttgccttcttcctctccgcgcacccccacgggcccgctggcaaagtggggtggggagcgaggcgcggggggcgggggccggcgccgcggccagggctgccgggcggccgagcatggaagaacagcagccggagcctaaaagtcagcgcgactcgggcctcggcgcggtggcagcggcggccccgagcggcctcagtctgagtctgagcccaggagccagcggcagcagcggcagcgatggagacagcgtgccggtgtccccgcagccagcgcccccgtcgcctcctgcggcaccctgtctgccgcccctggcccatcacccgcacctccccccgcatcccccgcccccgccgccgccgccgccgccgccaccgcagcatctcgcggcgcctgctcaccagccgcagcccgcggcccagctgcaccgcaccaccaactttttcatcgataacatcctaaggcccgatttcggttgcaaaaaggaacagcccctgcctcagctcctggtggcttcggctgcagccggaggaggcgcagcagcaggaggaggaagccgcgtggagcgtgaccgaggccagactggtgcaggtagagaccccgttcactctctgggcacacgagcttcgggggctgcctcgctcttgtgtgctccagatgcgaactgtggcccacccgacggctcccagcccgccaccgctgtcagcgccggcgcatccaaagccgggaacccggctgctgcggcggccgcggccgcagcagcggctgcagcggcagtggcggcagcggcggcagcagcctcgaagccctcggacagtggcggtggtagtggaggcaacgcggggagtcccggggcgcagggcgccaagttcccggaacacaaccctgcgatcctactcatgggttcggctaacggtgggccggtggtcaagactgactcacagcaacccctagtgtggcccgcctgggtctactgcacacgctattcggaccgtccgtcctctggtccacgcaccaggaagctaaagaagaaaaagaacgagaaggaagacaagcggccgcggacggcgttcacggccgagcagctgcagagactcaaggcggagttccaggcaaaccgctatatcacggagcagcggcgacagaccctcgcccaggagctcagcctgaatgagtcccagatcaagatctggttccaaaacaagcgtgccaagatcaagaaagccaccggcatcaagaacggcctggcgctgcacctcatggcccagggactgtacaaccactctaccaccacggttcaggacaaagacgagagcgagtagctgtggccagctccggggcccgcggtccaacggcgcccgtgccacctccaggctcctcggggctgccgcttcaccagccccacgcagagacgatcgctatggagggaggcatcaatcagggcgacagagaaagcgagcaagagaaagcaatcctccgagtggacattcacataggaacaaaacggtttttgaaacgggagtaagactcggacaggtgctatgggggaaaaataaacatctattctctaactcactgtataagatgaaactgcgaattccttaaagctctatctagccaaactgctttcgaccgtgtatatatttaatttcaggtaaggaaaacaaatatgtgtagcgatctctatttgctggacatttttattaatctcatttattattgttataattattataattattataattatttttcccctcctccctaccttgctgcaccccccccccccagcccagtttcgttttcgttgctcttttcctttgaatgtttttgcttctctgggtacctcctgcacccccaacgctggccctggtttctctgggacttttctttgtgtgagtgtgagtgtgtttccttgtgtgtctgcccctcgcctcttctctacccacccaggattcttctattggtcttgtctatccctcccgtaaatccccttccttttctggagactccttgagaaatacaaccccacagactgcgagactgaaccgccgctacaagccaaagattttattatgttcagaaacctgtagtctgaaataaaatgttcactgtgttcatgag

SEQ ID NO: 30 - protein, 401 aa, Mus musculus
MEEQQPEPKSQRDSGLGAVAAAAPSGLSLSLSPGASGSSGSDGDSVPVSPQPAPPSPPAAPCLPPLAHHPHLPPHPPPPPPPPPPPPQHLAAPAHQPQPAAQLHRTTNFFIDNILRPDFGCKKEQPLPQLLVASAAAGGGAAAGGGSRVERDRGQTGAGRDPVHSLGTRASGAASLLCAPDANCGPPDGSQPATAVSAGASKAGNPAAAAAAAAAAAAAAVAAAAAAASKPSDSGGGSGGNAGSPGAQGAKFPEHNPAILLMGSANGGPVVKTDSQQPLVWPAWVYCTRYSDRPSSGPRTRKLKKKKNEKEDKRPRTAFTAEQLQRLKAEFQANRYITEQRRQTLAQELSLNESQIKIWFQNKRAKIKKATGIKNGLALHLMAQGLYNHSTTTVQDKDESE

SEQ ID NO: 31 - DNA, 2962 bp, Homo sapiens
agctcacagacccataatcctgcatttctctaacaagttgtttatggagttgcttctccatttgcctacatcccaaaattcacccctcccgggtttcttctgccccctcctgagtcccggcctgaaggagggggagggacgcgggtgcgggcgcgggtgggggagggcggacccgacgcacagggccagcgccgaggcgccccctctccgccagcggttgacgcccccggattatttatccgcaaagtcccgcgcgcgcccattgggccgaggcccgagtgtcagcgcgagtcccggctcgccattggctccgcacacgtgcggccctgactcacgtgcttccggtttgaaggcaaaaagtgtgcctgggtgattttttttttaagcgagagagtttgtgcaaagatccgagctgtcagagatttgaaaaaaaaaaaaaaaaaaaaaaaaaaaaaaacagcccggcgctggcggagacgcgctctccctgcaaaaaaagcaaaggcgattaaaggcgctgccagcctcacgctctgggcacagctgagcgtgacactcggggaagtcaaacccctcactactgcctaggaagatggctagactttaaatactatttttttccctttaagaaaaaaattattggagctttttttcttgctttctttttccttttctttttctttttttccttcatttttttggccgtggcttactccccatttaaatcaaatcattgaatctggttgcagaaagaaaaaagaaatagccaagtgtctccatatctggatgtctacaaattagagagggagagacagcgagatctatctgctagataagaacgagcgatccaggccagacgcctgagcttttttcctgcacccgccccgtgccttcgctgaggcttcgcctgcctccttcctccgcgcacccccacgggccgctggcaaagtggggtggggagcgaggcggtgggggcgggggccggcgcggcggccggggcggcggggcggccgagcatggaagaacagcagccggaacctaaaagtcagcgcgactcggccctcggcgcggcggcggcggcgactccgggcggcctcagcctgagcctcagtccgggcgccagcggcagcagcggcagcggcagcgatggagacagcgtgccggtgtccccgcagcctgcgcccccctcgccgcccgcggcgccttgcctgccgcccctggcccaccacccgcacctccccccacaccccccgcccccgccgcctcagcatctcgcggcgcctgctcaccagccgcagccagcggcccagctgcaccgcaccaccaactttttcatcgacaacatcctgaggccggacttcggctgcaaaaaggagcagccgccaccgcagcttctggtggctgcggcggccagaggaggcgcaggaggaggaggccgggtcgagcgtgacagaggccagactgccgcaggtagagaccctgtccacccgttgggcacccgggcgccaggcgctgcctcgctcctgtgcgccccggacgcgaactgtggcccacccgacggctcccagccagccgccgccggcgcgggcgcgtctaaagctgggaacccggctgcggcggcggcggcggccgcggcggcagtggcggcggcggcggcggccgcagcagccaagccctcggacaccggtggcggcggcagtggaggcggcgcggggagccccggagcgcagggcaccaaatacccggagcacggcaacccggctatcctacttatgggctcagccaacggcgggcccgtggtcaaaactgactcgcagcagcctctcgtatggcccgcctgggtgtactgcacacgttattcggatcgtccatcctccggtccgcgcaccaggaagctgaagaagaagaagaacgagaaggaggacaagcggccgcggaccgcgttcacggccgagcagctgcagagactcaaggcggagttccaggcaaaccgctacatcacggagcagcggcggcagaccctggcccaggaactcagcctcaacgagtcccagatcaagatctggttccagaacaagcgcgccaagatcaagaaagccacaggcatcaagaacggcctggcgctgcacctcatggcccagggactgtacaaccactccaccaccacggtccaggacaaagacgagagcgagtagccgccacaggccggggccgcgcccgcgccccctcccggcaccgccgccgtcgtctcccggcccctcgctgggggagaaagcatctgctccaaggagggagggagcgcagggaaaagagcgagagagacagaaagagagcctcagaatggacaatgacgttgaaacgcagcatttttgaaaagggagaaagactcggacaggtgctatcgaaaaataagatccattctctattcccagtataagggacgaaactgcgaactccttaaagctctatctagccaaaccgcttacgaccttgtatatatttaatttcaggtaaggaaaacacatacgtgtagcgatctctatttgctggacatttttattaatctcctttattattattgttataattattataattattataattattttatcccctcccccaccgcctcgctgcccccgcccagtttcgttttcgttgcctttttcatttgaatgtcattgcttctccggtgcctcccgacccgcatcgccggccctggtttctctgggacttttctttgtgtgcgagagtgtgtttcctttcgtgtctgcccacctcttctcccccacctcccgggtcccttctgtcggtctgtctgttctgcccccctttcgttttccggagacttgttgagaaatacgaccccacagactgcgagactgaaccgccgctacaagccaaagattttattatgttcagaaacctgtagtctgaaataaagtgtacactgtgctcacga

SEQ ID NO: 32 - protein, 392 aa, Homo sapiens
MEEQQPEPKSQRDSALGAAAAATPGGLSLSLSPGASGSSGSGSDGDSVPVSPQPAPPSPPAAPCLPPLAHHPHLPPHPPPPPPQHLAAPAHQPQPAAQLHRTTNFFIDNILRPDFGCKKEQPPPQLLVAAAARGGAGGGGRVERDRGQTAAGRDPVHPLGTRAPGAASLLCAPDANCGPPDGSQPAAAGAGASKAGNPAAAAAAAAAAVAAAAAAAAAKPSDTGGGGSGGGAGSPGAQGTKYPEHGNPAILLMGSANGGPVVKTDSQQPLVWPAWVYCTRYSDRPSSGPRTRKLKKKKNEKEDKRPRTAFTAEQLQRLKAEFQANRYITEQRRQTLAQELSLNESQIKIWFQNKRAKIKKATGIKNGLALHLMAQGLYNHSTTTVQDKDESE

SEQ ID NO: 33 - DNA, 2127 bp, Mus musculus
ctgacgaccagggcggccagaccacgcgagtcctacgcgcctcctgaggccgccccgggacttaactgtaacggggaggggcctccggagcagcggccagcgagttaaagtatgctgggagccgtgaagatggaagggcacgagccatccgactggagcagctactacgcggagcccgagggctactcttccgtgagcaacatgaacgccggcctggggatgaatggcatgaacacatacatgagcatgtccgcggctgccatgggcggcggttccggcaacatgagcgcgggctccatgaacatgtcatcctatgtgggcgctggaatgagcccgtcgctagctggcatgtccccgggcgccggcgccatggcgggcatgagcggctcagccggggcggccggcgtggcgggcatgggacctcacctgagtccgagtctgagcccgctcgggggacaggcggccggggccatgggtggccttgccccctacgccaacatgaactcgatgagccccatgtacgggcaggccggcctgagccgcgctcgggaccccaagacataccgacgcagctacacacacgccaaacctccctactcgtacatctcgctcatcaccatggccatccagcagagccccaacaagatgctgacgctgagcgagatctatcagtggatcatggacctcttccctttctaccggcagaaccagcagcgctggcagaactccatccgccactctctctccttcaacgactgctttctcaaggtgccccgctcgccagacaagcctggcaagggctccttctggaccctgcacccagactcgggcaacatgttcgagaacggctgctacctgcgccgccagaagcgcttcaagtgtgagaagcaactggcactgaaggaagccgcgggtgcggccagtagcggaggcaagaagaccgctcctgggtcccaggcctctcaggctcagctcggggaggccgcgggctcggcctccgagactccggcgggcaccgagtccccccattccagcgcttctccgtgtcaggagcacaagcgaggtggcctaagcgagctaaagggagcacctgcctctgcgctgagtcctcccgagccggcgccctcgcctgggcagcagcagcaggctgcagcccacctgctgggcccacctcaccacccaggcctgccaccagaggcccacctgaagcccgagcaccattacgccttcaaccaccccttctctatcaacaacctcatgtcgtccgagcagcaacatcaccacagccaccaccaccatcagccccacaaaatggacctcaaggcctacgaacaggtcatgcactacccagggggctatggttcccccatgccaggcagcttggccatgggcccagtcacgaacaaagcgggcctggatgcctcgcccctggctgcagacacttcctactaccaaggagtgtactccaggcctattatgaactcatcctaagaagatggctttcaggccctgctagctctggtcactggggacaagggaaatgagaggctgagtggagactttgggagagctttgaggaaaagtagccaccacacttcaggcctcaagggagcagtctcacctgtctgtgtccctaaatagatgggccacagtgatctgtcattctaaatagggaagggaatggaaatatatatgtatacatataaacttgttttaaaggagcctttggtctcctctatgtagactactgcttctcaagacatctgcagagtttgatttttgttgttgttctctattgctgttgttgcagaaaagtctgactttaaaaacaaacaaacaaacaaaaaacttttgtgagtgacttggtgtaaaaccatgtagttttaacagaaaaccagagggttgtactgatgttgaaaagaggaaagaaaaataatgtaagagtctggtgtaccggaccaggagaaaggagaaaaacacatcccattctggacatggtgaaatccaggtctcgggtctgatttaatttatggtttctgcgtgctttatttatggcttataaatgtgtgttctggctagaatggccagaattccacaaatctatattaaagtgttattgccgatttt

SEQ ID NO: 34 - protein, 459 aa, Mus musculus
MLGAVKMEGHEPSDWSSYYAEPEGYSSVSNMNAGLGMNGMNTYMSMSAAAMGGGSGNMSAGSMNMSSYVGAGMSPSLAGMSPGAGAMAGMSGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAASSGGKKTAPGSQASQAQLGEAAGSASETPAGTESPHSSASPCQEHKRGGLSELKGAPASALSPPEPAPSPGQQQQAAAHLLGPPHHPGLPPEAHLKPEHHYAFNHPFSINNLMSSEQQHHHSHHHHQPHKMDLKAYEQVMHYPGGYGSPMPGSLAMGPVTNKAGLDASPLAADTSYYQGVYSRPIMNSS

SEQ ID NO: 35 - DNA, 2428 bp, Homo sapiens
cccgcccacttccaactaccgcctccggcctgcccagggagagagagggagtggagcccagggagagggagcgcgagagagggagggaggaggggacggtgctttggctgacttttttttaaaagagggtgggggtggggggtgattgctggtcgtttgttgtggctgttaaattttaaactgccatgcactcggcttccagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaagaagacgacggcttcaggcccggctaactctggcaccccggatcgaggacaagtgagagagcaagtgggggtcgagactttggggagacggtgttgcagagacgcaagggagaagaaatccataacacccccaccccaacacccccaagacagcagtcttcttcacccgctgcagccgttccgtcccaaacagagggccacacagataccccacgttctatataaggaggaaaacgggaaagaatataaagttaaaaaaaagcctccggtttccactactgtgtagactcctgcttcttcaagcacctgcagattctgatttttttgttgttgttgttctcctccattgctgttgttgcagggaagtcttacttaaaaaaaaaaaaaaattttgtgagtgactcggtgtaaaaccatgtagttttaacagaaccagagggttgtactattgtttaaaaacaggaaaaaaaataatgtaagggtctgttgtaaatgaccaagaaaaagaaaaaaaaagcattcccaatcttgacacggtgaaatccaggtctcgggtccgattaatttatggtttctgcgtgctttatttatggcttataaatgtgtattctggctgcaagggccagagttccacaaatctatattaaagtgttatacccggttttatcccttgaatcttttcttccagatttttcttttctttacttggcttacaaaatatacaggcttggaaattatttcaagaaggagggagggataccctgtctggttgcaggttgtattttattttggcccagggagtgttgctgttttcccaacattttattaataaaattttcagacataaaaaa

SEQ ID NO: 36 - protein, 463 aa, Homo sapiens
MHSASSMLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 37 - DNA, 1338 bp, Mus musculus
atggcgaccgcagcgtctaaccactacagcctgctcacctccagcgcctccatcgtacatgccgagccgcctggcggcatgcagcagggcgcagggggctaccgcgaggcgcagagcctggtgcagggcgactacggcgcgctgcagagcaacgggcacccgctcagccacgctcaccagtggatcaccgcgctgtcccacggcggcggcggcgggggcggcggcggcggtggaggaggcgggggaggcggcgggggaggcggcgacggctccccgtggtccaccagccccctaggccagccggacatcaagccctcggtggtggtacagcagggtggccgaggcgacgagctgcacgggccaggagcgctgcagcaacagcatcaacagcaacagcaacagcagcagcagcagcagcagcagcagcagcagcaacagcagcagcaacaacagcgaccgccacatctggtgcaccacgctgccaaccaccatcccgggcccggggcatggcggagtgcggcggctgcagctcacctccctccctccatgggagcttccaacggcggtttgctctattcgcagccgagcttcacggtgaacggcatgctgggcgcaggagggcagccggctgggctgcaccaccacggcctgagggacgcccacgatgagccacaccatgcagaccaccacccgcatccgcactctcacccacaccagcaaccgcccccgccacctcccccacaaggcccaccgggccacccaggcgcgcaccacgacccgcactcggacgaggacacgccgacctcagacgacctggagcagttcgccaagcaattcaagcagaggcggatcaaactcggatttactcaagcagacgtggggctggcgcttggcaccctgtacggcaacgtgttctcgcagaccaccatctgcaggtttgaggccctgcagctgagcttcaagaacatgtgcaagctgaagcctttgttgaacaagtggttggaagaggcagactcatcctcgggcagccccaccagcatagacaagatcgcagcgcaagggcgcaaacggaaaaagcggacctccatcgaggtgagcgtcaagggggctctggagagccatttcctcaaatgccctaagccctcggcccaggagatcacctccctcgcggacagcttacagctggagaaggaggtggtgagagtttggttttgtaacaggagacagaaagagaaaaggatgacccctcccggagggactctgccgggcgccgaggatgtgtatgggggtagtagggacacgccaccacaccacggggtgcagacgcccgtccagtga

SEQ ID NO: 38 - protein, 445 aa, Mus musculus
MATAASNHYSLLTSSASIVHAEPPGGMQQGAGGYREAQSLVQGDYGALQSNGHPLSHAHQWITALSHGGGGGGGGGGGGGGGGGGGGGDGSPWSTSPLGQPDIKPSVVVQQGGRGDELHGPGALQQQHQQQQQQQQQQQQQQQQQQQQQQQRPPHLVHHAANHHPGPGAWRSAAAAAHLPPSMGASNGGLLYSQPSFTVNGMLGAGGQPAGLHHHGLRDAHDEPHHADHHPHPHSHPHQQPPPPPPPQGPPGHPGAHHDPHSDEDTPTSDDLEQFAKQFKQRRIKLGFTQADVGLALGTLYGNVFSQTTICRFEALQLSFKNMCKLKPLLNKWLEEADSSSGSPTSIDKIAAQGRKRKKRTSIEVSVKGALESHFLKCPKPSAQEITSLADSLQLEKEVVRVWFCNRRQKEKRMTPPGGTLPGAEDVYGGSRDTPPHHGVQTPVQ

SEQ ID NO: 39 - DNA, 1332 bp, Homo sapiens
atggcgaccgcagcgtctaaccactacagcctgctcacctccagcgcctccatcgtgcacgccgagccgcccggcggcatgcagcagggcgcggggggctaccgcgaagcgcagagcctggtgcagggcgactacggcgctctgcagagcaacggacacccgctcagccacgctcaccagtggatcaccgcgctgtcccacggcggcggcggcgggggcggtggcggcggcggggggggcgggggcggcggcgggggcggcggcgacggctccccgtggtccaccagccccctgggccagccggacatcaagccctcggtggtggtgcagcagggcggccgcggagacgagctgcacgggccaggcgccctgcagcagcagcatcagcagcagcaacagcaacagcagcagcaacagcagcaacagcagcagcagcagcagcaacagcggccgccgcatctggtgcaccacgccgctaaccaccacccgggacccggggcatggcggagcgcggcggctgcagcgcacctcccaccctccatgggagcgtccaacggcggcttgctctactcgcagcccagcttcacggtgaacggcatgctgggcgccggcgggcagccggccgggctgcaccaccacggcctgcgggacgcgcacgacgagccacaccatgccgaccaccacccgcacccgcactcgcacccacaccagcagccgccgcccccgccgcccccgcagggtccgcctggccacccaggcgcgcaccacgacccgcactcggacgaggacacgccgacctcggacgacctggagcagttcgccaagcagttcaagcagcggcggatcaaactgggatttacccaagcggacgtggggctggctctgggcaccctgtatggcaacgtgttctcgcagaccaccatctgcaggtttgaggccctgcagctgagcttcaagaacatgtgcaagctgaagcctttgttgaacaagtggttggaggaggcggactcgtcctcgggcagccccacgagcatagacaagatcgcagcgcaagggcgcaagcggaaaaagcggacctccatcgaggtgagcgtcaagggggctctggagagccatttcctcaaatgccccaagccctcggcccaggagatcacctccctcgcggacagcttacagctggagaaggaggtggtgagagtttggttttgtaacaggagacagaaagagaaaaggatgacccctcccggagggactctgccgggcgccgaggatgtgtacggggggagtagggacactccaccacaccacggggtgcagacgcccgtccagtga

SEQ ID NO: 40 - protein, 443 aa, Homo sapiens
MATAASNHYSLLTSSASIVHAEPPGGMQQGAGGYREAQSLVQGDYGALQSNGHPLSHAHQWITALSHGGGGGGGGGGGGGGGGGGGGGDGSPWSTSPLGQPDIKPSVVVQQGGRGDELHGPGALQQQHQQQQQQQQQQQQQQQQQQQQQRPPHLVHHAANHHPGPGAWRSAAAAAHLPPSMGASNGGLLYSQPSFTVNGMLGAGGQPAGLHHHGLRDAHDEPHHADHHPHPHSHPHQQPPPPPPPQGPPGHPGAHHDPHSDEDTPTSDDLEQFAKQFKQRRIKLGFTQADVGLALGTLYGNVFSQTTICRFEALQLSFKNMCKLKPLLNKWLEEADSSSGSPTSIDKIAAQGRKRKKRTSIEVSVKGALESHFLKCPKPSAQEITSLADSLQLEKEVVRVWFCNRRQKEKRMTPPGGTLPGAEDVYGGSRDTPPHHGVQTPVQ

SEQ ID NO: 41 - DNA, 3564 bp, Mus musculus
atggacgtggactctgaggagaagcgccatcgcacacggtccaaaggggttcgagttcctgtggagccagccatacaagagctgttcagctgtcccactccaggctgcgacggcagtggtcacgtcagtggcaaatatgcacgacacagaagtgtatatggttgtcccttggctaaaaaaagaaaaacgcaagataaacagccccaagaacctgctcccaagcgaaaaccatttgcagtaaaagcagatagttcctcagtagacgaatgttatgagagtgatggtactgaagacatggatgataaggaggaagatgatgatgaggagttctctgaagacaatgatgagcaaggggatgatgacgacgaagatgaggtggatcgggaagacgaggaggagatcgaggaggaagatgatgaagaagatgatgatgatgaagatggtgacgatgtagaagaggaagaagaggatgatgatgaagaggaggaagaagaggaagaggaagaagaaaatgaagaccatcaaatgagttgtactcgaataatgcaggacacagacaaggatgataacaacaatgatgagtatgataactatgatgaactggtagctaagtcgctattaaatcttggcaaaattgctgaggatgcagcataccgagccaggactgaatcagagatgaacagcaatacctccaatagtctggaggacgatagtgacaaaaacgaaaacctcggtcggaaaagcgaactgagtctagacttagacagtgatgttgttagagaaacagtggactcccttaagctgttagcacaaggacatggtgttgtgctatcagagaatatcagtgacagaagttatgctgaggggatgtcacagcaggacagtagaaatatgaactatgtcatgctagggaagcccatgaacaatggactcatggagaagatggtggaggagagtgatgaggaagtgtgtctaagtagtctagagtgcctgaggaaccagtgctttgacctggccaggaaactcagcgagaccaacccacaggacaggagtcagccacccaacatgagtgtgcgccaacatgtccggcaagaggacgacttccctgggaggacgccagacaggagctactcggatatgatgaaccttatgcggctggaggagcagctcagtcccaggtctagaacgttctccagctgtgccaaggaggatgggtgtcatgagagggatgatgacaccacctcagtgaactcagacaggtctgaggaagtgtttgacatgaccaagggcaacctgactctgctagagaaagccattgccttggagacagagagagccaaggccatgcgggagaagatggccatggatgctgggagaagggataacctgagatcctatgaggaccagtctccaagacagctggctggggaagacagaaaatccaaatccagtgacagccatgtcaaaaagccatactatggtaaagatccctcaagaacagaaaagagagagagcaagtgtccaacccccgggtgtgatggaaccggccacgtaactgggctttacccgcatcaccgcagtctgtctggatgcccgcacaaagatagggtccctccagaaattcttgccatgcatgaaaatgttctcaagtgtcccactccaggctgcacagggcgagggcatgtgaatagcaacaggaactcgcacagaagcctctctggatgccccattgctgctgcagaaaaactggcaaaggcccaagagaaacaccagagctgtgatgtgtccaaatccaaccaggcctcagaccgagtcctcaggccaatgtgctttgtcaaacagcttgagattcctcagtatggctacagaaacaatgttcccacaaccacaccacgctccaacctggccaaggagcttgagaaatactccaagacttcgtttgagtacaacagttacgacaaccatacttatggcaaaagagccatagctcccaaggtgcaaaccagggacatatcccccaaaggatatgacgatgccaagcggtactgcaagaatgccagccccagcagcagcaccaccagcagctatgcacctagcagcagcagcaacctcagctgtggtggtggcagcagcgccagtagcacgtgtagcaagagcagctttgactacacacatgacatggaggccgcacacatggcagccacagccattctcaacctgtccacacgttgtcgtgaaatgccacagaacctgtccaccaagccacaggacctgtgtactgcccggaacccagacatggaggtggatgagaatggcaccctggacctgagcatgaacaagcagaggcctcgagacagctgctgcccagtcctgacacccctggaacccatgtctccgcagcagcaggccgtgatgagcagccgatgcttccagctgagcgagggggattgctgggacttgcctgtagactacaccaaaatgaagcctcggagggtagatgaggatgagcccaaagagattaccccagaagacttggacccattccaggaggctctggaagaaagacggtatccaggggaggtgaccatcccaagccccaaacccaagtaccctcagtgcaaggaaagcaaaaaggacttaataactctgtctggctgccccctggcggacaaaagcattcgaagtatgctggccaccagttcccaagagctcaagtgccccacccctggctgtgacggttctggacacatcactggcaattacgcttctcatcgaagcctttctgggtgcccgagagcaaagaagagtggcatccggatagcacagagcaaagaggacaaggaagaccaggagccaatcaggtgtccggtacctggctgtgacggtcagggacacatcactgggaagtatgcatcccaccgcagcgcctccgggtgtcccttggcagccaagaggcagaaagatgggtaccttaatggctcccagttctcctggaagtcggtcaagacggagggcatgtcctgccctacccccgggtgtgatgggtcaggacacgtcagtggcagcttcctcacacaccgcagcttgtcaggatgtccaagagccacatcagcaatgaagaaagcaaagctgtctggagaacagatgttgactatcaagcagcgagccagcaacggtatagaaaatgatgaagaaatcaagcagttagatgaagagatcaaggagcttaatgagtccaattcccagatggaggctgacatgatcaaactcagaactcagatcaccacaatggagagcaacctgaagacgattgaggaggagaacaaagtcattgaacagcagaatgagtcgctcttgcacgagttggccaacctgagccagtccctgatccacagcctcgccaacatccagctgcctcacatggatccaatcaatgaacaaaattttgatgcttacgtgactactttgacggaaatgtatacaaatcaagatcgttatcagagtccagaaaataaagccctactggaaaatataaagcaggctgtgagaggaattcaggtctga

SEQ ID NO: 42 - protein, 1187 aa, Mus musculus
MDVDSEEKRHRTRSKGVRVPVEPAIQELFSCPTPGCDGSGHVSGKYARHRSVYGCPLAKKRKTQDKQPQEPAPKRKPFAVKADSSSVDECYESDGTEDMDDKEEDDDEEFSEDNDEQGDDDDEDEVDREDEEEIEEEDDEEDDDDEDGDDVEEEEEDDDEEEEEEEEEEENEDHQMSCTRIMQDTDKDDNNNDEYDNYDELVAKSLLNLGKIAEDAAYRARTESEMNSNTSNSLEDDSDKNENLGRKSELSLDLDSDVVRETVDSLKLLAQGHGVVLSENISDRSYAEGMSQQDSRNMNYVMLGKPMNNGLMEKMVEESDEEVCLSSLECLRNQCFDLARKLSETNPQDRSQPPNMSVRQHVRQEDDFPGRTPDRSYSDMMNLMRLEEQLSPRSRTFSSCAKEDGCHERDDDTTSVNSDRSEEVFDMTKGNLTLLEKAIALETERAKAMREKMAMDAGRRDNLRSYEDQSPRQLAGEDRKSKSSDSHVKKPYYGKDPSRTEKRESKCPTPGCDGTGHVTGLYPHHRSLSGCPHKDRVPPEILAMHENVLKCPTPGCTGRGHVNSNRNSHRSLSGCPIAAAEKLAKAQEKHQSCDVSKSNQASDRVLRPMCFVKQLEIPQYGYRNNVPTTTPRSNLAKELEKYSKTSFEYNSYDNHTYGKRAIAPKVQTRDISPKGYDDAKRYCKNASPSSSTTSSYAPSSSSNLSCGGGSSASSTCSKSSFDYTHDMEAAHMAATAILNLSTRCREMPQNLSTKPQDLCTARNPDMEVDENGTLDLSMNKQRPRDSCCPVLTPLEPMSPQQQAVMSSRCFQLSEGDCWDLPVDYTKMKPRRVDEDEPKEITPEDLDPFQEALEERRYPGEVTIPSPKPKYPQCKESKKDLITLSGCPLADKSIRSMLATSSQELKCPTPGCDGSGHITGNYASHRSLSGCPRAKKSGIRIAQSKEDKEDQEPIRCPVPGCDGQGHITGKYASHRSASGCPLAAKRQKDGYLNGSQFSWKSVKTEGMSCPTPGCDGSGHVSGSFLTHRSLSGCPRATSAMKKAKLSGEQMLTIKQRASNGIENDEEIKQLDEEIKELNESNSQMEADMIKLRTQITTMESNLKTIEEENKVIEQQNESLLHELANLSQSLIHSLANIQLPHMDPINEQNFDAYVTTLTEMYTNQDRYQSPENKALLENIKQAVRGIQV

SEQ ID NO: 43 - DNA, 3555 bp, Homo sapiens
atggaggtggacaccgaggagaagcggcatcgcacgcggtccaaaggggttcgagttcccgtggaaccagccatacaagagctgttcagctgtcccacccctggctgtgacggcagtggtcatgtcagtggcaaatatgcaagacacagaagtgtatatggttgtcccttggcgaaaaaaagaaaaacacaagataaacagccccaggaacctgctcctaaacgaaagccatttgccgtgaaagcagacagctcctcagtggatgagtgtgacgacagtgatgggactgaggacatggatgagaaggaggaggatgagggggaggagtactccgaggacaatgacgagccaggggatgaggacgaggaggacgaggagggggaccgggaggaggaggaggagatcgaggaggaggatgaggacgatgacgaggatggagaagatgtggaggatgaagaagaggaagaggaggaggaggaggaggaggaagaggaagaagaaaacgaagaccatcaaatgaattgtcacaatactcgaataatgcaagacacagaaaaggatgataacaataatgacgaatatgacaattacgatgaactggtggccaagtcattgttaaacctcggcaaaatcgctgaggatgcagcctaccgggccaggactgagtcagaaatgaacagcaatacctccaatagtctggaagacgatagtgacaaaaacgaaaacctgggtcggaaaagtgagttgagtttagacttagacagtgatgttgttagagaaacagtggactcccttaaactattagcccaaggacacggtgttgtgctctcagaaaacatgaatgacagaaattatgcagacagcatgtcgcagcaagacagtagaaatatgaattacgtcatgttggggaagcccatgaacaacggactcatggaaaagatggtggaggagagcgatgaggaggtgtgtctgagcagtctggagtgtttgaggaatcagtgcttcgacctggccaggaagctcagtgagaccaacccgcaggagaggaatccgcagcagaacatgaacatccgtcagcatgtccggccagaagaggacttccccggaaggacgccggacagaaactactcggacatgctgaacctcatgcggctggaggagcagttgagcccccggtcgagagtgtttgccagctgtgcgaaggaggatgggtgtcatgagcgggacgacgataccacctctgtgaactcggacaggtctgaagaggtgttcgacatgaccaaggggaacctgaccctgctggagaaagccatcgctttggaaacggaaagagcaaaggccatgagggagaagatggccatggaagctgggaggagggacaatatgaggtcatatgaggaccagtctccgagacaacttcccggggaggacagaaagcctaaatccagtgacagccatgtcaaaaagccatactatgatccctcaagaacagaaaagaaagagagcaagtgtccaacccccgggtgtgatggaaccggccacgtaactgggctgtacccacatcaccgcagcctgtccggatgcccgcacaaagatagggtccctccagaaatccttgccatgcatgaaagtgtcctcaagtgccccactccgggctgcacggggcgcgggcatgtcaacagcaacaggaactcccaccgaagcctctccggatgcccgatcgctgcagcagagaaactggccaaggcacaggaaaagcaccagagctgcgacgtgtccaagtccagccaggcctcggaccgcgtgctcaggccaatgtgctttgtgaagcagctggagattcctcagtatggctacagaaacaatgtccccacaactacgccgcgttccaacctggccaaggagctcgagaaatattccaagacctcgtttgaatacaacagttacgacaaccatacttatggcaagcgagccatagctcccaaggtgcaaaccagggatatatcccccaaaggatatgatgatgcgaagcggtactgcaaggaccccagccccagcagcagcagcaccagcagctacgcgcccagcagcagcagcaacctgagctgcggcgggggcagcagcgccagcagcacgtgcagcaagagcagcttcgactacacgcacgacatggaggcggcccacatggcggccaccgccatcctcaacctgtccacgcgctgccgcgagatgccgcagaacctgagcaccaagccgcaggacctgtgcgccacgcggaaccctgacatggaggtggatgagaacgggaccctggacctcagcatgaacaagcagaggccgcgggacagctgctgccccatcctgacccctctggagcccatgtccccccagcagcaggcagtgatgaacaaccggtgtttccagctgggcgagggcgactgctgggacttgcccgtagactacaccaaaatgaaaccccggaggatagacgaggacgagtccaaagacattactccagaagacttggacccattccaggaggctctagaagaaagacggtatcccggggaggtgaccatcccaagtcccaaacccaagtaccctcagtgcaaggagagcaaaaaggacttaataactctgtctggctgccccctggcggacaaaagcattcgaagtatgctggccaccagctcccaagaactcaagtgccccacgcctggctgtgatggttctggacatatcaccggcaattatgcttctcatcggagcctttcaggttgcccaagagcaaagaaaagtggtatcaggatagcacagagcaaagaagataaagaagatcaagaacccatcaggtgtccggtccccgggtgcgacggccagggccacatcactgggaagtacgcgtcccatcgcagcgcctccgggtgccccttggcggccaagaggcagaaagacgggtacctgaatggctcccagttctcctggaagtcggtcaagacggaaggcatgtcctgccccacgccaggatgcgacggctcaggccacgtcagcggcagcttcctcacacaccgcagcttgtcaggatgcccgagagccacgtcagcgatgaagaaggcaaagctttctggagagcagatgctgaccatcaaacagcgggccagcaacggtatagaaaatgatgaagaaatcaaacagttagatgaagaaatcaaggagctaaatgaatccaattcccagatggaagccgatatgattaaactcagaactcagattaccacgatggagagcaacctgaagaccatcgaagaggagaacaaagtgattgagcagcagaacgagtctctcctccacgagctggcgaacctgagccagtctctgatccacagcctggctaacatccagctgccgcacatggatccaatcaatgaacaaaattttgatgcttacgtgactactttgacggaaatgtatacaaatcaagatcgttatcagagtccagaaaataaagccctactggaaaatataaagcaggctgtgagaggaattcaggtctga

SEQ ID NO: 44 - protein, 1184 aa, Homo sapiens
MEVDTEEKRHRTRSKGVRVPVEPAIQELFSCPTPGCDGSGHVSGKYARHRSVYGCPLAKKRKTQDKQPQEPAPKRKPFAVKADSSSVDECDDSDGTEDMDEKEEDEGEEYSEDNDEPGDEDEEDEEGDREEEEEIEEEDEDDDEDGEDVEDEEEEEEEEEEEEEEEENEDHQMNCHNTRIMQDTEKDDNNNDEYDNYDELVAKSLLNLGKIAEDAAYRARTESEMNSNTSNSLEDDSDKNENLGRKSELSLDLDSDVVRETVDSLKLLAQGHGVVLSENMNDRNYADSMSQQDSRNMNYVMLGKPMNNGLMEKMVEESDEEVCLSSLECLRNQCFDLARKLSETNPQERNPQQNMNIRQHVRPEEDFPGRTPDRNYSDMLNLMRLEEQLSPRSRVFASCAKEDGCHERDDDTTSVNSDRSEEVFDMTKGNLTLLEKAIALETERAKAMREKMAMEAGRRDNMRSYEDQSPRQLPGEDRKPKSSDSHVKKPYYDPSRTEKKESKCPTPGCDGTGHVTGLYPHHRSLSGCPHKDRVPPEILAMHESVLKCPTPGCTGRGHVNSNRNSHRSLSGCPIAAAEKLAKAQEKHQSCDVSKSSQASDRVLRPMCFVKQLEIPQYGYRNNVPTTTPRSNLAKELEKYSKTSFEYNSYDNHTYGKRAIAPKVQTRDISPKGYDDAKRYCKDPSPSSSSTSSYAPSSSSNLSCGGGSSASSTCSKSSFDYTHDMEAAHMAATAILNLSTRCREMPQNLSTKPQDLCATRNPDMEVDENGTLDLSMNKQRPRDSCCPILTPLEPMSPQQQAVMNNRCFQLGEGDCWDLPVDYTKMKPRRIDEDESKDITPEDLDPFQEALEERRYPGEVTIPSPKPKYPQCKESKKDLITLSGCPLADKSIRSMLATSSQELKCPTPGCDGSGHITGNYASHRSLSGCPRAKKSGIRIAQSKEDKEDQEPIRCPVPGCDGQGHITGKYASHRSASGCPLAAKRQKDGYLNGSQFSWKSVKTEGMSCPTPGCDGSGHVSGSFLTHRSLSGCPRATSAMKKAKLSGEQMLTIKQRASNGIENDEEIKQLDEEIKELNESNSQMEADMIKLRTQITTMESNLKTIEEENKVIEQQNESLLHELANLSQSLIHSLANIQLPHMDPINEQNFDAYVTTLTEMYTNQDRYQSPENKALLENIKQAVRGIQV

SEQ ID NO: 45 - DNA, 975 bp, Mus musculus
atggaggagaaggattccaagcccagcgagacggcggcggaggcgcagagacagccggaacccagctccggcggcggctctggcggcggcagcagcccgagcgactcggacaccggccgccggcgggctctgatgctgcccgaggtcctacaggcgccaggcaaccaccagcatccacatcgcatcaccaacttcttcatcgataacatcctgcggcctgagtttggccgccgaaaggacgcggggacttgctgtgcgggcgcgggcggagccaggggaggcgaaggcggcgctggcactaccgaaggaggcggcggcggcgcaggcggagccgagcagctactgggcgccagggagtcccgaccgaacccagcgtgcgcacccagcgcgggaggaacgctctccgccgccgctggcgaccctgcggtcgacggagaaggaggttccaagacgctatcacttcacggtggtgccaaaaaacccggcgatcctgggggttccttggatggagtgctcaaagcccggggcttgggcggcggtgacctgtcggtgagctccgactcggacagctctcaagccagcgccactctgggcgcgcagcccatgctctggcccgcttgggtctactgcacgcgctattctgaccggccttcttcaggtcccaggtcccgaaaaccaaagaagaagaaccctaacaaagaggacaagcggcctcgcacagccttcactgctgagcagctccagaggctcaaggctgagtttcagaccaacaggtacctgacagagcagcggcgccagagtctggcacaggagctcagcctgaacgagtctcagatcaagatttggttccagaacaagcgggccaaaatcaagaaagccacgggcaacaagaacactttggcggtgcacctcatggcacagggcctgtacaaccattccaccacggccaaggagggcaagtcggacagcgagtag

SEQ ID NO: 46 - protein, 324 aa, Mus musculus
MEEKDSKPSETAAEAQRQPEPSSGGGSGGGSSPSDSDTGRRRALMLPEVLQAPGNHQHPHRITNFFIDNILRPEFGRRKDAGTCCAGAGGARGGEGGAGTTEGGGGGAGGAEQLLGARESRPNPACAPSAGGTLSAAAGDPAVDGEGGSKTLSLHGGAKKPGDPGGSLDGVLKARGLGGGDLSVSSDSDSSQASATLGAQPMLWPAWVYCTRYSDRPSSGPRSRKPKKKNPNKEDKRPRTAFTAEQLQRLKAEFQTNRYLTEQRRQSLAQELSLNESQIKIWFQNKRAKIKKATGNKNTLAVHLMAQGLYNHSTTAKEGKSDSE

SEQ ID NO: 47 - DNA, 1002 bp, Homo sapiens
atggaggagaatgaccccaagcctggcgaagcagcggcggcggtggagggacagcggcagccggaatccagccccggcggcggctcgggcggcggcggcggtagcagcccgggcgaagcggacaccgggcgccggcgggctctgatgctgcccgcggtcctgcaggcgcccggcaaccaccagcacccgcaccgcatcaccaacttcttcatcgacaacatcctgcggcccgagttcggccggcgaaaggacgcggggacctgctgtgcgggcgcgggaggaggaaggggcggcggagccggcggcgaaggcggcgcgagcggtgcggagggaggcggcggcgcgggcggctcggagcagctcttgggctcgggctcccgagagccccggcagaacccgccatgtgcgcccggcgcgggcgggccgctcccagccgccggcagcgactctccgggtgacggggaaggcggctccaagacgctctcgctgcacggtggcgccaagaaaggcggcgaccccggcggccccctggacgggtcgctcaaggcccgcggcttgggcggcggcgacctgtcggtgagctcggactcggacagctcgcaagccggcgccaacctgggcgcgcagcccatgctctggccggcgtgggtctactgtacgcgctactcggaccggccttcttcaggtcccaggtctcgaaaaccaaagaagaagaacccgaacaaagaggacaagcggccgcgcacggcctttaccgccgagcagctgcagaggctcaaggccgagttccagaccaacaggtacctgacggagcagcggcgccagagcctggcgcaggagctgagcctcaacgagtcacagatcaagatttggttccagaacaagcgcgccaagatcaagaaggccacgggcaacaagaacacgctggccgtgcacctcatggcacagggcttgtacaaccactccaccacagccaaggagggcaagtcggacagcgagtag

SEQ ID NO: 48 - protein, 333 aa, Homo sapiens
MEENDPKPGEAAAAVEGQRQPESSPGGGSGGGGGSSPGEADTGRRRALMLPAVLQAPGNHQHPHRITNFFIDNILRPEFGRRKDAGTCCAGAGGGRGGGAGGEGGASGAEGGGGAGGSEQLLGSGSREPRQNPPCAPGAGGPLPAAGSDSPGDGEGGSKTLSLHGGAKKGGDPGGPLDGSLKARGLGGGDLSVSSDSDSSQAGANLGAQPMLWPAWVYCTRYSDRPSSGPRSRKPKKKNPNKEDKRPRTAFTAEQLQRLKAEFQTNRYLTEQRRQSLAQELSLNESQIKIWFQNKRAKIKKATGNKNTLAVHLMAQGLYNHSTTAKEGKSDSE

SEQ ID NO: 49 - DNA, 1407 bp, Mus musculus
atgttagggactgtgaagatggaagggcatgagagcaacgactggaacagctactacgcggacacgcaggaggcctactcctctgtccctgtcagcaacatgaactccggcctgggctctatgaactccatgaacacctacatgaccatgaacaccatgaccacgagcggcaacatgaccccggcttccttcaacatgtcctacgccaacacgggcttaggggccggcctgagtcccggtgctgtggctggcatgccaggggcctctgcaggcgccatgaacagcatgactgcggcgggcgtcacggccatgggtacggcgctgagcccgggaggcatgggctccatgggcgcgcagcccgccacctccatgaacggcctgggtccctacgccgccgccatgaacccgtgcatgagtcccatggcgtacgcgccgtccaacctgggccgcagccgcgcggggggcggcggcgacgccaagacattcaagcgcagctaccctcacgccaagccgccttactcctacatctcgctcatcacgatggccatccagcaggcgcccagcaagatgctcacgctgagcgagatctaccagtggatcatggacctcttcccctattaccgccagaaccagcagcgctggcagaactccatccgccactcgctgtccttcaacgattgtttcgtcaaggtggcacgatccccggacaagccaggcaagggctcctactggacgctgcacccggactccggcaacatgttcgagaacggctgctacttgcgccgccaaaagcgcttcaagtgtgagaagcagccgggggccggaggtgggagtgggggcggcggctccaaagggggcccagaaagtcgcaaggacccctcaggcccggggaaccccagcgccgagtcaccccttcaccggggtgtgcacggaaaggctagccagctagagggcgcgccggccccagggcccgccgccagcccccagactctggaccacagcggggccacggcgacagggggcgcttcggagttgaagtctccagcgtcttcatctgcgccccccataagctccgggccaggggcgctagcatctgtacccccctctcacccggctcacggcctggcaccccacgaatctcagctgcatctgaaaggggatccccactactcctttaatcaccccttctccatcaacaacctcatgtcctcctccgagcaacagcacaagctggacttcaaggcatacgagcaggcgctgcagtactctccttatggcgctaccttgcccgccagtctgccccttggcagcgcctcagtggccacgaggagccccatcgagccctcagccctggagccagcctactaccaaggtgtgtattccagacccgtgctaaatacttcctag

SEQ ID NO: 50 - protein, 468 aa, Mus musculus
MLGTVKMEGHESNDWNSYYADTQEAYSSVPVSNMNSGLGSMNSMNTYMTMNTMTTSGNMTPASFNMSYANTGLGAGLSPGAVAGMPGASAGAMNSMTAAGVTAMGTALSPGGMGSMGAQPATSMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGSGGGGSKGGPESRKDPSGPGNPSAESPLHRGVHGKASQLEGAPAPGPAASPQTLDHSGATATGGASELKSPASSSAPPISSGPGALASVPPSHPAHGLAPHESQLHLKGDPHYSFNHPFSINNLMSSSEQQHKLDFKAYEQALQYSPYGATLPASLPLGSASVATRSPIEPSALEPAYYQGVYSRPVLNTS

SEQ ID NO: 51 - DNA, 1419 bp, Homo sapiens
atgttaggaactgtgaagatggaagggcatgaaaccagcgactggaacagctactacgcagacacgcaggaggcctactcctccgtcccggtcagcaacatgaactcaggcctgggctccatgaactccatgaacacctacatgaccatgaacaccatgactacgagcggcaacatgaccccggcgtccttcaacatgtcctatgccaacccgggcctaggggccggcctgagtcccggcgcagtagccggcatgccggggggctcggcgggcgccatgaacagcatgactgcggccggcgtgacggccatgggtacggcgctgagcccgagcggcatgggcgccatgggtgcgcagcaggcggcctccatgaatggcctgggcccctacgcggccgccatgaacccgtgcatgagccccatggcgtacgcgccgtccaacctgggccgcagccgcgcgggcggcggcggcgacgccaagacgttcaagcgcagctacccgcacgccaagccgccctactcgtacatctcgctcatcaccatggccatccagcaggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggcccccgggcccgccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcctag

SEQ ID NO: 52 - protein, 472 aa, Homo sapiens
MLGTVKMEGHETSDWNSYYADTQEAYSSVPVSNMNSGLGSMNSMNTYMTMNTMTTSGNMTPASFNMSYANPGLGAGLSPGAVAGMPGGSAGAMNSMTAAGVTAMGTALSPSGMGAMGAQQAASMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGGGSGSGGSGAKGGPESRKDPSGASNPSADSPLHRGVHGKTGQLEGAPAPGPAASPQTLDHSGATATGGASELKTPASSTAPPISSGPGALASVPASHPAHGLAPHESQLHLKGDPHYSFNHPFSINNLMSSSEQQHKLDFKAYEQALQYSPYGSTLPASLPLGSASVTTRSPIEPSALEPAYYQGVYSRPVLNTS

SEQ ID NO: 53 - DNA, 912 bp, Mus musculus
atggccccggctgctgctatgacttctttgccactcggtgtcaaagtggaggactccgccttcgccaagcctgctgggggaggcgttggccaagcccccggggctgctgcggccaccgcaaccgccatgggcacagatgaggagggggccaagcccaaagtgcccgcttcactcctgcccttcagcgtggaggccctcatggccgatcacaggaagcccggggccaaggagagcgtcctggtggcctccgaaggggctcaggcagcgggtggctcggtgcagcacttgggcacccggcccgggtctctgggcgccccggatgcgccctcctcgccgcggcctctcggccatttctcagtcggaggactcctcaagctgccagaagatgctctggtgaaggccgaaagccccgagaaactagatcggaccccgtggatgcagagtccccgcttctccccgcccccagccagacggctgagtcccccagcatgcaccctacgcaagcacaagaccaaccgcaagcccaggacgcctttcaccacagctcagctgctggctctggagcgcaagttccgccagaagcagtacctgtctattgccgagcgcgcggaattctccagctcgctcagcctcaccgagacccaggtgaagatctggttccagaaccgtcgcgctaaggccaagagactgcaggaggcggagctggagaagctgaagatggccgcgaaacccatgttgccgcctgctgccttcggcctctcttttcctcttggcggtcctgcagcggtggctgcagctgcgggcgcctcactctacagtgcctctggccctttccagcgcgccgcgctgcctgtagcgcccgtgggactctacaccgcccatgtaggctacagcatgtaccacctgacttag

SEQ ID NO: 54 - protein, 303 aa, Mus musculus
MAPAAAMTSLPLGVKVEDSAFAKPAGGGVGQAPGAAAATATAMGTDEEGAKPKVPASLLPFSVEALMADHRKPGAKESVLVASEGAQAAGGSVQHLGTRPGSLGAPDAPSSPRPLGHFSVGGLLKLPEDALVKAESPEKLDRTPWMQSPRFSPPPARRLSPPACTLRKHKTNRKPRTPFTTAQLLALERKFRQKQYLSIAERAEFSSSLSLTETQVKIWFQNRRAKAKRLQEAELEKLKMAAKPMLPPAAFGLSFPLGGPAAVAAAAGASLYSASGPFQRAALPVAPVGLYTAHVGYSMYHLT

SEQ ID NO: 55 - DNA, 912 bp, Homo sapiens
atggccccggctgctgacatgacttctttgccactcggtgtcaaagtggaggactccgccttcggcaagccggcggggggaggcgcgggccaggcccccagcgccgccgcggccacggcagccgccatgggcgcggacgaggagggggccaagcccaaagtgtccccttcgctcctgcccttcagcgtggaggcgctcatggccgaccacaggaagccgggggccaaggagagcgccctggcgccctccgagggcgtgcaggcggcgggtggctcggcgcagccactgggcgtcccgccggggtcgctgggagccccggacgcgccctcttcgccgcggccgctcggccatttctcggtggggggactcctcaagctgccagaagatgcgctcgtcaaagccgagagccccgagaagcccgagaggaccccgtggatgcagagcccccgcttctccccgccgccggccaggcggctgagccccccagcctgcaccctccgcaaacacaagacgaaccgtaagccgcggacgcccttcaccaccgcgcagctgctggcgctggagcgcaagttccgccagaagcagtacctgtccatcgccgagcgcgcggagttctccagctcgctcagcctcactgagacgcaggtgaagatatggttccagaaccgccgcgccaaggcaaagagactacaagaggcagagctggagaagctgaagatggccgccaagcccatgctgccaccggctgccttcggcctctccttccctctcggcggccccgcagctgtagcggccgcggcgggtgcctcgctctacggtgcctctggccccttccagcgcgccgcgctgcctgtggcgcccgtgggactctacacggcccatgtgggctacagcatgtaccacctgacatag

SEQ ID NO: 56 - protein, 303 aa, Homo sapiens
MAPAADMTSLPLGVKVEDSAFGKPAGGGAGQAPSAAAATAAAMGADEEGAKPKVSPSLLPFSVEALMADHRKPGAKESALAPSEGVQAAGGSAQPLGVPPGSLGAPDAPSSPRPLGHFSVGGLLKLPEDALVKAESPEKPERTPWMQSPRFSPPPARRLSPPACTLRKHKTNRKPRTPFTTAQLLALERKFRQKQYLSIAERAEFSSSLSLTETQVKIWFQNRRAKAKRLQEAELEKLKMAAKPMLPPAAFGLSFPLGGPAAVAAAAGASLYGASGPFQRAALPVAPVGLYTAHVGYSMYHLT

SEQ ID NO: 57 - DNA, 63 bp, Artificial Sequence
  synthetic
aaacagactttgaattttgaccttctcaagttggcgggagacgtggagtccaacccagggccc

SEQ ID NO: 58 - DNA, 54 bp, Artificial Sequence
  synthetic
gagggcagaggaagtcttctaacatgcggtgacgtggaggagaatcccggccct

SEQ ID NO: 59 - DNA, 20 bp, Artificial Sequence
  synthetic
acgaccctcttagcccagag

SEQ ID NO: 60 - DNA, 19 bp, Artificial Sequence
  synthetic
ggccgaagggacgtagcag

SEQ ID NO: 61 - DNA, 20 bp, Artificial Sequence
  synthetic
ctgcaagtgaggaggtcatc

SEQ ID NO: 62 - DNA, 19 bp, Artificial Sequence
  synthetic
ctgctggcttgacaaccac

SEQ ID NO: 63 - DNA, 23 bp, Artificial Sequence
  synthetic
tttggaagatagggcctgcactg

SEQ ID NO: 64 - DNA, 24 bp, Artificial Sequence
  synthetic
cctggatgcggctatacaacactg

SEQ ID NO: 65 - DNA, 19 bp, Artificial Sequence
  synthetic
agcagaacggagtgcagct

SEQ ID NO: 66 - DNA, 19 bp, Artificial Sequence
  synthetic
gtatgctctgatgccgtct

SEQ ID NO: 67 - DNA, 22 bp, Artificial Sequence
  synthetic
cgtgggaccaatgtcttctgtg

SEQ ID NO: 68 - DNA, 22 bp, Artificial Sequence
  synthetic
atggtgaaggaggagaagaagt

SEQ ID NO: 69 - DNA, 24 bp, Artificial Sequence
  synthetic
acgcaagtgaattccgaaggagag

SEQ ID NO: 70 - DNA, 21 bp, Artificial Sequence
  synthetic
cagccgatggatcactttggt

SEQ ID NO: 71 - DNA, 21 bp, Artificial Sequence
  synthetic
cctactggctgctcggactaa

SEQ ID NO: 72 - DNA, 21 bp, Artificial Sequence
  synthetic
gcgtaccagtgactcaaactc

SEQ ID NO: 73 - DNA, 21 bp, Artificial Sequence
  synthetic
gcccttcatcgtcaccctgct

SEQ ID NO: 74 - DNA, 21 bp, Artificial Sequence
  synthetic
tgggcatggtctggatctcaa

SEQ ID NO: 75 - DNA, 24 bp, Artificial Sequence
  synthetic
tcaagactgactcacagcaacccc

SEQ ID NO: 76 - DNA, 24 bp, Artificial Sequence
  synthetic
ctttgtcctgaaccgtggtggtag

SEQ ID NO: 77 - DNA, 24 bp, Artificial Sequence
  synthetic
caagatcatcagcaatgcctcctg

SEQ ID NO: 78 - DNA, 22 bp, Artificial Sequence
  synthetic
gcctgcttcaccaccttcttga

SEQ ID NO: 79 - DNA, 20 bp, Artificial Sequence
  synthetic
ggcattgctctcaatgacaa

SEQ ID NO: 80 - DNA, 20 bp, Artificial Sequence
  synthetic
agggcctctctcttgctctc

SEQ ID NO: 81 - DNA, 19 bp, Artificial Sequence
  synthetic
cctcagcgtgcgtgtggtc

SEQ ID NO: 82 - DNA, 20 bp, Artificial Sequence
  synthetic
agcagtcgctgaggctggtg

SEQ ID NO: 83 - DNA, 21 bp, Artificial Sequence
  synthetic
ctcaccccaccccagatgcct

SEQ ID NO: 84 - DNA, 21 bp, Artificial Sequence
  synthetic
ctccctccccagcccacctct

SEQ ID NO: 85 - DNA, 20 bp, Artificial Sequence
  synthetic
ccccattgaccatctgtact

SEQ ID NO: 86 - DNA, 19 bp, Artificial Sequence
  synthetic
ggccgaagggacgtagcag

SEQ ID NO: 87 - DNA, 20 bp, Artificial Sequence
  synthetic
ggacattcccggacacacac

SEQ ID NO: 88 - DNA, 20 bp, Artificial Sequence
  synthetic
tctcgatcttcgtgagcttg

SEQ ID NO: 89 - DNA, 21 bp, Artificial Sequence
  synthetic
ttgctgccctggctatggtca

SEQ ID NO: 90 - DNA, 21 bp, Artificial Sequence
  synthetic
acaagcaagcatggccaaaca

SEQ ID NO: 91 - DNA, 20 bp, Artificial Sequence
  synthetic
tctacctgaaattggaagac

SEQ ID NO: 92 - DNA, 19 bp, Artificial Sequence
  synthetic
ggccgaagggacgtagcag

SEQ ID NO: 93 - DNA, 20 bp, Artificial Sequence
  synthetic
cgccttacgcagtcaatggg

SEQ ID NO: 94 - DNA, 21 bp, Artificial Sequence
  synthetic
gacagtggggagatggacgct

SEQ ID NO: 95 - DNA, 18 bp, Artificial Sequence
  synthetic
gcaactggccgcccaagg

SEQ ID NO: 96 - DNA, 18 bp, Artificial Sequence
  synthetic
aggccccacgttgaccga

SEQ ID NO: 97 - DNA, 20 bp, Artificial Sequence
  synthetic
ggcggcaaccagaagaacag

SEQ ID NO: 98 - DNA, 21 bp, Artificial Sequence
  synthetic
gcttggcctgcgtcgatgaac

SEQ ID NO: 99 - DNA, 22 bp, Artificial Sequence
  synthetic
gagtacaccgccgaggagattg

SEQ ID NO: 100 - DNA, 22 bp, Artificial Sequence
  synthetic
gcggatatactgggtgcactgg

SEQ ID NO: 101 - DNA, 21 bp, Artificial Sequence
  synthetic
tgtcacgtccccaaggttcat

SEQ ID NO: 102 - DNA, 21 bp, Artificial Sequence
  synthetic
gggcaggccgggtctctaagt

SEQ ID NO: 103 - DNA, 20 bp, Artificial Sequence
  synthetic
gattcagaggcaggtgcctg

SEQ ID NO: 104 - DNA, 21 bp, Artificial Sequence
  synthetic
gcatagtgcaagctggtggtc

SEQ ID NO: 105 - DNA, 24 bp, Artificial Sequence
  synthetic
ttggtctgttgtttgcctcgaaag

SEQ ID NO: 106 - DNA, 23 bp, Artificial Sequence
  synthetic
gggtccttcagcagcgtggttag

SEQ ID NO: 107 - DNA, 21 bp, Artificial Sequence
  synthetic
atccagactgccaggccagcg

SEQ ID NO: 108 - DNA, 21 bp, Artificial Sequence
  synthetic
ctccatccaagagcaccaagg

SEQ ID NO: 109 - DNA, 24 bp, Artificial Sequence
  synthetic
tgggctcctgtggctgtgttctag

SEQ ID NO: 110 - DNA, 22 bp, Artificial Sequence
  synthetic primer
ccggagcacaaggagtttcagt

(19) United States
(12) Patent Application Publication
(10) Pub. No.: US 2014/0315234 A1
(43) Pub. Date: Oct. 23, 2014

(54) METHOD TO GENERATE DOPAMINERGIC NEURONS FROM MOUSE AND HUMAN CELLS

(71) Applicants: Vania Broccoli, Milano (MI) (IT); Massimiliano Caiazzo, Milano (MI) (IT)

(72) Inventors: Vania Broccoli, Milano (MI) (IT); Massimiliano Caiazzo, Milano (MI) (IT)

(73) Assignee: OSPEDALE SAN RAFFAELE S.R.L., Milano (MI) (IT)

(21) Appl. No.: 13/991,837

(22) PCT Filed: Dec. 13, 2011
(86) PCT No.: PCT/EP2011/072608

§ 371 Date: Jun. 25, 2013

Related U.S. Application Data
(60) Provisional application No. 61/422,461, filed on Dec. 13, 2010.
(60) Provisional application No. 61/503,665, filed on Jul. 1, 2011.

(51) Int. Cl. C12N 5/0793

(52) U.S. Cl. CPC C12N 5/0619
USPC 435/29; 435/368; 435/354; 435/320.1

(57) ABSTRACT

The present invention relates to a method for reprogramming a differentiated non neuronal cell into a dopaminergic neuron comprising the step of inducing the expression in the differentiated non neuronal cell of at least the protein encoded by the Mash1 human gene or orthologues thereof and the protein encoded by the Nurr1 human gene or orthologues thereof, expression vectors, reprogrammed dopaminergic neuron and uses thereof.

### FIELD OF THE INVENTION

The present invention relates to a method for reprogramming a differentiated cell into a dopaminergic neuron by expressing specific proteins in the differentiated cell.

### BACKGROUND ART

Seminal studies have demonstrated that functional neurons can be generated independently of stem cells by direct cell conversion through genetic based approaches⁶. More recently, in a set of elegant experiments, fibroblasts have been directly converted into neuronal cells (iNs) by the forced expression of the three neurodevelopmental factors Mash1 (NCBI: Ascl1), Brn2 (NCBI: Pou3f2), and Myt1l⁷. However, iNs represent a heterogeneous population of glutamatergic and GABAergic neurons and their degree of global reprogramming remains to be properly characterized. It is thus unclear whether a specific neuronal subtype can be preferentially induced from direct reprogramming of heterologous cells. Therefore, the authors aimed to generate dopaminergic neurons (DA neurons) by direct conversion of somatic cells by forced expression of lineage-specific factors acting during brain development^{8,9}. Dopaminergic neurons are present in the CNS in ten distinct nuclei (A8-A17) where they preside the modulation of motor control, emotional behavior and sensory perception (e.g. retina and olfactory bulbs). Transplantation of DA neurons can potentially improve the clinical outcome of Parkinson's disease (PD), a neurological disorder resulting from degeneration of mesencephalic DA (mDA) neurons^{1,2}. In particular, transplantation of embryonic stem cell-derived DA neurons have shown to be efficient in restoring motor symptoms in conditions of DA deficiency^{3,4}. However, the use of pluripotent derived cells might lead to the development of tumors if not properly controlled⁵.

### SUMMARY OF THE INVENTION

In the present invention, the authors identified a minimal set of transcription factors which, upon their gene activation/expression, are able to generate directly functional DA neurons from non neuronal differentiated cells, i.e. fibroblasts, without reverting to a progenitor cell stage. Induced dopaminergic neuronal (iDAN) cells release dopamine and show spontaneous electrical activity organized in regular spikes consistent with the pacemaker activity featured by brain DA neurons. The identified transcription factors were able to elicit DA neuronal conversion in prenatal and adult fibroblasts from healthy donors and PD patients. Direct generation of induced dopaminergic (iDA) cells from somatic cells has significant implications for understanding critical processes of neuronal development, disease in vitro modeling and for cell replacement therapies.

One advantage of the present method is that it does not rely on pluripotent stem cells that are prone to generate tumors. Moreover, the process of the invention does not pass through proliferative progenitors that also might result tumorigenic²¹. Thus, the method of the invention avoid a dangerous drawback of stem cell therapies while providing enough number of functional DA neurons amenable for autologous cell replacement therapies.

It is therefore an object of the invention a method for reprogramming a differentiated non neuronal cell into a dopaminergic neuron comprising the step of inducing the expression in the differentiated non neuronal cell of at least the protein encoded by the Mash1 human gene or orthologues thereof and the protein encoded by the Nurr1 human gene or orthologues thereof. Preferably, the method further comprises the step of inducing the expression in the differentiated non neuronal cell of the protein encoded by the Lmx1a human gene and/or by the Lmx1b human gene or orthologues thereof.

Still preferably, the method further comprises the step of inducing the expression in the differentiated non neuronal cell of at least a protein encoded by a gene selected from the group of: Brn2, Myth1l, En-1, En-2, Pitx3, Foxa1, Foxa2, Otx2, Msx1 or Neurog2 human genes or orthologues thereof.

Yet preferably, the method comprises the step of inducing the expression in the differentiated non neuronal cell of proteins encoded by each of the following human genes or orthologues thereof: Mash1, Nurr1, Lmx1a, Lmx1b, Brn2, Myth1l, En-1, En-2, Pitx3, Foxa1, Foxa2, Otx2, Msx1 and Neurog2.

In a preferred embodiment the differentiated non neuronal cell is a mouse or a human cell.

In a preferred embodiment the differentiated non neuronal cell is selected from the group of: a cell of mesoderm origin or a cell of ectoderm origin, a fibroblast, an astroglial cell, a skin keratinocyte or an hematopoietic cell.

Preferably, the differentiated non neuronal cell is an adult cell.

Still preferably the differentiated non neuronal cell is an adult cell of an healthy subject or of a subject affected by a neurological disorder.

In a preferred embodiment the neurological disorder is characterized by dopaminergic system dysfunction. Preferably the neurological disorder characterized by dopaminergic system dysfunction is Parkinson's disease.

In a preferred embodiment the step of inducing the expression is obtained by genetically transforming the differentiated non neuronal cell with at least one vector containing and expressing the coding sequences of proteins as defined above.

Preferably the genetic transformation is performed by transfecting or infecting the differentiated non neuronal cell.

Still preferably the differentiated non neuronal cell is infected by a recombinant lentivirus.

In a preferred embodiment the step of inducing the expression is performed in hypoxia conditions.

Still preferably the step of inducing the expression is performed in the presence of 2 to 6% O₂. More preferably it is performed in the presence of 5% O₂.

It is a further object of the invention an eukaryotic vector comprising and expressing under appropriated promoter and regulatory sequences the coding sequences of the proteins as defined above.

Preferably, the eukaryotic vector comprises and expresses under appropriated promoter and regulatory sequences the coding sequences of the proteins Mash1, Nurr1 and either Lmx1 a or Lmx1b.

In a preferred embodiment the coding sequences of the proteins Mash1, Nurr1 and either Lmx1a or Lmx1b are in the following order: 5′ Mash1-Nurr1 and Lmx1a or Lmx1b 3′. Preferably, the vector of the invention is for use in the treatment of a neurological disorder. Preferably, the neurological disorder is characterized by dopaminergic system dysfunction. Still preferably the neurological disorder is Parkinson's disease.

It is a further object of the invention a dopaminergic neuron reprogrammed according to the method of the invention.

Preferably the reprogrammed dopaminergic neuron is for medical use. Still preferably it is for use in the treatment of a neurological disorder.

Yet preferably the neurological disorder is characterized by dopaminergic system dysfunction. Still preferably the neurological disorder is Parkinson's disease.

It is a further object of the invention a pharmaceutical composition comprising the reprogrammed dopaminergic neuron of the invention or the vector as defined above.

It is another object of the invention method for the screening of putative therapeutic agents comprising the step of:
incubating the reprogrammed dopaminergic neuron of the invention with the putative therapeutic agents;
measuring and/or observing an appropritate phenotype in said reprogrammed dopaminergic neuron; and
comparing said measured and/or observed phenotype with an appropriated control phenotype.

In the present invention it is possible to use iDAN cells for screening applications. Particularly, a library of pharmacological compounds may be tested on iDAN cells derived from healthy and/or Parkinson's disease (PD) patients in order to assess if it possible to rescue a potential phenotype identified in PD-iDAN cells. This phenotype could be related, as an example, to cell survival. The phenotype may be any output related to dopaminergic neurons, such as electrophysiological output or DA release or DA uptake. Further, in order to exacerbate the potential phenotype related to PD-iDAN cells, cells could be treated with a molecule that is able to induce oxidative stress, such as hydrogen peroxyde. Alternatively, iDAN cells could be engineered to overexpress alpha-synuclein in order to induce the formation of protein aggregates.

In the present invention a neurological disorder characterized by dopaminergic system dysfunction means a neurological disorder caused by a defect of dopaminergic neurons electrical activity and dopamine release, such as Parkinson's disease, attention deficit hyperactivity disorder (ADHD), addictive disorders, depression and schizophrenia.

The present invention will be described by means of non-limiting examples referring to the following figures and tables.

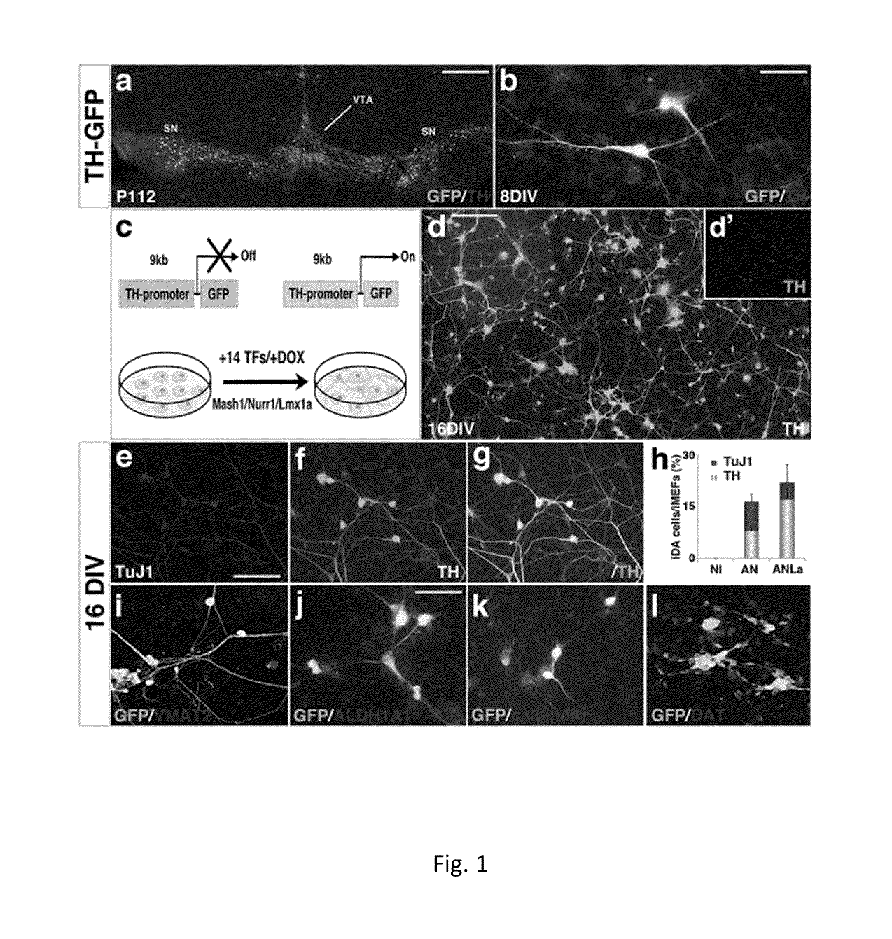

FIG. 1. Mash1, Nurr1 and Lmx1a reprogram mouse fibroblasts into iDA cells. TH and GFP detection in TH-GFP adult brain (a) and ventral midbrain primary cell culture (b). c, Scheme of DA transcription factors screening. TH staining in iDA cells (d) and in uninfected MEFs (d′) after 16 DIV (days in vitro). iDA cells are positive for the DA markers TH (e-g), VMAT2, ALDH1A1, calbindin and DAT (i-l). h, Quantification of TuJ1+ and TH+ cells. Scale bars: 20 μm (b, j), 50 μm (e-g, k, l), 100 μm (d, i), and 500 μm (a). SN, substantia nigra; VTA (ventral tegmental area). Data are presented with mean±s.e.m. NI (not infected), AN (Mash1, Nurr1), ANLa (Mash1, Nurr1, Lmx1a).

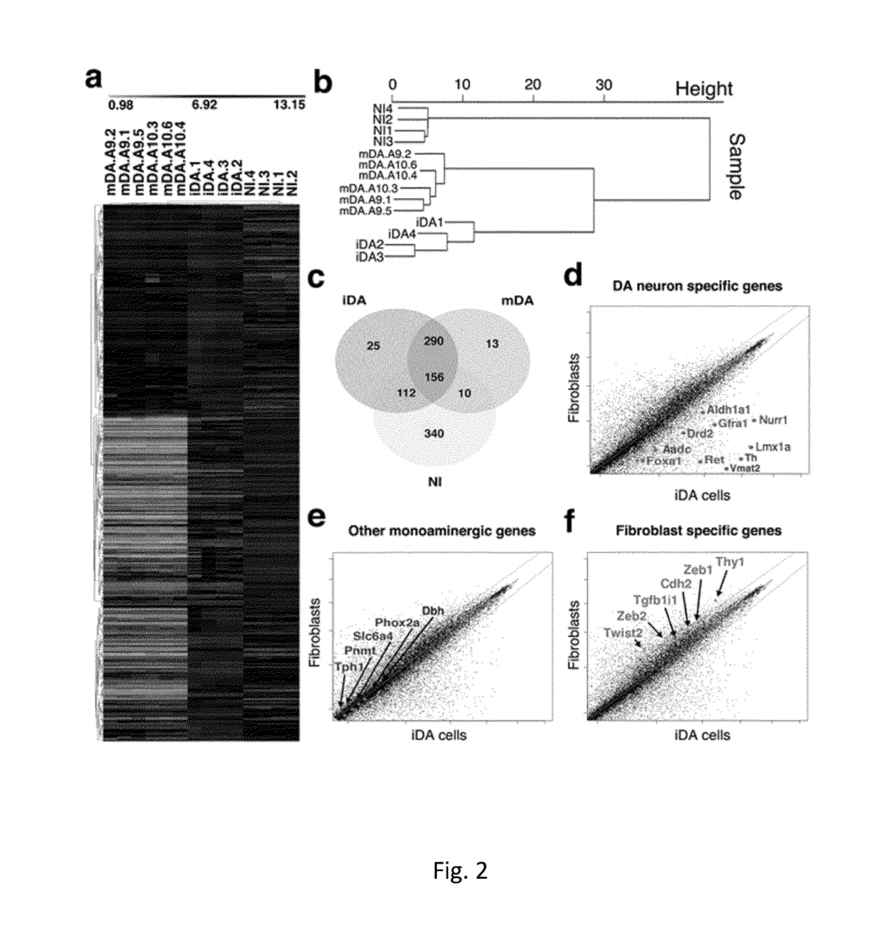

FIG. 2. Mouse iDA cells expression profiling. a, Heat-map of genes differentially expressed in RNA-microarray analysis performed on MEFs (NI), iDA cells and brain mesencephalic dopaminergic neurons (mDA A9-A10). Hierarchical clustering (b) and general degree of overlapping expression (c) among the three cell populations analyzed. d-f, Scatter plots show that in iDAN cells the majority of DA markers are increased, whereas other monoaminergic neuronal markers are not activated and fibroblasts markers are silenced.

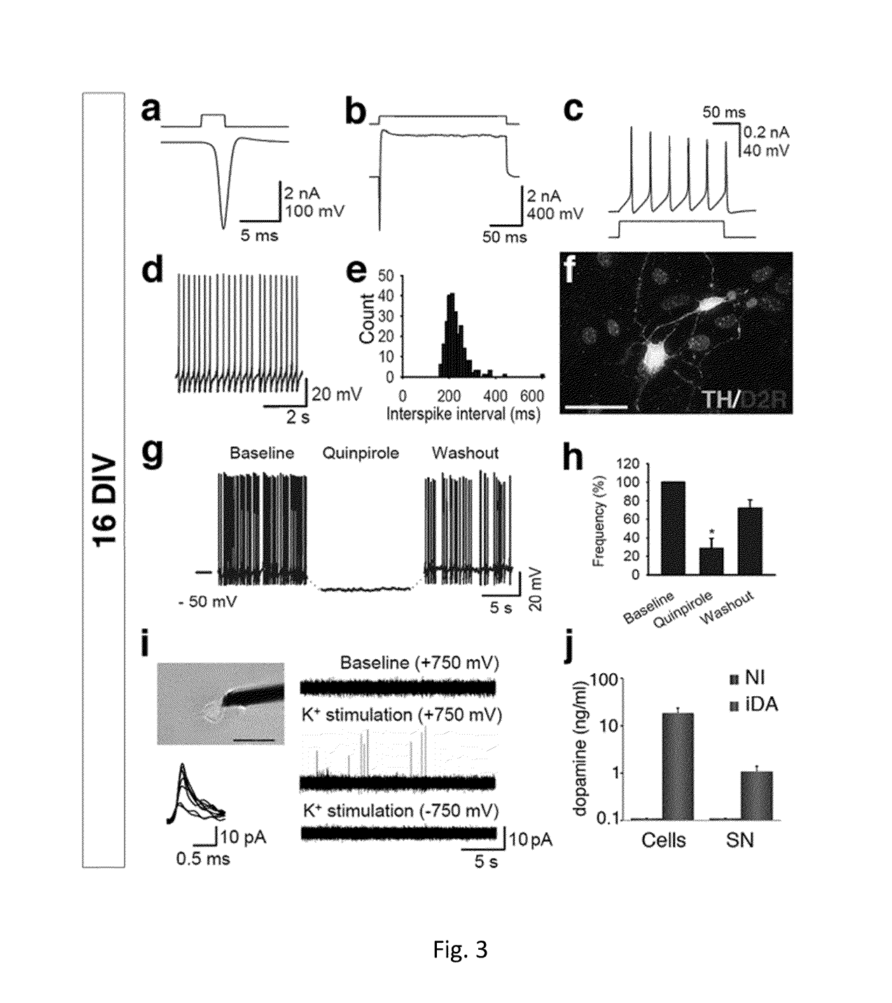

FIG. 3. Functional characterization of mouse iDAN cells. a, b, Whole-cell voltage-clamp recording of Na⁺ and K⁺ currents. c, Current-clamp recording of multiple action potentials evoked by current injection. d, e, Current-clamp recording and interspike interval frequency of spontaneous action potentials f, D2 receptor (D2R) staining. Effect of the D2/D3 agonist quinpirole on spiking frequency (g) and its statistical analysis (h) (*p=0.005, paired t-test, n=6). i, amperometric recordings after K⁺ stimulation; high resolution pattern is shown below the image of the recorded cell. j, dopamine content measured by HPLC in uninfected (NI) and iDAN cells, both in cell pellets and in the supernatant (SN) after K⁺ stimulation. Scale bars: 20 μm (i), and 50 μm (b). Data are presented with mean±s.e.m.

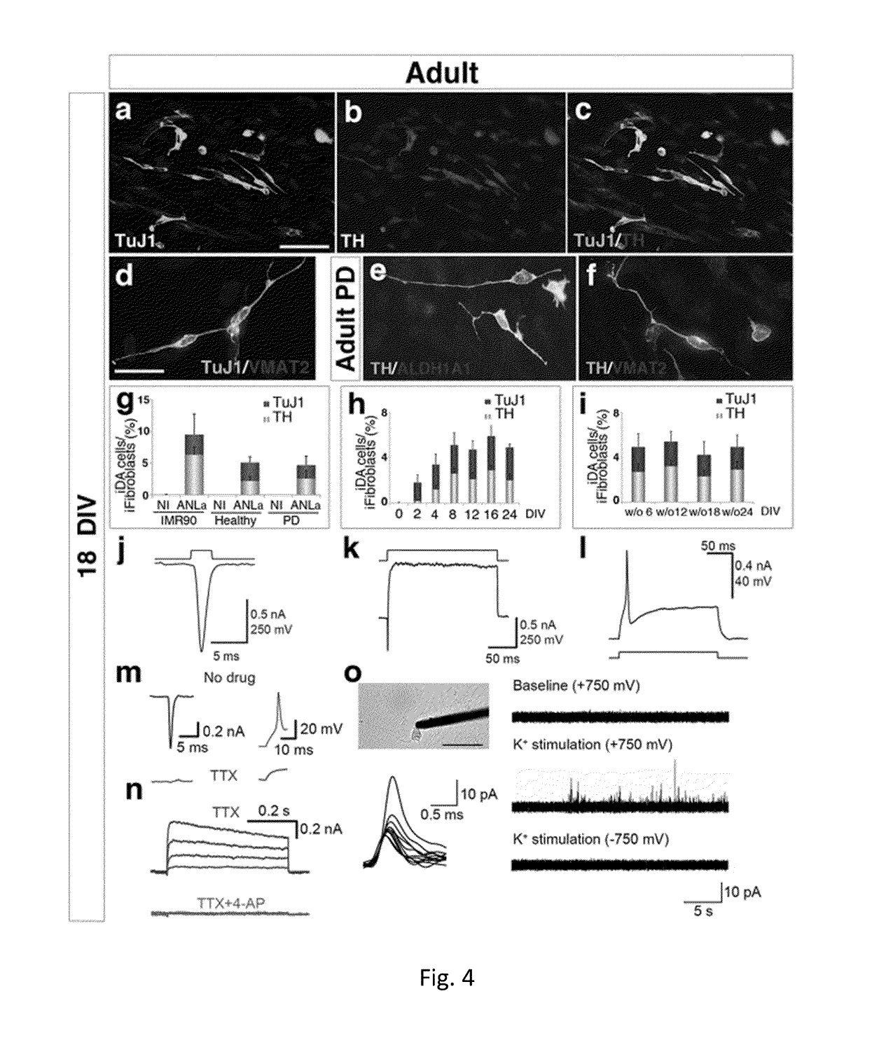

FIG. 4. Characterization of human fibroblasts reprogrammed into iDAN cells. Fibroblasts from healthy donor (a-d) and PD patient (e-f) show a comparable efficiency in DA neuronal conversion. g, Quantification of iDAN cells obtained from fetal (IMR90), healthy and PD adult fibroblasts. h, Quantification of TuJ1+ and TH+ cells in a time course study from 0 to 24 days in vitro (DIV). i, Quantification of TuJ1+ and TH+ reprogrammed cells kept with (w) or without (w/o) doxycycline for 6, 12, 18 or 24 DIV. j, k, Whole-cell voltage-clamp recording of Na⁺ and K⁺ currents. l, Whole-cell current-clamp recording of single action potential elicited by a minimal depolarization. Suppression of Na⁺ (m) and K⁺ (n) currents and action potentials by tetrodotoxin (TTX) and 4-AP. o, amperometric recordings after K⁺ stimulation; high resolution pattern is shown below the image of the recorded cell. Scale bars: 20 μm (d-f, o), and 50 μm (a-c). Data are presented with mean±s.e.m.

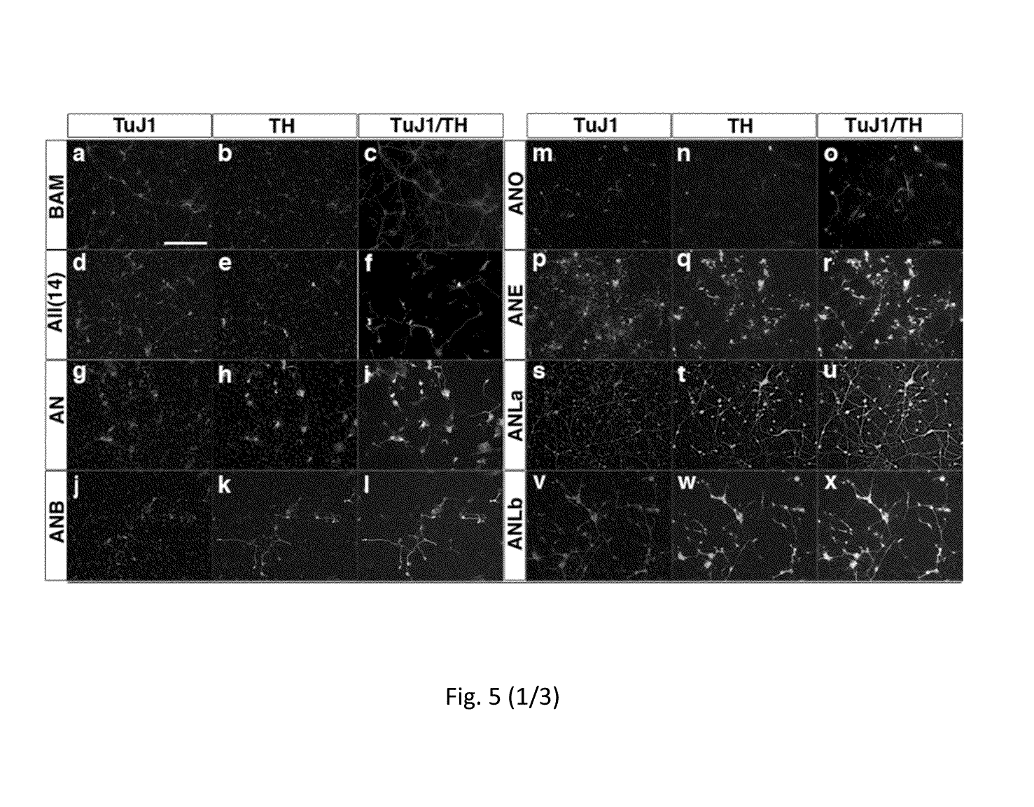

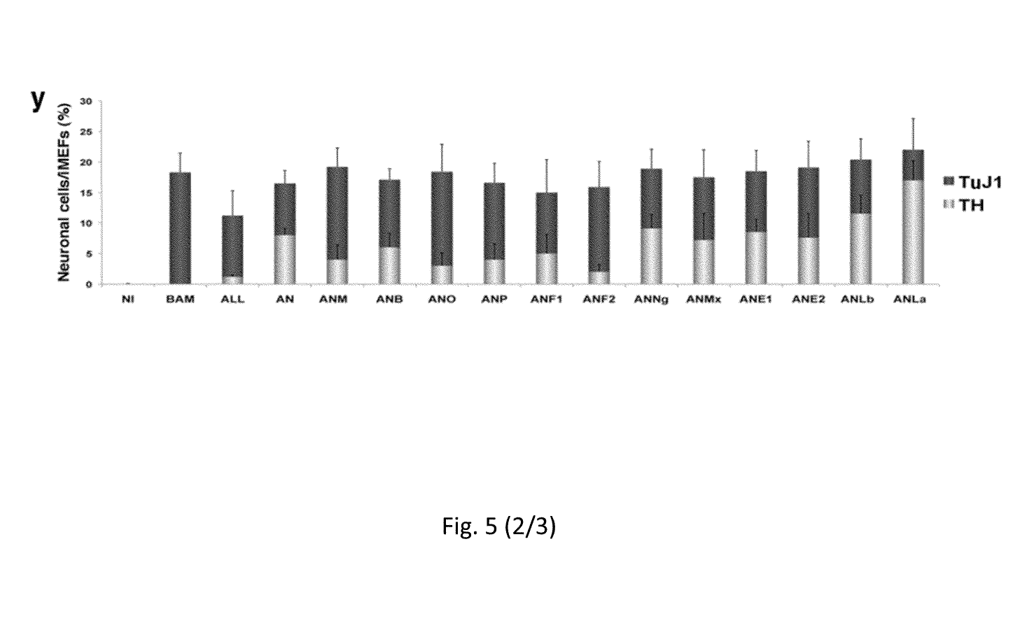

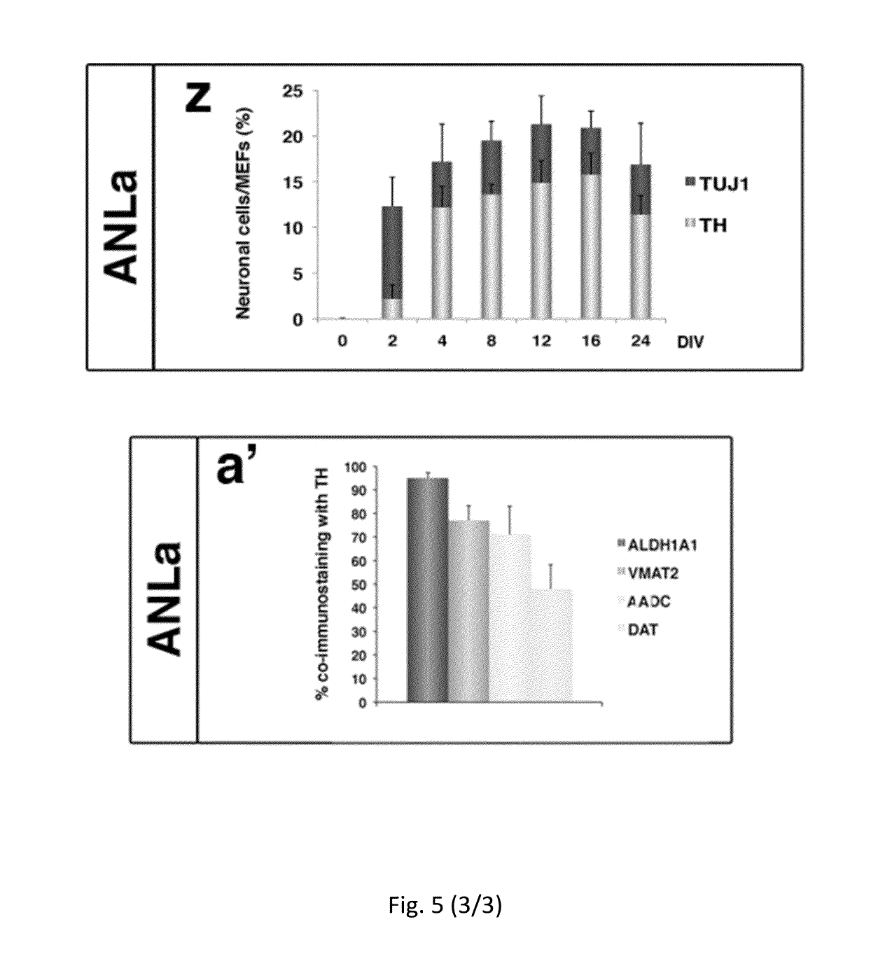

FIG. 5. Screening for the optimal combination of transcription factors inducing an efficient conversion of MEFs into iDAN cells. a-x, Efficiency of the pan-neuronal (TuJ1) and DA (TH) reprogramming of MEFs with some representative combinations of transcription factors evaluated through immunocytochemical analysis. The iN reprogramming factors (Mash1, Brn2 and Myt1l) promoted a robust neuronal induction, as proved by TuJ1+ cells (a-c), however this combination was not efficient for the generation of TH positive cells. y, Quantification of TuJ1+ and TH+ reprogrammed cells over the total of infected MEFs for all the viral combinations tested. z, Quantification of TuJ1+ and TH+ cells in a time course study of reprogramming from 0 to 24 days in vitro (DIV). The fraction of induced GFP+/TH+ remained constant up to 24 DIV. a′, Quantification of TH+ reprogrammed cells that co-express other DA markers. Letter code identifying gene combinations is reported in Table 1. Scale bar: 200 μm. Data are presented with mean±s.e.m.

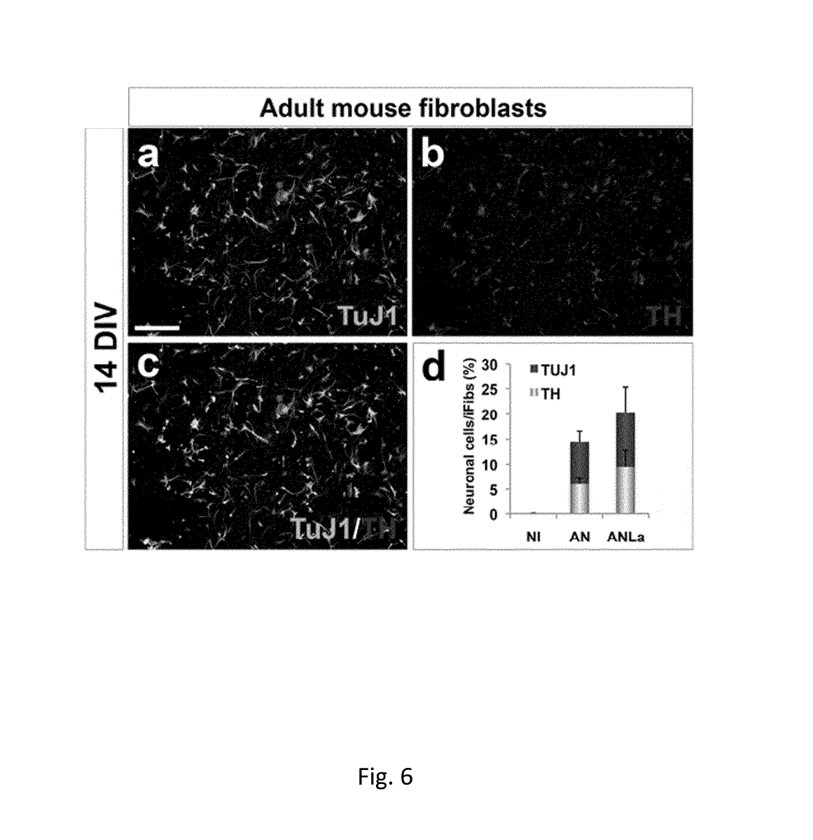

FIG. 6. Reprogramming of adult mouse tail fibroblasts into iDAN cells. a-c, Representative images of TuJ1 and TH immunostainings in reprogrammed adult tail fibroblasts. d, Quantification of TuJ1+ and TH+ neuronal cells following infection with AN and ANLa viral cocktails. Uninfected cells quantification (NI) is also shown. Immunocytochemical analysis was performed 14 days after lentiviral induction. Scale bar: 100 μm. Data are presented with mean±s.e.m.

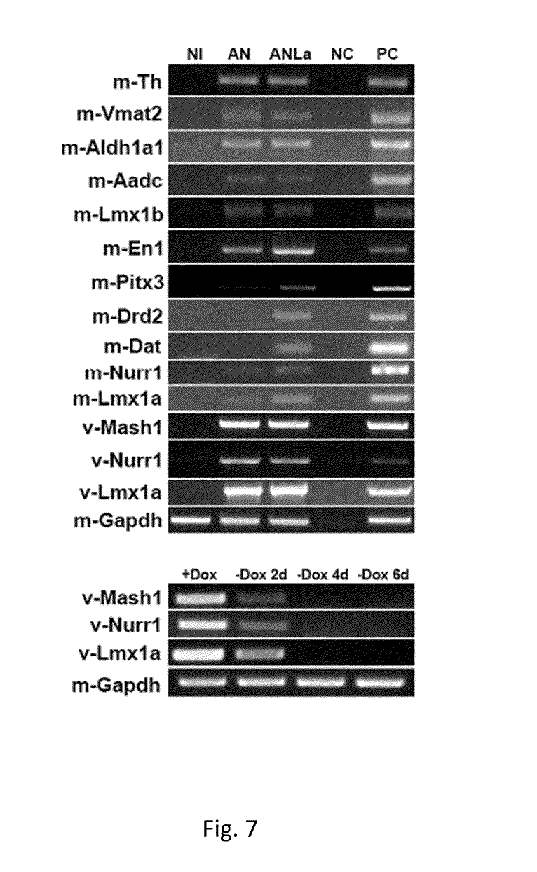

FIG. 7. RT-PCR analysis of DA neuronal markers in mouse iDAN cells. Expression profile of DA markers present in uninfected (NI) MEFs, or MEFs infected with the AN or ANLa lentiviral cocktails. RT-PCR analysis was performed after 16 days from lentiviral induction. Expression of the viral Mash1, Nurr1 and Lmx1a transgenes is also shown (v-Mash1, v-Nurr1 and v-Lmx1a). Viral transgene expression is silenced after 4 days (d) of doxycycline (dox) withdrawal.

E 14.5 mouse ventral midbrain tissue was used as positive control (PC) and not retro-transcripted samples as negative control (NC).

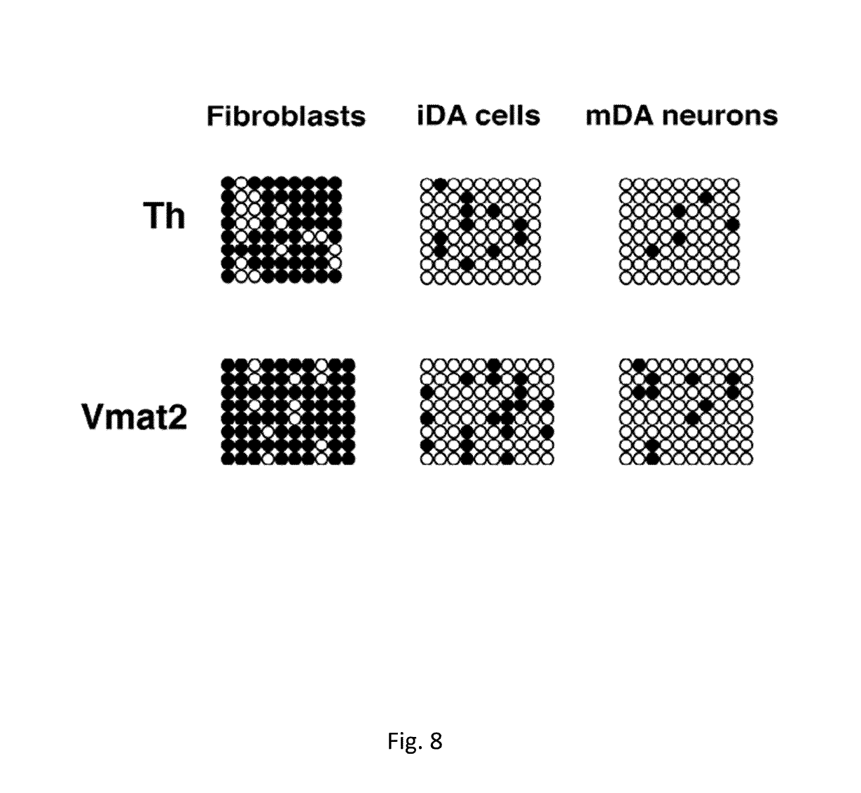

FIG. 8. Analysis of the epigenetic state of the Th and Vmat2 promoter regions. Analysis of the methylation state of the Th and Vmat2 promoters using bisulphite analysis in fibroblasts, reprogrammed iDAN cells and GFP+ sorted E14.5 brain DA neurons. Open circles indicate unmethylated CpG dinucleotides; closed circles indicate methylated CpGs.

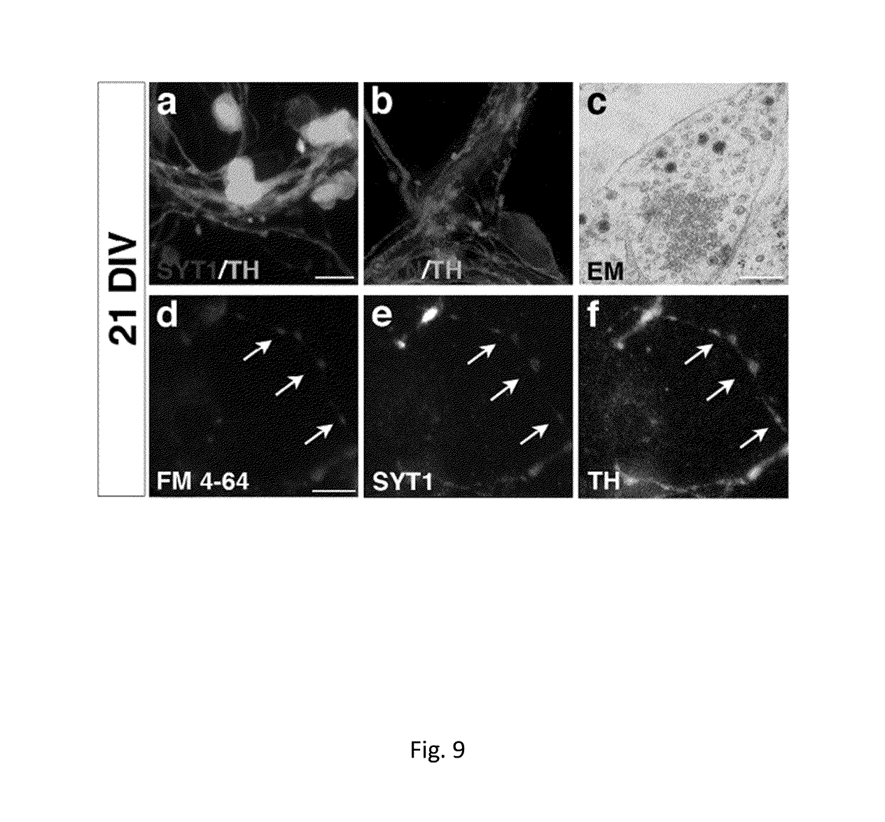

FIG. 9. Characterization of mouse iDAN cell synapses by FM 4-64 dye loading. a, b Immunocytochemical analysis for the synaptic markers Synaptotagmin 1 (SYT1) and Synapsin (SYN) in iDAN cells. C, Ultrastructure of a single iDAN synapse analyzed by electron microscopy (EM). d-f, Activity of iDAN synapses as proved by the co-localization of the FM4-64 dye (d) with SYT1 (e) and TH staining in iDA cells. The analysis was performed 21 days in vitro (DIV) after lentiviral infection. Arrows indicate TH+/SYT+ synaptic vesicles that show FM4-64 dye uptake. Scale bars: 500 nm (c), 10 μm (a, d).

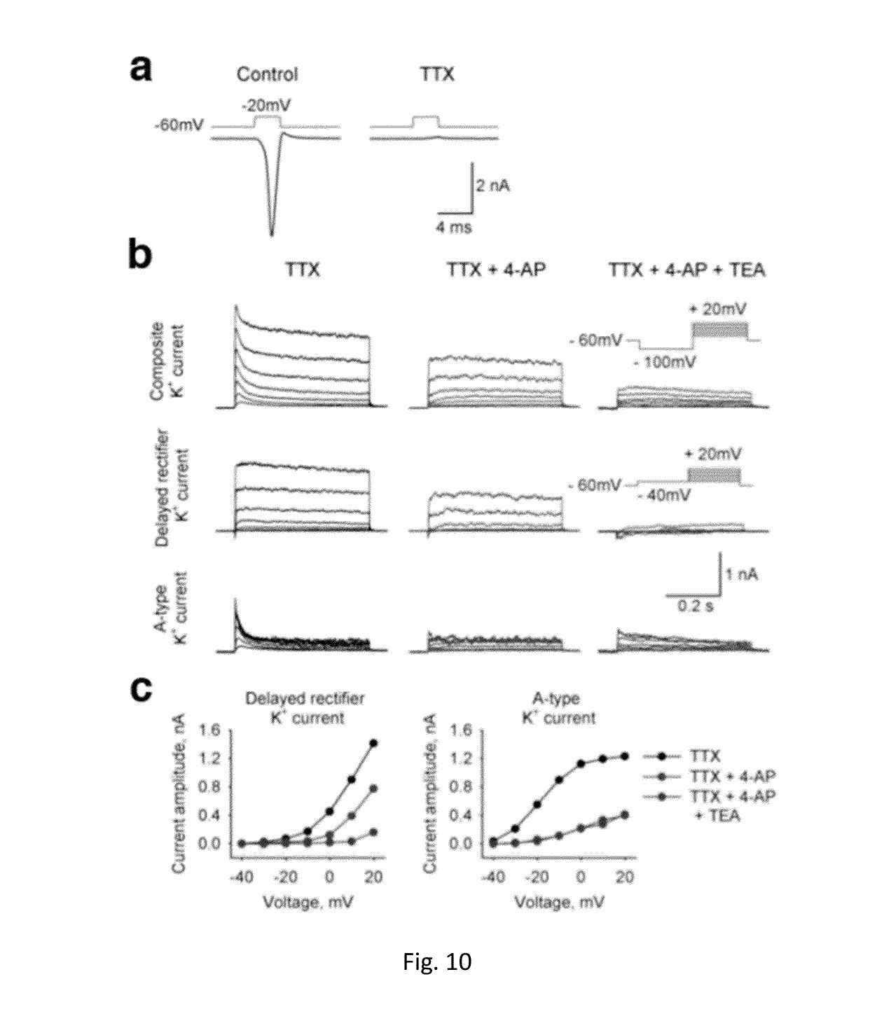

FIG. 10. Pharmacological analysis of Na⁺ and K⁺ currents in mouse iDA cells. a, Representative traces showing a complete block of the fast inward current by 0.5 μM tetrodotoxin (TTX). The same result was observed in 8 cells, confirming that this component is mediated by Na⁺ voltage-gated channels. b, Representative recordings showing the effects of K⁺ channel blockers 4-aminopyridine (4-AP, 3 mM) and tetraethylammonium (TEA, 10 mM) on outward K⁺ currents activated after a prepulse to −100 mV (top row, composite current) or −40 mV (delayed rectifier, middle row), and a difference between them (A-type current). The insets show the protocols used. All recordings were performed in the presence of TTX. c, Voltage-current curves showing the inhibition of outward currents by 4-AP and TEA (n=4).

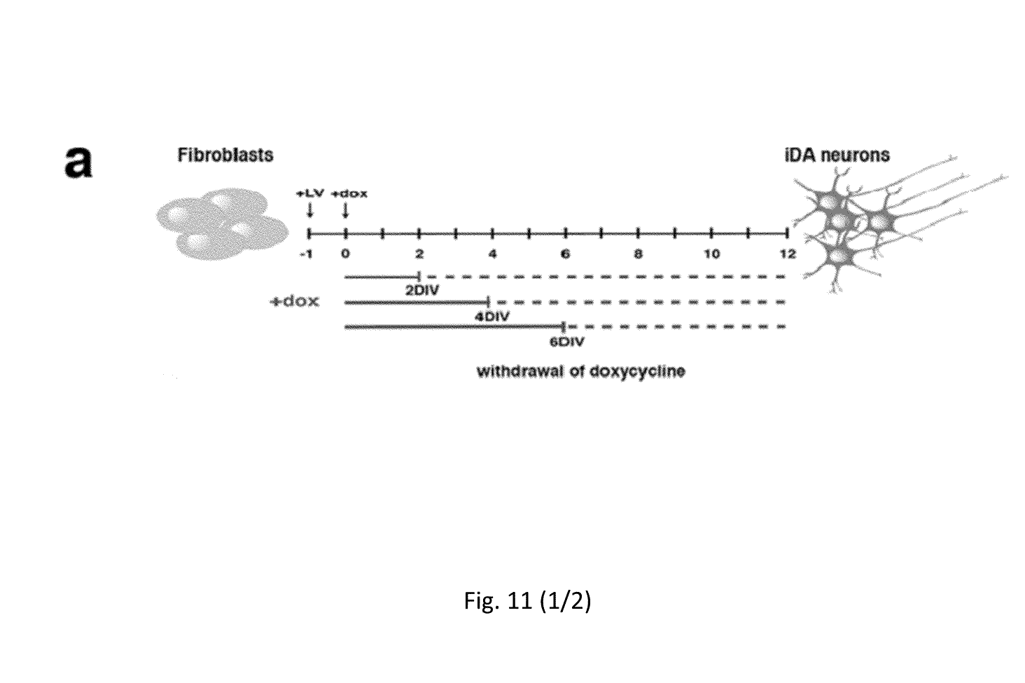

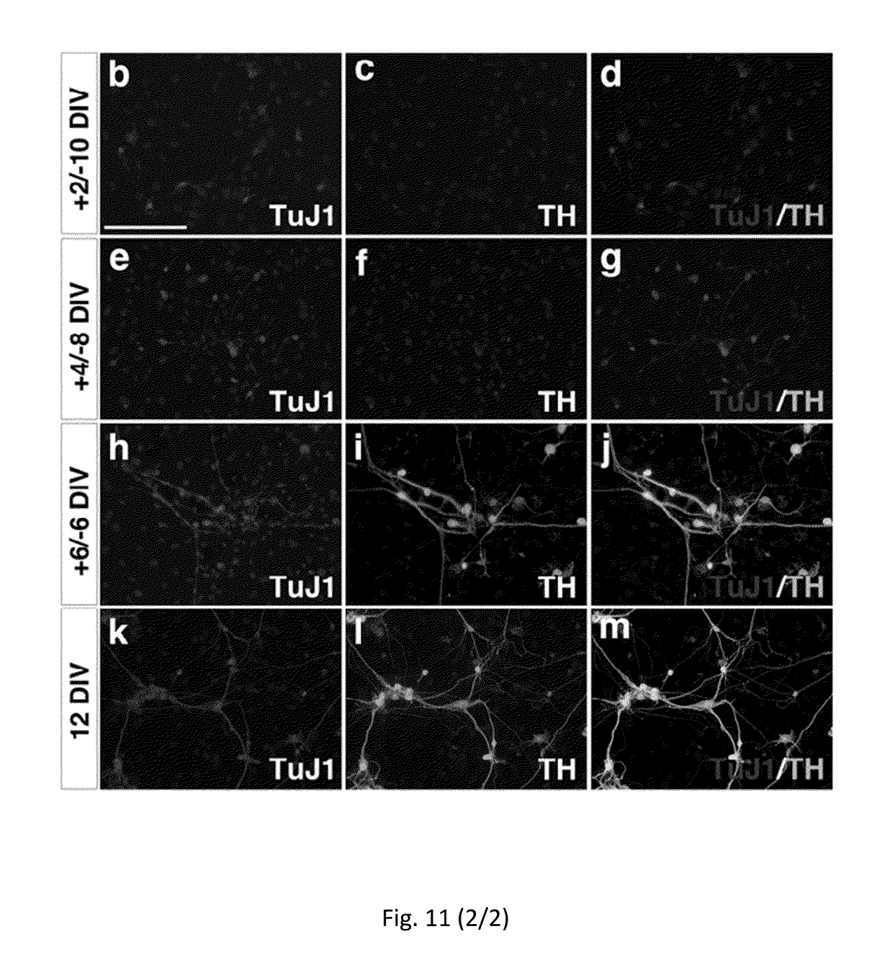

FIG. 11. Temporal requirement for the reprogramming factors to establish a stable induced cell conversion. a, Scheme of doxycycline (dox) treatment of DA reprogramming. Infected MEFs were treated with dox for different time windows to induce transcription factors expression, followed by dox withdrawal. b-m, TuJ1 and TH immunocytochemistry performed at 12 days in vitro (DIV) after 2, 4, 6 and 12 days of dox exposure reveals that a minimum of 6 days of lentiviral induction (LV) is required to obtain TuJ1+ and TH+ neuronal cells with a mature neuronal phenotype. Scale bar: 50 μm.

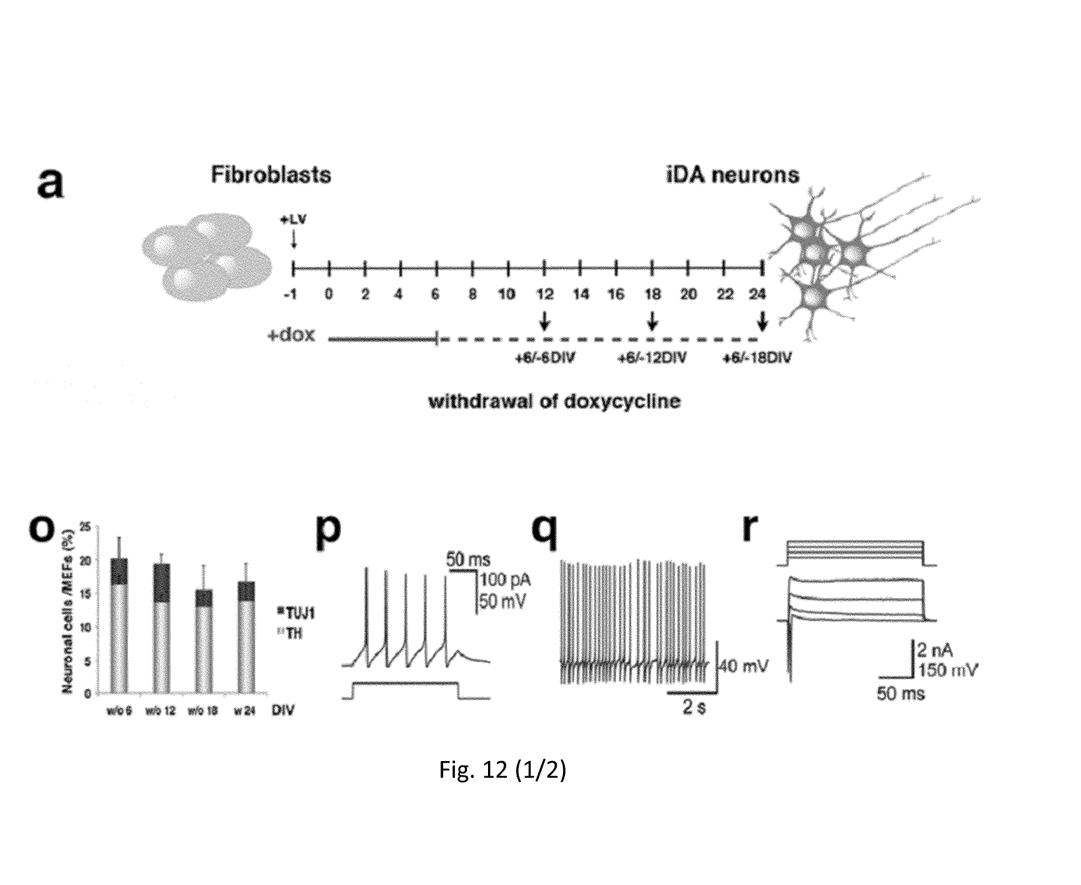

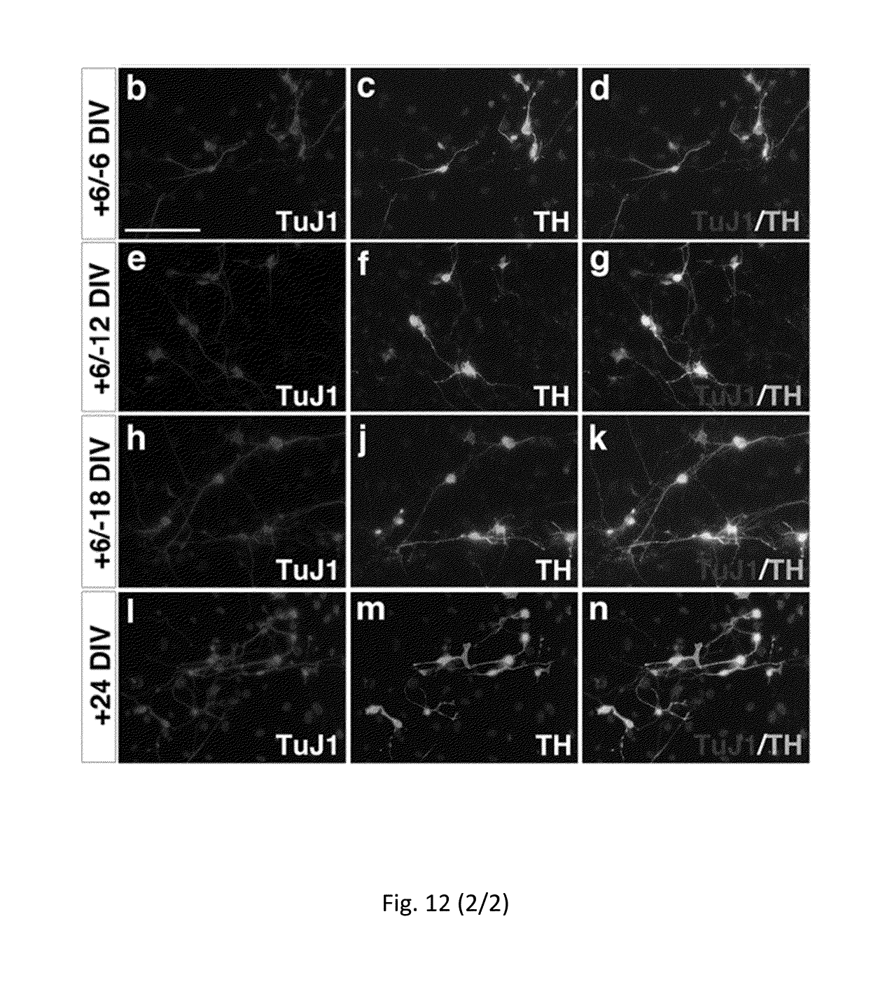

FIG. 12. iDAN cells are stable after doxycycline withdrawal for a long period in culture. a, Scheme of doxycycline (dox) treatment during DA reprogramming. Infected MEFs were treated with dox for 6 days in vitro (DIV), followed by dox withdrawal for different time windows. b-n, TuJ1 and TH immucytochemistry performed after 6 (b-d), 12 (e-g), 18 (h-k) DIV without dox or keeping dox for 24 DIV (l-n). o, Quantification of TuJ1+ and TH+ reprogrammed cells kept with (w) or without (w/o) dox for 6, 12, 18 or 24 DIV. p, Representative whole-cell current-clamp recording of multiple action potentials evoked by current injection, which shows spike amplitude attenuation during the burst. q, Current-clamp recording of spontaneous action potentials, which demonstrates rhythmic spiking of a iDAN cell. r, Whole-cell voltage-clamp recording of Na⁺ (rapid inward) and K⁺ (slow outward) voltage-gated currents. The cell was held at −60 mV and voltage steps to −10, 0, +10 and +20 mV were delivered to activate the currents. Scale bar: 50 μm. Data are presented with mean±s.e.m.

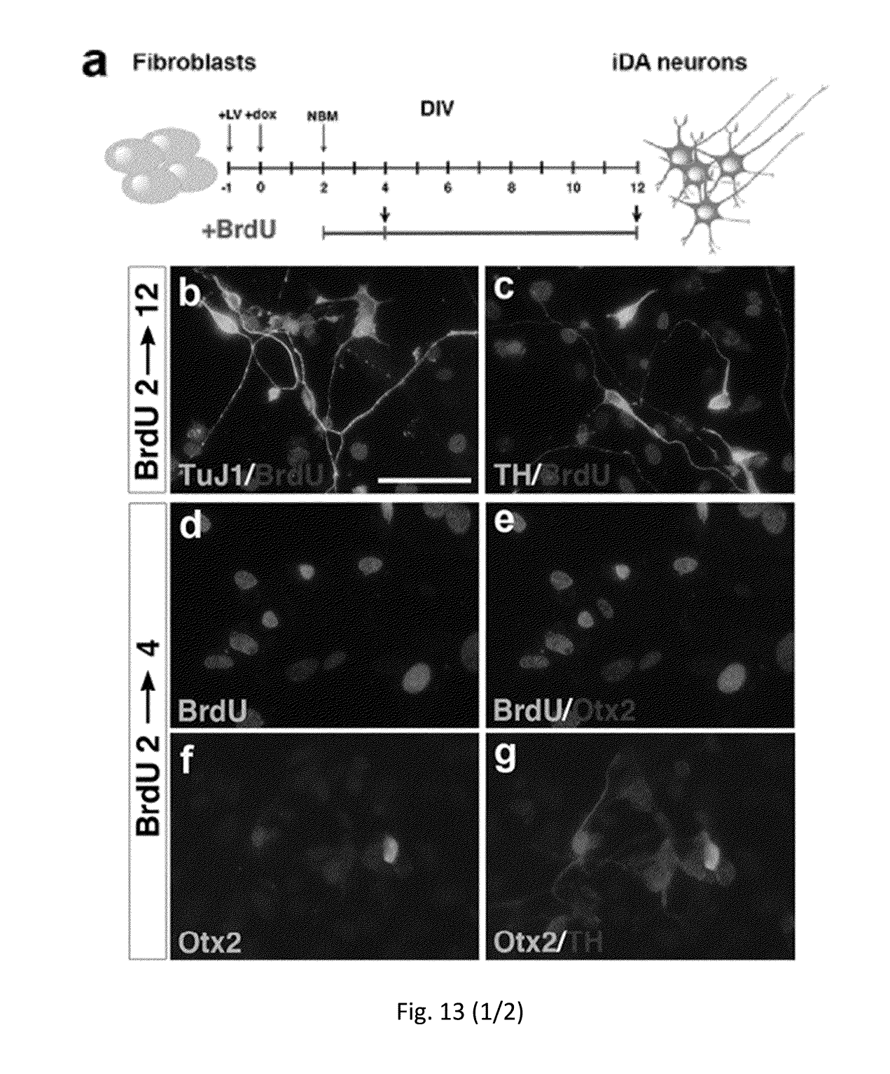

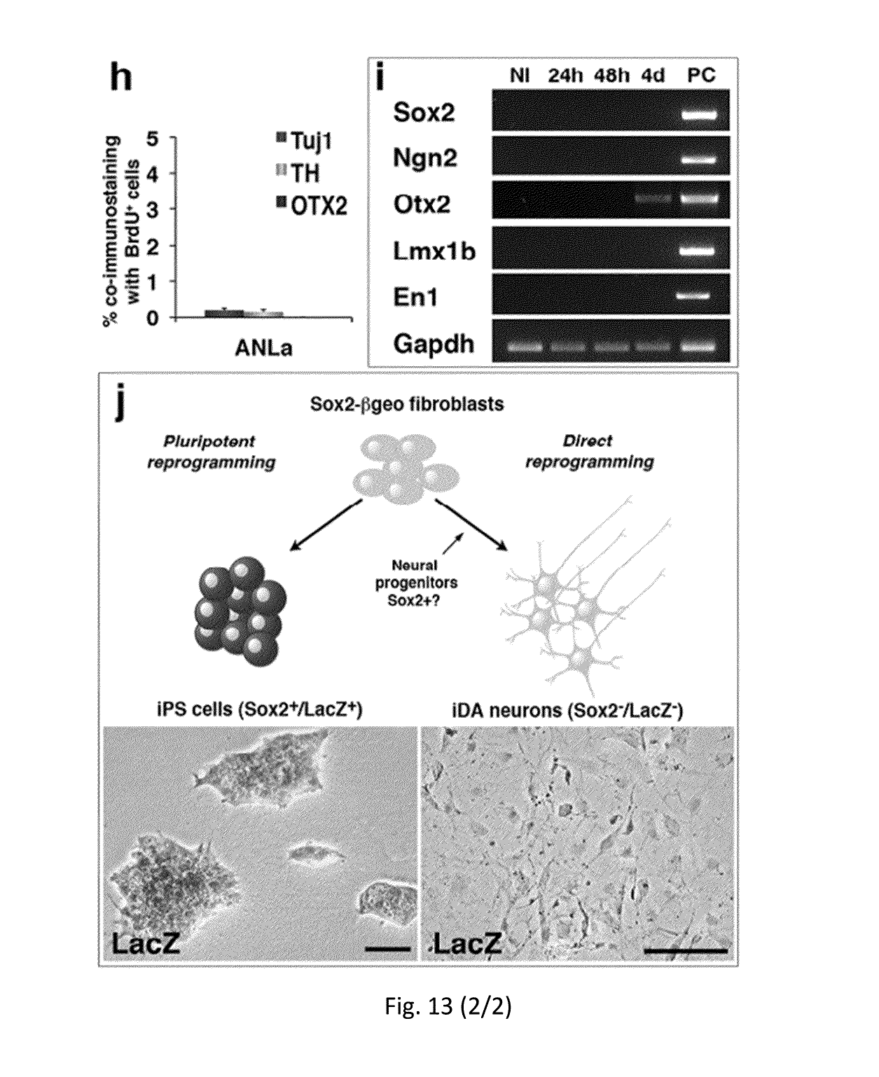

FIG. 13. Reprogramming of MEFs into iDAN cells does not require to pass through an intermediate neuronal progenitor state. a, Outline of the BrdU treatment on MEFs during reprogramming. Cells were treated with BrdU (10 μM) for different time windows after the activation of lentiviral vectors (LV). Two days after lentiviral infection cells were shifted to a neurobasal medium (NBM). b, c, TuJ1, TH and BrdU immunocytochemistry at 12 days in vitro (DIV) on iDAN cells treated from day 2 to 12 with BrdU reveals that the majority of TuJ1+ cells are post-mitotic. d-g, OTX2, TH and BrdU immunocytochemistry on iDAN cells treated from day 2 to 4 with BrdU and analyzed at 4 DIV shows that the few OTX2+ cells are postmitotic. h, Quantification of BrdU+ cells that coexpress TuJ1, TH and OTX2 showed in b-g panels. i, RT-PCRs at 24, 48 hours (h) and 4 days (d) of reprogramming. Not infected MEFs (NI) and E14.5 mouse ventral midbrain positive control (PC) are also shown. j, β-Gal staining on DA reprogrammed Sox2^{+/β-geo} MEFs shows no LacZ activity thus demonstrating lack of Sox2 expression during DA direct reprogramming. Conversely Sox2^{+/β-geo} MEFs reprogrammed to iPS cells show clear β-Gal staining. Scale bars: 20 μm (b) and 50 μm (j). Data are presented with mean±s.e.m.

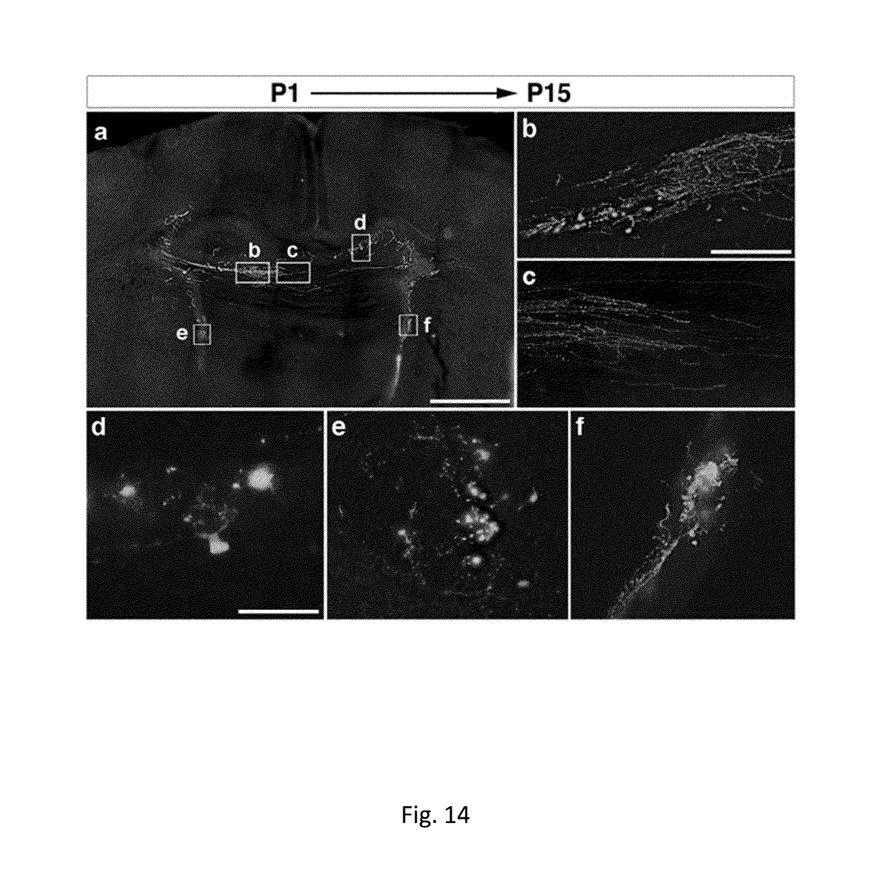

FIG. 14. In vivo transplantation of mouse iDAN cells. TH-GFP+ iDAN cells were injected in the ventricles of P1 mouse brains and immunohistochemistry analysis was performed 15 days post-transplantation. a, Transplanted cells integrate in multiple sites in the host brain and develop a complex pattern of long neurites some of which extend to the contralateral hemisphere. b-f, Higher power view of TH-GFP+ grafted cells integrated in different locations in the host neural tissue. Scale bars: 100 μm (d), 200 μm (c) and 1 mm (a).

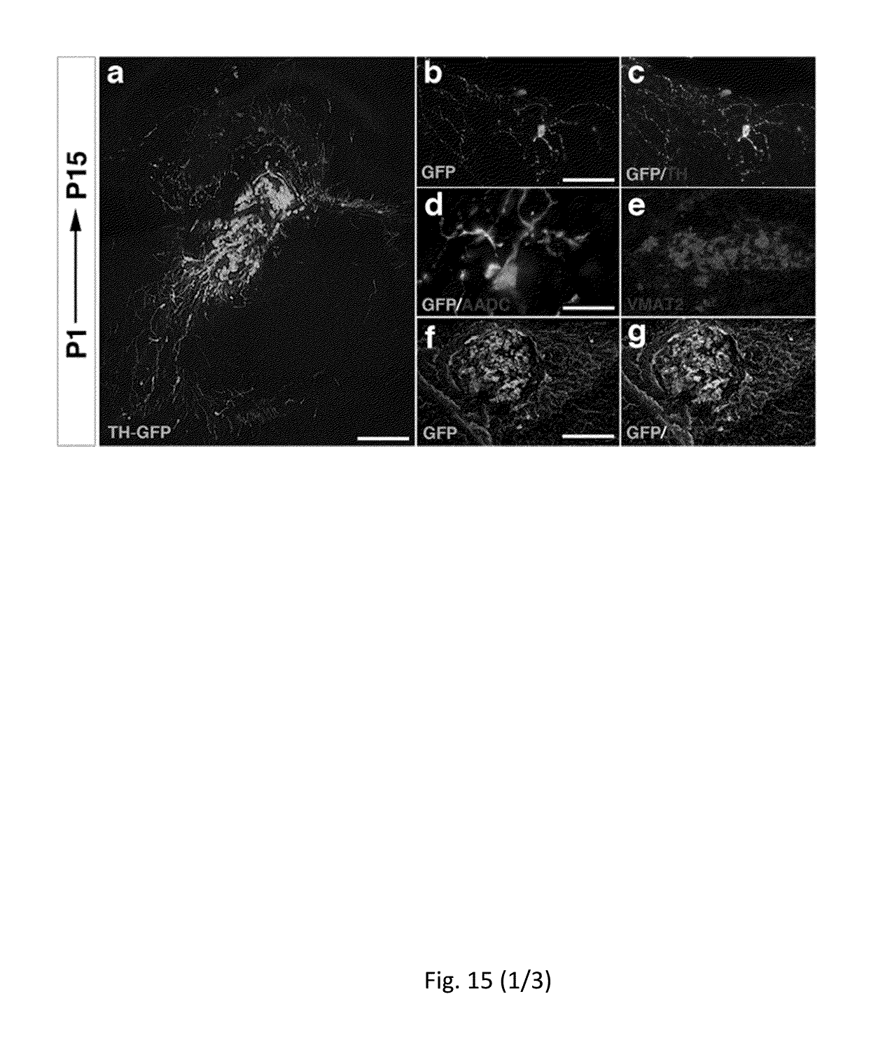

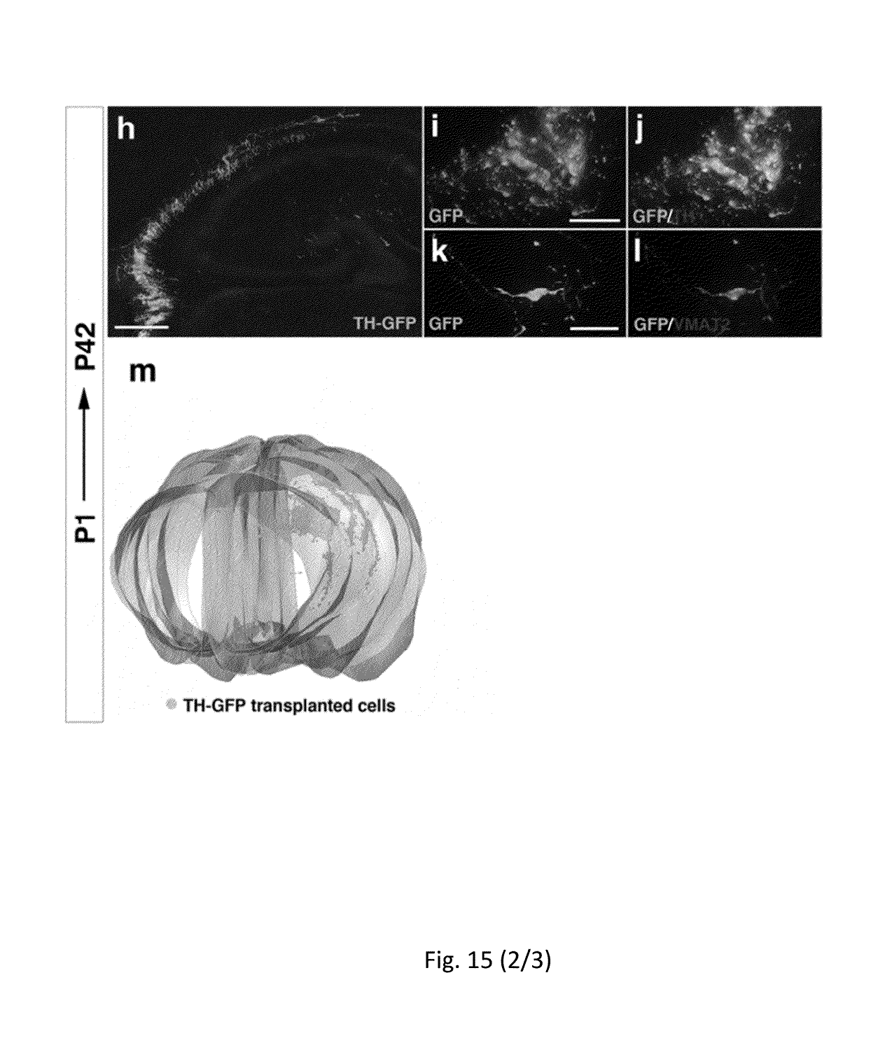

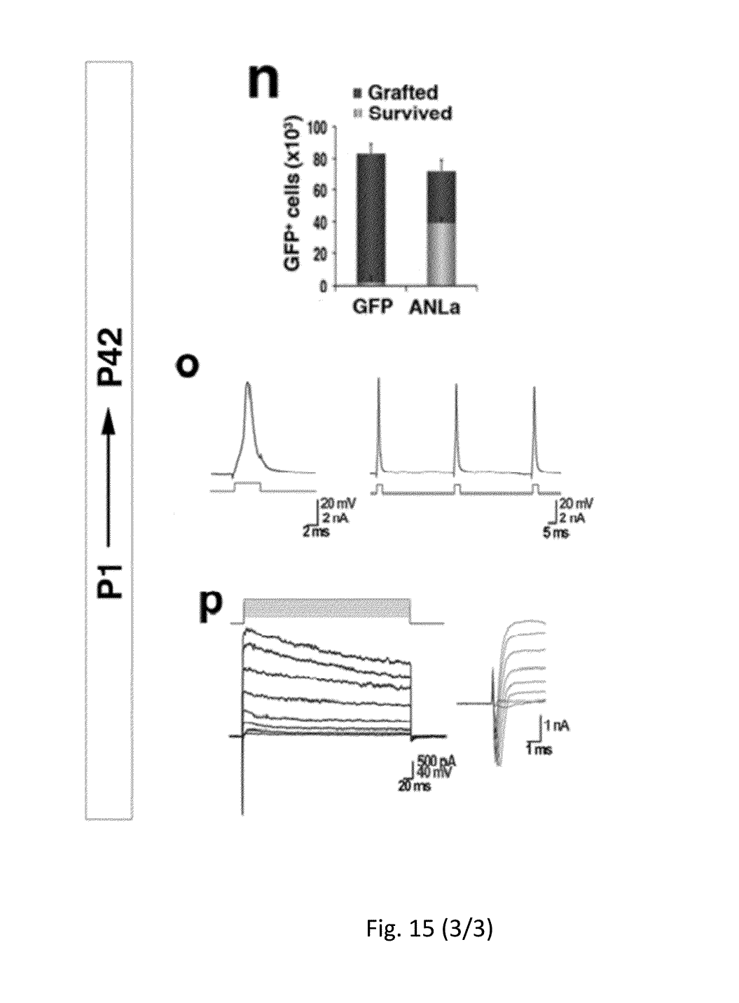

FIG. 15. Analysis of grafted mouse iDAN cells. a, Grafted mouse TH-GFP+ iDAN cells are integrated and display a mature morphology after 15 days from transplantation performed in P1 neonatal mouse brains. Grafted TH-GFP+ neuronal cells stain positive for the TH (b, c), AADC (d), VMAT2 (e) and DAT (f, g). h-p, iDAN cells grafted cells survive in the host brain also 42 days after transplantation, maintaining expression of DA markers (i-l). The distribution of grafted iDAN cells is shown in the 3D brain reconstruction (m). n, The diagram shows that about half of the grafted iDAN cells (ANLa) survive after 42 days, whereas only very few MEFs infected with a control GFP-expressing lentivirus are detectable at the same time point. o, Whole-cell current clamp recordings. The cell membrane potential was held at −65 mV. Action potentials were elicited by injection of supra-threshold current pulses (1 nA, 5 ms). p, Voltage-clamp recording of Na+ and K+ voltage-gated currents. Voltage steps (300 ms) up to +20 mV were delivered from a holding potential of −70 mV. The inset shows a magnification of Na+ currents. Scale bars: 20 μm (d, k), 50 μm (b, i), 200 μm (a, f) and 400 μm (h). Data are presented with mean±s.e.m.

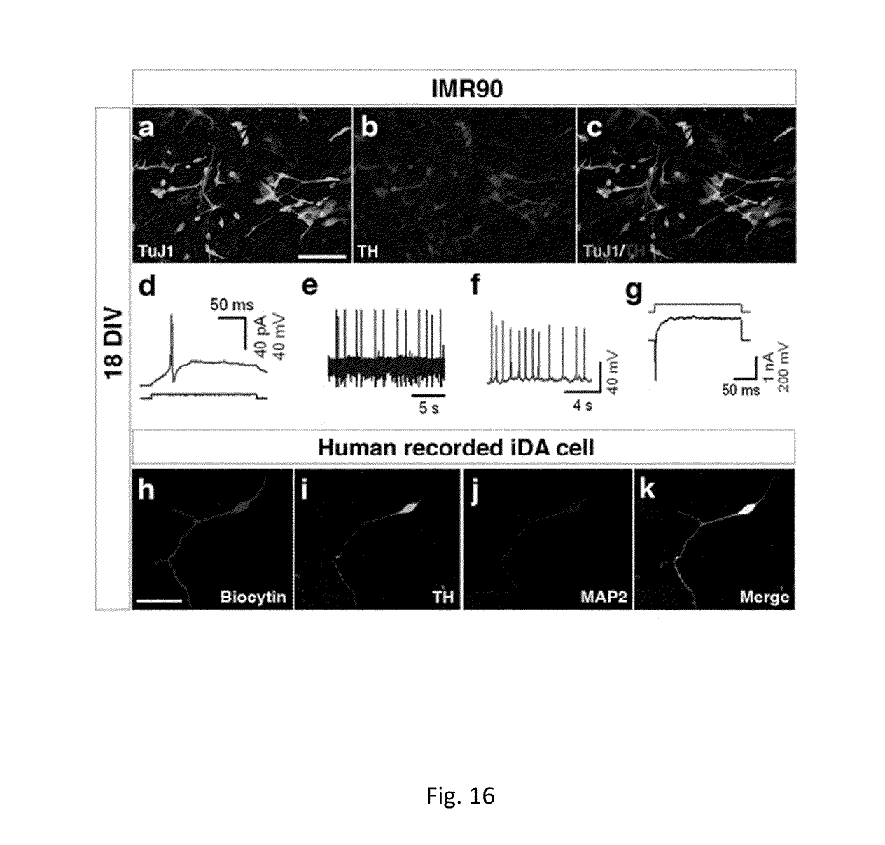

FIG. 16. Characterization of IMR90 human fetal fibroblasts reprogrammed into iDAN cells. a-c, TuJ1 and TH immunocytochemistry analysis on IMR90 cells reprogrammed into iDAN cells. d, Depolarization of a human fetal iDAN cell elicited an action potential which is followed by hyperpolarization. Spontaneous firing of the cell, as measured in whole-cell current-clamp (e) and on-cell (f) configurations. Rapidly inactivating Na+ currents and delayed rectifier K+ current in a human fetal iDAN cell (g). Reprogrammed human fetal iDAN cells were filled with biocytine (h) after successful recording and subjected retrospectively to immunocytochemistry for MAP2 (j) and TH (i). Co-staining of the three markers confirms the correct analysis of a reprogrammed iDAN cell (k). All analyses were performed at 18 days in vitro (DIV). Scale bar: 20 μm (h) and 50 μm (a).

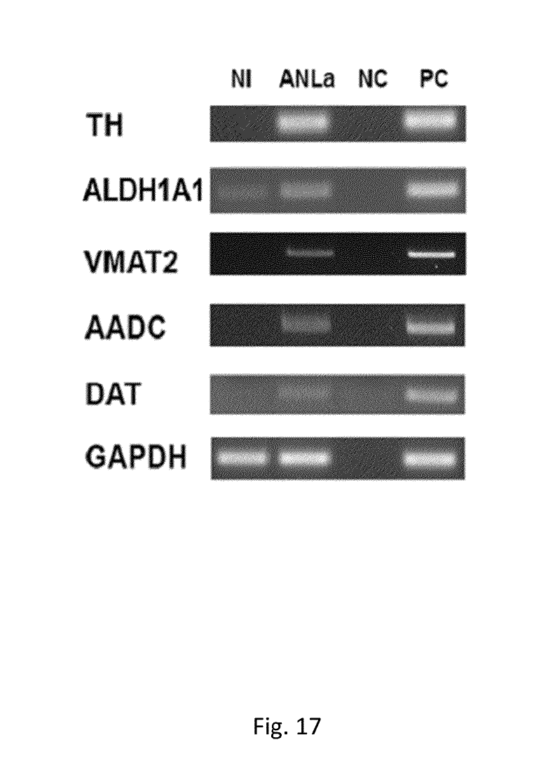

FIG. 17. RT-PCR analysis of DA neuronal markers in human reprogrammed adult fibroblasts. Analysis of DA molecular marker gene expression in human iDAN cells 18 days after infection. Cells were infected with ANLa viral cocktail. Not retro-transcripted samples as negative control (NC) and human iPS cells differentiated into DA neurons as positive control (PC) are also shown.

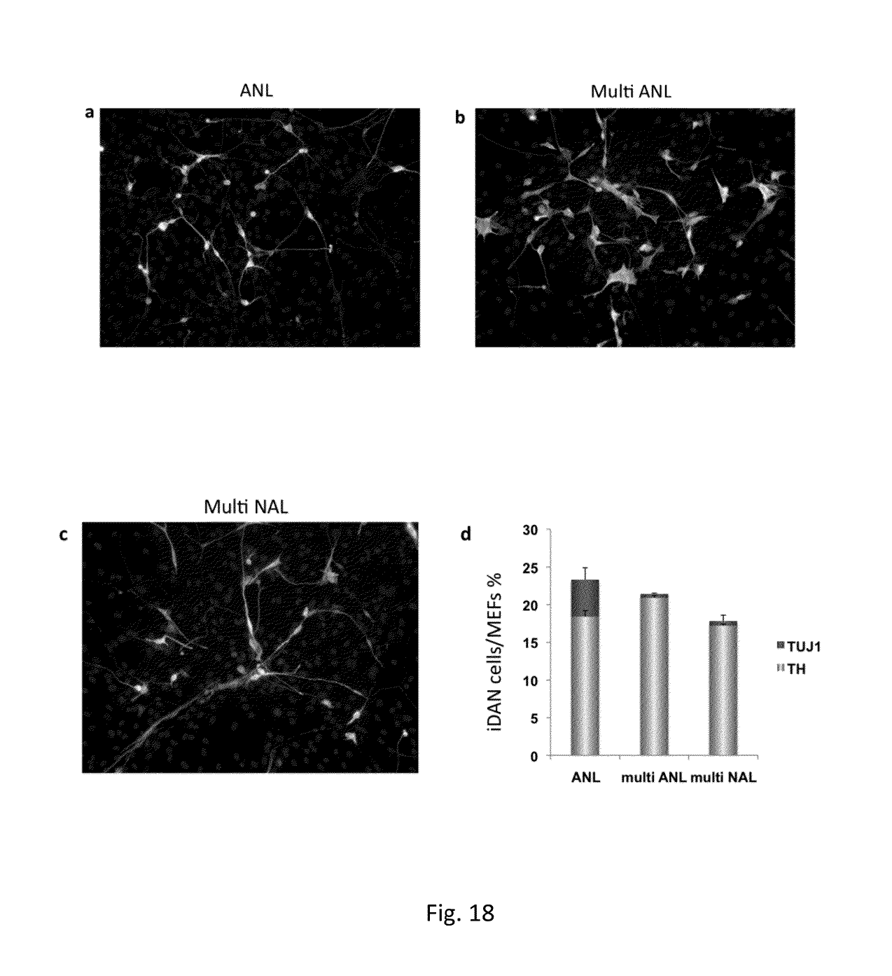

FIG. 18: Direct reprogramming of MEFs into dopaminergic neurons using a single multicistronic vector. Representative images of TuJ1 (red) and TH immunostainings of iDAN cells reprogrammed with the three single Mash1, Nurr1, Lmx1a viruses (ANL, a) the multicistronic virus ANL (b) or the multicistronic virus NAL (c). (d) Quantification of TuJ1+ and TH+ neuronal. Immunocytochemical analysis was performed 14 days after lentiviral induction.

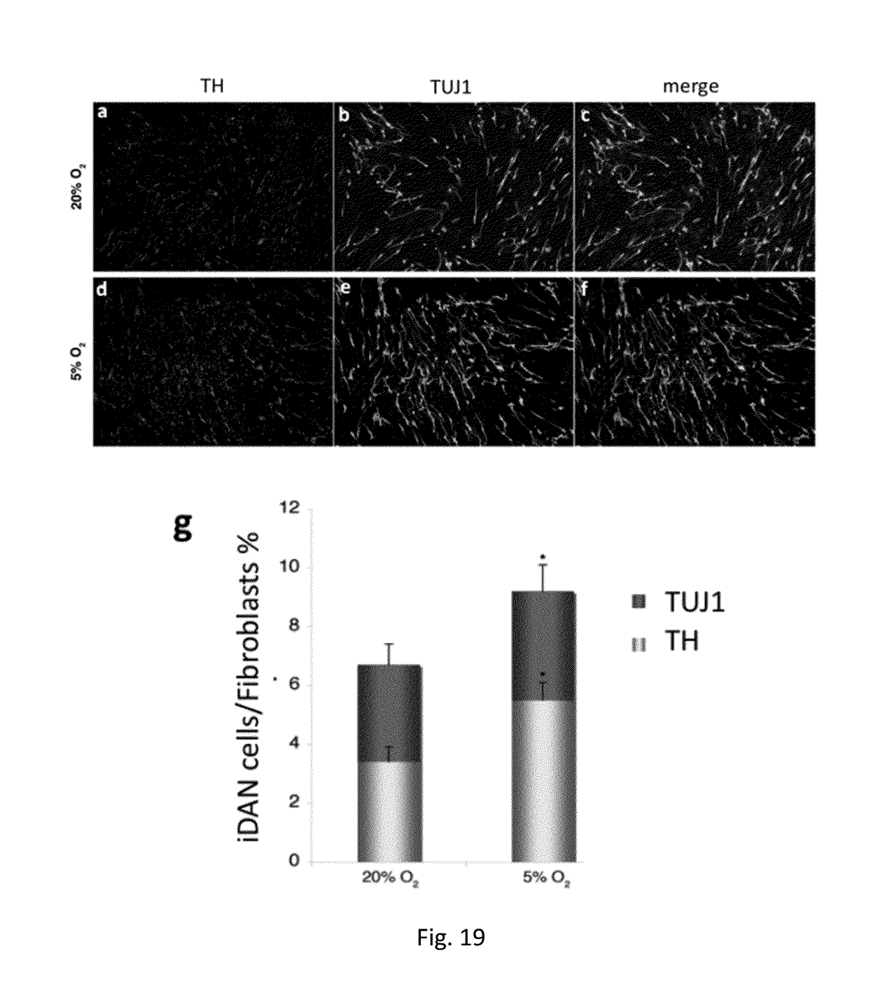

FIG. 19: Direct reprogramming of human adult fibroblasts in hypoxia condition. Representative images of TuJ1 (red) and TH immunostainings of human iDAN cells reprogrammed with the three single Mash1, Nurr1, Lmx1a viruses in 20% O₂ (a-c) or 5% O₂ (d-f). g, Quantification of TuJ1+ and TH+ neuronal. Immunocytochemical analysis was performed 21 days after lentiviral induction.

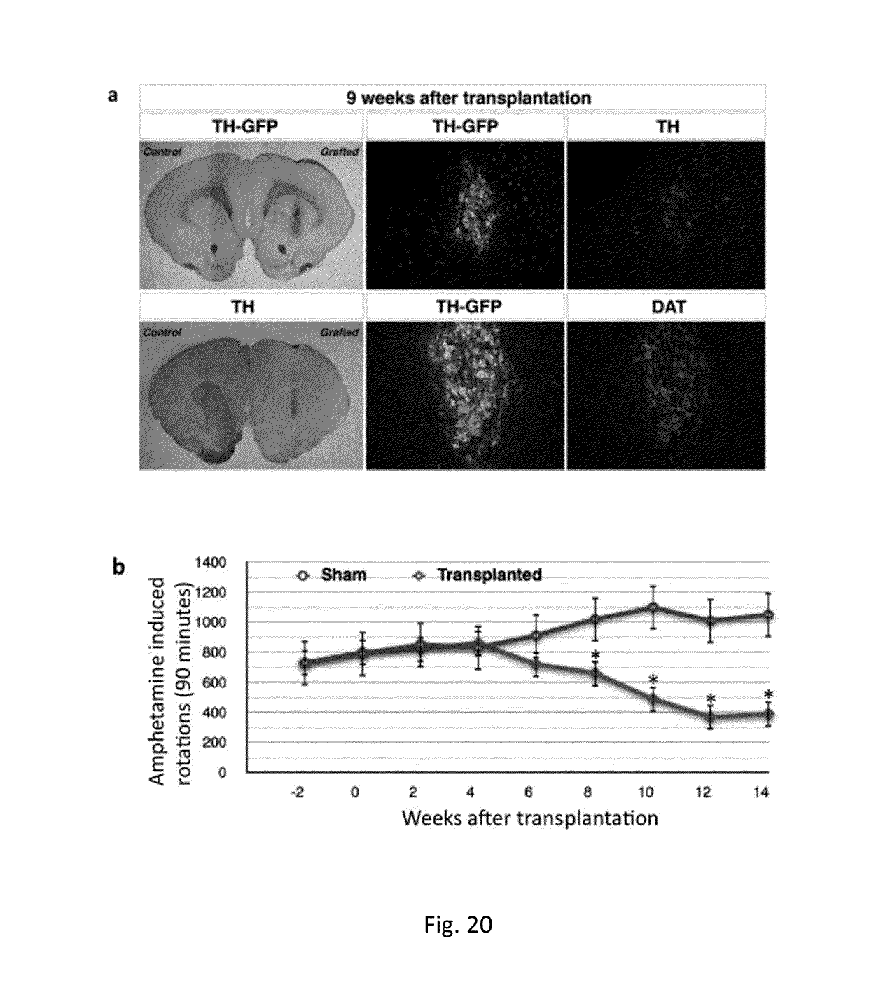

FIG. 20. Graft of iDAN cells in a rat model of Parkinson's disease. The histological analysis of 6OHDA lesioned rats transplanted with iDAN cells show clear integration in the striatum, 9 weeks after transplantation. b) Amphetamine-induced rotations for 90 min in 6OHDA lesioned mice before the cell transplantation, and 4 and 8 weeks after the transplantation of TH-GFP+ cells, into the lesioned striatum. Transplantation of reprogrammed TH-GFP+ cells led to a significant reduction in amphetamine-induced rotation scores in 6OHDA lesioned rats since 8 weeks after transplantation. n=12, data represent mean±SEM; ANOVA test, *p<0.05.

### DETAILED DESCRIPTION OF THE INVENTION

### Material and Methods

Cell Culture and Viral Infection.

MEFs were isolated from E14.5 wild type or TH-GFP mice embryos. Adult human fibroblasts isolated from healthy subjects and PD patients as well as human fetal lung fibroblasts (IMR90) were grown in MEF media. Cells were infected with dox-inducible lentiviruses as previously reported⁷.

Electrophysiology and Amperometry.

Electrophysiological recordings were performed in on-cell and whole-cell configurations. Carbon-fiber microelectrodes were used for amperometric recordings¹⁵.

Cell Culture.

MEFs were isolated from E14.5 wild-type or TH-GFP knock-in mice embryos. Head, vertebral column, dorsal root ganglia and all internal organs were removed and discarded and the remaining embryonic tissue was manually dissociated and incubated in 0.25% trypsin (Sigma) for 10-15 min. Cells from each embryo were plated onto a 15-cm tissue culture dish in MEF media [Dulbecco's Modified Eagle Medium; (Invitrogen) containing 10% fetal bovine serum (FBS; Hyclone), β-mercaptoethanol (Sigma), non-essential amino acids (Invitrogen), sodium pyruvate and penicillin/streptomycin (Invitrogen). In all experiments cells were not splitted more than four times. Mouse adult fibroblasts were isolated from tail tip samples. Tails were peeled, minced into 1 cm pieces, placed on culture dishes, and incubated in MEF media for 5 days. Adult human fibroblasts were isolated from skin biopsy samples of healthy and PD patients^{22,23} provided from the “Cell Line and DNA Biobank from Patients affected by Genetic Diseases” (G. Gaslini Institute) and “Parkinson Institute Biobank” (Milan, http://www.parkinson.it/dnabank.html) of the Telethon Genetic Biobank Network (http://www.biobanknetwork.org). The informed consent as issued by the ICP Ethical committee was obtained by healthy and PD patients enrolled for the DNA and cell biobank collection.

Human skin samples were mechanically dissociated and plated on matrigel coated dishes. Human fibroblasts were cultured as MEFs. Mouse and adult fibroblasts were grown in MEF media as well as human fetal lung fibroblasts IMR90 (ATCC). Mesencephalic DA primary cell cultures from TH-GFP mice were prepared as previously described²⁴. Mice were maintained at San Raffaele Scientific Institute Institutional mouse facility and experiments were performed in accordance with experimental protocols approved by local Institutional animal care and use committees (IACUC).

Molecular Cloning and Viral Infection.

cDNAs for the DA transcription factors were cloned into lentiviral vectors under the control of the tetracycline operator. Each gene was cloned independently in a lentiviral vector.

| Mash1-Mouse-Gene (underline letters denote cDNA boundaries) | |
|---|---|
| (SEQ ID No. 1) | |
| atggagag ctctggcaag atggagagtggagccggcca gcagccgcag cccccgcagc ccttcctgcc | |
| tcccgcagcc tgcttctttgcgaccgcggc ggcggcggca gcggcggcgg ccgcggcagc tcagagcgcg | |
| cagcagcaacagccgcaggc gccgccgcag caggcgccgc agctgagccc ggtggccgac | |
| agccagccctcagggggcgg tcacaagtca gcggccaagc aggtcaagcg ccagcgctcg | |
| tcctctccggaactgatgcg ctgcaaacgc cggctcaact tcagcggctt cggctacagc | |
| ctgccacagcagcagccggc cgccgtggcg cgccgcaacg agcgcgagcg caaccgggtc | |
| aagttggtcaacctgggttt tgccaccctc cgggagcatg tccccaacgg cgcggccaac | |
| aagaagatgagcaaggtgga gacgctgcgc tcggcggtcg agtacatccg cgcgctgcag | |
| cagctgctggacgagcacga cgcggtgagc gctgcctttc aggcgggcgt cctgtcgccc | |
| accatctcccccaactactc caacgacttg aactctatgg cgggttctcc ggtctcgtcc | |
| tactcctccgacgagggatc ctacgaccct cttagcccag aggaacaaga gctgctggac | |
| tttaccaactggttctga | |
| Mash1-Mouse-Protein | |
| (SEQ ID No. 2) | |
| MESSGKMESGAGQQPQPPQPFLPPAACFFATAAAAAAAAAAAAQSAQQQQPQAPPQQAPQLSPVADSQPSGGGHKS | |
| AAKQVKRQRSSSPELMRCKRRLNFSGFGYSLPQQQPAAVARRNERERNRVKLVNLGFATLREHVPNGAANKKMSKV | |
| ETLRSAVEYIRALQQLLDEHDAVSAAFQAGVLSPTISPNYSNDLNSMAGSPVSSYSSDEGSYDPLSPEEQELLDFT | |
| NWF | |
| Mash1-Human-gene (underline letters denote cDNA boundaries) | |
| (SEQ ID No. 3) | |
| 1 | agcactctct cacttctggc cagggaacgt ggaaggcgca ccgacaggga tccggccagg |
| 61 | gagggcgagt gaaagaagga aatcagaaag gaagggagtt aacaaaataa taaaaacagc |
| 121 | ctgagccacg gctggagaga ccgagacccg gcgcaagaga gcgcagcctt agtaggagag |
| 181 | gaacgcgaga cgcggcagag cgcgttcagc actgactttt gctgctgctt ctgctttttt |
| 241 | ttttcttaga aacaagaagg cgccagcggc agcctcacac gcgagcgcca cgcgaggctc |
| 301 | ccgaagccaa cccgcgaagg gaggagggga gggaggagga ggcggcgtgc agggaggaga |
| 361 | aaaagcattt tcactttttt tgctcccact ctaagaagtc tcccggggat tttgtatata |
| 421 | ttttttaact tccgtcaggg ctcccgcttc atatttcctt ttctttccct ctctgttcct |
| 481 | gcacccaagt tctctctgtg tccccctcgc gggccccgca cctcgcgtcc cggatcgctc |
| 541 | tgattccgcg actccttggc cgccgctgcg catggaaagc tctgccaaga tggagagcgg |
| 601 | cggcgccggc cagcagcccc agccgcagcc ccagcagccc ttcctgccgc ccgcagcctg |
| 661 | tttctttgcc acggccgcag ccgcggcggc cgcagccgcc gcagcggcag cgcagagcgc |
| 721 | gcagcagcag cagcagcagc agcagcagca gcagcaggcg ccgcagctga gaccggcggc |
| 781 | cgacggccag ccctcagggg gcggtcacaa gtcagcgccc aagcaagtca agcgacagcg |
| 841 | ctcgtcttcg cccgaactga tgcgctgcaa acgccggctc aacttcagcg gctttggcta |
| 901 | cagcctgccg cagcagcagc cggccgccgt ggcgcgccgc aacgagcgcg agcgcaaccg |
| 961 | cgtcaagttg gtcaacctgg gctttgccac ccttcgggag cacgtcccca acggcgcggc |
| 1021 | caacaagaag atgagtaagg tggagacact gcgctcggcg gtcgagtaca tccgcgcgct |
| 1081 | gcagcagctg ctggacgagc atgacgcggt gagcgccgcc ttccaggcag gcgtcctgtc |
| 1141 | gcccaccatc tcccccaact actccaacga cttgaactcc atggccggct cgccggtctc |
| 1201 | atcctactcg tcggacgagg gctcttacga cccgctcagc cccgaggagc aggagcttct |
| 1261 | cgacttcacc aactggttct gaggggctcg gcctggtcag gccctggtgc gaatggactt |
| 1321 | tggaagcagg gtgatcgcac aacctgcatc tttagtgctt tcttgtcagt ggcgttggga |
| 1381 | gggggagaaa aggaaaagaa aaaaaaaaga agaagaagaa gaaaagagaa gaagaaaaaa |
| 1441 | acgaaaacag tcaaccaacc ccatcgccaa ctaagcgagg catgcctgag agacatggct |
| 1501 | ttcagaaaac gggaagcgct cagaacagta tctttgcact ccaatcattc acggagatat |
| 1561 | gaagagcaac tgggacctga gtcaatgcgc aaaatgcagc ttgtgtgcaa aagcagtggg |
| 1621 | ctcctggcag aagggagcag cacacgcgtt atagtaactc ccatcacctc taacacgcac |
| 1681 | agctgaaagt tcttgctcgg gtcccttcac ctcctcgccc tttcttaaag tgcagttctt |
| 1741 | agccctctag aaacgagttg gtgtctttcg tctcagtagc ccccacccca ataagctgta |
| 1801 | gacattggtt tacagtgaaa ctatgctatt ctcagccctt tgaaactctg cttctcctcc |
| 1861 | agggcccgat tcccaaaccc catggcttcc ctcacactgt cttttctacc attttcatta |
| 1921 | tagaatgctt ccaatctttt gtgaattttt tattataaaa aatctatttg tatctatcct |
| 1981 | aaccagttcg gggatatatt aagatatttt tgtacataag agagaaagag agagaaaaat |
| 2041 | ttatagaagt tttgtacaaa tggtttaaaa tgtgtatatc ttgatacttt aacatgtaat |
| 2101 | gctattacct ctgcatattt tagatgtgta gttcacctta caactgcaat tttccctatg |
| 2161 | tggttttgta aagaactctc ctcataggtg agatcaagag gccaccagtt gtacttcagc |
| 2221 | accaatgtgt cttactttat agaaatgttg ttaatgtatt aatgatgtta ttaaatactg |
| 2281 | ttcaagaaga acaaagttta tgcagctact gtccaaactc aaagtggcag ccagttggtt |
| 2341 | ttgataggtt gccttttgga gatttctatt actgcctttt tttttcttac tgttttatta |
| 2401 | caaacttaca aaaatatgta taaccctgtt ttatacaaac tagtttcgta ataaaacttt |
| 2461 | ttcctttttt taaaatgaaa ataaaaaaaa |
| Mash1 Human-Protein | |
| (SEQ ID No. 4) | |
| MESSAKMESGGAGQQPQPQPQQPFLPPAACFFATAAAAAAAAAAAAAQSAQQQQQQQQQQQQAPQLRPAADGQPSG | |
| GGHKSAPKQVKRQRSSSPELMRCKRRLNFSGFGYSLPQQQPAAVARRNERERNRVKLVNLGFATLREHVPNGAANK | |
| KMSKVETLRSAVEYIRALQQLLDEHDAVSAAFQAGVLSPTISPNYSNDLNSMAGSPVSSYSSDEGS | |
| YDPLSPEEQELLDFTNWF | |
| Nurr1 (Nr4a2) -Mouse-Gene (underline letters denote cDNA boundaries) | |
| (SEQ ID No. 5) | |
| atgccttgtgt tcaggcgcag tatgggtcct cgcctcaagg agccagcccc gcttctcagagctacagtta | |
| ccactcttcg ggagaataca gctccgattt cttaactcca gagtttgtcaagtttagcat ggacctcacc | |
| aacactgaaa ttactgccac cacttctctc cccagcttca gtacctttat ggacaactac agcacaggct | |
| acgacgtcaa gccaccttgc ttgtaccaaatgcccctgtc cggacagcag tcctccatta aggtagaaga | |
| cattcagatg cacaactaccagcaacacag ccacctgccc cctcagtccg aggagatgat gccacacagc | |
| gggtcggttt actacaagcc ctcttcgccc ccgacaccca gcaccccgag cttccaggtg | |
| cagcatagcccgatgtggga cgatccgggc tcccttcaca acttccacca gaactacgtg | |
| gccactacgcatatgatcga gcagaggaag acacctgtct cccgcctgtc actcttctcc | |
| tttaagcagtcgcccccggg cactcctgtg tctagctgcc agatgcgctt cgacgggcct | |
| ctgcacgtccccatgaaccc ggagcccgcg ggcagccacc acgtagtgga tgggcagacc | |
| ttcgccgtgcccaaccccat tcgcaagccg gcatccatgg gcttcccggg cctgcagatc | |
| ggccacgcatcgcagttgct tgacacgcag gtgccctcgc cgccgtcccg gggctctccc | |
| tccaatgagggtctgtgcgc tgtttgcggt gacaacgcgg cctgtcagca ctacggtgtt | |
| cgcacttgtgagggctgcaa aggtttcttt aagcgcacgg tgcaaaaaaa cgcgaaatat | |
| gtgtgtttagcaaataaaaa ctgcccagtg gacaagcgcc gccgaaatcg ttgtcagtac | |
| tgtcggtttcagaagtgcct agctgttggg atggttaaag aagtggttcg cacggacagt | |
| ttaaaaggccggagaggtcg tttaccctcg aagccgaaga gcccacagga tccctctccc | |
| ccctcacctccggtgagtct gatcagtgcc ctcgtcagag cccacgtcga ttccaatccg | |
| gcaatgaccagcctggacta ttccaggttc caggcaaacc ctgactatca gatgagtgga | |
| gatgatacccaacatatcca gcagttctac gatctcctga ccggctctat ggagatcatc | |
| agagggtgggcagagaagat ccctggcttt gctgacctgc ccaaagccga ccaggacctg | |
| ctttttgaatcagctttctt agaattattt gttctgcgct tagcatacag gtccaaccca | |
| gtggagggtaaactcatctt ttgcaatggg gtggtcttgc acaggttgca atgcgtgcgt | |
| ggctttggggaatggattga ttccattgtt gaattctcct ccaacttgca gaatatgaac | |
| atcgacatttctgccttctc ctgcattgct gccctggcta tggtcacaga gagacacggg | |
| ctcaaggaacccaagagagt ggaagagcta caaaacaaaa ttgtaaattg tcttaaagac | |
| catgtgactttcaataatgg gggtttgaac cgacccaact acctgtctaa actgttgggg | |
| aagctgccagaactccgcac cctttgcaca cagggcctcc agcgcatttt ctacctgaaa | |
| ttggaagacttggtaccacc accagcaata attgacaaac ttttcctgga caccttacct ttctaa | |
| Nurr1 (Nr4a2) -Mouse protein | |
| (SEQ ID No. 6) | |
| MPCVQAQYGSSPQGASPASQSYSYHSSGEYSSDFLTPEFVKFSMDLTNTEITATTSLPSFSTFMDNYSTGYDVKPP | |
| CLYQMPLSGQQSSIKVEDIQMHNYQQHSHLPPQSEEMMPHSGSVYYKPSSPPTPSTPSFQVQHSPMWDDPGSLHNF | |
| HQNYVATTHMIEQRKTPVSRLSLFSFKQSPPGTPVSSCQMRFDGPLHVPMNPEPAGSHHVVDGQTFAVPNPIRKPA | |
| SMGFPGLQIGHASQLLDTQVPSPPSRGSPSNEGLCAVCGDNAACQHYGVRTCEGCKGFFKRTVQKNAKYVCLANKN | |
| CPVDKRRRNRCQYCRFQKCLAVGMVKEVVRTDSLKGRRGRLPSKPKSPQDPSPPSPPVSLISALVRAHVDSNPAMT | |
| SLDYSRFQANPDYQMSGDDTQHIQQFYDLLTGSMEIIRGWAEKIPGFADLPKADQDLLFESAFLELFVLRLAYRSN | |
| PVEGKLIFCNGVVLHRLQCVRGFGEWIDSIVEFSSNLQNMNIDISAFSCIAALAMVTERHGLKEPKRVEELQNKIV | |
| NCLKDHVTFNNGGLNRPNYLSKLLGKLPELRTLCTQGLQRIFYLKLEDLVPPPAIIDKLFLDTLPF | |
| Nurr1-Human-gene (underline letters denote cDNA boundaries) | |
| (SEQ ID No. 7) | |
| 1 | gctgacgcgc gctgacgcgc ggagacttta ggtgcatgtt ggcagcggca gcgcaagcca |
| 61 | cataaacaaa ggcacattgg cggccagggc cagtccgccc ggcggctcgc gcacggctcc |
| 121 | gcggtccctt ttgcctgtcc agccggccgc ctgtccctgc tccctccctc cgtgaggtgt |
| 181 | ccgggttccc ttcgcccagc tctcccaccc ctacccgacc ccggcgcccg ggctcccaga |
| 241 | gggaactgca cttcggcaga gttgaatgaa tgaagagaga cgcggagaac tcctaaggag |
| 301 | gagattggac aggctggact ccccattgct tttctaaaaa tcttggaaac tttgtccttc |
| 361 | attgaattac gacactgtcc acctttaatt tcctcgaaaa cgcctgtaac tcggctgaag |
| 421 | ccatgccttg tgttcaggcg cagtatgggt cctcgcctca aggagccagc cccgcttctc |
| 481 | agagctacag ttaccactct tcgggagaat acagctccga tttcttaact ccagagtttg |
| 541 | tcaagtttag catggacctc accaacactg aaatcactgc caccacttct ctccccagct |
| 601 | tcagtacctt tatggacaac tacagcacag gctacgacgt caagccacct tgcttgtacc |
| 661 | aaatgcccct gtccggacag cagtcctcca ttaaggtaga agacattcag atgcacaact |
| 721 | accagcaaca cagccacctg cccccccagt ctgaggagat gatgccgcac tccgggtcgg |
| 781 | tttactacaa gccctcctcg cccccgacgc ccaccacccc gggcttccag gtgcagcaca |
| 841 | gccccatgtg ggacgacccg ggatctctcc acaacttcca ccagaactac gtggccacta |
| 901 | cgcacatgat cgagcagagg aaaacgccag tctcccgcct ctccctcttc tcctttaagc |
| 961 | aatcgccccc tggcaccccg gtgtctagtt gccagatgcg cttcgacggg cccctgcacg |
| 1021 | tccccatgaa cccggagccc gccggcagcc accacgtggt ggacgggcag accttcgctg |
| 1081 | tgcccaaccc cattcgcaag cccgcgtcca tgggcttccc gggcctgcag atcggccacg |
| 1141 | cgtctcagct gctcgacacg caggtgccct caccgccgtc gcggggctcc ccctccaacg |
| 1201 | aggggctgtg cgctgtgtgt ggggacaacg cggcctgcca acactacggc gtgcgcacct |
| 1261 | gtgagggctg caaaggcttc tttaagcgca cagtgcaaaa aaatgcaaaa tacgtgtgtt |
| 1321 | tagcaaataa aaactgccca gtggacaagc gtcgccggaa tcgctgtcag tactgccgat |
| 1381 | ttcagaagtg cctggctgtt gggatggtca aagaagtggt tcgcacagac agtttaaaag |
| 1441 | gccggagagg tcgtttgccc tcgaaaccga agagcccaca ggagccctct cccccttcgc |
| 1501 | ccccggtgag tctgatcagt gccctcgtca gggcccatgt cgactccaac ccggctatga |
| 1561 | ccagcctgga ctattccagg ttccaggcga accctgacta tcaaatgagt ggagatgaca |
| 1621 | cccagcatat ccagcaattc tatgatctcc tgactggctc catggagatc atccggggct |
| 1681 | gggcagagaa gatccctggc ttcgcagacc tgcccaaagc cgaccaagac ctgctttttg |
| 1741 | aatcagcttt cttagaactg tttgtccttc gattagcata caggtccaac ccagtggagg |
| 1801 | gtaaactcat cttttgcaat ggggtggtct tgcacaggtt gcaatgcgtt cgtggctttg |
| 1861 | gggaatggat tgattccatt gttgaattct cctccaactt gcagaatatg aacatcgaca |
| 1921 | tttctgcctt ctcctgcatt gctgccctgg ctatggtcac agagagacac gggctcaagg |
| 1981 | aacccaagag agtggaagaa ctgcaaaaca agattgtaaa ttgtctcaaa gaccacgtga |
| 2041 | ctttcaacaa tggggggttg aaccgcccca attatttgtc caaactgttg gggaagctcc |
| 2101 | cagaacttcg taccctttgc acacaggggc tacagcgcat tttctacctg aaattggaag |
| 2161 | acttggtgcc accgccagca ataattgaca aacttttcct ggacacttta cctttctaag |
| 2221 | acctcctccc aagcacttca aaggaactgg aatgataatg gaaactgtca agagggggca |
| 2281 | agtcacatgg gcagagatag ccgtgtgagc agtctcagct caagctgccc cccatttctg |
| 2341 | taaccctcct agcccccttg atccctaaag aaaacaaaca aacaaacaaa aactgttgct |
| 2401 | atttcctaac ctgcaggcag aacctgaaag ggcattttgg ctccggggca tcctggattt |
| 2461 | agaacatgga ctacacacaa tacagtggta taaacttttt attctcagtt taaaaatcag |
| 2521 | tttgttgttc agaagaaaga ttgctataat gtataatggg aaatgtttgg ccatgcttgg |
| 2581 | ttgttgcagt tcagacaaat gtaacacaca cacacataca cacacacacacacacacaga |
| 2641 | gacacatctt aaggggaccc acaagtattg ccctttaaca agacttcaaa gttttctgct |
| 2701 | gtaaagaaag ctgtaatata tagtaaaact aaatgttgcg tgggtggcat gagttgaaga |
| 2761 | aggcaaaggc ttgtaaattt acccaatgca gtttggcttt ttaaattatt ttgtgcctat |
| 2821 | ttatgaataa atattacaaa ttctaaaaga taagtgtgtt tgcaaaaaaa aagaaaataa |
| 2881 | atacataaaa aagggacaag catgttgatt ctaggttgaa aatgttatag gcacttgcta |
| 2941 | cttcagtaat gtctatatta tataaatagt atttcagaca ctatgtagtc tgttagattt |
| 3001 | tataaagatt ggtagttatc tgagcttaaa cattttctca attgtaaaat aggtgggcac |
| 3061 | aagtattaca catcagaaaa tcctgacaaa agggacacat agtgtttgta acaccgtcca |
| 3121 | acattccttg tttgtaagtg ttgtatgtac cgttgatgtt gataaaaaga aagtttatat |
| 3181 | cttgattatt ttgttgtcta aagctaaaca aaacttgcat gcagcagctt ttgactgttt |
| 3241 | ccagagtgct tataatatac ataactccct ggaaataact gagcactttg aatttttttt |
| 3301 | atgtctaaaa ttgtcagtta atttattatt ttgtttgagt aagaatttta atattgccat |
| 3361 | attctgtagt atttttcttt gtatatttct agtatggcac atgatatgag tcactgcctt |
| 3421 | tttttctatg gtgtatgaca gttagagatg ctgatttttt ttctgataaa ttctttcttt |
| 3481 | gagaaagaca attttaatgt ttacaacaat aaaccatgta aatgaacaga aaaaaaaaaa |
| 3541 | aaaaaa |
| Nurr1 Human-Protein | |
| (SEQ ID No. 8) | |
| MPCVQAQYGSSPQGASPASQSYSYHSSGEYSSDFLTPEFVKFSMDLTNTEITATTSLPSFSTFMDNYSTGYDVKPP | |
| CLYQMPLSGQQSSIKVEDIQMHNYQQHSHLPPQSEEMMPHSGSVYYKPSSPPTPTTPGFQVQHSPMWDDPGSLHNF | |
| HQNYVATTHMIEQRKTPVSRLSLFSFKQSPPGTPVSSCQMRFDGPLHVPMNPEPAGSHHVVDGQTF | |
| AVPNPIRKPASMGFPGLQIGHASQLLDTQVPSPPSRGSPSNEGLCAVCGDNAACQHYGVRTCEGCKGFFKRTVQKN | |
| AKYVCLANKNCPVDKRRRNRCQYCRFQKCLAVGMVKEVVRTDSLKGRRGRLPSKPKSPQEPSPPSPPVSLISALVR | |
| AHVDSNPAMTSLDYSRFQANPDYQMSGDDTQHIQQFYDLLTGSMEIIRGWAEKIPGFADLPKADQDLLFESAFLEL | |
| FVLRLAYRSNPVEGKLIFCNGVVLHRLQCVRGFGEWIDSIVEFSSNLQNMNIDISAFSCIAALAMVTERHGLKEPK | |
| RVEELQNKIVNCLKDHVTFNNGGLNRPNYLSKLLGKLPELRTLCTQGLQRIFYLKLEDLVPPPAIIDKLFLDTLPF | |
| Lmx1a- Mouse gene (underline letters denote cDNA boundaries) | |
| (SEQ ID No. 9) | |
| atg ttggacggcc tgaagatggaggagaacttt caaagtgcga ttgagacctc ggcatctttc | |
| tcctctttgc tgggcagagc ggtgagcccc aagtctgtct gcgagggctg tcagcgggtc atctcggaca | |
| ggtttctgctgcggctcaac gacagcttct ggcacgagca atgcgtgcag tgtgcctcct | |
| gcaaagagcccctggagacc acctgcttct accgggacaa gaagctctac tgcaagtacc actacgagaa | |
| actgtttgct gtcaaatgtg ggggctgctt cgaggccatt gcgcccaatg agtttgtcatgcgtgcccag | |
| aagagcgtat accacctgag ctgcttctgc tgctgcgtct gtgagcgacagctgcagaag ggtgacgagt | |
| ttgtcctgaa ggagggccag ctgctctgca aaggggacta tgagaaagaa cgggagctgc tgagcctggt | |
| gagccctgcg gcctcagact caggcaaaagcgatgatgag gagagccttt gcaagtcagc ccatggggca | |
| ggaaaaggag catcagaggacggcaaggac cataagcgac ccaaacgtcc cagaaccatc ctgaccactc | |
| agcagaggag agcattcaag gcctcgtttg aagtatcctc caagccctgc agaaaggtga | |
| gggagactctggctgcggag acagggctga gtgtccgtgt ggttcaggtg tggttccaga | |
| accagcgagccaagatgaag aagctggccc ggcgacagca gcaacagcaa caggaccaac agaacaccca | |
| gaggctgact tctgctcaga caaatggtag tgggaatgcg ggcatggaag ggatcatgaacccctataca | |
| acgttgccca ccccacagca gctgctggcc attgaacaga gcgtctacaactctgatccc ttccgacagg | |
| gtctcacccc accccagatg cctggagatc acatgcaccc ctatggtgct gaacctcttt tccatgactt | |
| ggatagtgat gacacatctc tcagtaacctgggagactgc ttcctggcaa cctcagaagc tgggcccctg | |
| cagtccagag tgggaaaccccattgaccat ctgtactcca tgcagaattc ctatttcacc tcttga | |
| Lmx1a- Mouse protein | |
| (SEQ ID No. 10) | |
| MLDGLKMEENFQSAIETSASFSSLLGRAVSPKSVCEGCQRVISDRFLLRLNDSFWHEQCVQCASCKEPLETTCFYR | |
| DKKLYCKYHYEKLFAVKCGGCFEATAPNEFVMRAQKSVYHLSCFCCCVCERQLQKGDEFVLKEGQLLCKGDYEKER | |
| ELLSLVSPAASDSGKSDDEESLCKSAHGAGKGASEDGKDHKRPKRPRTILTTQQRRAFKASFEVSSKPCRKVRETL | |
| AAETGLSVRVVQVWFQNQRAKMKKLARRQQQQQQDQQNTQRLTSAQTNGSGNAGMEGIMNPYTTLPTPQQLLAIEQ | |
| SVYNSDPFRQGLTPPQMPGDHMHPYGAEPLFHDLDSDDTSLSNLGDCFLATSEAGPLQSRVGNPIDHLYSMQNSYF | |
| T | |
| Lmx1a-Human-gene (underline letters denote cDNA boundaries) | |
| (SEQ ID No. 11) | |
| 1 | agaagctgca ggatccgccc cggcgaagca gggccgactc gcacccagga ccctgggcct |
| 61 | ctgccttccc tcctagcctt ggagaagcaa ctggccctct cctcccgctg aggagcgacg |
| 121 | cgggctggta ggacgtcccg ggaaggccgg cagctcgcga ccacgtcccg gcccagcctg |
| 181 | ggcgcgccga ggagcagagc cagcggccgg cgttcgctcc ggctccctcc ccggcgctcc |
| 241 | gaagccgagg gcggctcctc cggctgcagt ctcgggggcg acgccttccc gggcagaagc |
| 301 | ttccagcagc gctccgcaac ttctctctgc tccagtcact gggagagagc tcgcctacca |
| 361 | ggtcctcccg gcccggcccg aacatgctgg acggcctaaa gatggaggag aacttccaaa |
| 421 | gcgcgatcga cacctcggcc tccttctcct cgctgctggg cagagcggtg agccccaagt |
| 481 | ctgtctgcga gggctgtcag cgggtcatct tggacaggtt tctgctgcgg ctcaacgaca |
| 541 | gcttctggca tgagcagtgc gtgcagtgcg cctcctgcaa agagcccctg gagaccacct |
| 601 | gcttctaccg ggacaagaag ctgtactgca agtatgacta cgagaagctg tttgctgtta |
| 661 | aatgtggggg ctgcttcgag gccatcgctc ccaatgagtt tgttatgcgg gcccagaaga |
| 721 | gtgtatacca cctgagctgc ttctgctgct gtgtctgcga gcgacagctt cagaagggtg |
| 781 | atgagtttgt cctgaaggag gggcagctgc tctgcaaagg ggactatgag aaggagcggg |
| 841 | agctgctcag cctggtgagc ccagcagcct cagactcagg taaaagtgat gatgaagaaa |
| 901 | gtctctgcaa gtcagcccat ggggcaggga aaggaactgc tgaggaaggc aaggaccata |
| 961 | agcgccccaa acgtccgaga accatcttga caactcaaca gaggcgagca ttcaaggcct |
| 1021 | catttgaagt atcctccaag ccctgcagga aggtgagaga gactctggct gcagagacag |
| 1081 | ggctgagtgt ccgtgtcgtc caggtgtggt tccaaaacca gagagcgaag atgaagaagc |
| 1141 | tggccaggcg acagcagcag cagcagcaag atcagcagaa cacccagagg ctgagctctg |
| 1201 | ctcagacaaa cggtggtggg agtgctggga tggaaggaat catgaacccc tacacggctc |
| 1261 | tgcccacccc acagcagctc ctggccatcg agcagagtgt ctacagctca gatcccttcc |
| 1321 | gacagggtct caccccaccc cagatgcctg gagaccacat gcacccttat ggtgccgagc |
| 1381 | cccttttcca tgacctggat agcgacgaca cctccctcag taacctgggt gattgtttcc |
| 1441 | tagcaacctc agaagctggg cctctgcagt ccagagtggg aaaccccatt gaccatctgt |
| 1501 | actccatgca gaattcttac ttcacatctt gagtcttccc ctagagttct gtgactaggc |
| 1561 | tcccatatgg aacaaccata ttctttgagg ggtcactggc tttaggacag ggaggccagg |
| 1621 | gaagaggtgg gttggggagg gagttttgtt ggggatgctg ttgtataatg atatggtgta |
| 1681 | gctcagcatt tccaaagact gaatacatta tggattgcat agtttaatgt ttctaataag |
| 1741 | agtcttagca ttagatatga agacgtgttt atcattaagg acagagactt ttaatataga |
| 1801 | cattctcatg caaactagat acttagggac tcctaacaac ttcccaccat gtcggggaag |
| 1861 | ctcttgtcaa gaggtgcata tgtctatcca tctacacacc aatagacaga aggacagata |
| 1921 | gatagatgtg tgtgtgtgag tgtgtaacct ttcgtatttt accctcaaag tttattccta |
| 1981 | attataacag acaccaactg tacagcaaaa gtaactttat tttcagtgtg aactatattt |
| 2041 | aaggaaatgc ttgatgcact taagttataa aatgagataa tttactttta taaactttat |
| 2101 | ttttagcttg acaagacttg tcagcagggc agagagggct gctccaccta gccccatagc |
| 2161 | tttgagtgct ggggttcatt ctgttttcag agtgtctttc agatctggaa agaaattctg |
| 2221 | tgtggctgat ggtgttctct cttgcattct tgctctcttt ggggttgaat cactgggcag |
| 2281 | gggtgggaca gaataatctc tgatcatgtt ctgagaaaat gtaaagccca gactcctggg |
| 2341 | ctttctttta aattctgaca agtggttgtt gggcagtgct aggatgattg gttcagctct |
| 2401 | tgagcttcag catctgcaaa tgtggatgag gctaatagta tgtacctacc tcactgggaa |
| 2461 | acaccaaggc ttaattcatt cccaggacac atgagcaggg ctgagactaa tatctgatat |
| 2521 | ttgtttaaga tacaaccagg ccactcactt ggcaaaggag ggtacatagg gttgcagagc |
| 2581 | aggagggctc ctgaactcca gagggcagtt ctgcctgctg aagtccctct gcaaagcctg |
| 2641 | tgctgaagga gacaccagct cagagcagtt cagagggatc ccagagtccc agagtgggga |
| 2701 | ggaggtgaag gctgagggga tagaggaggg cctggtggtg ttctagagca gggttgggca |
| 2761 | aactcctgct tgcgggcctg ctttctatgg cttgccagca aagaatggtt tttacttttt |
| 2821 | ttttgaggtc attaaaaaaa aggagaagaa gaatatataa caggctgtct gtggcctgga |
| 2881 | aagcctgaaa tatttgctat ctgtattgtc tggcccttac agaaaaagtt tggggcccct |
| 2941 | tgttttagag ggtctgtttc taaagaacct catggcgctc atagaggcag aaggttccag |
| 3001 | tggaaaccct tggctcttcc ttccaactca ctcctctgat cctcggcaca gaagacccag |
| 3061 | cagccattgt acatggggac agttccacac cctggtctcc agttgcggtg ctaggatggt |
| 3121 | attgttctgt gctaggaagt ctcctgggaa cccagaatga gttggtgggg aagacagcgg |
| 3181 | gtcactgtgg acccatccag gaggggccag gataggcttg gcctcatttc tggggacatc |
| 3241 | attggagact tgaacacaga gacacgtccc tatcactctg gcaaggccag agggaacatg |
| 3301 | tccccttatg gtagagtcta tgttgtgtga tttttgtgct cttgtttata atttatgcaa |
| 3361 | accaccaaga aacccaaacc agtctgatga gcgaaaatta tgcagatgct gtatggcccc |
| 3421 | acaggtttct gtggtaaaga ccagttggag aatgtaggag atactatgtg agtgaaaatg |
| 3481 | aatagagatc cttattccac tccttaatgg cataccaaga tgaaattaaa atctcttaca |
| 3541 | aatgaaaaaa aaaaa |
| Lmx1a-Human-Protein | |
| (SEQ ID No. 12) | |
| MLDGLKMEENFQSAIDTSASFSSLLGRAVSPKSVCEGCQRVILDRFLLRLNDSFWHEQCVQCASCKEPLETTCFYR | |
| DKKLYCKYDYEKLFAVKCGGCFEAIAPNEFVMRAQKSVYHLSCFCCCVCERQLQKGDEFVLKEGQLLCKGDYEKER | |
| ELLSLVSPAASDSGKSDDEESLCKSAHGAGKGTAEEGKDHKRPKRPRTILTTQQRRAFKASFEVSS | |
| KPCRKVRETIAAETGLSVRVVQVWFQNQRAKMKKLARRQQQQQQDQQNTQRLSSAQTNGGGSAGMEGIMNPYTALP | |
| TPQQLLAIEQSVYSSDPFRQGLTPPQMPGDHMHPYGAEPLFHDLDSDDTSLSNLGDCFLATSEAGPLQSRVGNPID | |
| HLYSMQNSYFTS | |
| Lmx1b mouse gene (underline letters denote cDNA boundaries) | |
| (SEQ ID No. 13) | |
| 1 | atg ttggacg gcatcaagat ggaggagcac gcccttcgcc ccgggcccgc caccctgggg |
| 61 | gtgctgctgg gctccgactg cccgcatccc gccgtctgcg agggctgcca gcggcccatc |
| 121 | tccgaccgct tcctgatgcg agtcaacgag tcgtcctggc acgaggagtg tttgcagtgc |
| 181 | gcggcatgtc agcaagccct caccaccagc tgctacttcc gggatcggaa actgtactgc |
| 241 | aaacaagact accaacagct cttcgcggca aagtgcagcg gctgcatgga gaagatcgcc |
| 301 | cctaccgagt tcgtcatgcg ggcgctggag tgtgtgtacc acttgggctg tttctgctgc |
| 361 | tgtgtgtgcg agaggcaact gcgcaagggg gacgagttcg tgctcaagga gggccagctg |
| 421 | ctgtgcaagg gtgactatga gaaggagaaa gacctgctca gctccgtgag cccggacgag |
| 481 | tctgactctg tgaagagtga ggatgaagat ggagacatga agccggccaa ggggcagggc |
| 541 | agccagagta aaggcagtgg agatgacggg aaagacccga gaaggcccaa acggccccga |
| 601 | accatcctca ccacacagca gcgaagagct ttcaaggcat cctttgaggt ctcctccaag |
| 661 | ccctgtcgga aggtccgaga gacattggca gcagagacag gcctcagcgt gcgtgtggtc |
| 721 | caggtctggt ttcagaacca aagagcaaag atgaagaagc tggcccggag acaccagcaa |
| 781 | cagcaggagc agcagaactc ccagcggctg ggccaagagg ttctgtcaag ccgcatggag |
| 841 | ggcatgatgg cctcctacac gccgctggcc cctccgcagc agcagatcgt ggccatggag |
| 901 | cagagcccct acggaagtag cgaccccttc caacagggcc tcacgccgcc ccaaatgcca |
| 961 | gggaacgact ccatcttcca cgatatcgat agtgatacct ccctcaccag cctcagcgac |
| 1021 | tgcttcctcg gctcttccga cgtgggctcc ctgcaggccc gcgtggggaa ccccattgac |
| 1081 | cggctctact ccatgcagag ctcctactct gcctcctgag agccagccgg gccgcatgga |
| 1141 | cgcttgggcc tgggcctagg gtggagccac aggcctctgc agccagccgg ccccccagcc |
| 1201 | caccacccgc tcagactct |
| Lmx1b mouse protein | |
| (SEQ ID No. 14) | |
| MLDGIKMEEHALRPGPATLGVLLGSDCPHPAVCEGCQRPISDRFLMRVNESSWHEECLQCAACQQALTTSCYFRDR | |
| KLYCKQDYQQLFAAKCSGCMEKIAPTEFVMRALECVYHLGCFCCCVCERQLRKGDEEVLKEGQLLCKGDYEKEKDL | |
| LSSVSPDESDSVKSEDEDGDMKPAKGQGSQSKGSGDDGKDPRRPKRPRTILTTQQRRAFKASFEVS | |
| SKPCRKVRETLAAETGLSVRVVQVWFQNQRAKMKKLARRHQQQQEQQNSQRLGQEVLSSRMEGMMASYTPLAPPQQ | |
| QIVAMEQSPYGSSDPFQQGLTPPQMPGNDSIFHDIDSDTSLTSLSDCFLGSSDVGSLQARVGNPIDRLYSMQSSYF | |
| AS | |
| Lmx1b human gene (underline letters denote cDNA boundaries) | |
| (SEQ ID No. 15) | |
| 1 | gcgtcccatg gatatagcaa caggtcccga gtcgctggag aggtgcttcc ctcgcgggca |
| 61 | gacggactgc gccaagatgt tggacggcat caagatggag gagcacgccc tgcgccccgg |
| 121 | gcccgccact ctgggggtgc tgctgggctc cgactgcccg catcccgccg tctgcgaggg |
| 181 | ctgccagcgg cccatctccg accgcttcct gatgcgagtc aacgagtcgt cctggcacga |
| 241 | ggagtgtttg cagtgcgcgg cgtgtcagca agccctcacc accagctgct acttccggga |
| 301 | tcggaaactg tactgcaaac aagactacca acagctcttc gcggccaagt gcagcggctg |
| 361 | catggagaag atcgccccca ccgagttcgt gatgcgggcg ctggagtgcg tgtaccacct |
| 421 | gggctgcttc tgctgctgcg tgtgtgaacg gcagctacgc aagggcgacg aattcgtgct |
| 481 | caaggagggc cagctgctgt gcaagggtga ctacgagaag gagaaggacc tgctcagctc |
| 541 | cgtgagcccc gacgagtccg actccgtgaa gagcgaggat gaagatgggg acatgaagcc |
| 601 | ggccaagggg cagggcagtc agagcaaggg cagcggggat gacgggaagg acccgcggag |
| 661 | gcccaagcga ccccggacca tcctcaccac gcagcagcga agagccttca aggcctcctt |
| 721 | cgaggtctcg tcgaagcctt gccgaaaggt ccgagagaca ctggcagctg agacgggcct |
| 781 | cagtgtgcgc gtggtccagg tctggtttca gaaccaaaga gcaaagatga agaagctggc |
| 841 | gcggcggcac cagcagcagc aggagcagca gaactcccag cggctgggcc aggaggtcct |
| 901 | gtccagccgc atggagggca tgatggcttc ctacacgccg ctggccccac cacagcagca |
| 961 | gatcgtggcc atggaacaga gcccctacgg cagcagcgac cccttccagc agggcctcac |
| 1021 | gccgccccaa atgccaggga acgactccat cttccatgac atcgacagcg atacctcctt |
| 1081 | aaccagcctc agcgactgct tcctcggctc ctcagacgtg ggctccctgc aggcccgcgt |
| 1141 | ggggaacccc atcgaccggc tctactccat gcagagttcc tacttcgcct cctgagagcc |
| 1201 | agccaggcgc acggacgctt gggcaggggc ctggggggga ctgccagcct ctgcggccag |
| 1261 | cctggccacc cccgccctgc tctccgcaca gactacagac agccatacgg tgccctcccc |
| 1321 | tcggccagct gggcctgacc actgtgcccg ttgggtacag ccagaccggt agatgggcac |
| 1381 | agcctgggca ggggctgtgt cctgcccaca gagaccttgt catccccagg gacccagagc |
| 1441 | tctcggacgg ccactcgcct cccagcccca cctcggcctc catcgcctcc tccccatctc |
| 1501 | ttttttggga agcttaaatt ctctctattt ttttaaatgt cctctctgtg tccatggccc |
| 1561 | tccatgcaag ccccaggaca atggtgtcat gaggcggtga cctgagaagc gtgtgtacct |
| 1621 | gtgccccagc aagggcaggg gtggcctctg ggggcaggcc cactgcctgg aaccgcacac |
| 1681 | ccctcagcct gagtctggag cagcagtgga gaggggcctg aggggaggca ctgtcaggag |
| 1741 | gcgggctcgg agcctgagcc tgggcaggcg caaagggaca gagaggcacg tgcagacaca |
| 1801 | tgcacacttg cagacaaacc cacgcaaaca cacacacagc tgtatgggga caccagaagg |
| 1861 | gacagggatg ctcagcgggt ctgtcctgcc ttgtcagaaa gagaaaagga ggccaggcag |
| 1921 | gggacccccc agttcttaag agcgattgga aagggaggaa ggggagagga agaggcgaac |
| 1981 | ttgaagcatc ggacccagtt gtatcccagc ctgggcccaa atgggggcag cctgggcagg |
| 2041 | gagggcagcc ccaggcccca ccaactctag aggcagatgg agcccccaga accaggtagc |
| 2101 | atcagaccag acaacagagc ctccaggggt cagggacttc agaagcacct gctgggcacc |
| 2161 | ccatctgcaa tgtggtcctc tccccagcca cctctgcctc ccctcacata cctccagtga |
| 2221 | caaggagctc actaggtcag cgagcccaca gcagctgtgc tgtcctgcat cccagagcca |
| 2281 | ggcttcccca gctctccctc ttaacactgt cccccagcag gcctccggct gtccctctaa |
| 2341 | aggtgtgggg caggtatcac ttcaccttcc cactgatgtc agccggccag aagtgagcag |
| 2401 | gcacatcacc tctcctgctg tggcaccctt cctctgttaa tttggcccaa aagacaatga |
| 2461 | tttggccaca tgaccttaga gattcaccct gccctgctgt agctaaatcc ctgggcccca |
| 2521 | cacgcaagtg acagctaagc cacatctgtt ttctgtgtat atgcaggatg ggggcaccta |
| 2581 | ctgttttgtt ttgttttgtt ttgttttgtt ttgttttgtt ttgttttgtt ttgtttgaga |
| 2641 | cggagtttcg ctcttgttgc ccaggctgga gtgcaatggc gcgatctcgg ctcaccacaa |
| 2701 | cctccgcctc ccaggttcaa gtgattctga tgcctcagcc tccctagtag ctgagattac |
| 2761 | aggcatgcgc caccacaccc agctaatttt gtatttttag tagcaacggg gtttctccat |
| 2821 | gttggtcagg ctggtctcca acccccgacc tcaggtgatc cgcctgcctc ggcctcccaa |
| 2881 | agtgctggga ttacaggcgt gagccaccgc acccagtctg cacttactgt ttagactgaa |
| 2941 | tgagggaccg tgacctcttt ccttttccat tccttcttac tcgattcatt ccagcctgtg |
| 3001 | gaatttctct gcaccctgat tcagtgacca ctgctctcct ctctcccagc acatctgccc |
| 3061 | agtgaggagt tggccctggg tctcacctga ggtgtgtgga ccgggctggc ctctccctgt |
| 3121 | ttgacattgg cccattaatg catcctcttt gggggacaca ttccaattgc atttcctgcc |
| 3181 | cccttctccc agggcaattg cagaagattg tgtcaggcgc cctgctggaa gtcaggtgca |
| 3241 | ctagatccat ccccagcccc agtctgctca actctatccc tgtcagagca aggaggctgg |
| 3301 | gctgctgggg cctgactggt gagcccaccc tgtcccctgg tgatcactgt gtccccttgt |
| 3361 | tcaggtgctc acaaccctac ctttaactct gaggtcaagc cctaggccac caccctaaag |
| 3421 | tctgcctggt ccaacctttg agcaagtaag gataatgaat gtcccttttc cacctttggg |
| 3481 | gccctctgcc tggatctctg gaatcctcta agttcaacct gttctgtggt tttgctcccg |
| 3541 | tttgctggga aattcagtcc ccccagaatg tcctgggcca acctccttgc ctgacatgtg |
| 3601 | gcctcgtgtc acccattggg ccccagcagc cagctagccc ttctgcagct cttcttacaa |
| 3661 | acagagcctc tccaaggacc tcagttgatg ttctggtcct tctgccgcct cagcccacca |
| 3721 | gggtccgtgc caccatgggt ctcttgagca gcagctgcac tggcttctgg agagacaccc |
| 3781 | ctctttctcc ttttgcacat gcaccatctg aatcgtgcca gggacatcct gggcagattc |
| 3841 | aggggcagat gccctatccc ccaggagacc tggcccttct ctctcagacc caataagttg |
| 3901 | gaagggacgt cagaagcggt catctcatct gccccttatt ttatagttgg aaaccctgag |
| 3961 | gcaagagagg gaaagaggcc tgtccaaggt ccgggttagt gacagagctg agctgagaac |
| 4021 | agggacgttg tgccccactg tcccctgtgg tttgtgaatg acctccaggt cagggggtca |
| 4081 | caacttgttc ttagtaaact tgccagctgt tggggtcaca tattcccatt ctggggcctc |
| 4141 | acaaaccccc gaatccagcc gggaccccat gccaggagct ggtctaggga cagcatgctt |
| 4201 | gtgacccaca gactgttaaa gccagaaggg acctcagaga gtcccttatg ctggaggcgc |
| 4261 | cctgtcagcc gtggctaggg gccccttgct ctatgctgtg ccttgctgcc cacaggctcc |
| 4321 | cagacaccag tgcccactct gcccagcccc ggactgggtg tggctcgcag atgaacaaga |
| 4381 | tgcagggcct gccttgaggg gtgtctccta gaaggaaagc cagactctcc ggcccagcca |
| 4441 | gagagtccag acatggcagg gacccgtttc tcagatgagg agcctgaggc tcagagaagg |
| 4501 | gaggcgatgt gttcagggcc acccagcaga agcctgtggg gctgggcaac cttctcccac |
| 4561 | tttatgggag gagctgcagc cttggctggg agctgggcgg ggagtagcca ggaccacccc |
| 4621 | ttgcccgtgc cgtgacatgg aaccttcatc actaaggggg ctggagtggg aagagggaga |
| 4681 | taactgtgtg gtctccagag caaaagagaa tgagaggtgg gcagggggag tcttggcaaa |
| 4741 | agaccaagtt ccacttccct gctggggaag tcaaggctca gaaagaggaa ataattgccc |
| 4801 | caggtaacac agggcagagg agggacaaaa agctgggcat ggccccagcc agagcctcat |
| 4861 | ctgcctactc cgtgaagcct cccaggtact ctgctatcct gggaaacgca cagggaggcc |
| 4921 | acacagagac actgctcaca agagtcagac caaggtgcca gcacagcctg gaaagagctc |
| 4981 | agaaaggggg ttggtgcacg tggctgggca tcttaggagg cttcctgagg gtgggtaaag |
| 5041 | gtgggaaggc cctggcgctg catcagatga gcagggcctg gcagggacaa gcctcttctc |
| 5101 | ctttgggaag ccctgcagcc tcctagcaag aggctgattc cccactctgc ccccatctga |
| 5161 | atgtcctttt catgttgcac gcagggaacc tcaggaagga ggattgcctg atgcctgcct |
| 5221 | ggctccatcc ttgagctctg ggcaccacct agggtgaggg agagcctgca gctctggggc |
| 5281 | taagtctgcc ctggggggaa agggctccac gctcacacgc acgcgctcgc acacacacac |
| 5341 | tcacacctgg acgcacacgg aggcttgcgg acccatactc acaggcacat gtggcctggg |
| 5401 | gactggggga gcaggaaaga cccctccaac atttggccct tggaaggcac cattgccaat |
| 5461 | gagcctcttt gctggttccc ccgaccccac ctgggggtcc catgggagcc cagcccagcc |
| 5521 | aggtgtgggg atgggccacc ggccattcct gttttccttg tacagacaga ttctcactac |
| 5581 | ccacccgcca tccccagaca cattttattt aataacttgt cattgttaaa ttatttatta |
| 5641 | gcgtttacca caccaccacc cccaccctgc cctccactct caccttccac ctcttcccac |
| 5701 | aacagcagaa aatggaaaca acaacaaaaa aagatgagac atcagtatat ttgtaaataa |
| 5761 | accgacctgt acactcaaaa aaa |
| Lmx1b human protein | |
| (SEQ ID No. 16) | |
| MDIATGPESLERCFPRGQTDCAKMLDGIKMEEHALRPGPATLGVLLGSDCPHPAVCEGCQRPISDRFLMRVNESSW | |
| HEECLQCAACQQALTTSCYFRDRKLYCKQDYQQLFAAKCSGCMEKIAPTEFVMRALECVYHLGCFCCCVCERQLRK | |
| GDEFVLKEGQLLCKGDYEKEKDLLSSVSPDESDSVKSEDEDGDMKPAKGQGSQSKGSGDDGKDPRR | |
| PKRPRTILTTQQRRAFKASFEVSSKPCRKVRETLAAETGLSVRVVQVWFQNQRAKMKKLARRHQQQQEQQNSQRLG | |
| QEVLSSRMEGMMASYTPLAPPQQQIVAMEQSPYGSSDPFQQGLTPPQMPGNDSIFHDIDSDTSLTSLSDCFLGSSD | |
| VGSLQARVGNPIDRLYSMQSSYFAS | |
| Otx2 mouse gene (underline letters denote cDNA boundaries) | |
| (SEQ ID No. 17) | |
| 1 | caggtttatc tggtctcact ccatcccctc tagttttgga gctgctgggg ggtggggggg |
| 61 | acggcggggg tgggggacgc atctgcaact cctttaaaag cctgtgccca gcgtctcccg |
| 121 | ggttcttttt agttagtgct ggaacgtgga ggaagctgct ccctccgaag cagtaaacca |
| 181 | gcatttctgt ttgtttgttt gctttgccct tagttccgtc actccaaatc tacccaccaa |
| 241 | ggaccctgac cctgtccact ccaggcgaat cgagaccgtc cggctgggtc cccccaattt |
| 301 | gggccgactt tgcgcctcca aacaacctta gcatgatgtc ttatctaaag caaccgcctt |
| 361 | acgcagtcaa tgggctgagt ctgaccactt cgggtatgga cttgctgcat ccctccgtgg |
| 421 | gctaccccgc caccccccgg aaacagcgaa gggagaggac gacatttact agggcacagc |
| 481 | tcgacgttct ggaagctctg tttgccaaga cccggtaccc agacatcttc atgagggaag |
| 541 | aggtggcact gaaaatcaac ttgccagaat ccagggtgca ggtatggttt aagaatcgaa |
| 601 | gagctaagtg ccgccaacag cagcagcagc agcagaatgg aggtcagaac aaagtgaggc |
| 661 | ctgccaagaa gaagagctct ccagctcggg aagtgagttc agagagtgga acaagtggcc |
| 721 | agttcagtcc cccctctagt acctcagtcc caaccattgc cagcagcagt gctccagtgt |
| 781 | ctatctggag cccagcgtcc atctccccac tgtctgaccc cttgtccact tcctcctcct |
| 841 | gcatgcagag gtcctatccc atgacctata ctcaggcttc aggttatagt caaggctatg |
| 901 | ctggctcaac ttcctacttt gggggcatgg actgtggatc ttatttgacc cctatgcatc |
| 961 | accagcttcc tggaccaggg gccacactca gtcccatggg taccaatgct gttaccagcc |
| 1021 | atctcaatca gtccccagct tctctttcca cccagggata tggagcttca agcttgggtt |
| 1081 | ttaactcaac cactgattgc ttggattata aggaccaaac tgcctcttgg aagcttaact |
| 1141 | tcaatgctga ctgcttggat tataaagatc agacgtcctc atggaaattc caggttttgt |
| 1201 | gaagacctgt agaagctatt tttgtgggtg atttttaaat atgctgggct gaacattcca |
| 1261 | gttttagcca ggcattggtt aaaaaagtta gatggaacga tgctctcaga ctcctgatca |
| 1321 | aagttaccga gaggcataga aggaaaaagg aaggggcctt agaagggtcc atcaaccagc |
| 1381 | aacctgaaat ggacaaacca atctacttaa gattctgtta tagttctaga tcattggttt |
| 1441 | cctgatttgc aaatgattga tcaaatatat tctagcgaca tgcaaccaaa taccactcaa |
| 1501 | aacaaaaatc cagcaaaact gagttgtgag ggaagggagg gaaggtcatg gccttcaaag |
| 1561 | cagaggtgat ccggtgtttt agccaatctt tggttgaatc ttaggaatgg acaatgtccc |
| 1621 | aggctcattc acgtttcatg accaacaggt agttggcact gaaaaacttt tcagggctgt |
| 1681 | gtggattgtg cgactgattg tcctagatgc actactttat ttaaaaaaaa aaaaaaa |
| Otx2 mouse protein | |
| (SEQ ID No. 18) | |
| MMSYLKQPPYAVNGLSLTTSGMDLLHPSVGYPATPRKQRRERTTFTRAQLDVLEALFAKTRYPDIFMREEVALKIN | |
| LPESRVQVWFKNRRAKCRQQQQQQQNGGQNKVRPAKKKSSPAREVSSESGTSGQFSPPSSTSVPTIASSSAPVSIW | |
| SPASISPLSDPLSTSSSCMQRSYPMTYTQASGYSQGYAGSTSYFGGMDCGSYLTPMHHQLPGPGAT | |
| LSPMGTNAVTSHLNQSPASLSTQGYGASSLGFNSTTDCLDYKDQTASWKLNFNADCLDYKDQTSSWKFQVL | |
| Otx2 gene human (underline letters denote cDNA boundaries) | |
| (SEQ ID No. 19) | |
| 1 | gagagcggga ccggcctcag ctccaacaca gcctccactg tgattaaaaa taaaaattgc |
| 61 | tagagcagcc ctcactcgcc acatctactt tgatagctgg ctatttggaa tttaaaggat |
| 121 | atttgacttt ttctaacctc ccatgaggct gtaagttcca ctgctccaaa cccacccacc |
| 181 | aaggactctg aacctgtcca ccccgggcgc atcaagatct tccagctggg tacccccgat |
| 241 | ttgggccgac tttgcacctc caaacaacct tagcatgatg tcttatctta agcaaccgcc |
| 301 | ttacgcagtc aatgggctga gtctgaccac ttcgggtatg gacttgctgc acccctccgt |
| 361 | gggctacccg gggccctggg cttcttgtcc cgcagccacc ccccggaaac agcgccggga |
| 421 | gaggacgacg ttcactcggg cgcagctaga tgtgctggaa gcactgtttg ccaagacccg |
| 481 | gtacccagac atcttcatgc gagaggaggt ggcactgaaa atcaacttgc ccgagtcgag |
| 541 | ggtgcaggta tggtttaaga atcgaagagc taagtgccgc caacaacagc aacaacagca |
| 601 | gaatggaggt caaaacaaag tgagacctgc caaaaagaag acatctccag ctcgggaagt |
| 661 | gagttcagag agtggaacaa gtggccaatt cactcccccc tctagcacct cagtcccgac |
| 721 | cattgccagc agcagtgctc ctgtgtctat ctggagccca gcttccatct ccccactgtc |
| 781 | agatcccttg tccacctcct cttcctgcat gcagaggtcc tatcccatga cctatactca |
| 841 | ggcttcaggt tatagtcaag gatatgctgg ctcaacttcc tactttgggg gcatggactg |
| 901 | tggatcatat ttgaccccta tgcatcacca gcttcccgga ccaggggcca cactcagtcc |
| 961 | catgggtacc aatgcagtca ccagccatct caatcagtcc ccagcttctc tttccaccca |
| 1021 | gggatatgga gcttcaagct tgggttttaa ctcaaccact gattgcttgg attataagga |
| 1081 | ccaaactgcc tcctggaagc ttaacttcaa tgctgactgc ttggattata aagatcagac |
| 1141 | atcctcgtgg aaattccagg ttttgtgaag acctgtagaa cctctttttg tgggtgattt |
| 1201 | ttaaatatac tgggctggac attccagttt tagccaggca ttggttaaaa gagttagatg |
| 1261 | ggatgatgct cagactcatc tgatcaaagt tccgagaggc atagaaggaa aaacgaaggg |
| 1321 | ccttagaggg gcctacaaac cagcaacatg aaatggacaa accaatctgc ttaagatcct |
| 1381 | gtcatagttt tagatcattg gttatcctga tttgcaaagt gatcaaaagc attctagcca |
| 1441 | tgtgcaacca aacaccacca aaaataaaat caaacaaaac taagttgtga aggaagggag |
| 1501 | ggaaggtcat agccttctta agcagaggtg ttccattgtt ttagccaatc cttggttgaa |
| 1561 | tcttaggaat gaacagtgtc tcaagctcat tcacgtttca tgaccaactg gtagttggca |
| 1621 | ctgaaaaaac ttttcagggc tgtgtgaatt gtgtgactga ttgtcctaga tgcactactt |
| 1681 | tatttaaaaa ataatgttca taaggagtca atatgtagtt taagagacaa tcagtgtgtg |
| 1741 | tcttataaat ggtacatctg tggtttttaa tctgtgctag acttcaaaac tgtgatctcc |
| 1801 | tgttattgta tgcaaccttg aactccacct ctgcaggggt tcttctgtga ttaaataggt |
| 1861 | tataattata agcaaaattc agagcaactg agtactgatc taaaaagatt acctttggct |
| 1921 | ggaggtgagc tgcactgaaa ctttacgaca aaatgtctct ggacaaagag agtcagagaa |
| 1981 | gagaagcaaa aggacactaa ttcatctgta atttactgtt ggtaagccta gcagtaaaga |
| 2041 | gacattggtc aattgctctg accctgatga attattaaac tgagatcatt gtcgtttatg |
| 2101 | cttgcagatg ttaaatggaa aagttatata tgcataaacc ttttcttcct ggatttggca |
| 2161 | gatatgtata attatattaa aatggttcta gcacaaaaaa aaaaaaaaa |
| Otx2 protein human | |
| (SEQ ID No. 20) | |
| MMSYLKQPPYAVNGLSLTTSGMDLLHPSVGYPGPWASCPAATPRKQRRERTTFTRAQLDVLEALFAKTRYPDIFMR | |
| EEVALKINLPESRVQVWFKNRRAKCRQQQQQQQNGGQNKVRPAKKKTSPAREVSSESGTSGQFTPPSSTSVPTIAS | |
| SSAPVSIWSPASISPLSDPLSTSSSCMQRSYPMTYTQASGYSQGYAGSTSYFGGMDCGSYLTPMHH | |
| QLPGPGATLSPMGTNAVTSHLNQSPASLSTQGYGASSLGFNSTTDCLDYKDQTASWKLNFNADCLDYKDQTSSWKF | |
| QVL | |
| Pitx3 mouse gene (underline letters denote cDNA boundaries) | |
| (SEQ ID No. 21) | |
| 1 | gcggccgccc cagagcaggg gggcggcccc caccccgcag ggtgcctggc ccctggcccc |
| 61 | tgcctgcgct ccagaacgcc gccgccacag ccaccacccg gagtctgcct gctgcgggac |
| 121 | gcactagacc tccctccatg gagtttgggc tgcttggtga ggcagaggcg cgaagccctg |
| 181 | cgctgtcgtt atcggacgca ggcactccac accctccgct tccagaacat ggctgcaagg |
| 241 | ggcaggagca cagtgactcg gagaaggcct cggcctcact gccggggggc tcccccgagg |
| 301 | acggctctct gaagaagaag cagcggcggc agcgcacgca cttcaccagc cagcagctgc |
| 361 | aggagctgga ggccaccttc cagaggaatc gctaccctga catgagcacc cgcgaagaga |
| 421 | tcgcggtgtg gaccaacctc actgaggccc gcgtgcgggt gtggttcaag aaccggcgcg |
| 481 | ccaagtggcg gaagcgggag cgcagccagc aggcggagct gtgcaaaggt ggcttcgcag |
| 541 | ccccgctcgg gggcctggtg ccaccctacg aggaggtgta cccgggctac tcgtacggca |
| 601 | actggccgcc caaggctctc gccccgccgc tcgccgccaa gaccttcccg ttcgccttca |
| 661 | actcggtcaa cgtggggcct ctggcttcac agcctgtatt ctcaccgccc agctccatcg |
| 721 | ccgcttctat ggtgccctcg gccgccgctg ccccgggcac cgtaccaggt cccggagcct |
| 781 | tgcagggcct gggcggggca ccccccgggc tggctccagc cgccgtgtcc tccggggcag |
| 841 | tgtcctgccc ttacgcctcg gccgccgcag ccgccgctgc agccgcctcc tccccctatg |
| 901 | tataccggga cccgtgtaac tcgagcctgg ctagcctgcg gctcaaagcc aagcagcacg |
| 961 | cctctttcag ctatcccgcc gtgcccgggc cgccgccggc cgctaacctt agcccctgcc |
| 1021 | agtacgccgt ggaacggccg gtgtgagccg caggtctgtg gatccatccc cgagggcggg |
| 1081 | gcagtaattc acagcctctc cggacagggg tcgcctagac tggcttgccc tcgtcccagg |
| 1141 | gtctgaaagg ggtgccagag cacccgggaa gaggccgcgg gcttcgaaga gggccttttc |
| 1201 | cctcgcagcc cccgagcggt ggtctgaccc ctatgcggag accgcgcccc taggactaag |
| 1261 | gccaggaaca gggaccagct cccccagggc caattcaccc ttggctcacc ccgccttctc |
| 1321 | cagactcccc ctatcccatt ttcaaagatc aatgaaataa acgtgcgcgg actgtcaaa |
| Pitx3 mouse protein | |
| (SEQ ID No. 22) | |
| MEFGLLGEAEARSPALSLSDAGTPHPPLPEHGCKGQEHSDSEKASASLPGGSPEDGSLKKKQRRQRTHFTSQQLQE | |
| LEATFQRNRYPDMSTREEIAVWTNLTEARVRVWFKNRRAKWRKREPSQQAELCKGGFAAPLGGLVPPYEEVYPGYS | |
| YGNWPPKALAPPLAAKTFPFAFNSVNVGPLASQPVFSPPSSIAASMVPSAAAAPGTVPGPGALQGL | |
| GGAPPGLAPAAVSSGAVSCPYASAAAAAAAAASSPYVYRDPCNSSLASLRLKAKQHASFSYPAVPGPPPAANLSPC | |
| QYAVERPV | |
| Pitx3 gene human (underline letters denote cDNA boundaries) | |
| (SEQ ID No. 23) | |
| 1 | ggagcgcccg agcggagagg cggcccggga gcaggggggc ggcccccact ccggccgggt |
| 61 | gcccggcccc tggcccctgc ctgccctcta gatcgccgcc gcagccgccg ctactgggag |
| 121 | tctgcctgtt gcaggacgca ctagccctcc ctccatggag ttcggcctgc tcagcgaggc |
| 181 | agaggcccgg agccctgccc tgtcgctgcc agacgctggc actccgcacc cccagctccc |
| 241 | agagcacggc tgcaagggcc aggagcacag cgactcagaa aaggcctcgg cttcgctgcc |
| 301 | cggcggctcc ccagaggacg gttcgctgaa aaagaagcag cggcggcagc gcacgcactt |
| 361 | caccagccag cagctacagg agctagaggc gaccttccag aggaaccgct accccgacat |
| 421 | gagcacgcgc gaggagatcg ccgtgtggac caacctcacc gaggcccgcg tgcgggtgtg |
| 481 | gttcaagaac cggcgcgcca aatggcggaa gcgcgagcgc agccagcagg ccgagctatg |
| 541 | caaaggcagc ttcgcggcgc cgctcggggg gctggtgccg ccctacgagg aggtgtaccc |
| 601 | cggctacccg tacggcaact ggccgcccaa ggctcttgcc ccgccgctcg ccgccaagac |
| 661 | ctttccattc gccttcaact cggtcaacgt ggggcctctg gcttcgcagc ccgtcttctc |
| 721 | gccacccagc tccatcgccg cctccatggt gccctccgcc gcggctgccc cgggcaccgt |
| 781 | gccagggcct ggggccctgc agggcctggg cgggggcccc cccgggctgg ctccggccgc |
| 841 | cgtgtcctcc ggggccgtgt cctgccctta tgcctcggcc gccgccgccg ccgcggctgc |
| 901 | cgcctcttcc ccctacgtct atcgggaccc gtgtaactcg agcctggcca gcctgcggct |
| 961 | caaagccaaa cagcacgcct ccttcagcta ccccgctgtg cacgggccgc ccccggcagc |
| 1021 | caaccttagt ccgtgccagt acgccgtgga aaggcccgta tgagcggccc cgcccgtaga |
| 1081 | tcatccccga gggcgggggc aacgattcac agcctccgcg gactggggtc attttgactg |
| 1141 | gcttgctccc gccccagggt ctgaaagggg tgtttgggca gctggggggc accggctcag |
| 1201 | gagagggcct tcccctccca gccctgaggg gtggactagg ccctacacac agaccgcgcc |
| 1261 | cctgggacta aagccaggaa cagggaccag ctccccgggg gccaactcac ccttggccca |
| 1321 | tcccgccttc tccaggcttc ccctccctcg ttttcaaaga taaatgaaat aaacgtgcgc |
| 1381 | ggactgtcaa aaaaaaaaaa aaaaaaa |
| Pitx3 protein human | |
| (SEQ ID No. 24) | |
| MEFGLLSEAEARSPALSLSDAGTPHPQLPEHGCKGQEHSDSEKASASLPGGSPEDGSLKKKQRRQRTHFTSQQLQE | |
| LEATFQRNRYPDMSTREEIAVWTNLTEARVRVWFKNRRAKWRKRERSQQAELCKGSFAAPLGGLVPPYEEVYPGYS | |
| YGNWPPKALAPPLAAKTFPFAFNSVNVGPLASQPVFSPPSSIAASMVPSAAAAPGTVPGPGALQGL | |
| GGGPPGLAPAAVSSGAVSCPYASAAAAAAAAASSPYVYRDPCNSSLASLRLKAKQHASFSYPAVHGPPPAANLSPC | |
| QYAVERPV | |
| Ngn2 (Neurog2) mouse gene (underline letters denote cDNA boundaries) | |
| (SEQ ID No. 25) | |
| 1 | gcagccactg aaccacaagc agctcggctt taactggagt gccttggagt cgcgtgccag |
| 61 | cagccacacg gccagggact gactgacaga caaccacgca cgagaacgac aacacacgag |
| 121 | actcgggcga gctgccgcgg tcgtccgggc tcttggcaaa gtcgcccagc cgagaggccc |
| 181 | ccccgcggag gtgcgcctag gaagcgccaa gcccgcggcg cggaggacac cgtgctcggt |
| 241 | tccgggctgc ggggacattc ccggacacac accggagcag cagctgcgcc gcgacacatc |
| 301 | tggagccgcg taggatgttc gtcaaatctg agactctgga gttgaaggag gaagaggagg |
| 361 | tactgatgct gctgggctcg gcttccccgg cctcggcgac cctgaccccg atgtcctcca |
| 421 | gcgcggacga ggaggaggac gaggagctgc gccggccggg ctccgcgcgt gggcagcgtg |
| 481 | gagcggaagc cgggcagggg gtgcagggca gtccggcgtc gggtgccggg ggttgccggc |
| 541 | cagggcggct gctgggcctg atgcacgagt gcaagcgtcg cccgtcgcgc tcacgggccg |
| 601 | tctcccgagg tgccaagacg gcggagacgg tgcagcgcat caagaagacc cgcaggctca |
| 661 | aggccaacaa ccgcgagcgc aaccgcatgc acaacctaaa cgccgcgctg gacgcgctgc |
| 721 | gcgaggtgct gcccaccttc cccgaggatg ccaagctcac gaagatcgag acgctgcgct |
| 781 | tcgcccacaa ttacatctgg gcgctcaccg agactctgcg cctggcggac cactgcgccg |
| 841 | gcgccggtgg cctccagggg gcgctcttca cggaggcggt gctcctgagc ccgggagctg |
| 901 | cgctcggcgc cagcggggac agcccttctc caccttcctc ctggagctgc accaacagcc |
| 961 | cggcgtcatc ctccaactcc acgtccccac acagctgcac tttatcgccc gccagccccg |
| 1021 | ggtcagacgt ggactactgg cagcccccac ctccggagaa gcatcgttat gcgcctcacc |
| 1081 | tgcccctcgc cagggactgt atctagagct gcgggtctcc ctctctcgtc ctctacccgg |
| 1141 | ccctcttccc atccttctcc cgcccctcac cctccacgcc ccggactcca cttcacagag |
| 1201 | cagaggtggc ccttgcaatc ccctcggcgg ctggtgcatt cgggggtgga gaccagctct |
| 1261 | ggtttattga agatgtgagg atttatggtc aaagaggact atggcgtgtg ggagtggggg |
| 1321 | ctggcgtggg gaacctcgta agactgtaaa agacactgag aaaaagtacc ataactaacg |
| 1381 | agtgtgcaga gcagactgac gctcctcccc tctctcagag ctgctggagg agaactccgg |
| 1441 | gcaggcagtt cgtgtgaatc tctcagaggg aatgcaactg gtccctgtga tcttttcacc |
| 1501 | ttcgtttcta catagagatg ttaatgtcag tcgaaagaaa tgtattttag catctgaatg |
| 1561 | aatttactgg taataatatt atccacacat ttgcaatggc tggcatctgc tctattccca |
| 1621 | ttgctgtctg caggctgtgg gaatttcacc tgtcaaacca aactttccct ctctgatgtg |
| 1681 | cactttgttt ttttcccaga ttcgtcacaa tgcctattgt cccgcccttc tttttgcttt |
| 1741 | ttttctccat tttgccatct gtctcttatg atttataagg gggaaaaact tgttttgtta |
| 1801 | gagggccagg ttagaagtca ttgtataatt tgtaggcttt tgtaagggtt gaatgcaagc |
| 1861 | gtggaaattt aggctgaatt ctctatcaaa agaaaaaatg tgaaggaaaa aggaaaaatc |
| 1921 | aggagggagg attgcttcat gcattattta tctcgacctt ttaggggaga aggaactccc |
| 1981 | ccatcctttc aagagattaa aaataaatca acagtctgaa aacctaagca gacacggggc |
| 2041 | attgccagga tcagccacac acgtgtttcc ttctatttat tttgaagaaa aatttcatgg |
| 2101 | gaaagtatgt atttttttgt atattctaca gagtttattc tagtatgtat ttacatcccg |
| 2161 | aagaataaga aaattgtttt gtgattaagc tataaataaa gtatctaatt ttcataaaaa |
| 2221 | aaaaaaaaaa aaaaaaaaaa aaaa |
| Ngn2 mouse protein | |
| (SEQ ID No. 26) | |
| MFVKSETLELKEEEEVLMLLGSASPASATLTPMSSSADEEEDEELRRPGSARGQRGAEAGQGVQGSPASGAGGCRP | |
| GRLLGLMHECKRRPSRSRAVSRGAKTAETVQRIKKTRRLKANNRERNRMHNLNAALDALREVLPTFPEDAKLTKIE | |
| TLRFAHNYIWALTETLRLADHCAGAGGLQGALFTEAVLLSPGAALGASGDSPSPPSSWSCTNSPAS | |
| SSNSTSPYSCTLSPASPGSDVDYWQPPPPEKHRYAPHLPLARDCI | |
| Ngn2 (Neurog2) gene human (underline letters denote cDNA boundaries) | |
| (SEQ ID No. 27) | |
| 1 | cgcagccact gaaccacaag cagcttcgcg ttaactggag tgcctgggag tcgcgtgcca |
| 61 | ggagccgcac ggccagggac tgactgacag acagacacgc accaccacca caacacacga |
| 121 | gacccgggcg ggccgccgcc gccgccgccg gggctcttgg caaactcgcc ggtcgcagag |
| 181 | gtcccccgcg gagctgcgcc acagtagcgc cgggcttgca gctttcacgc cgggcgaagg |
| 241 | acccggcgct gcgctcgcag ctgcgcggag attcccggca caggccaaag tcacagcaac |
| 301 | gctgaggcac agttagagcc aactaagatg ttcgtcaaat ccgagacctt ggagttgaag |
| 361 | gaggaagagg acgtgttagt gctgctcgga tcggcctccc ccgccttggc ggccctgacc |
| 421 | ccgctgtcat ccagcgccga cgaagaagag gaggaggagc cgggcgcgtc aggcggggcg |
| 481 | cgtcggcagc gcggggctga ggccgggcag ggggcgcggg gcggcgtggc tgcgggtgcg |
| 541 | gagggctgcc ggcccgcacg gctgctgggt ctggtacacg attgcaaacg gcgcccttcc |
| 601 | cgggcgcggg ccgtctcccg aggcgccaag acggccgaga cggtgcagcg catcaagaag |
| 661 | acccgtagac tgaaggccaa caaccgcgag cgaaaccgca tgcacaacct caacgcggca |
| 721 | ctggacgcgc tgcgcgaggt gctccccacg ttccccgagg acgccaagct caccaagatc |
| 781 | gagaccctgc gcttcgccca caactacatc tgggcactca ccgagaccct gcgcctggcg |
| 841 | gatcactgcg ggggcggcgg cgggggcctg ccgggggcgc tcttctccga ggcagtgttg |
| 901 | ctgagcccgg gaggcgccag cgccgccctg agcagcagcg gagacagccc ctcgcccgcc |
| 961 | tccacgtgga gttgcaccaa cagccccgcg ccgtcctcct ccgtgtcctc caattccacc |
| 1021 | tccccctaca gctgcacttt atcgcccgcc agcccggccg ggtcagacat ggactattgg |
| 1081 | cagcccccac ctcccgacaa gcaccgctat gcacctcacc tccccatagc cagggattgt |
| 1141 | atctagagct gccatttctg ctacccacgc caggccttag tgggttccct ttcctgtccc |
| 1201 | cagtcgagcc ctcctccctt cccctgcccc tcctttccac gccctggaaa ccatctcact |
| 1261 | tcacagggca ggtgtagcct ttctgattcc tcggttgttt cttgcatttc ttggctttgg |
| 1321 | gtatccttca ttcagacggg ctctgattta ctgaaggtgt gatggagctt attgtcaaag |
| 1381 | ccaagggtgg cgttttgggg gcgcttcttg agacgaaaaa gaccctggga agagatgatg |
| 1441 | gtggcatatc taaagagttt gcagagcgga ctgacgctcc tcccctttct ctttaacgcc |
| 1501 | gaaggacttg gtgcagttcg tgtgaatctc acagggggaa tgcaactggt tcctgtgatc |
| 1561 | tcttcacctt tgcttctaca tagagatgtt aatgtcgagt agaaagaaat gtatcttagc |
| 1621 | atctgaatga ttttgctggt aataatatta tccacagatt tgcaatggct ggcatctgct |
| 1681 | ttattcccat tgctgtctgc aggctgtggg aatttcacct gtcaaaccaa acttccctct |
| 1741 | ctgatgtgca ctttgttctg tttcccagat tcgtcacaat gcctattgtc ctgtccttct |
| 1801 | ctttcctttt tcttccccat tttgccatct gtctcttatg atttataagg ggaaaaaaac |
| 1861 | ttgttttgtt agaggggcag gttagaagtc attgtataat ttgtaggctt tgtaatgatt |
| 1921 | gaatgcaagc gtggaaattt aggctgaact ctctatcaaa aggaaaaatg tggaggaaaa |
| 1981 | gggaaaaatc aggagggagg attgcctcat gtattattta tttcgacctt ttaggggaga |
| 2041 | aggaactccc ccattctttc aagagattaa aaataaatca acagtctgaa aacctaagca |
| 2101 | gacacggagc attatccgga tcagccacac acgtgttccc ttctatttat tataaagaaa |
| 2161 | tttttcatgg gaaaatatgt attttttgta tattctacag agtttattct agtatgtatt |
| 2221 | tacatcttga agaacaagaa agttgttctt gtgattaaac tataaataaa ctatctaatt |
| 2281 | ttcataaaaa aaaaaaaaaa aaaaaaaaaa aaaaaaaaaa aaaaaaaaaa aaaaaaaaaa |
| 2341 | aaaaaaaaaa aaaaaaaaaa aaaaaaaaaa |
| Ngn2 (Neurog2) protein human | |
| (SEQ ID No. 28) | |
| MFVKSETLELKEEEDVLVLLGSASPALAALTPLSSSADEEEEEEPGASGGARRQRGAEAGQGARGGVAAGAEGCRP | |
| ARLLGLVHDCKRRPSRARAVSRGAKTAETVQRIKKTRRLKANNRERNRMHNLNAALDALREVLPTFPEDAKLTKIE | |
| TLRFAHNYIWALTETLRLADHCGGGGGGLPGALFSEAVLLSPGGASAALSSSGDSPSPASTWSCTN | |
| SPAPSSSVSSNSTSPYSCTLSPASPAGSDMDYWQPPPPDKHRYAPHLPIARDCI | |
| En1 mouse gene (underline letters denote cDNA boundaries) | |
| (SEQ ID No. 29) | |
| 1 | cgattaaagg cgctgccagc ctcgctctct gggcacagct gagcgtgaca ctggggaagt |
| 61 | caaacccctc tactgcctag gaagatggct agactttaaa gattattttt ttccctttaa |
| 121 | ggaaaaagtc tcggagcttt aaaaaaaatt cctttttctc tttttttttc tcccctcttt |
| 181 | tttttttttt ctgagccgtg gcttatcccc ccattaagac caatcactga aatcttgttg |
| 241 | ctgaaagaaa aaaaagaaag aaagaaaaag aaaagaaaat aatagccaag tgtcttcact |
| 301 | gtatctggat gtctacaaat tagagagagg gagagagcga gatttgctcc accagagcgg |
| 361 | gcgagagcca ggccagacgc tcgcctttct tttttccgcc tgcatccgcc ctgtgccttc |
| 421 | gctgaggctt cgctttgcct tcttcctctc cgcgcacccc cacgggcccg ctggcaaagt |
| 481 | ggggtgggga gcgaggcgcg gggggcgggg gccggcgccg cggccagggc tgccgggcgg |
| 541 | ccgagcatgg aagaacagca gccggagcct aaaagtcagc gcgactcggg cctcggcgcg |
| 601 | gtggcagcgg cggccccgag cggcctcagt ctgagtctga gcccaggagc cagcggcagc |
| 661 | agcggcagcg atggagacag cgtgccggtg tccccgcagc cagcgccccc gtcgcctcct |
| 721 | gcggcaccct gtctgccgcc cctggcccat cacccgcacc tccccccgca tcccccgccc |
| 781 | ccgccgccgc cgccgccgcc gccaccgcag catctcgcgg cgcctgctca ccagccgcag |
| 841 | cccgcggccc agctgcaccg caccaccaac tttttcatcg ataacatcct aaggcccgat |
| 901 | ttcggttgca aaaaggaaca gcccctgcct cagctcctgg tggcttcggc tgcagccgga |
| 961 | ggaggcgcag cagcaggagg aggaagccgc gtggagcgtg accgaggcca gactggtgca |
| 1021 | ggtagagacc ccgttcactc tctgggcaca cgagcttcgg gggctgcctc gctcttgtgt |
| 1081 | gctccagatg cgaactgtgg cccacccgac ggctcccagc ccgccaccgc tgtcagcgcc |
| 1141 | ggcgcatcca aagccgggaa cccggctgct gcggcggccg cggccgcagc agcggctgca |
| 1201 | gcggcagtgg cggcagcggc ggcagcagcc tcgaagccct cggacagtgg cggtggtagt |
| 1261 | ggaggcaacg cggggagtcc cggggcgcag ggcgccaagt tcccggaaca caaccctgcg |
| 1321 | atcctactca tgggttcggc taacggtggg ccggtggtca agactgactc acagcaaccc |
| 1381 | ctagtgtggc ccgcctgggt ctactgcaca cgctattcgg accgtccgtc ctctggtcca |
| 1441 | cgcaccagga agctaaagaa gaaaaagaac gagaaggaag acaagcggcc gcggacggcg |
| 1501 | ttcacggccg agcagctgca gagactcaag gcggagttcc aggcaaaccg ctatatcacg |
| 1561 | gagcagcggc gacagaccct cgcccaggag ctcagcctga atgagtccca gatcaagatc |
| 1621 | tggttccaaa acaagcgtgc caagatcaag aaagccaccg gcatcaagaa cggcctggcg |
| 1681 | ctgcacctca tggcccaggg actgtacaac cactctacca ccacggttca ggacaaagac |
| 1741 | gagagcgagt agctgtggcc agctccgggg cccgcggtcc aacggcgccc gtgccacctc |
| 1801 | caggctcctc ggggctgccg cttcaccagc cccacgcaga gacgatcgct atggagggag |
| 1861 | gcatcaatca gggcgacaga gaaagcgagc aagagaaagc aatcctccga gtggacattc |
| 1921 | acataggaac aaaacggttt ttgaaacggg agtaagactc ggacaggtgc tatgggggaa |
| 1981 | aaataaacat ctattctcta actcactgta taagatgaaa ctgcgaattc cttaaagctc |
| 2041 | tatctagcca aactgctttc gaccgcgtat acatctaatt tcaggtaagg aaaacaaata |
| 2101 | tgtgtagcga tctctatttg ctggacattt ttattaatct catttattat tgttataatt |
| 2161 | attataatta ttataattat ttttcccctc ctccctacct tgctgcaccc ccccccccca |
| 2221 | gcccagtttc gttttcgttg ctcttttcct ttgaatgttt ttgcttctct gggtacctcc |
| 2281 | tgcaccccca acgctggccc tggtttctct gggacttttc tttgtgtgag tgtgagtgtg |
| 2341 | tttccttgtg tgtctgcccc tcgcctcttc tctacccacc caggattctt ctattggtct |
| 2401 | tgtctatccc tcccgtaaat ccccttcctt ttctggagac tccttgagaa atacaacccc |
| 2461 | acagactgcg agactgaacc gccgctacaa gccaaagatt ttattatgtt cagaaacctg |
| 2521 | tagtctgaaa taaaatgttc actgtgttca tgag |
| En1 mouse protein | |
| (SEQ ID No. 30) | |
| MEEQQPEPKSQRDSGLGAVAAAAPSGLSLSLSPGASGSSGSDGDSVPVSPQPAPPSPPAAPCLPPLAHHPHLPPHP | |
| PPPPPPPPPPPQHLAAPAHQPQPAAQLHRTTNFFIDNILRPDFGCKKEQPLPQLLVASAAAGGGAAAGGGSRVERD | |
| RGQTGAGRDPVHSLGTRASGAASLLCAPDANCGPPDGSQPATAVSAGASKAGNPAAAAAAAAAAAA | |
| AAVAAAAAAASKPSDSGGGSGGNAGSPGAQGAKFPEHNPAILLMGSANGGPVVKTDSQQPLVWPAWVYCTRYSDRP | |
| SSGPRTRKLKKKKNEKEDKRPRTAFTAEQLQRLKAEFQANRYITEQRRQTLAQELSLNESQIKIWFQNKRAKIKKA | |
| TGIKNGLALHLMAQGLYNHSTTTVQDKDESE | |
| En1 gene human (underline letters denote cDNA boundaries) | |
| (SEQ ID No. 31) | |
| 1 | agctcacaga cccataatcc tgcatttctc taacaagttg tttatggagt tgcttctcca |
| 61 | tttgcctaca tcccaaaatt cacccctccc gggtttcttc tgccccctcc tgagtcccgg |
| 121 | cctgaaggag ggggagggac gcgggtgcgg gcgcgggtgg gggagggcgg acccgacgca |
| 181 | cagggccagc gccgaggcgc cccctctccg ccagcggttg acgcccccgg attatttatc |
| 241 | cgcaaagtcc cgcgcgcgcc cattgggccg aggcccgagt gtcagcgcga gtcccggctc |
| 301 | gccattggct ccgcacacgt gcggccctga ctcacgtgct tccggtttga aggcaaaaag |
| 361 | tgtgcctggg tgattttttt tttaagcgag agagtttgtg caaagatccg agctgtcaga |
| 421 | gatttgaaaa aaaaaaaaaa aaaaaaaaaa aaaaaaacag cccggcgctg gcggagacgc |
| 481 | gctctccctg caaaaaaagc aaaggcgact aaaggcgctg ccagcctcac gctctgggca |
| 541 | cagctgagcg tgacactcgg ggaagtcaaa cccctcacta ctgcctagga agatggctag |
| 601 | actttaaata ctattttttt ccctttaaga aaaaaattat tggagctttt tttcttgctt |
| 661 | tctttttcct tttctttttc tttttttcct tcattttttt ggccgtggct tactccccat |
| 721 | ttaaatcaaa tcattgaatc tggttgcaga aagaaaaaag aaatagccaa gtgtctccat |
| 781 | atctggatgt ctacaaatta gagagggaga gacagcgaga tctatctgct agataagaac |
| 841 | gagcgatcca ggccagacgc ctgagctttt ttcctgcacc cgccccgtgc cttcgctgag |
| 901 | gcttcgcctg cctccttcct ccgcgcaccc ccacgggccg ctggcaaagt ggggtgggga |
| 961 | gcgaggcggt gggggcgggg gccggcgcgg cggccggggc ggcggggcgg ccgagcatgg |
| 1021 | aagaacagca gccggaacct aaaagtcagc gcgactcggc cctcggcgcg gcggcggcgg |
| 1081 | cgactccggg cggcctcagc ctgagcctca gtccgggcgc cagcggcagc agcggcagcg |
| 1141 | gcagcgatgg agacagcgtg ccggtgtccc cgcagcctgc gcccccctcg ccgcccgcgg |
| 1201 | cgccttgcct gccgcccctg gcccaccacc cgcacctccc cccacacccc ccgcccccgc |
| 1261 | cgcctcagca tctcgcggcg cctgctcacc agccgcagcc agcggcccag ctgcaccgca |
| 1321 | ccaccaactt tttcatcgac aacatcctga ggccggactt cggctgcaaa aaggagcagc |
| 1381 | cgccaccgca gcttctggtg gctgcggcgg ccagaggagg cgcaggagga ggaggccggg |
| 1441 | tcgagcgtga cagaggccag actgccgcag gtagagaccc tgtccacccg ttgggcaccc |
| 1501 | gggcgccagg cgctgcctcg ctcctgtgcg ccccggacgc gaactgtggc ccacccgacg |
| 1561 | gctcccagcc agccgccgcc ggcgcgggcg cgtctaaagc tgggaacccg gctgcggcgg |
| 1621 | cggcggcggc cgcggcggca gtggcggcgg cggcggcggc cgcagcagcc aagccctcgg |
| 1681 | acaccggtgg cggcggcagt ggaggcggcg cggggagccc cggagcgcag ggcaccaaat |
| 1741 | acccggagca cggcaacccg gctatcctac ttatgggctc agccaacggc gggcccgtgg |
| 1801 | tcaaaactga ctcgcagcag cctctcgtat ggcccgcctg ggtgtactgc acacgttatt |
| 1861 | cggatcgtcc atcctccggt ccgcgcacca ggaagctgaa gaagaagaag aacgagaagg |
| 1921 | aggacaagcg gccgcggacc gcgttcacgg ccgagcagct gcagagactc aaggcggagt |
| 1981 | tccaggcaaa ccgctacatc acggagcagc ggcggcagac cctggcccag gaactcagcc |
| 2041 | tcaacgagtc ccagatcaag atctggttcc agaacaagcg cgccaagatc aagaaagcca |
| 2101 | caggcatcaa gaacggcctg gcgctgcacc tcatggccca gggactgtac aaccactcca |
| 2161 | ccaccacggt ccaggacaaa gacgagagcg agtagccgcc acaggccggg gccgcgcccg |
| 2221 | cgccccctcc cggcaccgcc gccgtcgtct cccggcccct cgctggggga gaaagcatct |
| 2281 | gctccaagga gggagggagc gcagggaaaa gagcgagaga gacagaaaga gagcctcaga |
| 2341 | atggacaatg acgttgaaac gcagcatttt tgaaaaggga gaaagactcg gacaggtgct |
| 2401 | atcgaaaaat aagatccatt ctctattccc agtataaggg acgaaactgc gaactcctta |
| 2461 | aagctctatc tagccaaacc gcttacgacc ttgtatatat ttaatttcag gtaaggaaaa |
| 2521 | cacatacgtg tagcgatctc tatttgctgg acatttttat taatctcctt tattattatt |
| 2581 | gttataatta ttataattat tataattatt ttatcccctc ccccaccgcc tcgctgcccc |
| 2641 | cgcccagttt cgttttcgtt gcctttttca tttgaatgtc attgcttctc cggtgcctcc |
| 2701 | cgacccgcat cgccggccct ggtttctctg ggacttttct ttgtgtgcga gagtgtgttt |
| 2761 | cctttcgtgt ctgcccacct cttctccccc acctcccggg tcccttctgt cggtctgtct |
| 2821 | gttctgcccc cctttcgttt tccggagact tgttgagaaa tacgacccca cagactgcga |
| 2881 | gactgaaccg ccgctacaag ccaaagattt tattatgttc agaaacctgt agtctgaaat |
| 2941 | aaagtgtaca ctgtgctcac ga |
| En1 protein human | |
| (SEQ ID No. 32) | |
| MEEQQPEPKSQRDSALGAAAAATPGGLSLSLSPGASGSSGSGSDGDSVPVSPQPAPPSPPAAPCLPPLAHHPHLPP | |
| HPPPPPPQHLAAPAHQPQPAAQLHRTTNFFIDNILRPDFGCKKEQPPPQLLVAAAARGGAGGGGRVERDRGQTAAG | |
| RDPVHPLGTRAPGAASLLCAPDANCGPPDGSQPAAAGAGASKAGNPAAAAAAAAAAVAAAAAAAAA | |
| KPSDTGGGGSGGGAGSPGAQGTKYPEHGNPAILLMGSANGGPVVKTDSQQPLVWPAWVYCTRYSDRPSSGPRTRKL | |
| KKKKNEKEDKRPRTAFTAEQLQRLKAEFQANRYITEQRRQTLAQELSLNESQIKIWFQNKRAKIKKATGIKNGLAL | |
| HLMAQGLYNHSTTTVQDKDESE | |
| Foxa2 mouse gene (underline letters denote cDNA boundaries) | |
| (SEQ ID No. 33) | |
| 1 | ctgacgacca gggcggccag accacgcgag tcctacgcgc ctcctgaggc cgccccggga |
| 61 | cttaactgta acggggaggg gcctccggag cagcggccag cgagttaaag tatgctggga |
| 121 | gccgtgaaga tggaagggca cgagccatcc gactggagca gctactacgc ggagcccgag |
| 181 | ggctactctt ccgtgagcaa catgaacgcc ggcctgggga tgaatggcat gaacacatac |
| 241 | atgagcatgt ccgcggctgc catgggcggc ggttccggca acatgagcgc gggctccatg |
| 301 | aacatgtcat cctatgtggg cgctggaatg agcccgtcgc tagctggcat gtccccgggc |
| 361 | gccggcgcca tggcgggcat gagcggctca gccggggcgg ccggcgtggc gggcatggga |
| 421 | cctcacctga gtccgagtct gagcccgctc gggggacagg cggccggggc catgggtggc |
| 481 | cttgccccct acgccaacat gaactcgatg agccccatgt acgggcaggc cggcctgagc |
| 541 | cgcgctcggg accccaagac ataccgacgc agctacacac acgccaaacc tccctactcg |
| 601 | tacatctcgc tcatcaccat ggccatccag cagagcccca acaagatgct gacgctgagc |
| 661 | gagatctatc agtggatcat ggacctcttc cctttctacc ggcagaacca gcagcgctgg |
| 721 | cagaactcca tccgccactc tctctccttc aacgactgct ttctcaaggt gccccgctcg |
| 781 | ccagacaagc ctggcaaggg ctccttctgg accctgcacc cagactcggg caacatgttc |
| 841 | gagaacggct gctacctgcg ccgccagaag cgcttcaagt gtgagaagca actggcactg |
| 901 | aaggaagccg cgggtgcggc cagtagcgga ggcaagaaga ccgctcctgg gtcccaggcc |
| 961 | tctcaggctc agctcgggga ggccgcgggc tcggcctccg agactccggc gggcaccgag |
| 1021 | tccccccatt ccagcgcttc tccgtgtcag gagcacaagc gaggtggcct aagcgagcta |
| 1081 | aagggagcac ctgcctctgc gctgagtcct cccgagccgg cgccctcgcc tgggcagcag |
| 1141 | cagcaggctg cagcccacct gctgggccca cctcaccacc caggcctgcc accagaggcc |
| 1201 | cacctgaagc ccgagcacca ttacgccttc aaccacccct tctctatcaa caacctcatg |
| 1261 | tcgtccgagc agcaacatca ccacagccac caccaccatc agccccacaa aatggacctc |
| 1321 | aaggcctacg aacaggtcat gcactaccca gggggctatg gttcccccat gccaggcagc |
| 1381 | ttggccatgg gcccagtcac gaacaaagcg ggcctggatg cctcgcccct ggctgcagac |
| 1441 | acttcctact accaaggagt gtactccagg cctattatga actcatccta agaagatggc |
| 1501 | tttcaggccc tgctagctct ggtcactggg gacaagggaa atgagaggct gagtggagac |
| 1561 | tttgggagag ctttgaggaa aagtagccac cacacttcag gcctcaaggg agcagtctca |
| 1621 | cctgtctgtg tccctaaata gatgggccac agtgatctgt cattctaaat agggaaggga |
| 1681 | atggaaatat atatgtatac atataaactt gttttaaagg agcctttggt ctcctctatg |
| 1741 | tagactactg cttctcaaga catctgcaga gtttgatttt tgttgttgtt ctctattgct |
| 1801 | gttgttgcag aaaagtctga ctttaaaaac aaacaaacaa acaaaaaact tttgtgagtg |
| 1861 | acttggtgta aaaccatgta gttttaacag aaaaccagag ggttgtactg atgttgaaaa |
| 1921 | gaggaaagaa aaataatgta agagtctggt gtaccggacc aggagaaagg agaaaaacac |
| 1981 | atcccattct ggacatggtg aaatccaggt ctcgggtctg atttaattta tggtttctgc |
| 2041 | gtgctttatt tatggcttat aaatgtgtgt tctggctaga atggccagaa ttccacaaat |
| 2101 | ctatattaaa gtgttattgc cgatttt |
| Foxa2 mouse protein | |
| (SEQ ID No. 34) | |
| MLGAVKMEGHEPSDWSSYYAEPEGYSSVSNMNAGLGMNGMNTYMSMSAAAMGGGSGNMSAGSMNMSSYVGAGMSPS | |
| LAGMSPGAGAMAGMSGSAGAAGVAGMGPHLSPSLSPLGGQAAGAMGGLAPYANMNSMSPMYGQAGLSRARDPKTYR | |
| RSYTHAKPPYSYISLITMAIQQSPNKMLTLSEIYQWIMDLFPFYRQNQQRWQNSIRHSLSFNDCFL | |
| KVPRSPDKPGKGSFWTLHPDSGNMFENGCYLRRQKRFKCEKQLALKEAAGAASSGGKKTAPGSQASQAQLGEAAGS | |
| ASETPAGTESPHSSASPCQEHKRGGLSELKGAPASALSPPEPAPSPGQQQQAAAHLLGPPHHPGLPPEAHLKPEHH | |
| YAFNHPFSINNLMSSEQQHHHSHHHHQPHKMDLKAYEQVMHYPGGYGSPMPGSLAMGPVTNKAGLDASPLAADTSY | |
| YQGVYSRPIMNSS | |
| Foxa2 human gene (underline letters denote cDNA boundaries) | |
| (SEQ ID No. 35) | |
| 1 | cccgcccact tccaactacc gcctccggcc tgcccaggga gagagaggga gtggagccca |
| 61 | gggagaggga gcgcgagaga gggagggagg aggggacggt gctttggctg actttttttt |
| 121 | aaaagagggt gggggtgggg ggtgattgct ggtcgtttgt tgtggctgtt aaattttaaa |
| 181 | ctgccatgca ctcggcttcc agtatgctgg gagcggtgaa gatggaaggg cacgagccgt |
| 241 | ccgactggag cagctactat gcagagcccg agggctactc ctccgtgagc aacatgaacg |
| 301 | ccggcctggg gatgaacggc atgaacacgt acatgagcat gtcggcggcc gccatgggca |
| 361 | gcggctcggg caacatgagc gcgggctcca tgaacatgtc gtcgtacgtg ggcgctggca |
| 421 | tgagcccgtc cctggcgggg atgtcccccg gcgcgggcgc catggcgggc atgggcggct |
| 481 | cggccggggc ggccggcgtg gcgggcatgg ggccgcactt gagtcccagc ctgagcccgc |
| 541 | tcggggggca ggcggccggg gccatgggcg gcctggcccc ctacgccaac atgaactcca |
| 601 | tgagccccat gtacgggcag gcgggcctga gccgcgcccg cgaccccaag acctacaggc |
| 661 | gcagctacac gcacgcaaag ccgccctact cgtacatctc gctcatcacc atggccatcc |
| 721 | agcagagccc caacaagatg ctgacgctga gcgagatcta ccagtggatc atggacctct |
| 781 | tccccttcta ccggcagaac cagcagcgct ggcagaactc catccgccac tcgctctcct |
| 841 | tcaacgactg ccucctgaag gcgccccgcc cgcccgacaa gcccggcaag ggctccttct |
| 901 | ggaccctgca ccctgactcg ggcaacatgt tcgagaacgg ctgctacctg cgccgccaga |
| 961 | agcgcttcaa gtgcgagaag cagctggcgc tgaaggaggc cgcaggcgcc gccggcagcg |
| 1021 | gcaagaaggc ggccgccgga gcccaggcct cacaggctca actcggggag gccgccgggc |
| 1081 | cggcctccga gactccggcg ggcaccgagt cgcctcactc gagcgcctcc ccgtgccagg |
| 1141 | agcacaagcg agggggcctg ggagagctga aggggacgcc ggctgcggcg ctgagccccc |
| 1201 | cagagccggc gccctctccc gggcagcagc agcaggccgc ggcccacctg ctgggcccgc |
| 1261 | cccaccaccc gggcctgccg cctgaggccc acctgaagcc ggaacaccac tacgccttca |
| 1321 | accacccgtt ctccatcaac aacctcatgt cctcggagca gcagcaccac cacagccacc |
| 1381 | accaccacca accccacaaa atggacctca aggcctacga acaggtgatg cactaccccg |
| 1441 | gctacggttc ccccatgcct ggcagcttgg ccatgggccc ggtcacgaac aaaacgggcc |
| 1501 | tggacgcctc gcccctggcc gcagatacct cctactacca gggggtgtac tcccggccca |
| 1561 | ttatgaactc ctcttaagaa gacgacggct tcaggcccgg ctaactctgg caccccggat |
| 1621 | cgaggacaag tgagagagca agtgggggtc gagactttgg ggagacggtg ttgcagagac |
| 1681 | gcaagggaga agaaatccat aacaccccca ccccaacacc cccaagacag cagtcttctt |
| 1741 | cacccgctgc agccgttccg tcccaaacag agggccacac agatacccca cgttctatat |
| 1801 | aaggaggaaa acgggaaaga atataaagtt aaaaaaaagc ctccggtttc cactactgtg |
| 1861 | tagactcctg cttcttcaag cacctgcaga ttctgatttt tttgttgttg ttgttctcct |
| 1921 | ccattgctgt tgttgcaggg aagtcttact taaaaaaaaa aaaaaatttt gtgagtgact |
| 1981 | cggtgtaaaa ccatgtagtt ttaacagaac cagagggttg tactattgtt taaaaacagg |
| 2041 | aaaaaaaata atgtaagggt ctgttgtaaa tgaccaagaa aaagaaaaaa aaagcattcc |
| 2101 | caatcttgac acggtgaaat ccaggtctcg ggtccgatta atttatggtt tctgcgtgct |
| 2161 | ttatttatgg cttataaatg tgtattctgg ctgcaagggc cagagttcca caaatctata |
| 2221 | ttaaagtgtt atacccggtt ttatcccttg aatcttttct tccagatttt tcttttcttt |
| 2281 | acttggctta caaaatatac aggcttggaa attatttcaa gaaggaggga gggataccct |
| 2341 | gtctggttgc aggttgtatt ttattttggc ccagggagtg ttgctgtttt cccaacattt |
| 2401 | tattaataaa attttcagac ataaaaaa |

Foxa2 human protein
(SEQ ID No. 36)
MHSASSMLGAVKMEGHEPSDWSSYYAEPEGYSSVSNMNAGLGMNGMNTYMSMSAAAMGSGSGNMSAGSMNMSSYVG
AGMSPSLAGMSPGAGAMAGMGGSAGAAGVAGMGPHLSPSLSPLGGQAAGAMGGLAPYANMNSMSPMYGQAGLSRAR
DPKTYRRSYTHAKPPYSYISLITMAIQQSPNKMLTLSEIYQWIMDLFPFYRQNQQRWQNSIRHSLS
FNDCFLKVPRSPDKPGKGSFWTLHPDSGNMFENGCYLRRQKRFKCEKQLALKEAAGAAGSGKKAAAGAQASQAQLG
EAAGPASETPAGTESPHSSASPCQEHKRGGLGELKGTPAAALSPPEPAPSPGQQQQAAAHLLGPPHHPGLPPEAHL
KPEHHYAFNHPFSINNLMSSEQQHHHSHHHHQPHKMDLKAYEQVMHYPGYGSPMPGSLAMGPVTNKTGLDASPLAA
DTSYYQGVYSRPIMNSS
Brn2 mouse cDNA
(SEQ ID No. 37)
atggc
gaccgcagcg tctaaccact acagcctgct cacctccagc gcctccatcg tacatgccga
gccgcctggc ggcatgcagc agggcgcagg gggctaccgc gaggcgcaga gcctggtgca
gggcgaccac ggcgcgctgc agagcaacgg gcacccgctc agccacgctc accagtggat
caccgcgctg tcccacggcg gcggcggcgg gggcggcggc ggcggtggag gaggcggggg
aggcggcggg ggaggcggcg acggctcccc gtggtccacc agccccctag gccagccgga
catcaagccc tcggtggtgg tacagcaggg tggccgaggc gacgagctgc acgggccagg
agcgctgcag caacagcatc aacagcaaca gcaacagcag cagcagcagc agcagcagca
gcagcagcaa cagcagcagc aacaacagcg accgccacat ctggtgcacc acgctgccaa
ccaccatccc gggcccgggg catggcggag tgcggcggct gcagctcacc tccctccctc
catgggagct tccaacggcg gtttgctcta ttcgcagccg agcttcacgg tgaacggcat
gctgggcgca ggagggcagc cggctgggct gcaccaccac ggcctgaggg acgcccacga
tgagccacac catgcagacc accacccgca tccgcactct cacccacacc agcaaccgcc
cccgccacct cccccacaag gcccaccggg ccacccaggc gcgcaccacg acccgcactc
ggacgaggac acgccgacct cagacgacct ggagcagttc gccaagcaat tcaagcagag
gcggatcaaa ctcggattta ctcaagcaga cgtggggctg gcgcttggca ccctgtacgg
caacgtgttc tcgcagacca ccatctgcag gtttgaggcc ctgcagctga gcttcaagaa
catgtgcaag ctgaagcctt tgttgaacaa gtggttggaa gaggcagact catcctcggg
cagccccacc agcatagaca agatcgcagc gcaagggcgc aaacggaaaa agcggacctc
catcgaggtg agcgtcaagg gggctctgga gagccatttc ctcaaatgcc ctaagccctc
ggcccaggag atcacctccc tcgcggacag cttacagctg gagaaggagg tggtgagagt
ttggttttgt aacaggagac agaaagagaa aaggatgacc cctcccggag ggactctgcc
gggcgccgag gatgtgtatg ggggtagtag ggacacgcca ccacaccacg gggtgcagac
gcccgtccag tga
Brn2 mouse protein
(SEQ ID No. 38)
MATAASNHYSLLTSSASIVHAEPPGGMQQGAGGYREAQSLVQGDYGALQSNGHPLSHAHQWITALSHGGGGGGGGG
GGGGGGGGGGGGDGSPWSTSPLGQPDIKPSVVVQQGGRGDELHGPGALQQQHQQQQQQQQQQQQQQQQQQQQQQQR
PPHLVHHAANHHPGPGAWRSAAAAAHLPPSMGASNGGLLYSQPSFTVNGMLGAGGQPAGLHHHGLR
DAHDEPHHADHHPHPHSHPHQQPPPPPPPQGPPGHPGAHHDPHSDEDTPTSDDLEQFAKQFKQRRIKLGFTQADVG
LALGTLYGNVFSQTTICRFEALQLSFKNMCKLKPLLNKWLEEADSSSGSPTSIDKIAAQGRKRKKRTSIEVSVKGA
LESHFLKCPKPSAQEITSLADSLQLEKEVVRVWFCNRRQKEKRMTPPGGTLPGAEDVYGGSRDTPPHHGVQTPVQ
Brn2 human cDNA
(SEQ ID No. 39)
atggcgaccg cagcgtctaa ccactacagc ctgctcacct ccagcgcctc catcgtgcac gccgagccgc
ccggcggcat gcagcagggc gcggggggct accgcgaagc gcagagcctg gtgcagggcg actacggcgc
tctgcagagc aacggacacc cgctcagcca cgctcaccag tggatcaccg cgctgtccca cggcggcggc
ggcgggggcg gtggcggcgg cggggggggc gggggcggcg gcgggggcgg cggcgacggc tccccgtggt
ccaccagccc cctgggccag ccggacatca agccctcggt ggtggtgcag cagggcggcc gcggagacga
gctgcacggg ccaggcgccc tgcagcagca gcatcagcag cagcaacagc aacagcagca gcaacagcag
caacagcagc agcagcagca gcaacagcgg ccgccgcatc tggtgcacca cgccgctaac caccacccgg
gacccggggc atggcggagc gcggcggctg cagcgcacct cccaccctcc atgggagcgt ccaacggcgg
cttgctctac tcgcagccca gcttcacggt gaacggcatg ctgggcgccg gcgggcagcc ggccgggctg
caccaccacg gcctgcggga cgcgcacgac gagccacacc atgccgacca ccacccgcac ccgcactcgc
acccacacca gcagccgccg cccccgccgc ccccgcaggg tccgcctggc cacccaggcg cgcaccacga
cccgcactcg gacgaggaca cgccgacctc ggacgacctg gagcagttcg ccaagcagtt caagcagcgg
cggatcaaac tgggatttac ccaagcggac gtggggctgg ctctgggcac cctgtatggc aacgtgttct
cgcagaccac catctgcagg tttgaggccc tgcagctgag cttcaagaac atgtgcaagc tgaagccttt
gttgaacaag tggttggagg aggcggactc gtcctcgggc agccccacga gcatagacaa gatcgcagcg
caagggcgca agcggaaaaa gcggacctcc atcgaggtga gcgtcaaggg ggctctggag agccatttcc
tcaaatgccc caagccctcg gcccaggaga tcacctccct cgcggacagc ttacagctgg agaaggaggt
ggtgagagtt tggttttgta acaggagaca gaaagagaaa aggatgaccc ctcccggagg gactctgccg
ggcgccgagg atgtgtacgg ggggagtagg gacactccac cacaccacgg ggtgcagacg cccgtccagt
ga
Brn2 human protein
(SEQ ID No. 40)
MATAASNHYSLLTSSASIVHAEPPGGMQQGAGGYREAQSLVQGDYGALQSNGHPLSHAHQWITALSHGGGGGGGGG
GGGGGGGGGGGGDGSPWSTSPLGQPDIKPSVVVQQGGRGDELHGPGALQQQHQQQQQQQQQQQQQQQQQQQQQRPP
HLVHHAANHHPGPGAWRSAAAAAHLPPSMGASNGGLLYSQPSFTVNGMLGAGGQPAGLHHHGLRDA
HDEPHHADHHPHPHSHPHQQPPPPPPPQGPPGHPGAHHDPHSDEDTPTSDDLEQFAKQFKQRRIKLGFTQADVGLA
LGTLYGNVFSQTTICRFEALQLSFKNMCKLKPLLNKWLEEADSSSGSPTSIDKIAAQGRKRKKRTSIEVSVKGALE
SHFLKCPKPSAQEITSLADSLQLEKEVVRVWFCNRRQKEKPMTPPGGTLPGAEDVYGGSRDTPPHHGVQTPVQ
Myt11 mouse cDNA
(SEQ ID No. 41)
atgg acgtggactc tgaggagaag cgccatcgca cacggtccaa aggggttcga
gttcctgtgg agccagccat acaagagctg ttcagctgtc ccactccagg ctgcgacggc
agtggtcacg tcagtggcaa atatgcacga cacagaagtg tatatggttg tcccttggct
aaaaaaagaa aaacgcaaga taaacagccc caagaacctg ctcccaagcg aaaaccattt
gcagtaaaag cagatagttc ctcagtagac gaatgttatg agagtgatgg tactgaagac
atggatgata aggaggaaga tgatgatgag gagttctctg aagacaatga tgagcaaggg
gatgatgacg acgaagatga ggtggatcgg gaagacgagg aggagatcga ggaggaagat
gatgaagaag atgatgatga tgaagatggt gacgatgtag aagaggaaga agaggatgat
gatgaagagg aggaagaaga ggaagaggaa gaagaaaatg aagaccatca aatgagttgt
actcgaataa tgcaggacac agacaaggat gataacaaca atgatgagta tgataactat
gatgaactgg tagctaagtc gctattaaat cttggcaaaa ttgctgagga tgcagcatac
cgagccagga ctgaatcaga gatgaacagc aatacctcca atagtctgga ggacgatagt
gacaaaaacg aaaacctcgg tcggaaaagc gaactgagtc tagacttaga cagtgatgtt
gttagagaaa cagtggactc ccttaagctg ttagcacaag gacatggtgt tgtgctatca
gagaatatca gtgacagaag ttatgctgag gggatgtcac agcaggacag tagaaatatg
aactatgtca tgctagggaa gcccatgaac aatggactca tggagaagat ggtggaggag
agtgatgagg aagtgtgtct aagtagtcta gagtgcctga ggaaccagtg ctttgacctg
gccaggaaac tcagcgagac caacccacag gacaggagtc agccacccaa catgagtgtg
cgccaacatg tccggcaaga ggacgacttc cctgggagga cgccagacag gagctactcg
gatatgatga accttatgcg gctggaggag cagctcagtc ccaggtctag aacgttctcc
agctgtgcca aggaggatgg gtgtcatgag agggatgatg acaccacctc agtgaactca
gacaggtctg aggaagtgtt tgacatgacc aagggcaacc tgactctgct agagaaagcc
attgccttgg agacagagag agccaaggcc atgcgggaga agatggccat ggatgctggg
agaagggata acctgagatc ctatgaggac cagtctccaa gacagctggc tggggaagac
agaaaatcca aatccagtga cagccatgtc aaaaagccat actatggtaa agatccctca
agaacagaaa agagagagag caagtgtcca acccccgggt gtgatggaac cggccacgta
actgggcttt acccgcatca ccgcagtctg tctggatgcc cgcacaaaga tagggtccct
ccagaaattc ttgccatgca tgaaaatgtt ctcaagtgtc ccactccagg ctgcacaggg
cgagggcatg tgaatagcaa caggaactcg cacagaagcc tctctggatg ccccattgct
gctgcagaaa aactggcaaa ggcccaagag aaacaccaga gctgtgatgt gtccaaatcc
aaccaggcct cagaccgagt cctcaggcca atgtgctttg tcaaacagct tgagattcct
cagtatggct acagaaacaa tgttcccaca accacaccac gctccaacct ggccaaggag
cttgagaaat actccaagac ttcgtttgag tacaacagtt acgacaacca tacttatggc
aaaagagcca tagctcccaa ggtgcaaacc agggacatat cccccaaagg atatgacgat
gccaagcggt actgcaagaa tgccagcccc agcagcagca ccaccagcag ctatgcacct
agcagcagca gcaacctcag ctgtggtggt ggcagcagcg ccagtagcac gtgtagcaag
agcagctttg actacacaca tgacatggag gccgcacaca tggcagccac agccattctc
aacctgtcca cacgttgtcg tgaaatgcca cagaacctgt ccaccaagcc acaggacctg
tgtactgccc ggaacccaga catggaggtg gatgagaatg gcaccctgga cctgagcatg
aacaagcaga ggcctcgaga cagctgctgc ccagtcctga cacccctgga acccatgtct
ccgcagcagc aggccgtgat gagcagccga tgcttccagc tgagcgaggg ggattgctgg
gacttgcctg tagactacac caaaatgaag cctcggaggg tagatgagga tgagcccaaa
gagattaccc cagaagactt ggacccattc caggaggctc tggaagaaag acggtatcca
ggggaggtga ccatcccaag ccccaaaccc aagtaccctc agtgcaagga aagcaaaaag
gacttaataa ctctgtctgg ctgccccctg gcggacaaaa gcattcgaag tatgctggcc
accagttccc aagagctcaa gtgccccacc cctggctgtg acggttctgg acacatcact
ggcaattacg cttctcatcg aagcctttct gggtgcccga gagcaaagaa gagtggcatc
cggatagcac agagcaaaga ggacaaggaa gaccaggagc caatcaggtg tccggtacct
ggctgtgacg gtcagggaca catcactggg aagtatgcat cccaccgcag cgcctccggg
tgtcccttgg cagccaagag gcagaaagat gggtacctta atggctccca gttctcctgg
aagtcggtca agacggaggg catgtcctgc cctacccccg ggtgtgatgg gtcaggacac
gtcagtggca gcttcctcac acaccgcagc ttgtcaggat gtccaagagc cacatcagca
atgaagaaag caaagctgtc tggagaacag atgttgacta tcaagcagcg agccagcaac
ggtatagaaa atgatgaaga aatcaagcag ttagatgaag agatcaagga gcttaatgag
tccaattccc agatggaggc tgacatgatc aaactcagaa ctcagatcac cacaatggag
agcaacctga agacgattga ggaggagaac aaagtcattg aacagcagaa tgagtcgctc
ttgcacgagt tggccaacct gagccagtcc ctgatccaca gcctcgccaa catccagctg
cctcacatgg atccaatcaa tgaacaaaat tttgatgctt acgtgactac tttgacggaa
atgtatacaa atcaagatcg ttatcagagt ccagaaaata aagccctact ggaaaatata
aagcaggctg tgagaggaat tcaggtctga
Myt11 mouse protein
(SEQ ID No. 42)
MDVDSEEKRHRTRSKGVRVPVEPAIQELFSCPTPGCDGSGHVSGKYARHRSVYGCPLAKKRKTQDKQPQEPAPKRK
PFAVKADSSSVDECYESDGTEDMDDKEEDDDEEFSEDNDEQGDDDDEDEVDREDEEEIEEEDDEEDDDDEDGDDVE
EEEEDDDEEEEEEEEEEENEDHQMSCTRIMQDTDKDDNNNDEYDNYDELVAKSLLNLGKIAEDAAY
RARTESEMNSNTSNSLEDDSDKNENLGRKSELSLDLDSDVVRETVDSLKLLAQGHGVVLSENISDRSYAEGMSQQD
SRNMNYVMLGKPMNNGLMEKMVEESDEEVCLSSLECLRNQCFDLARKLSETNPQDRSQPPNMSVPQHVPQEDDFPG
RTPDRSYSDMMNLMRLEEQLSPRSRTFSSCAKEDGCHERDDDTTSVNSDRSEEVFDMTKGNLTLLEKAIALETERA
KAMREKMAMDAGRRDNLRSYEDQSPRQIAGEDRKSKSSDSHVKKPYYGKDPSRTEKRESKCP
TPGCDGTGHVTGLYPHHRSLSGCPHKDRVPPEILAMHENVLKCPTPGCTGRGHVNSNRNSHRSLSGCPIAAAEKLA
KAQEKHQSCDVSKSNQASDRVLRPMCFVKQLEIPQYGYRNNVPTTTPRSNLAKELEKYSKTSFEYNSYDNHTYGKR
AIAPKVQTRDISPKGYDDAKRYCKNASPSSSTTSSYAPSSSSNLSCGGGSSASSTCSKSSFDYTHDMEAAHMAATA
ILNLSTRCREMPQNLSTKPQDLCTARNPDMEVDENGTLDLSMNKQRPRDSCCPVLTPLEPMS
PQQQAVMSSRCFQLSEGDCWDLPVDYTKMKPRRVDEDEPKEITPEDLDPFQEALEERRYPGEVTIPSPKPKYPQCK
ESKKDLITLSGCPLADKSIRSMLATSSQELKCPTPGCDGSGHITGNYASHRSLSGCPRAKKSGIRIAQSKEDKEDQ
EPIRCPVPGCDGQGHITGKYASHRSASGCPLAAKRQKDGYLNGSQFSWKSVKTEGMSCPTPGCDGSGHVSGSFLTH
RSLSGCPRATSAMKKAKLSGEQMLTIKQRASNGIENDEEIKQLDEEIKELNESNSQMEADMIKLRTQITTMESNLK
TIEEENKVIEQQNESLLHELANLSQSLIHSLANIQLPHMDPINEQNFDAYVTTLTEMYTNQDRYQSPENKALLENI
KQAVRGIQV
Myt11 human cDNA
(SEQ ID No. 43)
atg gaggtggaca ccgaggagaa gcggcatcgc acgcggtcca aaggggttcg agttcccgtg
gaaccagcca tacaagagct gttcagctgt cccacccctg gctgtgacgg cagtggtcat gtcagtggca
aatatgcaag acacagaagt gtatatggtt gtcccttggc gaaaaaaaga aaaacacaag ataaacagcc
ccaggaacct gctcctaaac gaaagccatt tgccgtgaaa gcagacagct cctcagtgga tgagtgtgac
gacagtgatg ggactgagga catggatgag aaggaggagg atgaggggga ggagtactcc gaggacaatg
acgagccagg ggatgaggac gaggaggacg aggaggggga ccgggaggag gaggaggaga tcgaggagga
ggatgaggac gatgacgagg atggagaaga tgtggaggat gaagaagagg aagaggagga ggaggaggag
gaggaagagg aagaagaaaa cgaagaccat caaatgaatt gtcacaatac tcgaataatg caagacacag
aaaaggatga taacaataat gacgaatatg acaattacga tgaactggtg gccaagtcat tgttaaacct
cggcaaaatc gctgaggatg cagcctaccg ggccaggact gagtcagaaa tgaacagcaa tacctccaat
agtctggaag acgatagcga caaaaacgaa aacctgggtc ggaaaagtga gttgagttta gacttagaca
gtgatgttgt tagagaaaca gtggactccc ttaaactatt agcccaagga cacggtgtcg tgctctcaga
aaacatgaat gacagaaatt atgcagacag catgtcgcag caagacagta gaaatatgaa ttacgtcatg
ttggggaagc ccatgaacaa cggactcatg gaaaagatgg tggaggagag cgatgaggag gtgtgtctga
gcagtctgga gtgtttgagg aatcagtgct tcgacctggc caggaagctc agtgagacca acccgcagga
gaggaatccg cagcagaaca tgaacatccg tcagcatgtc cggccagaag aggacttccc cggaaggacg
ccggacagaa actactcgga catgctgaac ctcatgcggc tggaggagca gttgagcccc cggtcgagag
tgtttgccag ctgtgcgaag gaggatgggt gtcatgagcg ggacgacgat accacctctg tgaactcgga
caggtctgaa gaggtgttcg acatgaccaa ggggaacctg accctgctgg agaaagccat cgctttggaa
acggaaagag caaaggccat gagggagaag atggccatgg aagctgggag gagggacaat atgaggtcat
atgaggacca gtctccgaga caacttcccg gggaggacag aaagcctaaa tccagtgaca gccatgtcaa
aaagccatac tatgatccct caagaacaga aaagaaagag agcaagtgtc caacccccgg gtgtgatgga
accggccacg taactgggct gtacccacat caccgcagcc tgtccggatg cccgcacaaa gatagggtcc
ctccagaaat ccttgccatg catgaaagtg tcctcaagtg ccccactccg ggctgcacgg ggcgcgggca
tgtcaacagc aacaggaact cccaccgaag cctctccgga tgcccgatcg ctgcagcaga gaaactggcc
aaggcacagg aaaagcacca gagctgcgac gtgtccaagt ccagccaggc ctcggaccgc gtgctcaggc
caatgtgctt tgtgaagcag ctggagattc ctcagtatgg ctacagaaac aatgtcccca caactacgcc
gcgttccaac ctggccaagg agctcgagaa atattccaag acctcgtttg aatacaacag ttacgacaac
catacttatg gcaagcgagc catagctccc aaggtgcaaa ccagggatat atcccccaaa ggatatgatg
atgcgaagcg gtactgcaag gaccccagcc ccagcagcag cagcaccagc agctacgcgc ccagcagcag
cagcaacctg agctgcggcg ggggcagcag cgccagcagc acgtgcagca agagcagctt cgactacacg
cacgacatgg aggcggccca catggcggcc accgccatcc tcaacctgtc cacgcgctgc cgcgagatgc
cgcagaacct gagcaccaag ccgcaggacc tgtgcgccac gcggaaccct gacatggagg tggatgagaa
cgggaccctg gacctcagca tgaacaagca gaggccgcgg gacagctgct gccccatcct gacccctctg
gagcccatgt ccccccagca gcaggcagtg atgaacaacc ggtgtttcca gctgggcgag ggcgactgct
gggacttgcc cgtagactac accaaaatga aaccccggag gatagacgag gacgagtcca aagacattac
tccagaagac ttggacccat tccaggaggc tctagaagaa agacggtatc ccggggaggt gaccatccca
agtcccaaac ccaagtaccc tcagtgcaag gagagcaaaa aggacttaat aactctgtct ggctgccccc
tggcggacaa aagcattcga agtatgctgg ccaccagctc ccaagaactc aagtgcccca cgcctggctg
tgatggttct ggacatatca ccggcaatta tgcttctcat cggagccttt caggttgccc aagagcaaag
aaaagtggta tcaggatagc acagagcaaa gaagataaag aagatcaaga acccatcagg tgtccggtcc
ccgggtgcga cggccagggc cacatcactg ggaagtacgc gtcccatcgc agcgcctccg ggtgcccctt
ggcggccaag aggcagaaag acgggtacct gaatggctcc cagttctcct ggaagtcggt caagacggaa
ggcatgtcct gccccacgcc aggatgcgac ggctcaggcc acgtcagcgg cagcttcctc acacaccgca
gcttgtcagg atgcccgaga gccacgtcag cgatgaagaa ggcaaagctt tctggagagc agatgctgac
catcaaacag cgggccagca acggtataga aaatgatgaa gaaatcaaac agttagatga agaaatcaag
gagctaaatg aatccaattc ccagatggaa gccgatatga ttaaactcag aactcagatt accacgatgg
agagcaacct gaagaccatc gaagaggaga acaaagtgat tgagcagcag aacgagtctc tcctccacga
gctggcgaac ctgagccagt ctctgatcca cagcctggct aacatccagc tgccgcacat ggatccaatc
aatgaacaaa attttgatgc ttacgtgact actttgacgg aaatgtatac aaatcaagat cgttatcaga
gtccagaaaa taaagcccta ctggaaaata taaagcaggc tgtgagagga attcaggtct ga
Myt11 human protein
(SEQ ID No. 44)
MEVDTEEKRHRTRSKGVRVPVEPAIQELFSCPTPGCDGSGHVSGKYARHRSVYGCPLAKKRKTQDKQPQEPAPKRK
PFAVKADSSSVDECDDSDGTEDMDEKEEDEGEEYSEDNDEPGDEDEEDEEGDREEEEEIEEEDEDDDEDGEDVEDE
EEEEEEEEEEEEEEENEDHQMNCHNTRIMQDTEKDDNNNDEYDNYDELVAKSLLNLGKIAEDAAYR
ARTESEMNSNTSNSLEDDSDKNENLGRKSELSLDLDSDVVRETVDSLKLLAQGHGVVLSENMNDRNYADSMSQQDS
RNMNYVMLGKPMNNGLMEKMVEESDEEVCLSSLECLRNQCFDLARKLSETNPQERNPQQNMNIRQHVRPEEDFPGR
TPDRNYSDMLNLMRLEEQLSPRSRVFASCAKEDGCHERDDDTTSVNSDRSEEVFDMTKGNLTLLEKAIALETERAK
AMREKMAMEAGRRDNMRSYEDQSPRQLPGEDRKPKSSDSHVKKPYYDPSRTEKKESKCPTPG
CDGTGHVTGLYPHHRSLSGCPHKDRVPPEILAMHESVLKCPTPGCTGRGHVNSNRNSHRSLSGCPIAAAEKLAKAQ
EKHQSCDVSKSSQASDRVLRPMCFVKQLEIPQYGYRNNVPTTTPRSNLAKELEKYSKTSFEYNSYDNHTYGKRAIA
PKVQTRDISPKGYDDAKRYCKDPSPSSSSTSSYAPSSSSNLSCGGGSSASSTCSKSSFDYTHDMEAAHMAATAILN
LSTRCREMPQNLSTKPQDLCATRNPDMEVDENGTLDLSMNKQRPRDSCCPILTPLEPMSPQQ
QAVMNNRCFQLGEGDCWDLPVDYTKMKPRRIDEDESKDITPEDLDPFQEALEERRYPGEVTIPSPKPKYPQCKESK
KDLITLSGCPLADKSIRSMLATSSQELKCPTPGCDGSGHITGNYASHRSLSGCPRAKKSGIRIAQSKEDKEDQEPI
RCPVPGCDGQGHITGKYASHRSASGCPLAAKRQKDGYLNGSQFSWKSVKTEGMSCPTPGCDGSGHVSGSFLTHRSL
SGCPRATSAMKKAKLSGEQMLTIKQRASNGIENDEEIKQLDEEIKELNESNSQMEADMIKLR
TQITTMESNLKTIEEENKVIEQQNESLLHELANLSQSLIHSLANIQLPHMDPINEQNFDAYVTTLTEMYTNQDRYQ
SPENKALLENIKQAVRGIQV
En2 mouse cDNA
(SEQ ID No. 45)
atggaggag aaggattcca agcccagcga gacggcggcg gaggcgcaga gacagccgga acccagctcc
ggcggcggct ctggcggcgg cagcagcccg agcgactcgg acaccggccg ccggcgggct ctgatgctgc
ccgaggtcct acaggcgcca ggcaaccacc agcatccaca tcgcatcacc aacttcttca tcgataacat
cctgcggcct gagtttggcc gccgaaagga cgcggggact tgctgtgcgg gcgcgggcgg agccagggga
ggcgaaggcg gcgctggcac taccgaagga ggcggcggcg gcgcaggcgg agccgagcag ctactgggcg
ccagggagtc ccgaccgaac ccagcgtgcg cacccagcgc gggaggaacg ctctccgccg ccgctggcga
ccctgcggtc gacggagaag gaggttccaa gacgctatca cttcacggtg gtgccaaaaa acccggcgat
cctgggggtt ccttggatgg agtgctcaaa gcccggggct tgggcggcgg tgacctgtcg gtgagctccg
actcggacag ctctcaagcc agcgccactc tgggcgcgca gcccatgctc tggcccgctt gggtctactg
cacgcgctat tctgaccggc cttcttcagg tcccaggtcc cgaaaaccaa agaagaagaa ccctaacaaa
gaggacaagc ggcctcgcac agccttcact gctgagcagc tccagaggct caaggctgag tttcagacca
acaggtacct gacagagcag cggcgccaga gtctggcaca ggagctcagc ctgaacgagt ctcagatcaa
gatttggttc cagaacaagc gggccaaaat caagaaagcc acgggcaaca agaacacttt ggcggtgcac
ctcatggcac agggcctgta caaccattcc accacggcca aggagggcaa gtcggacagc gagtag
En2 mouse protein
(SEQ ID No. 46)
MEEKDSKPSETAAEAQRQPEPSSGGGSGGGSSPSDSDTGRRRALMLPEVLQAPGNHQHPHRITNFFIDNILRPEFG
RRKDAGTCCAGAGGARGGEGGAGTTEGGGGGAGGAEQLLGARESRPNPACAPSAGGTLSAAAGDPAVDGEGGSKTL
SLHGGAKKPGDPGGSLDGVLKARGLGGGDLSVSSDSDSSQASATLGAQPMLWPAWFYCTRYSDRPS
SGPRSRKPKKKNPNKEDKRPRTAFTAEQLQRLKAEFQTNRYLTEQRRQSLAQELSLNESQIKIWFQNKRAKIKKAT
GNKNTLAVHLMAQGLYNHSTTAKEGKSDSE
En2 human cDNA
(SEQ ID No. 47)
a tggaggagaa tgaccccaag cctggcgaag cagcggcggc ggtggaggga cagcggcagc
cggaatccag ccccggcggc ggctcgggcg gcggcggcgg tagcagcccg ggcgaagcgg acaccgggcg
ccggcgggct ctgatgctgc ccgcggtcct gcaggcgccc ggcaaccacc agcacccgca ccgcatcacc
aacttcttca tcgacaacat cctgcggccc gagttcggcc ggcgaaagga cgcggggacc tgctgtgcgg
gcgcgggagg aggaaggggc ggcggagccg gcggcgaagg cggcgcgagc ggtgcggagg gaggcggcgg
cgcgggcggc tcggagcagc tcttgggctc gggctcccga gagccccggc agaacccgcc atgtgcgccc
ggcgcgggcg ggccgctccc agccgccggc agcgactctc cgggtgacgg ggaaggcggc tccaagacgc
tctcgctgca cggtggcgcc aagaaaggcg gcgaccccgg cggccccctg gacgggtcgc tcaaggcccg
cggcttgggc ggcggcgacc tgtcggtgag ctcggactcg gacagctcgc aagccggcgc caacctgggc
gcgcagccca tgctctggcc ggcgtgggtc tactgtacgc gctactcgga ccggccttct tcaggtccca
ggtctcgaaa accaaagaag aagaacccga acaaagagga caagcggccg cgcacggcct ttaccgccga
gcagctgcag aggctcaagg ccgagttcca gaccaacagg tacctgacgg agcagcggcg ccagagcctg
gcgcaggagc tgagcctcaa cgagtcacag atcaagattt ggttccagaa caagcgcgcc aagatcaaga
aggccacggg caacaagaac acgctggccg tgcacctcat ggcacagggc ttgtacaacc actccaccac
agccaaggag ggcaagtcgg acagcgagta g
Hn2 human protein
(SEQ ID No. 48)
MEENDPKPGEAAAAVEGQRQPESSPGGGSGGGGGSSPGEADTGRRRALMLPAVLQAPGNHQHPHRITNFFIDNILR
PEFGRRKDAGTCCAGAGGGRGGGAGGEGGASGAEGGGGAGGSEQLLGSGSREPRQNPPCAPGAGGPLPAAGSDSPG
DGEGGSKTLSLHGGAKKGGDPGGPLDGSLKARGLGGGDLSVSSDSDSSQAGANLGAQPMLWPAWVY
CTRYSDRPSSGPRSRKPKKKNPNKEDKRPRTAFTAEQLQRLKAEFQTNRYLTEQRRQSLAQELSLNESQIKIWFQN
KRAKIKKATGNKNTLAVHLMAQGLYNHSTTAKEGKSDSE
Foxa1 mouse cDNA
(SEQ ID No. 49)
atgt tagggactgt gaagatggaa gggcatgaga gcaacgactg gaacagctac tacgcggaca
cgcaggaggc ctactcctct gtccctgtca gcaacatgaa ctccggcctg ggctctatga actccatgaa
cacctacatg accatgaaca ccatgaccac gagcggcaac atgaccccgg cttccttcaa catgtcctac
gccaacacgg gcttaggggc cggcctgagt cccggtgctg tggctggcat gccaggggcc tctgcaggcg
ccatgaacag catgactgcg gcgggcgtca cggccatggg tacggcgctg agcccgggag gcatgggctc
catgggcgcg cagcccgcca cctccatgaa cggcctgggt ccctacgccg ccgccatgaa cccgtgcatg
agtcccatgg cgtacgcgcc gtccaacctg ggccgcagcc gcgcgggggg cggcggcgac gccaagacat
tcaagcgcag ctaccctcac gccaagccgc cttactccta catctcgctc atcacgatgg ccatccagca
ggcgcccagc aagatgctca cgctgagcga gatctaccag tggatcatgg acctcttccc ctattaccgc
cagaaccagc agcgctggca gaactccatc cgccactcgc tgtccttcaa cgattgtttc gtcaaggtgg
cacgatcccc ggacaagcca ggcaagggct cctactggac gctgcacccg gactccggca acatgttcga
gaacggctgc tacttgcgcc gccaaaagcg cttcaagtgt gagaagcagc cgggggccgg aggtgggagt
gggggcggcg gctccaaagg gggcccagaa agtcgcaagg acccctcagg cccggggaac cccagcgccg
agtcacccct tcaccggggt gtgcacggaa aggctagcca gctagagggc gcgccggccc cagggcccgc
cgccagcccc cagactctgg accacagcgg ggccacggcg acagggggcg cttcggagtt gaagtctcca
gcgtcttcat ctgcgccccc cataagctcc gggccagggg cgctagcatc tgtacccccc tctcacccgg
ctcacggcct ggcaccccac gaatctcagc tgcatctgaa aggggatccc cactactcct ttaatcaccc
cttctccatc aacaacctca tgtcctcctc cgagcaacag cacaagctgg acttcaaggc atacgagcag
gcgctgcagt actctcctta tggcgctacc ttgcccgcca gtctgcccct tggcagcgcc tcagtggcca
cgaggagccc catcgagccc tcagccctgg agccagccta ctaccaaggt gtgtattcca gacccgtgct
aaatacttcc tag
Foxa1 mouse protein
(SEQ ID No. 50)
MLGTVKMEGHESNDWNSYYADTQEAYSSVPVSNMNSGLGSMNSMNTYMTMNTMTTSGNMTPASFWMSYANTGLGAG
LSPGAVAGMPGASAGAMNSMTAAGVTAMGTALSPGGMGSMGAQPATSMNGLGPYAAAMNPCMSPMAYAPSNLGRSR
AGGGGDAKTFKRSYPHAKPPYSYISLITMAIQQAPSKMLTLSEIYQWIMDLFPYYRQNQQRWQNSI
RHSLSFNDCFVKVARSPDKPGKGSYWTLHPDSGNMFENGCYLRRQKRFKCEKQPGAGGGSGGGGSKGGPESRKDPS
GPGNPSAESPLHRGVHGKASQLEGAPAPGPAASPQTLDHSGATATGGASELKSPASSSAPPISSGPGALASVPPSH
PAHGLAPHESQLHLKGDPHYSFNHPFSINNLMSSSEQQHKLDFKAYEQALQYSPYGATLPASLPLGSASVATRSPI
EPSALEPAYYQGVYSRPVLNTS
Foxa1 human cDNA
(SEQ ID No. 51)
atgttagg aactgtgaag atggaagggc atgaaaccag cgactggaac agctactacg cagacacgca
ggaggcctac tcctccgtcc cggtcagcaa catgaactca ggcctgggct ccatgaactc catgaacacc
tacatgacca tgaacaccat gactacgagc ggcaacatga ccccggcgtc cttcaacatg tcctatgcca
acccgggcct aggggccggc ctgagtcccg gcgcagtagc cggcatgccg gggggctcgg cgggcgccat
gaacagcatg actgcggccg gcgtgacggc catgggtacg gcgctgagcc cgagcggcat gggcgccatg
ggtgcgcagc aggcggcctc catgaatggc ctgggcccct acgcggccgc catgaacccg tgcatgagcc
ccatggcgta cgcgccgtcc aacctgggcc gcagccgcgc gggcggcggc ggcgacgcca agacgttcaa
gcgcagctac ccgcacgcca agccgcccta ctcgtacatc tcgctcatca ccatggccat ccagcaggcg
cccagcaaga tgctcacgct gagcgagatc taccagtgga tcatggacct cttcccctat taccggcaga
accagcagcg ctggcagaac tccatccgcc actcgctgtc cttcaatgac tgcttcgtca aggtggcacg
ctccccggac aagccgggca agggctccta ctggacgctg cacccggact ccggcaacat gttcgagaac
ggctgctact tgcgccgcca gaagcgcttc aagtgcgaga agcagccggg ggccggcggc gggggcggga
gcggaagcgg gggcagcggc gccaagggcg gccctgagag ccgcaaggac ccctctggcg cctctaaccc
cagcgccgac tcgcccctcc atcggggtgt gcacgggaag accggccagc tagagggcgc gccggccccc
gggcccgccg ccagccccca gactctggac cacagtgggg cgacggcgac agggggcgcc tcggagttga
agactccagc ctcctcaact gcgcccccca taagctccgg gcccggggcg ctggcctctg tgcccgcctc
tcacccggca cacggcttgg caccccacga gtcccagctg cacctgaaag gggaccccca ctactccttc
aaccacccgt tctccatcaa caacctcatg tcctcctcgg agcagcagca taagctggac ttcaaggcat
acgaacaggc actgcaatac tcgccttacg gctctacgtt gcccgccagc ctgcctctag gcagcgcctc
ggtgaccacc aggagcccca tcgagccctc agccctggag ccggcgtact accaaggtgt gtattccaga
cccgtcctaa acacttccta g
Foxa1 human protein
(SEQ ID No. 52)
MLGTVKMEGHETSDWNSYYADTQEAYSSVPVSNMNSGLGSMNSMNTYMTMNTMTTSGNMTPASFNMSYANPGLGAG
LSPGAVAGMPGGSAGAMNSMTAAGVTAMGTALSPSGMGAMGAQQAASMNGLGPYAAAMNPCMSPMAYAPSNLGRSR
AGGGGDAKTFKRSYPHAKPPYSYISLITMAIQQAPSKMLTLSEIYQWIMDLFPYYRQNQQRWQNSI
RHSLSFNDCFVKVARSPDKPGKGSYWTLHPDSGNMFENGCYLRRQKRFKCEKQPGAGGGGGSGSGGSGAKGGPESR
KDPSGASNPSADSPLHRGVHGKTGQLEGAPAPGPAASPQTLDHSGATATGGASELKTPASSTAPPISSGPGALASV
PASHPAHGLAPHESQLHLKGDPHYSFNHPFSINNLMSSSEQQHKLDFKAYEQALQYSPYGSTLPASLPLGSASVTT
RSPIEPSALEPAYYQGVYSRPVLNTS
Msx1 mouse cDNA
(SEQ ID No. 53)
atggcccc ggctgctgct atgacttctt tgccactcgg tgtcaaagtg gaggactccg ccttcgccaa
gcctgctggg ggaggcgttg gccaagcccc cggggctgct gcggccaccg caaccgccat gggcacagat
gaggaggggg ccaagcccaa agtgcccgct tcactcctgc ccttcagcgt ggaggccctc atggccgatc
acaggaagcc cggggccaag gagagcgtcc tggtggcctc cgaaggggct caggcagcgg gtggctcggt
gcagcacttg ggcacccggc ccgggtctct gggcgccccg gacgcgccct cctcgccgcg gcctctcggc
catttctcag tcggaggact cctcaagctg ccagaagatg ctctggtgaa ggccgaaagc cccgagaaac
tagatcggac cccgtggatg cagagtcccc gcttctcccc gcccccagcc agacggctga gtcccccagc
atgcacccta cgcaagcaca agaccaaccg caagcccagg acgcctttca ccacagctca gctgctggct
ctggagcgca agttccgcca gaagcagtac ctgtctattg ccgagcgcgc ggaattctcc agctcgctca
gcctcaccga gacccaggtg aagatctggt tccagaaccg tcgcgctaag gccaagagac tgcaggaggc
ggagctggag aagctgaaga tggccgcgaa acccatgttg ccgcctgctg ccttcggcct ctcttttcct
cttggcggtc ctgcagcggt ggctgcagct gcgggcgcct cactctacag tgcctctggc cctttccagc
gcgccgcgct gcctgtagcg cccgtgggac tctacaccgc ccatgtaggc tacagcatgt accacctgac
ttag
Msx1 mouse protein
(SEQ ID No. 54)
MAPAAAMTSLPLGVKVEDSAFAKPAGGGVGQAPGAAAATATAMGTDEEGAKPKVPASLLPFSVEALMADHRKPGAK
ESVLVASEGAQAAGGSVQHLGTRPGSLGAPDAPSSPRPLGHFSVGGLLKLPEDALVKAESPEKLDRTPWMQSPRFS
PPPARRLSPPACTLRKHKTNRKPRTPFTTAQLLALERKFRQKQYLSIAERAEFSSSLSLTETQVKI
WFQNRRAKAKRLQEAELEKLKMAAKPMLPPAAFGLSFPLGGPAAVAAAAGASLYSASGPFQRAALPVAPVGLYTAH
VGYSMYHLT
Msx1 human cDNA
(SEQ ID No. 55)
atggc cccggctgct gacatgactt ctttgccact cggtgtcaaa gtggaggact ccgccttcgg
caagccggcg gggggaggcg cgggccaggc ccccagcgcc gccgcggcca cggcagccgc
catgggcgcg gacgaggagg gggccaagcc caaagtgtcc ccttcgctcc tgcccttcag
cgtggaggcg ctcatggccg accacaggaa gccgggggcc aaggagagcg ccctggcgcc
ctccgagggc gtgcaggcgg cgggtggctc ggcgcagcca ctgggcgtcc cgccggggtc
gctgggagcc ccggacgcgc cctcttcgcc gcggccgctc ggccatttct cggtgggggg
actcctcaag ctgccagaag atgcgctcgt caaagccgag agccccgaga agcccgagag
gaccccgtgg atgcagagcc cccgcttctc cccgccgccg gccaggcggc tgagcccccc
agcctgcacc ctccgcaaac acaagacgaa ccgtaagccg cggacgccct tcaccaccgc
gcagctgctg gcgctggagc gcaagttccg ccagaagcag tacctgtcca tcgccgagcg
cgcggagttc tccagctcgc tcagcctcac tgagacgcag gtgaagatat ggttccagaa
ccgccgcgcc aaggcaaaga gactacaaga ggcagagctg gagaagctga agatggccgc
caagcccatg ctgccaccgg ctgccttcgg cctctccttc cctctcggcg gccccgcagc
tgtagcggcc gcggcgggtg cctcgctcta cggtgcctct ggccccttcc agcgcgccgc
gctgcctgtg gcgcccgtgg gactctacac ggcccatgtg ggctacagca tgtaccacct
gacatag
Msx1 human protein
(SEQ ID No. 56)
MAPAADMTSLPLGVKVEDSAFGKPAGGGAGQAPSAAAATAAAMGADEEGAKPKVSPSLLPFSVEALMADHRKPGAK
ESALAPSEGVQAAGGSAQPLGVPPGSLGAPDAPSSPRPLGHFSVGGLLKLPEDALVKAESPEKPERTPWMQSPRFS
PPPARRLSPPACTLRKHKTNRKPRTPFTTAQLLALERKFRQKQYLSIAERAEFSSSLSLTETQVKI
WFQNRRAKAKRLQEAELEKLKMAAKPMLPPAAFGLSFPLGGPAAVAAAAGASLYGASGPFQRAALPVAPVGLYTAH
VGYSMYHLT

The authors also generated multi-cistronic 2A peptide vectors expressing the three factors (Mash1 (A), Nurr1 (N) and Lmx1a (L)) in order to co-express in the same cells all of them. The authors cloned the three factors in the following order: ANL or NAL. ANL and NAL multicistronic cassettes were constructed separating the cDNAs (full length cDNA as reported above) with the 2A peptide sequences (SEQ ID No. 57 and 58) as follows from the 5′ end to the 3′ end for ANL:

Human Mash1: nucleotide 572 to nucleotide 1282 of Seq ID No. 3

F2A:
(SEQ ID No. 57)
AAACAGACTTTGAATTTTGACCTTCTCAAGTTGGCGGGA
GACGTGGAGTCCAACCCAGGGCCC

Human Nurr 1: nucleotide 423 to nucleotide 2219 of SEQ ID No. 7

T2A:
(SEQ ID No. 58)
GAGGGCAGAGGAAGTCTTCTAACATGCGGTGACGTGGA
GGAGAATCCCGGCCCT.
and

Human Lmx1a: nucleotide 384 to nucleotide 1532 of SEQ ID No. 11 (ANL vector: 5′ A+F2A+N+T2A+L 3′)

Similarly, the NAL vector contains the sequences: 5′ N+F2A+A+T2A+L 3′. Replication-incompetent, VSVg-coated lentiviral particles were packaged in 293T cells. MEFs, IMR90 and adult mouse and human fibroblasts were infected in MEF media. 16-20 h after infection cells were switched into fresh MEF media containing doxycycline (2 mg/ml, Sigma). After 48 h medium was replaced with neuronal inducing media RDMEM/F12 (Invitrogen), 25 μg/ml insulin (Sigma), 50 μg/ml transferrin (Sigma), 30 nM sodium selenite, 20 nM progesterone (Sigma), 100 nM putrescine (Sigma) and penicillin/streptomycin (Sigma) containing doxycycline. The medium was changed every 2-3 days for further 10-22 days. For proliferation assay, MEFs were treated with a 48 h pulse of 10 μM BrdU. In the experiments performed in hypoxia condition, cells were kept at 5% O₂ (instead of about 20%) since the infection day.

As negative control we used DsRed cDNA cloned in the TET-O-FUW lentiviral vector as done for the dopaminergic cDNAs. The corresponding lentivirus was used to infect MEF.

Immunohistochemistry.

For immucytochemical analysis 5×10⁴ mouse or human fibroblasts were plated on matrigel-coated glass coverslips the day before the infection. 10-28 days following viral infection cells were fixed for 20 min at RT in 4% paraformaldehyde in PBS, permeabilized for 30 min in PBS containing 0.1% Triton X-100 and 10% normal goat serum (NGS), and incubated o/n at 4° C. in PBS containing 10% NGS and primary antibodies. Then cells were washed three times with PBS and incubated for 2 h at RT with anti-rabbit or anti-mouse secondary antibodies Alexa Fluor-488 or Alexa Fluor-594 (1:500, Invitrogen). For immunohistochemical analysis P15 or adult mouse brains were fixed o/n at 4° C. with 4% paraformaldehyde, buffered in 30% sucrose and embedded in OCT. Frozen brains were sectioned into 15- or 40-μm thick sections with a cryostat and processed for immunostaining. Sections were boiled 3 min in 10 mM citrate buffer solution pH 6 for antigen retrieval and permeabilized for 1 h at RT in PBS containing 0.1% or 0.25% Triton X-100 and 10% NGS. Primary antibodies were as follows: mouse anti-TH (1:200, Millipore), rabbit anti-TH (1:200, Immunological Sciences), mouse anti-βIII-tubulin (1:500, Covance), rabbit anti-βIII-tubulin (TuJ1) (1:500, Covance), rabbit anti-PITX3 (1:200, Zymed), rabbit anti-VMAT2 (1:200, Chemicon), rat anti-DAT (1:500, Millipore), rabbit anti-D2 receptor (1:100, Millipore), rabbit anti-calbindin (1:200, Swant), rabbit anti-AADC (1:100, Novus Biologicals), rabbit anti-ALDH1A1 (1:200, Abcam), mouse anti-synaptotagmin I (1:200, Synaptic Systems), mouse anti-synapsin (1:200; Synaptic Systems), chicken anti-GFP (1:2000, Molecular Probes), rat anti-BrdU (1:200, BD), mouse anti-MAP2 (1:500, Immunological Sciences), rabbit anti-Otx2 (1:100 R&D). Beta-galactosiDANse staining was performed as previously described²⁵.

Statistical Analysis.

The total numbers of Th+ and Tuj1+ cells were quantified 12-24 days after infection. Cell counting was performed on ten fields from three replicates for each condition and normalized with the number of cells plated before the infection. Data were expressed as mean±SE.

RT-PCR.

RNA was extracted from single cultures, using Trizol isolation system (Invitrogen) according to manufacturer's instructions. The yield and integrity of the RNA were determined by the spectrophotometric measurement of A260 and by agarose-gel electrophoresis, respectively. Total RNA was treated with DNAse I (Qiagen) to prevent DNA contamination. Two micrograms of RNA were reverse transcribed Transcriptor High Fidelity cDNA Synthesis Kit (Roche). One twentieth of the reverse transcribed cDNA was amplified in a 25 microliters of reaction mixture containing Taq polymerase buffer (Fisher BioReagents), 0.2 mM dNTPs (Finnzymes OY, Espoo, Finland), 0.4 micromolar each primer, 1 U Taq polymerase (Fisher BioReagents). Primers used to amplify cDNA samples are listed in Table I.

TABLE I
Table of nucleotide primers.

| | | | Annealing |
|---|---|---|---|
| Primer | Forward sequence 5′-3′ | Reverse sequence 5′-3′ | Temperature (° C.) |
| v-Ascl1 | ACGACCCTCTTAGCCCAGAG | GGCCGAAGGGACGTAGCAG | |
| | (SEQ ID No. 59) | (SEQ ID No. 60) | 60 |
| m-Aldh1a1 | CTGCAAGTGAGGAGGTCATC | CTGCTGGCTTGACAACCAC | |
| | (SEQ ID No. 61) | (SEQ No. 62) | 59 |
| h-ALDH1A1 | TTTGGAAGATAGGGCCTGCACTG | CCTGGATGCGGCTATACAACACTG | |
| | (SEQ ID No. 63) | (SEQ ID No. 64) | 58 |
| h-DAT | AGCAGAACGGAGTGCAGCT | GTATGCTCTGATGCCGTCT | |
| | (SEQ ID No. 65) | (SEQ ID No. 66) | 55 |
| m-Dat | CGTGGGACCAATGTCTTCTGTG | ATGGTGAAGGAGGAGAAGAAGT | |
| | (SEQ ID No. 67) | (SEQ ID No. 68) | 58 |
| h-AADC | ACGCAAGTGAATTCCGAAGGAGAG | CAGCCGATGGATCACTTTGGT | |
| | (SEQ ID No. 69) | (SEQ ID No. 70) | 60 |
| m-Aadc | CCTACTGGCTGCTCGGACTAA | GCGTACCAGTGACTCAAACTC | |
| | (SEQ ID No. 71) | (SEQ ID No. 72) | 60 |
| m-Drd2 | GCCCTTCATCGTCACCCTGCT | TGGGCATGGTCTGGATCTCAA | |
| | (SEQ ID No. 73) | (SEQ ID No. 74) | 60 |
| m-En1 | TCAAGACTGACTCACAGCAACCCC | CTTTGTCCTGAACCGTGGTGGTAG | |
| | (SEQ ID No. 75) | (SEQ ID No. 76) | 60 |
| h-GAPDH | CAAGATCATCAGCAATGCCTCCTG | GCCTGCTTCACCACCTTCTTGA | |
| | (SEQ ID No. 77) | (SEQ ID No. 78) | 60 |
| m-Gapdh | GGCATTGCTCTCAATGACAA | AGGGCCTCTCTCTTGCTCTC | |
| | (SEQ ID No. 79) | (SEQ ID No. 80) | 60 |
| m-Lmx1b | CCTCAGCGTGCGTGTGGTC | AGCAGTCGCTGAGGCTGGTG | |
| | (SEQ ID No. 81) | (SEQ ID No. 82) | 62 |
| m-Lmx1a | CTCACCCCACCCCAGATGCCT | CTCCCTCCCCAGCCCACCTCT | |
| | (SEQ ID No. 83) | (SEQ ID No. 84) | 60 |
| v-Lmx1a | CCCCATTGACCATCTGTACT | GGCCGAAGGGACGTAGCAG | |
| | (SEQ ID No. 85) | (SEQ ID No. 86) | 60 |
| m-Ngn2 | GGACATTCCCGGACACACAC | TCTCGATCTTCGTGAGCTTG | |
| | (SEQ ID No. 87) | (SEQ ID No. 88) | 60 |
| m-Nurr1 | TTGCTGCCCTGGCTATGGTCA | ACAAGCAAGCATGGCCAAACA | |
| | (SEQ ID No. 89) | (SEQ ID No. 90) | 58 |
| v-Nurr1 | TCTACCTGAAATTGGAAGAC | GGCCGAAGGGACGTAGCAG | |
| | (SEQ ID No. 91) | (SEQ ID No. 92) | 60 |
| m-Otx2 | CGCCTTACGCAGTCAATGGG | GACAGTGGGGAGATGGACGCT | |
| | (SEQ ID No. 93) | (SEQ ID No. 94) | 57 |
| m-Pitx3 | GCAACTGGCCGCCCAAGG | AGGCCCCACGTTGACCGA | |
| | (SEQ ID No. 95) | (SEQ ID No. 96) | 58 |
| m-Sox2 | GGCGGCAACCAGAAGAACAG | GCTTGGCCTGCGTCGATGAAC | |
| | (SEQ ID No. 97) | (SEQ ID No. 98) | 62 |
| h-TH | GAGTACACCGCCGAGGAGATTG | GCGGATATACTGGGTGCACTGG | |
| | (SEQ ID No. 99) | (SEQ ID No. 100) | 60 |
| m-Th | TGTCACGTCCCCAAGGTTCAT | GGGCAGGCCGGGTCTCTAAGT | |
| | (SEQ ID No. 101) | (SEQ ID No. 102) | 57 |
| m-Th | GATTCAGAGGCAGGTGCCTG | GCATAGTGCAAGCTGGTGGTC | |
| promoter | (SEQ ID No. 103) | (SEQ ID No. 104) | 60 |
| h-VMAT2 | TTGGTCTGTTGTTTGCCTCGAAAG | GGGTCCTTCAGCAGCGTGGTTAG | |
| | (SEQ ID No. 105) | (SEQ ID No. 106) | 60 |
| m-Vmat2 | ATCCAGACTGCCAGGCCAGCG | CTCCATCCAAGAGCACCAAGG | |
| | (SEQ ID No. 107) | (SEQ ID No. 108) | 58 |
| m-Vmat2 | TGGGCTCCTGTGGCTGTGTTCTAG | CCGGAGCACAAGGAGTTTCGT | |
| promoter | (SEQ ID No. 109) | (SEQ ID No. 110) | 62 |

Cell Sorting, Laser Capture Microdissection and Microarray Analysis.

TH-GFP positive iDAN cells were directly sorted in Trizol (Invitrogen) using the cell sorter FACSVantage SE DiVa (Becton-Dickinson). Thus RNA was extracted as reported above and biotin-labeled cRNA was obtained using the Ovation kit (NuGEN). Labeled cRNA was hybridized (CBM genexpression facility, SISSA) on Affymetrix Mouse Gene 1.0 ST Arrays, containing 35,557 probe sets corresponding to 28,853 genes. Hybridized arrays were stained and washed (GeneChip Fluidics Station 450) and scanned (GeneChip Scanner 3000 7G). Cell intensity values were computed using the Affymetrix GeneChip Operating Software (GCOS). Further data processing was performed in the R computing environment (http://www.r-project.org/) version 2.8.0 with BioConductor packages (http://www.bioconductor.org/). Robust Multi-Array Average (RMA) normalization was applied²⁶. Data were then filtered based on probe set intensity, so that only probe sets that had intensity value >50 in at least half the arrays were retained. Statistical analysis was performed with limma²⁷. P-values were adjusted for multiple testing using Benjamini and Hochberg's method to control the false discovery rate²⁶. Genes with adjusted P values below 0.01 were considered differentially expressed. Furthermore, a fold-change threshold cutoff was set to focus on genes whose expression level changes at least 2 times. Data were analyzed through DAVID Bioinformatics Resources v6.7²⁸.

Gene expression profiles of adult A9 and A10 DA neurons were obtained as previously described²⁹. In brief, adult TH-GFP female mice were sacrificed by cervical dislocation. The brains were rapidly cut to isolate midbrain region, and immediately immerged in 1× zinc fixative (BD Pharmingen) for 4-6 hours at +4° C. Once fixed, tissues were moved to 30% sucrose in 1× zinc fixative solution at +4° C. o/n. Following inclusion in OCT, tissues were frozen in iso-pentan (Sigma) and percooled with liquid nitrogen. 14-mm cryosections were mounted on SuperFrost plus glass slides (Menzel-Glaser) and air-dried. mDA A9 and A10 neurons (each one from three different mice) were isolated from cryosections by using a PALM LCM microdissection system (PALM Microlaser Technology, Bernried, Germany). To facilitate detection of fluorescent neurons, a drop of 1× zinc fixative was applied to the section during cell selection. The sections were air-dried, neurons were dissected and catapulted onto PALM adhesive caps (Zeiss). Total RNA from 2500 pooled neurons was isolated by using the Nano RNA extraction kit (Stratagene) and contaminating genomic DNA was removed through on-column DNase digestion step. The common reference RNA was generated from three midbrain regions of age-matched female mice. Midbrain RNA was isolated using RNeasy Mini kit (Qiagen), followed by DNase treatment. RNA from dissected neurons and all midbrains was amplified and labeled by Ovation Pico kit, WT exon and Encore biotin labeling kit (Nugene), following manufacturer's instructions. Once prepared, each target was hybridizated on MoExon 1.0ST GeneChip (Affymetrix). Statistical analysis was performed by oneChannelGUI R package. All hierarchical clusters were generated by TMEV software. The data discussed in this publication have been deposited in NCBI's Gene Expression Omnibus and are accessible through GEO series accession number GSE27174 (http://www.ncbi.nlm.nih.gov/geo/query/acc.cgi?acc=GSE27174).

Bisulfite Genomic Sequencing.

DNA from sorted TH-GFP+ reprogrammed MEFs was modified using the CpGenome modification kit (Chemicon) according to the manufacturer's recommendations. Thus Th and Vmat2 promoters CpG-rich selected regions were amplified using PCR primers listed in Table S5.

HPLC.

To quantify dopamine level in reprogrammed cells, cell pellets were homogenized in 100 μl 0.1 N HClO₄ and analyzed by using high performance liquid chromatography (HPLC) with electrochemical detection (Alexis 100, Antec Leyden, NV Zoeterwoude, Netherlands). To measure dopamine concentrations in the supernatants, cells were exposed to media with or without 50 mM KCl for 30 min, then 0.9 ml of supernatants were collected with addition of 0.1 ml of 1N HClO₄, filtered and analyzed by HPLC. Dopamine was separated on a reverse-phase column (ALB-105, 3 μm, 50×1 mm) with a mobile phase consisting of 50 mM phosphate buffer, 8 mM KCl, 500 mg/L octyl sodium sulfate, 0.1 mM EDTA, and 3% methanol (pH 6.0) at a flow rate of 50 μl/min. Dopamine was detected by a Decade II electrochemical detector equipped with two micro VT-03 electrochemical flow cells and 0.7 mm diameter glassy carbon electrode (Alexis 100, Antec Leyden, NV Zoeterwoude, Netherlands). The volume of injection was 5 μl. The detection limit established as 3:1 signal-to-noise ratio was below 0.5 nM.

Electrophysiology.

Recordings were performed on reprogrammed mouse and human fibroblasts and primary mesencephalic DA neurons. The mouse TH-GFP+ cells selected for the electrophysiological analysis were not so flat as the fibroblasts, and had several well developed neurites. The human cells selected for the electrophysiological analysis also had neuron-like shape with clearly distinguishable neurites by phase contrast microscopy. Only cells without signs of detachment from the substrate were used for recordings. Cells were perfused continuously with HEPES-buffered saline (HBS) of the following composition (in mM): 140 NaCl, 5 KCl, 2 CaCl₂, 2 MgCl₂, 15 HEPES, and 25 glucose, pH 7.4. The patch pipette solution contained (in mM): 130 K-gluconate, 10 KCl, 0.5 CaCl₂, 15 HEPES, 5 EGTA, 8 NaCl, 2 MgATP, 0.3 Na₂GTP, and 10 glucose, pH adjusted to 7.2 with KOH. Action potentials were recorded in the on-cell and the current-clamp whole-cell configuration. A current was injected to have membrane potentials around −60 mV, and step currents from −50 pA to 40 pA were injected to elicit action potentials. Na⁺ currents and composite K⁺ currents were recorded in the voltage-clamp configuration by delivering voltage steps ranging from −100 mV to +20 mV in cells held at −60 mV. Delayed rectifier K+ currents were activated by 0.5 s voltage steps from −40 mV to +20 mV after a 0.5 s-long step to −40 mV. A-type K⁺ currents were isolated by subtraction of delayed rectifier K⁺ currents from those activated by voltage steps after a 0.5 s-long step to −100 mV. Recordings were performed using an EPC10 USB patch clamp amplifier and PATCHMASTER software (HEKA Elektronik). Data were digitized at 10 kHz and analyzed with FITMASTER Software (HEKA Elektronik). Detection and measurements of action potentials were performed using MiniAnalysis software (Synaptosoft, Leonia, N.J.).

In Vivo Electrophysiology.

Slices were obtained from transplanted mice at postnatal day 42. The brains were quickly removed from the skull in ice-cold artificial cerebrospinal fluid (ACSF) containing the following (in mM): 125 NaCl, 25 NaHCO₃, 2.5 KCl, 1.25 NaH2PO₄, 2 CaCl₂, 1 MgCl₂, and 25 glucose, pH 7.4 (bubbled with 95% O₂ and 5% CO₂). Coronal slices (300 μm thick) were cut using a vibratome (VT1000S; Leica, Germany) and stored in ACSF at 25-28° C. For recording, slices were transferred to a recording chamber continuously superfused with ACSF (1-2 ml/min at 30-32° C.). Whole-cell recordings were performed in both current- and voltage-clamp configurations. Recording pipettes (3-5 MΩ of resistance) contained the following solution (in mM): 124 KH₂PO₄, 10 NaCl, 2 MgCl₂, 0.5 EGTA, 10 HEPES, 2 Na₂-ATP, 0.03 Na-GTP (pH 7.2, adjusted with KOH). Signals were sampled at 10 kHz, filtered at 2 kHz, and acquired using a MultiClamp 700A amplifier and pClamp 10 software (Molecular Devices, Sunnyvale, Calif.).

Amperometric Recording.

Amperometry was used to detect the evoked dopamine exocytosis from single cells³¹. Carbon-fiber microelectrodes were fabricated from 5 μm carbon fibers (Goodfellow, Oakdale, USA) inserted in a 1.2×0.68 mm glass capillary (A-M system, Sequim, USA) and pulled with a PE-22 micropipette puller (Narishige, London). Electrodes were sealed by dipping in Epoxy resin (Epo-Tek 301, Epoxy Technology, USA) and cured at 100° C. for 24 hours. They were backfilled with 3M KCl and trimmed to obtain a basal current between 140 and 180 nA. Electrode's response was tested by cyclic voltammetry and those with unstable cyclic voltammograms, when tested in a solution of 10 μM dopamine, were rejected. A voltage was applied to the carbon fiber using an EPC10 USB patch clamp amplifier (HEKA Elektronik). The signal was low-pass filtered at 10 kHz using a 4-pole Bessel filter, digitalized at 50 kHz and digitally refiltered at 1-1000 Hz. The latter resulted in slightly longer responses, but significantly improved visualization of secretion events that was the only aim of these experiments. The electrode was positioned adjacent to individual cells and lowered to approach somatodendritic domain of iDAN cells³², using Olympus BX50 microscope with ×40 water immersion objective. To increase signal-to-noise ratio, cells were pretreated with 100 μM L-DOPA (Sigma-Aldrich) for 30 minutes although the authors were able to resolve single spike-like release events in two untreated cells. The experiments consisted of current recordings at +750 mV during a brief baseline period, during which cells were perfused with standard external medium containing 5 mM K⁺. It was then exchanged for a stimulation solution (25 mM K⁺), and amperometric signals were recorded for a further period of 7 min. Catecholamine secretion was apparent as discrete spike-like events, each corresponding to vesicular catecholamine release. Most events were detected during 2 min after 25 mM K⁺ stimulation, but occasional events were observed also during baseline recordings. No vesicular release of dopamine was recorded at the electrode placed adjacent to a cell when the applied potential was 0 mV or −750 mV, or at +750 mV when electrode was placed remotely from cells.

FM4-64 Assay.

FM4-64 dye uptake experiments were performed as previously reported³³. Briefly, 21 DIV TH-GFP+ iDAN cells were stimulated for 1 min with 55 mM KCl, in the presence of FM4-64 (10 μM). After FM4-64 loading, neuronal cells were washed and perfused for 10 min with warmed Krebs buffer (37° C.) supplemented with TTX (1 μM) and CNQX (10 μM). After live fluorescent FM4-64 signals were acquired, cells were fixed and immonostained for TH and SYT1.

Electron Microscopy.

For ultrastructural immunocytochemistry, 21 DIV infected MEFs were fixed in 2% glutaraldehyde in PBS, washed in PBS, postfixed in 2% OsO₄ in PBS, and embedded in Epon. Ultrathin sections, prepared from these samples were analyzed with electron microscope (H-7000; Hitachi).

Cell Transplantation.

After 4 days of infection, TH-GFP MEFs were trypsinized and resuspended at 2×10⁵ cells/μl in fresh prepared Krebs buffer containing the following (in mM): 126 NaCl, 2.5 KCl, 1.2 NaH₂PO₄, 1.2 MgCl₂, 2.1 CaCl₂, 11 glucose, 4.2 NaHCO₃, 1 HEPES, and 1% vital dye Fast Green. P1 mice pups were anesthetized by hypothermia (4 min) and fixed to a support using band-aid. The skin and the skull overlying the lateral ventricle were opened over about 2 mm using an ophthalmic scalpel. Subsequently, the animal was placed in a stereotaxic rig (Kopff, Germany) under a Hamilton syringe containing 2 μl of cells suspension. The syringe was placed over the incision, positioned at the level of the skull, then lowered into the lumen of the right lateral ventricle (LV, 2.5 mm) or in the somatosensory cortex (1.5 mm) and cell solution was injected. Animals were left on a 37° C. heating blanket for several minutes after surgical manipulation to avoid fatal hypothermia.

6-Hydroxydopamine (6-OHDA) Lesion and Behavioral Analysis.

6-OHDA-lesioned adult male Sprague-Dawley rats (300-350 g) were purchased Charles River. The animals were unilaterally lesioned by 6-OHDA injection into the substantia nigra. 4 weeks after 6OHDA lesioned, the mice were injected with Amphetamine HCl (Sigma, 4 mg/kg i.p. injection) and Amphetamine-induced rotations were assessed before the cell grafting. The FACS sorted TH-GFP+ iDAN cells were resuspended in Krebs buffer at a density of about 100,000 cells per ul, and rats were grafted into the lesioned striatum (AP: +0.4 mm; ML: ±3.8 mm; DV: −3.3 mm) with 2 or 3-ul of cell suspension. Amphetamine-induced rotational behavior was measured again at 4, 8 and 14 weeks after grafting.

Stereological Analysis.

Three animals transplanted with reprogrammed cells were used for stereological analysis. Three weeks after transplantation animals were anaesthetized and sacrificed by transcardiac perfusion with 0.1 M PBS followed by 4% paraformaldehyde. Brains were cryoprotected through incubation in an ice-cold solution of 30% sucrose in 0.1M PBS and cut in coronal 40 μm-thick cryostat sections. From these sections, one systematic random series of sections was stained for GFP, so that sections were spaced at 7 section intervals (total of 16 sections per mouse). GFP immunoperoxiDANse staining was performed as described elsewere³². Cells were quantified using the assistance of the Stereo Investigator v 3.0 software (MicroBrightField, Inc., Colchester, Vt.) and a personal computer running the software connected to a color video camera mounted on a Leica microscope^{35,36}. The motorized stage of the microscope, allowed precise and well-defined movements along the x-, y- and z-axes. Images were first acquired with a CCD-IRIS color video camera and the cerebral hemispheres were interactively delineated at low magnification on a video image of the section. Counting of cells was performed manually on every 7th section using a 40× lens. To estimate the total number of GFP positive cells the total number of neurons counted on the sections was multiplied by 7.

### Results

Initially, the authors transduced mouse embryonic fibroblasts (MEFs) from TH-GFP transgenic animals¹⁰ with a mixture of doxycycline (dox)-inducible lentiviruses expressing all selected factors (11 DA and 3 iN (first three genes), Table II) or with DsRed retrovirus (negative control) (FIG. 1a-d′).

TABLE II
List of the transcription factors included in the functional screening with
their relative NCBI access number and abbreviation letter
throughout the text.

| | Gene Name | Gene ID | Letter |
|---|---|---|---|
| 1 | Mash1 | 11924 | A |
| 2 | Brn2 | 18992 | B |
| 3 | Myt1l | 17933 | M |
| 4 | En-1 | 13798 | E1 |
| 5 | En-2 | 13799 | E2 |
| 6 | Pitx3 | 18742 | P |
| 7 | Foxa1 | 15375 | F1 |
| 8 | Foxa2 | 15376 | F2 |
| 9 | Nurr1 | 18227 | N |
| 10 | Otx2 | 18424 | O |
| 11 | Lmx1a | 110648 | La |
| 12 | Lmx1b | 16917 | Lb |
| 13 | Msx1 | 17701 | Mx |
| 14 | Neurog2 | 11924 | Ng |

The authors did not observe any GFP+ cells in MEFs 10 days after Ds-Red retrovirus infection or in culture without any viral infection (FIG. 1d′). In contrast, transduction of all factors resulted in the generation of a small number of bright GFP+ cells (1.8±0.8%) (FIG. 5d-f). The authors next sought to determine the minimal set of genes required for DA neuronal induction. Given its essential role as a proneural gene during neurogenesis, Mash1 was introduced into MEFs together with each other single DA factor. Reporter gene expression was elicited only when Mash1 was combined with Nurr1 (NCBI: Nr4a2), a critical determinant of the DA neuronal specification and survival during development and in adulthood¹¹. However, Mash1/Nurr1 combined activation elicited a true though modest increase of GFP+ cells (8±2%) (FIG. 5g-i). Therefore, the authors added a third molecule of the 12 remaining and scored for the rate and morphology of GFP+ cells in each combination. Surprisingly, only Lmx1a and in part Lmx1b (18±3% vs 13±3% of GFP+ cells, respectively) were able to synergize with Mash1/Nurr1, robustly increasing the generation of GFP+ cells with an evident complex neuronal morphology (FIG. 1h and FIG. 5s-y). Thus, the viral cocktail Lmx1a/Mash1/Nurr1 leads to an efficiency of TH+ cells of 18±3% (FIG. 1).

Using Mash1/Nurr1/Lmx1a factor combination the double GFP+/TH+ cells represented the majority of the induced TuJ1 neuronal cells (85±4%). Supplementation of a fourth factor among the remaining ones failed to produce any further increase in GFP+ cells, with Brn2 and Myt1l, the other two iN factors, even reducing the overall reprogramming efficiency (data not shown). For these reasons the authors focused on cells reprogrammed exclusively with the Mash1/Nurr1/Lmx1a factor combination. The same gene cocktail was also proficient in reprogramming adult mouse fibroblasts with high efficiency (FIG. 6). The authors also generated multi-cistronic 2A peptide vectors expressing the three factors (Mash1, Nurr1 and Lmx1a) in order to co-express in the same cells all of them. The authors cloned the three factors in the following order: ANL or NAL. When dopaminergic reprogramming experiments on MEFs are performed using lentiviruses expressing ANL- or NAL-multi-cistronic factor, there is clear increase of the TH/TUJ1+ cells (97±0.5% and 96±0.9% versus 85±4% obtained with the three single lentiviruses; FIG. 18).

Sixteen days after reprogramming, a large number of GFP+ cells expressed many of the distinctive components of the DA machinery like TH, vesicular monoamine transporter 2 (VMAT2, NCBI: SlC18A2), dopamine transporter (DAT, NCBI: SLC6A3), as well as aldehyde dehydrogenase 1a1 (ALDH1A1) and calbindin (FIG. 1e-l). Conversely, markers associated with adrenergic (dopamine-beta-hydroxylase, DBH) or serotonergic (tryptophan hydroxylase 1 or 2, TPH1/2; serotonin transporter, SERT, NCBI: SLC6A4) neurons were not induced (data not shown). Transcriptional analysis by RT-PCR confirmed the activation of the DA-specific gene network including the endogenous expression of Nurr1 and Lmx1a (FIG. 7). Global expression analysis showed that iDAN cells clustered with A9 and A10 adult mDA neurons rather than with fibroblasts of origin as illustrated by hierarchical clustering (FIG. 2a, b) and the general degree of gene expression overlap (FIG. 2c).

The list of genes present in table III indicates that the transcriptional profile of iDAN cells presents all the major features that characterize generic mouse dopaminergic neurons. Moreover iDAN cells do not share transcriptional marks of other catecholaminergic neurons.

TABLE III
List of all genes differentially expressed (>5 fold change) between 16 DIV
iDAN cells and MEFs.

| AFFY ID | SYMBOL | DESCRIPTION |
|---|---|---|
| 10593233 | Htr3a | 5-hydroxytryptamine (serotonin) receptor 3A |
| 10440522 | Adamts1 | a disintegrin-like and metallopeptidase (reprolysin type) with thrombospondin type 1 motif, 1 |
| 10531191 | Adamts3 | a disintegrin-like and metallopeptidase (reprolysin type) with thrombospondin type 1 motif, 3 |
| 10531195 | Adamts3 | a disintegrin-like and metallopeptidase (reprolysin type) with thrombospondin type 1 motif, 3 |
| 10412607 | Abhd6 | abhydrolase domain containing 6 |
| 10485982 | Actc1 | actin, alpha, cardiac |
| 10429029 | Adcy8 | adenylate cyclase 8 |
| 10402783 | Ahnak2 | AHNAK nucleoprotein 2 |
| 10450484 | Aif1 | allograft inflammatory factor 1 |
| 10417315 | LOC100041306 | alpha23-takusan |
| 10522934 | Amtn | amelotin |
| 10403796 | Amph | amphiphysin |
| 10603066 | Ace2 | angiotensin I converting enzyme (peptidyl-dipeptidase A) 2 |
| 10591781 | Anln | anillin, actin binding protein (scraps homolog, Drosophila) |
| 10536324 | Asb4 | ankyrin repeat and SOCS box-containing protein 4 |
| 10523451 | Anxa3 | annexin A3 |
| 10414065 | Anxa8 | annexin A8 |
| 10439009 | Apod | apolipoprotein D |
| 10527638 | Alox5ap | arachidonate 5-lipoxygenase activating protein |
| 10568109 | Asphd1 | aspartate beta-hydroxylase domain containing 1 |
| 10405047 | Aspn | asporin |
| 10350896 | Astn1 | astrotactin 1 |
| 10371092 | Atcay | ataxia, cerebellar, Cayman type homolog (human) |
| 10519555 | Abcb1b | ATP-binding cassette, sub-family B (MDR/TAP), member 1B |
| 10530319 | Atp8a1 | ATPase, aminophospholipid transporter (APLT), class I, type 8A, member 1 |
| 10538082 | Atp6v0e2 | ATPase, H+ transporting, lysosomal V0 subunit E2 |
| 10444756 | Atp6v1g2 | ATPase, H+ transporting, lysosomal V1 subunit G2 |
| 10560919 | Atp1a3 | ATPase, Na+/K+ transporting, alpha 3 polypeptide |
| 10595633 | Bcl2a1b | B-cell leukemia/lymphoma 2 related protein A1b |
| 10587683 | Bcl2a1b | B-cell leukemia/lymphoma 2 related protein A1b |
| 10587690 | Bcl2a1b | B-cell leukemia/lymphoma 2 related protein A1b |
| 10366528 | Best3 | bestrophin 3 |
| 10362968 | Bves | blood vessel epicardial substance |
| 10499358 | Bglap2 | bone gamma-carboxyglutamate protein 2 |
| 10606868 | Bex1 | brain expressed gene 1 |
| 10358457 | Bex4 | brain expressed gene 4 |
| 10601850 | Bex4 | brain expressed gene 4 |
| 10606835 | Bex2 | brain expressed X-linked 2 |
| 10487480 | Bub1 | budding uninhibited by benzimidazoles 1 homolog (S. cerevisiae) |
| 10542164 | Clec12a | C-type lectin domain family 12, member a |
| 10541614 | Clec4d | C-type lectin domain family 4, member d |
| 10423080 | C1qtnf3 | C1q and tumor necrosis factor related protein 3 |
| 10543369 | Cadps2 | Ca2+-dependent activator protein for secretion 2 |
| 10417628 | Cadps | Ca2+-dependent secretion activator |
| 10575034 | Cdh3 | cadherin 3 |
| 10423230 | Cdh9 | cadherin 9 |
| 10503416 | Calb1 | calbindin-28K |
| 10579649 | Cib3 | calcium and integrin binding family member 3 |
| 10588592 | Cacna2d2 | calcium channel, voltage-dependent, alpha 2/delta subunit 2 |
| 10430282 | Cacng2 | calcium channel, voltage-dependent, gamma subunit 2 |
| 10474875 | Casc5 | cancer susceptibility candidate 5 |
| 10503902 | Cnr1 | cannabinoid receptor 1 (brain) |
| 10528864 | Cnpy1 | canopy 1 homolog (zebrafish) |
| 10490913 | Car3 | carbonic anhydrase 3 |
| 10353102 | Cpa6 | carboxypeptidase A6 |
| 10411527 | Cartpt | CART prepropeptide |
| 10583008 | Casp12 | caspase 12 |
| 10554789 | Ctsc | cathepsin C |
| 10494271 | Ctss | cathepsin S |
| 10587383 | Cd109 | CD109 antigen |
| 10406928 | Cd180 | CD180 antigen |
| 10439651 | Cd200 | Cd200 antigen |
| 10528207 | Cd36 | CD36 antigen |
| 10351658 | Cd48 | CD48 antigen |
| 10501063 | Cd53 | CD53 antigen |
| 10387536 | Cd68 | CD68 antigen |
| 10435704 | Cd80 | CD80 antigen |
| 10351679 | Cd84 | CD84 antigen |
| 10488382 | Cd93 | CD93 antigen |
| 10347073 | BC042720 | cDNA sequence BC042720 |
| 10569163 | Cend1 | cell cycle exit and neuronal differentiation 1 |
| 10496204 | Cenpe | centromere protein E |
| 10360985 | Cenpf | centromere protein F |
| 10580469 | Cbln1 | cerebellin 1 precursor protein |
| 10379530 | Ccl12 | chemokine (C-C motif) ligand 12 |
| 10512372 | Ccl19 | chemokine (C-C motif) ligand 19 |
| 10512322 | Ccl19 | chemokine (C-C motif) ligand 19 |
| 10504159 | Ccl19 | chemokine (C-C motif) ligand 19 |
| 10504188 | Ccl19 | chemokine (C-C motif) ligand 19 |
| 10504132 | Ccl19 | chemokine (C-C motif) ligand 19 |
| 10523145 | Cxcl15 | chemokine (C—X—C motif) ligand 15 |
| 10502552 | Clca1 | chloride channel calcium activated 1 |
| 10502575 | Clca4 | chloride channel calcium activated 4 |
| 10593756 | Chrna3 | cholinergic receptor, nicotinic, alpha polypeptide 3 |
| 10585484 | Chrna5 | cholinergic receptor, nicotinic, alpha polypeptide 5 |
| 10593767 | Chrnb4 | cholinergic receptor, nicotinic, beta polypeptide 4 |
| 10503196 | Chd7 | chromodomain helicase DNA binding protein 7 |
| 10397882 | Chga | chromogranin A |
| 10476355 | Chgb | chromogranin B |
| 10512279 | Cntfr | ciliary neurotrophic factor receptor |
| 10498337 | Clrn1 | clarin 1 |
| 10495675 | F3 | coagulation factor III |
| 10595211 | Col12a1 | collagen, type XII, alpha 1 |
| 10521498 | Crmp1 | collapsin response mediator protein 1 |
| 10517517 | C1qa | complement component 1, q subcomponent, alpha polypeptide |
| 10517508 | C1qb | complement component 1, q subcomponent, beta polypeptide |
| 10517513 | C1qc | complement component 1, q subcomponent, C chain |
| 10452316 | C3 | complement component 3 |
| 10547657 | C3ar1 | complement component 3a receptor 1 |
| 10560242 | C5ar1 | complement component 5a receptor 1 |
| 10358339 | Cfh | complement component factor h |
| 10450325 | Cfb | complement factor B |
| 10532180 | Cplx1 | complexin 1 |
| 10607562 | Cnksr2 | connector enhancer of kinase suppressor of Ras 2 |
| 10426397 | Cntn1 | contactin 1 |
| 10540333 | Cntn6 | contactin 6 |
| 10537851 | Cntnap2 | contactin associated protein-like 2 |
| 10594301 | Coro2b | coronin, actin binding protein, 2B |
| 10475324 | Ckmt1 | creatine kinase, mitochondrial 1, ubiquitous |
| 10533401 | Cux2 | cut-like homeobox 2 |
| 10597323 | Arpp21 | cyclic AMP-regulated phosphoprotein, 21 |
| 10603551 | Cybb | cytochrome b-245, beta polypeptide |
| 10551836 | Cox7a1 | cytochrome c oxidase, subunit VIIa 1 |
| 10569008 | Cox8b | cytochrome c oxidase, subunit VIIIb |
| 10385391 | Cyfip2 | cytoplasmic FMR1 interacting protein 2 |
| 10409876 | Ctla2a | cytotoxic T lymphocyte-associated protein 2 alpha |
| 10592140 | Ddx25 | DEAD (Asp-Glu-Ala-Asp) box polypeptide 25 |
| 10356177 | Dner | delta/notch-like EGF-related receptor |
| 10381666 | Dcakd | dephospho-CoA kinase domain containing |
| 10395428 | Dgkb | diacylglycerol kinase, beta |
| 10496125 | Dkk2 | dickkopf homolog 2 (Xenopus laevis) |
| 10520527 | Dpysl5 | dihydropyrimidinase-like 5 |
| 10520318 | Dpp6 | dipeptidylpeptidase 6 |
| 10474814 | Disp2 | dispatched homolog 2 (Drosophila) |
| 10393594 | D11Bwg0517e | DNA segment, Chr 11, Brigham & Women's Genetics 0517 expressed |
| 10501754 | D3Bwg0562e | DNA segment, Chr 3, Brigham & Women's Genetics 0562 expressed |
| 10506274 | Dnajc6 | DnaJ (Hsp40) homolog, subfamily C, member 6 |
| 10607156 | Dcx | doublecortin |
| 10499138 | Dclk2 | doublecortin-like kinase 2 |
| 10441195 | Dscam | Down syndrome cell adhesion molecule |
| 10351111 | Dnm3os | dynamin 3, opposite strand |
| 10536334 | Dync1i1 | dynein cytoplasmic 1 intermediate chain 1 |
| 10497590 | Evi1 | ecotropic viral integration site 1 |
| 10368289 | Enpp1 | ectonucleotide pyrophosphatase/phosphodiesterase 1 |
| 10446282 | Emr1 | EGF-like module containing, mucin-like, hormone receptor-like sequence 1 |
| 10515095 | Elavl4 | ELAV (embryonic lethal, abnormal vision, Drosophila)-like 4 (Hu antigen D) |
| 10520368 | En2 | engrailed 2 |
| 10377938 | Eno3 | enolase 3, beta muscle |
| 10523175 | Ereg | epiregulin |
| 10542355 | Emp1 | epithelial membrane protein 1 |
| 10490602 | Eef1a2 | eukaryotic translation elongation factor 1 alpha 2 |
| 10575693 | AI427515 | expressed sequence AI427515 |
| 10495186 | AI504432 | expressed sequence AI504432 |
| 10593499 | AI593442 | expressed sequence AI593442 |
| 10475578 | BB181834 | expressed sequence BB181834 |
| 10500204 | Ecm1 | extracellular matrix protein 1 |
| 10491732 | Fat4 | FAT tumor suppressor homolog 4 (Drosophila) |
| 10363224 | Fabp7 | fatty acid binding protein 7, brain |
| 10499189 | Fcrls | Fc receptor-like S, scavenger receptor |
| 10360070 | Fcer1g | Fc receptor, IgE, high affinity I, gamma polypeptide |
| 10360040 | Fcgr3 | Fc receptor, IgG, low affinity III |
| 10475643 | Fgf7 | fibroblast growth factor 7 |
| 10397633 | Flrt2 | fibronectin leucine rich transmembrane protein 2 |
| 10540085 | Fbln2 | fibulin 2 |
| 10595298 | Filip1 | filamin A interacting protein 1 |
| 10492640 | Fstl5 | follistatin-like 5 |
| 10351971 | Fmn2 | formin 2 |
| 10485402 | Fjx1 | four jointed box 1 (Drosophila) |
| 10409999 | Fbp2 | fructose bisphosphatase 2 |
| 10527732 | Fry | furry homolog (Drosophila) |
| 10562192 | Fxyd5 | FXYD domain-containing ion transport regulator 5 |
| 10422760 | Fyb | FYN binding protein |
| 10358023 | Gpr37l1 | G protein-coupled receptor 37-like 1 |
| 10602896 | Gpr64 | G protein-coupled receptor 64 |
| 10601834 | Gprasp2 | G protein-coupled receptor associated sorting protein 2 |
| 10397645 | Gpr65 | G-protein coupled receptor 65 |
| 10464471 | Gal | galanin |
| 10385283 | Gabrg2 | gamma-aminobutyric acid (GABA-A) receptor, subunit gamma 2 |
| 10512807 | Gabbr2 | gamma-aminobutyric acid (GABA) B receptor 2 |
| 10478374 | Gdap1l1 | ganglioside-induced differentiation-associated protein 1-like 1 |
| 10344973 | Gdap1 | ganglioside-induced differentiation-associated-protein 1 |
| 10456353 | Grp | gastrin releasing peptide |
| 10406777 | Gm73 | gene model 73, (NCBI) |
| 10468722 | Gfra1 | glial cell line derived neurotrophic factor family receptor alpha 1 |
| 10498885 | Gria2 | glutamate receptor, ionotropic, AMPA2 (alpha 2) |
| 10599348 | Gria3 | glutamate receptor, ionotropic, AMPA3 (alpha 3) |
| 10368999 | Grik2 | glutamate receptor, ionotropic, kainate 2 (beta 2) |
| 10480676 | Grin1 | glutamate receptor, ionotropic, NMDA1 (zeta 1) |
| 10469672 | Gad2 | glutamic acid decarboxylase 2 |
| 10426812 | Gpd1 | glycerol-3-phosphate dehydrogenase 1 (soluble) |
| 10573054 | Gypa | glycophorin A |
| 10363070 | Gp49a | glycoprotein 49 A |
| 10571815 | Gpm6a | glycoprotein m6a |
| 10439514 | Gap43 | growth associated protein 43 |
| 10489179 | Ghrh | growth hormone releasing hormone |
| 10465820 | Gng3 | guanine nucleotide binding protein (G protein), gamma 3 |
| 10380571 | Gngt2 | guanine nucleotide binding protein (G protein), gamma transducing activity polypeptide 2 |
| 10573979 | Gnao1 | guanine nucleotide binding protein, alpha O |
| 10456363 | Gnal | guanine nucleotide binding protein, alpha stimulating, olfactory type |
| 10569341 | H19 | H19 fetal liver mRNA |
| 10581605 | Hp | haptoglobin |
| 10417421 | Hn1l | hematological and neurological expressed 1-like |
| 10417373 | Hn1l | hematological and neurological expressed 1-like |
| 10358609 | Hmcn1 | hemicentin 1 |
| 10358575 | Hmcn1 | hemicentin 1 |
| 10358585 | Hmcn1 | hemicentin 1 |
| 10358637 | Hmcn1 | hemicentin 1 |
| 10358601 | Hmcn1 | hemicentin 1 |
| 10358569 | Hmcn1 | hemicentin 1 |
| 10358593 | Hmcn1 | hemicentin 1 |
| 10358579 | Hmcn1 | hemicentin 1 |
| 10358607 | Hmcn1 | hemicentin 1 |
| 10358613 | Hmcn1 | hemicentin 1 |
| 10358615 | Hmcn1 | hemicentin 1 |
| 10358527 | Hmcn1 | hemicentin 1 |
| 10358553 | Hmcn1 | hemicentin 1 |
| 10358648 | Hmcn1 | hemicentin 1 |
| 10358549 | Hmcn1 | hemicentin 1 |
| 10358599 | Hmcn1 | hemicentin 1 |
| 10358611 | Hmcn1 | hemicentin 1 |
| 10358531 | Hmcn1 | hemicentin 1 |
| 10358517 | Hmcn1 | hemicentin 1 |
| 10358573 | Hmcn1 | hemicentin 1 |
| 10358597 | Hmcn1 | hemicentin 1 |
| 10358513 | Hmcn1 | hemicentin 1 |
| 10358555 | Hmcn1 | hemicentin 1 |
| 10358557 | Hmcn1 | hemicentin 1 |
| 10358529 | Hmcn1 | hemicentin 1 |
| 10358605 | Hmcn1 | hemicentin 1 |
| 10358567 | Hmcn1 | hemicentin 1 |
| 10358563 | Hmcn1 | hemicentin 1 |
| 10358559 | Hmcn1 | hemicentin 1 |
| 10358623 | Hmcn1 | hemicentin 1 |
| 10358589 | Hmcn1 | hemicentin 1 |
| 10358519 | Hmcn1 | hemicentin 1 |
| 10358571 | Hmcn1 | hemicentin 1 |
| 10358662 | Hmcn1 | hemicentin 1 |
| 10358515 | Hmcn1 | hemicentin 1 |
| 10358619 | Hmcn1 | hemicentin 1 |
| 10358660 | Hmcn1 | hemicentin 1 |
| 10358591 | Hmcn1 | hemicentin 1 |
| 10358587 | Hmcn1 | hemicentin 1 |
| 10358658 | Hmcn1 | hemicentin 1 |
| 10358650 | Hmcn1 | hemicentin 1 |
| 10358617 | Hmcn1 | hemicentin 1 |
| 10358656 | Hmcn1 | hemicentin 1 |
| 10358577 | Hmcn1 | hemicentin 1 |
| 10358525 | Hmcn1 | hemicentin 1 |
| 10358666 | Hmcn1 | hemicentin 1 |
| 10358521 | Hmcn1 | hemicentin 1 |
| 10358533 | Hmcn1 | hemicentin 1 |
| 10358654 | Hmcn1 | hemicentin 1 |
| 10358670 | Hmcn1 | hemicentin 1 |
| 10358561 | Hmcn1 | hemicentin 1 |
| 10358668 | Hmcn1 | hemicentin 1 |
| 10358535 | Hmcn1 | hemicentin 1 |
| 10358652 | Hmcn1 | hemicentin 1 |
| 10358583 | Hmcn1 | hemicentin 1 |
| 10358581 | Hmcn1 | hemicentin 1 |
| 10358664 | Hmcn1 | hemicentin 1 |
| 10358565 | Hmcn1 | hemicentin 1 |
| 10531737 | Hpse | heparanase |
| 10389786 | Hlf | hepatic leukemia factor |
| 10565156 | Homer2 | homer homolog 2 (Drosophila) |
| 10391084 | Hap1 | huntingtin-associated protein 1 |
| 10385248 | Hmmr | hyaluronan mediated motility receptor (RHAMM) |
| 10571840 | Hpgd | hydroxyprostaglandin dehydrogenase 15 (NAD) |
| 10383198 | LOC672511 | hypothetical LOC672511 |
| 10498383 | Igsf10 | immunoglobulin superfamily, member 10 |
| 10498386 | Igsf10 | immunoglobulin superfamily, member 10 |
| 10498379 | Igsf10 | immunoglobulin superfamily, member 10 |
| 10403743 | Inhba | inhibin beta-A |
| 10352234 | Itpkb | inositol 1,4,5-trisphosphate 3-kinase B |
| 10390211 | Igf2bp1 | insulin-like growth factor 2 mRNA binding protein 1 |
| 10480090 | Itga8 | integrin alpha 8 |
| 10586079 | Itga11 | integrin, alpha 11 |
| 10480003 | Itih2 | inter-alpha trypsin inhibitor, heavy chain 2 |
| 10462623 | Ifit1 | interferon-induced protein with tetratricopeptide repeats 1 |
| 10462618 | Ifit3 | interferon-induced protein with tetratricopeptide repeats 3 |
| 10469816 | Il1rn | interleukin 1 receptor antagonist |
| 10345791 | Il1rl1 | interleukin 1 receptor-like 1 |
| 10463737 | Ina | internexin neuronal intermediate filament protein, alpha |
| 10463732 | Ina | internexin neuronal intermediate filament protein, alpha |
| 10543120 | Ica1 | islet cell autoantigen 1 |
| 10458685 | Jakmip2 | janus kinase and microtubule interacting protein 2 |
| 10472562 | Kbtbd10 | kelch repeat and BTB (POZ) domain containing 10 |
| 10390860 | Krt23 | keratin 23 |
| 10462632 | Kif20b | kinesin family member 20B |
| 10471994 | Kif5c | kinesin family member 5C |
| 10434719 | Kng1 | kininogen 1 |
| 10487392 | Kcnip3 | Kv channel interacting protein 3, calsenilin |
| 10529937 | Kcnip4 | Kv channel interacting protein 4 |
| 10385096 | Kcnip1 | Kv channel-interacting protein 1 |
| 10401527 | Ltbp2 | latent transforming growth factor beta binding protein 2 |
| 10593449 | Layn | layilin |
| 10536711 | Lmod2 | leiomodin 2 (cardiac) |
| 10540401 | Lrrn1 | leucine rich repeat protein 1, neuronal |
| 10369752 | Lrrtm3 | leucine rich repeat transmembrane neuronal 3 |
| 10363082 | Lilrb4 | leukocyte immunoglobulin-like receptor, subfamily B, member 4 |
| 10548940 | Lmo3 | LIM domain only 3 |
| 10358272 | Lhx9 | LIM homeobox protein 9 |
| 10351443 | Lmx1a | LIM homeobox transcription factor 1 alpha |
| 10435752 | Lsamp | limbic system-associated membrane protein |
| 10366229 | Lin7a | lin-7 homolog A (C. elegans) |
| 10470175 | Lcn13 | lipocalin 13 |
| 10481627 | Lcn2 | lipocalin 2 |
| 10354141 | Lonrf2 | LON peptidase N-terminal domain and ring finger 2 |
| 10444674 | Ly6g6c | lymphocyte antigen 6 complex, locus G6C |
| 10416437 | Lcp1 | lymphocyte cytosolic protein 1 |
| 10601412 | Lpar4 | lysophosphatidic acid receptor 4 |
| 10508663 | Laptm5 | lysosomal-associated protein transmembrane 5 |
| 10372648 | Lyz2 | lysozyme 2 |
| 10458894 | Lox | lysyl oxidase |
| 10476594 | Macrod2 | MACRO domain containing 2 |
| 10476592 | Macrod2 | MACRO domain containing 2 |
| 10476588 | Macrod2 | MACRO domain containing 2 |
| 10476590 | Macrod2 | MACRO domain containing 2 |
| 10476582 | Macrod2 | MACRO domain containing 2 |
| 10461721 | Mpeg1 | macrophage expressed gene 1 |
| 10578264 | Msr1 | macrophage scavenger receptor 1 |
| 10513455 | Mup2 | major urinary protein 2 |
| 10513467 | Mup2 | major urinary protein 2 |
| 10513437 | Mup2 | major urinary protein 2 |
| 10513472 | Mup2 | major urinary protein 2 |
| 10513428 | Mup2 | major urinary protein 2 |
| 10513504 | Mup2 | major urinary protein 2 |
| 10513497 | Mup2 | major urinary protein 2 |
| 10513512 | Mup2 | major urinary protein 2 |
| 10513514 | Mup5 | major urinary protein 5 |
| 10469358 | Mrc1 | mannose receptor, C type 1 |
| 10602805 | Mtap7d2 | MAP7 domain containing 2 |
| 10492231 | Med12l | mediator of RNA polymerase II transcription, subunit 12 homolog (yeast)-like |
| 10561187 | Mia1 | melanoma inhibitory activity 1 |
| 10492355 | Mme | membrane metallo endopeptidase |
| 10466190 | Ms4a14 | membrane-spanning 4-domains, subfamily A, member 14 |
| 10461622 | Ms4a6b | membrane-spanning 4-domains, subfamily A, member 6B |
| 10461614 | Ms4a6c | membrane-spanning 4-domains, subfamily A, member 6C |
| 10466210 | Ms4a6d | membrane-spanning 4-domains, subfamily A, member 6D |
| 10466200 | Ms4a7 | membrane-spanning 4-domains, subfamily A, member 7 |
| 10580247 | Mast1 | microtubule associated serine/threonine kinase 1 |
| 10480432 | Mastl | microtubule associated serine/threonine kinase-like |
| 10531869 | Mapk10 | mitogen-activated protein kinase 10 |
| 10485151 | Mapk8ip1 | mitogen-activated protein kinase 8 interacting protein 1 |
| 10426244 | Mapk8ip2 | mitogen-activated protein kinase 8 interacting protein 2 |
| 10460947 | Pygm | muscle glycogen phosphorylase |
| 10479698 | Myt1 | myelin transcription factor 1 |
| 10488387 | Napb | N-ethylmaleimide sensitive fusion protein attachment protein beta |
| 10575880 | Necab2 | N-terminal EF-hand calcium binding protein 2 |
| 10510265 | Nppa | natriuretic peptide precursor type A |
| 10510260 | Nppb | natriuretic peptide precursor type B |
| 10356345 | Nppc | natriuretic peptide precursor type C |
| 10553450 | Nell1 | NEL-like 1 (chicken) |
| 10408359 | Nrsn1 | neurensin 1 |
| 10453518 | Nrxn1 | neurexin I |
| 10357736 | Nfasc | neurofascin |
| 10383920 | Nefh | neurofilament, heavy polypeptide |
| 10416175 | Nefl | neurofilament, light polypeptide |
| 10421100 | Nefm | neurofilament, medium polypeptide |
| 10565067 | Nmb | neuromedin B |
| 10375019 | Nsg2 | neuron specific gene family member 2 |
| 10477986 | Nnat | neuronatin |
| 10558400 | Nps | neuropeptide S |
| 10544704 | Npvf | neuropeptide VF precursor |
| 10601942 | Nrk | Nik related kinase |
| 10600707 | Nr0b1 | nuclear receptor subfamily 0, group B, member 1 |
| 10482772 | Nr4a2 | nuclear receptor subfamily 4, group A, member 2 |
| 10606174 | Nap1l2 | nucleosome assembly protein 1-like 2 |
| 10545041 | Nap1l5 | nucleosome assembly protein 1-like 5 |
| 10603266 | Nudt10 | nudix (nucleoside diphosphate linked moiety X)-type motif 10 |
| 10598236 | Nudt11 | nudix (nucleoside diphosphate linked moiety X)-type motif 11 |
| 10364102 | Ndg2 | Nur77 downstream gene 2 |
| 10470529 | Olfm1 | olfactomedin 1 |
| 10485813 | Olfr1314 | olfactory receptor 1314 |
| 10474524 | Olfr1318 | olfactory receptor 1318 |
| 10521731 | Ncapg | on-SMC condensin I complex, subunit G |
| 10427471 | Osmr | oncostatin M receptor |
| 10584024 | Opcml | opioid binding protein/cell adhesion molecule-like |
| 10405063 | Ogn | osteoglycin |
| 10476628 | Otor | otoraplin |
| 10607484 | Ptchd1 | patched domain containing 1 |
| 10426425 | Pdzrn4 | PDZ domain containing RING finger 4 |
| 10497713 | Pex5l | peroxisomal biogenesis factor 5-like |
| 10529979 | Ppargc1a | peroxisome proliferative activated receptor, gamma, coactivator 1 alpha |
| 10529977 | Ppargc1a | peroxisome proliferative activated receptor, gamma, coactivator 1 alpha |
| 10544941 | Pde1c | phosphodiesterase 1C |
| 10555510 | Pde2a | phosphodiesterase 2A, cGMP-stimulated |
| 10443786 | Pde9a | phosphodiesterase 9A |
| 10384015 | Pgam2 | phosphoglycerate mutase 2 |
| 10358434 | Pla2g4a | phospholipase A2, group IVA (cytosolic, calcium-dependent) |
| 10462922 | Plce1 | phospholipase C, epsilon 1 |
| 10360463 | Pld5 | phospholipase D family, member 5 |
| 10470959 | Phyhd1 | phytanoyl-CoA dioxygenase domain containing 1 |
| 10369835 | Phyhipl | phytanoyl-CoA hydroxylase interacting protein-like |
| 10413047 | Plau | plasminogen activator, urokinase |
| 10523134 | Pf4 | platelet factor 4 |
| 10492689 | Pdgfc | platelet-derived growth factor, C polypeptide |
| 10384458 | Plek | pleckstrin |
| 10482802 | Pscdbp | pleckstrin homology, Sec7 and coiled-coil domains, binding protein |
| 10550400 | Pnmal2 | PNMA-like 2 |
| 10366391 | Kcnc2 | potassium voltage gated channel, Shaw-related subfamily, member 2 |
| 10537458 | EG434008 | predicted gene, EG434008 |
| 10344674 | EG620393 | predicted gene, EG620393 |
| 10566571 | EG668108 | predicted gene, EG668108 |
| 10566578 | EG668108 | predicted gene, EG668108 |
| 10578017 | ENSMUSG00000053570 | predicted gene, ENSMUSG00000053570 |
| 10454441 | ENSMUSG00000053802 | predicted gene, ENSMUSG00000053802 |
| 10417302 | ENSMUSG00000063277 | predicted gene, ENSMUSG00000063277 |
| 10417258 | ENSMUSG00000063277 | predicted gene, ENSMUSG00000063277 |
| 10417264 | ENSMUSG00000063277 | predicted gene, ENSMUSG00000063277 |
| 10417235 | ENSMUSG00000068790 | predicted gene, ENSMUSG00000068790 |
| 10417366 | ENSMUSG00000068790 | predicted gene, ENSMUSG00000068790 |
| 10417461 | ENSMUSG00000072735 | predicted gene, ENSMUSG00000072735 |
| 10578950 | ENSMUSG00000074303 | predicted gene, ENSMUSG00000074303 |
| 10379727 | OTTMUSG00000000971 | predicted gene, OTTMUSG00000000971 |
| 10510215 | OTTMUSG00000010657 | predicted gene, OTTMUSG00000010657 |
| 10487506 | OTTMUSG00000015351 | predicted gene, OTTMUSG00000015351 |
| 10486201 | OTTMUSG00000015946 | predicted gene, OTTMUSG00000015946 |
| 10465424 | OTTMUSG00000018617 | predicted gene, OTTMUSG00000018617 |
| 10605067 | Pnck | pregnancy upregulated non-ubiquitously expressed CaM kinase |
| 10505489 | Pappa | pregnancy-associated plasma protein A |
| 10511363 | Penk1 | preproenkephalin 1 |
| 10394240 | Pomc | pro-opiomelanocortin-alpha |
| 10523128 | Ppbp | pro-platelet basic protein |
| 10555323 | P4ha3 | procollagen-proline, 2-oxoglutarate 4-dioxygenase (proline 4-hydroxylase), alpha polypeptide |
| | | III |
| 10406229 | Pcsk1 | proprotein convertase subtilisin/kexin type 1 |
| 10598493 | Pcsk1n | proprotein convertase subtilisin/kexin type 1 inhibitor |
| 10476633 | Pcsk2 | proprotein convertase subtilisin/kexin type 2 |
| 10361023 | Prox1 | prospero-related homeobox 1 |
| 10565456 | Prss23 | protease, serine, 23 |
| 10469255 | Prkcq | protein kinase C, theta |
| 10521471 | Ppp2r2c | protein phosphatase 2 (formerly 2A), regulatory subunit B (PR 52), gamma isoform |
| 10358224 | Ptprc | protein tyrosine phosphatase, receptor type, C |
| 10399121 | Ptprn2 | protein tyrosine phosphatase, receptor type, N polypeptide 2 |
| 10601569 | Pcdh11x | protocadherin 11 X-linked |
| 10498018 | Pcdh18 | protocadherin 18 |
| 10455084 | Pcdhb10 | protocadherin beta 10 |
| 10461869 | Prune2 | prune homolog 2 (Drosophila) |
| 10363455 | Pcbd1 | pterin 4 alpha carbinolamine dehydratase/dimerization cofactor of hepatocyte nuclear factor |
| | | 1 alpha (TCF1) 1 |
| 10437205 | Pcp4 | Purkinje cell protein 4 |
| 10425158 | Pdxp | pyridoxal (pyridoxine, vitamin B6) phosphatase |
| 10428388 | Rspo2 | R-spondin 2 homolog (Xenopus laevis) |
| 10368495 | Rspo3 | R-spondin 3 homolog (Xenopus laevis) |
| 10572485 | Rab3a | RAB3A, member RAS oncogene family |
| 10412066 | Rab3c | RAB3C, member RAS oncogene family |
| 10588283 | Rab6b | RAB6B, member RAS oncogene family |
| 10472860 | Rapgef4 | Rap guanine nucleotide exchange factor (GEF) 4 |
| 10410995 | Rasgrf2 | RAS protein-specific guanine nucleotide-releasing factor 2 |
| 10457929 | Rit2 | Ras-like without CAAX 2 |
| 10446965 | Rasgrp3 | RAS, guanyl releasing protein 3 |
| 10531610 | Rasgef1b | RasGEF domain family, member 1B |
| 10539200 | Reg1 | regenerating islet-derived 1 |
| 10545569 | Reg3g | regenerating islet-derived 3 gamma |
| 10355836 | Resp18 | regulated endocrine-specific protein 18 |
| 10360418 | Rgs7 | regulator of G protein signaling 7 |
| 10547227 | Ret | ret proto-oncogene |
| 10400926 | Rtn1 | reticulon 1 |
| 10362091 | Raet1d | retinoic acid early transcript delta |
| 10427744 | Rai14 | retinoic acid induced 14 |
| 10485963 | Arhgap11a | Rho GTPase activating protein 11A |
| 10492682 | 1110032E23Rik | RIKEN cDNA 1110032E23 gene |
| 10577641 | 1810011O10Rik | RIKEN cDNA 1810011O10 gene |
| 10354506 | 2210010L05Rik | RIKEN cDNA 2210010L05 gene |
| 10513420 | 2610016E04Rik | RIKEN cDNA 2610016E04 gene |
| 10386211 | 3100002J23Rik | RIKEN cDNA 3100002J23 gene |
| 10410460 | 3110006E14Rik | RIKEN cDNA 3110006E14 gene |
| 10436363 | 4631422O05Rik | RIKEN cDNA 4631422O05 gene |
| 10461723 | 4632417K18Rik | RIKEN cDNA 4632417K18 gene |
| 10593460 | 4833427G06Rik | RIKEN cDNA 4833427G06 gene |
| 10476795 | 4930529M08Rik | RIKEN cDNA 4930529M08 gene |
| 10369132 | 4930589M24Rik | RIKEN cDNA 4930589M24 gene |
| 10607945 | 4933400A11Rik | RIKEN cDNA 4933400A11 gene |
| 10362363 | 6330407J23Rik | RIKEN cDNA 6330407J23 gene |
| 10363161 | 6330442E10Rik | RIKEN cDNA 6330442E10 gene |
| 10604175 | 6430550H21Rik | RIKEN cDNA 6430550H21 gene |
| 10371627 | 8030451F13Rik | RIKEN cDNA 8030451F13 gene |
| 10565152 | 9330120H11Rik | RIKEN cDNA 9330120H11 gene |
| 10362372 | 9330159F19Rik | RIKEN cDNA 9330159F19 gene |
| 10461878 | A230083H22Rik | RIKEN cDNA A230083H22 gene |
| 10455942 | A730017C20Rik | RIKEN cDNA A730017C20 gene |
| 10418092 | A830039N20Rik | RIKEN cDNA A830039N20 gene |
| 10412537 | B930046C15Rik | RIKEN cDNA B930046C15 gene |
| 10392910 | C630004H02Rik | RIKEN cDNA C630004H02 gene |
| 10485550 | D430041D05Rik | RIKEN cDNA D430041D05 gene |
| 10485546 | D430041D05Rik | RIKEN cDNA D430041D05 gene |
| 10417319 | D830030K20Rik | RIKEN cDNA D830030K20 gene |
| 10412549 | D830030K20Rik | RIKEN cDNA D830030K20 gene |
| 10474129 | E430002G05Rik | RIKEN cDNA E430002G05 gene |
| 10469951 | Rnf208 | ring finger protein 208 |
| 10381574 | Rundc3a | RUN domain containing 3A |
| 10607705 | S100g | S100 calcium binding protein G |
| 10385466 | Sgcd | sarcoglycan, delta (dystrophin-associated glycoprotein) |
| 10395389 | Sostdc1 | sclerostin domain containing 1 |
| 10569129 | Sct | secretin |
| 10355960 | Scg2 | secretogranin II |
| 10595033 | Scg3 | secretogranin III |
| 10485955 | Scg5 | secretogranin V |
| 10489463 | Slpi | secretory leukocyte peptidase inhibitor |
| 10557535 | Sez6l2 | seizure related 6 homolog like 2 |
| 10519717 | Sema3a | sema domain, immunoglobulin domain (Ig), short basic domain, secreted, (semaphorin) 3A |
| 10519693 | Sema3d | sema domain, immunoglobulin domain (Ig), short basic domain, secreted, (semaphorin) 3D |
| 10425726 | Sept3 | septin 3 |
| 10380067 | Sept4 | septin 4 |
| 10349174 | Serpinb8 | serine (or cysteine) peptdiase inhibitor, clade B, member 8 |
| 10408557 | Serpinb1a | serine (or cysteine) peptidase inhibitor, clade B, member 1a |
| 10534667 | Serpine1 | serine (or cysteine) peptidase inhibitor, clade E, member 1 |
| 10484463 | Serping1 | serine (or cysteine) peptidase inhibitor, clade G, member 1 |
| 10492628 | Serpini1 | serine (or cysteine) peptidase inhibitor, clade I, member 1 |
| 10566730 | Stk33 | serine/threonine kinase 33 |
| 10563597 | Saa3 | serum amyloid A 3 |
| 10506360 | Sgip1 | SH3-domain GRB2-like (endophilin) interacting protein 1 |
| 10505705 | Sh3gl2 | SH3-domain GRB2-like 2 |
| 10431051 | Scube1 | signal peptide, CUB domain, EGF-like 1 |
| 10416887 | Slain1 | SLAIN motif family, member 1 |
| 10564205 | Snord116 | small nucleolar RNA, C/D box 116 |
| 10564187 | Snord116 | small nucleolar RNA, C/D box 116 |
| 10564195 | Snord116 | small nucleolar RNA, C/D box 116 |
| 10564193 | Snord116 | small nucleolar RNA, C/D box 116 |
| 10564179 | Snord116 | small nucleolar RNA, C/D box 116 |
| 10564163 | Snord116 | small nucleolar RNA, C/D box 116 |
| 10564173 | Snord116 | small nucleolar RNA, C/D box 116 |
| 10564167 | Snord116 | small nucleolar RNA, C/D box 116 |
| 10564201 | Snord116 | small nucleolar RNA, C/D box 116 |
| 10564199 | Snord116 | small nucleolar RNA, C/D box 116 |
| 10564189 | Snord116 | small nucleolar RNA, C/D box 116 |
| 10564207 | Snord116 | small nucleolar RNA, C/D box 116 |
| 10564185 | Snord116 | small nucleolar RNA, C/D box 116 |
| 10564191 | Snord116 | small nucleolar RNA, C/D box 116 |
| 10564171 | Snord116 | small nucleolar RNA, C/D box 116 |
| 10564181 | Snord116 | small nucleolar RNA, C/D box 116 |
| 10564197 | Snord116 | small nucleolar RNA, C/D box 116 |
| 10564175 | Snord116 | small nucleolar RNA, C/D box 116 |
| 10564161 | Snord116 | small nucleolar RNA, C/D box 116 |
| 10564177 | Snord116 | small nucleolar RNA, C/D box 116 |
| 10472374 | Scn2a1 | sodium channel, voltage-gated, type II, alpha 1 |
| 10472378 | Scn2a1 | sodium channel, voltage-gated, type II, alpha 1 |
| 10483215 | Scn3a | sodium channel, voltage-gated, type III, alpha |
| 10483228 | Scn3a | sodium channel, voltage-gated, type III, alpha |
| 10584549 | Scn3b | sodium channel, voltage-gated, type III, beta |
| 10522388 | Slc10a4 | solute carrier family 10 (sodium/bile acid cotransporter family), member 4 |
| 10530499 | Slc10a4 | solute carrier family 10 (sodium/bile acid cotransporter family), member 4 |
| 10553501 | Slc17a6 | solute carrier family 17 (sodium-dependent inorganic phosphate cotransporter), member 6 |
| 10464370 | Slc18a2 | solute carrier family 18 (vesicular monoamine), member 2 |
| 10431711 | Slc2a13 | solute carrier family 2 (facilitated glucose transporter), member 13 |
| 10514240 | Slc24a2 | solute carrier family 24 (sodium/potassium/calcium exchanger), member 2 |
| 10598507 | Slc38a5 | solute carrier family 38, member 5 |
| 10496975 | Slc44a5 | solute carrier family 44, member 5 |
| 10451838 | Slc5a7 | solute carrier family 5 (choline transporter), member 7 |
| 10553430 | Slc6a5 | solute carrier family 6 (neurotransmitter transporter, glycine), member 5 |
| 10419854 | Slc7a8 | solute carrier family 7 (cationic amino acid transporter, y+ system), member 8 |
| 10603814 | Slc9a7 | solute carrier family 9 (sodium/hydrogen exchanger), member 7 |
| 10382341 | Sstr2 | somatostatin receptor 2 |
| 10396936 | Smoc1 | SPARC related modular calcium binding 1 |
| 10409616 | Spock1 | sparc/osteonectin, cwcv and kazal-like domains proteoglycan 1 |
| 10451763 | Satb1 | special AT-rich sequence binding protein 1 |
| 10502881 | St6galnac5 | ST6 (alpha-N-acetyl-neuraminyl-2,3-beta-galactosyl-1,3)-N-acetylgalactosaminide alpha-2,6- |
| | | sialyltransferase 5 |
| 10456237 | St8sia3 | ST8 alpha-N-acetyl-neuraminide alpha-2,8-sialyltransferase 3 |
| 10490665 | Stmn3 | stathmin-like 3 |
| 10416090 | Stmn4 | stathmin-like 4 |
| 10519497 | Steap4 | STEAP family member 4 |
| 10492558 | Smc4 | structural maintenance of chromosomes 4 |
| 10344897 | Sulf1 | sulfatase 1 |
| 10352439 | Susd4 | sushi domain containing 4 |
| 10603843 | Syn1 | synapsin I |
| 10540880 | Syn2 | synapsin II |
| 10494372 | Sv2a | synaptic vesicle glycoprotein 2 a |
| 10598359 | Syp | synaptophysin |
| 10476512 | Snap25 | synaptosomal-associated protein 25 |
| 10595496 | Snap91 | synaptosomal-associated protein 91 |
| 10372324 | Syt1 | synaptotagmin I |
| 10457942 | Syt4 | synaptotagmin IV |
| 10431625 | Syt10 | synaptotagmin X |
| 10567289 | Syt17 | synaptotagmin XVII |
| 10545086 | Snca | synuclein, alpha |
| 10536363 | Tac1 | tachykinin 1 |
| 10598626 | Tspan7 | tetraspanin 7 |
| 10474700 | Thbs1 | thrombospondin 1 |
| 10598976 | Timp1 | tissue inhibitor of metalloproteinase 1 |
| 10601385 | Tlr13 | toll-like receptor 13 |
| 10580522 | Tox3 | TOX high mobility group box family member 3 |
| 10601874 | Tceal3 | transcription elongation factor A (SII)-like 3 |
| 10606864 | Tceal5 | transcription elongation factor A (SII)-like 5 |
| 10606789 | Tceal6 | transcription elongation factor A (SII)-like 6 |
| 10439695 | Tagln3 | transgelin 3 |
| 10485745 | Tmem16c | transmembrane protein 16C |
| 10601701 | Tmem35 | transmembrane protein 35 |
| 10494069 | Tnrc4 | trinucleotide repeat containing 4 |
| 10498620 | Trim59 | tripartite motif-containing 59 |
| 10489545 | Tnnc2 | troponin C2, fast |
| 10576332 | Tubb3 | tubulin, beta 3 |
| 10452295 | Tubb4 | tubulin, beta 4 |
| 10416230 | Tnfrsf10b | tumor necrosis factor receptor superfamily, member 10b |
| 10551883 | Tyrobp | TYRO protein tyrosine kinase binding protein |
| 10505614 | Tyrp1 | tyrosinase-related protein 1 |
| 10569370 | Th | tyrosine hydroxylase |
| 10522208 | Uchl1 | ubiquitin carboxy-terminal hydrolase L1 |
| 10472136 | Galnt13 | UDP-N-acetyl-alpha-D-galactosamine:polypeptide N-acetylgalactosaminyltransferase 13 |
| 10577996 | Unc5d | unc-5 homolog D (C. elegans) |
| 10555389 | Ucp2 | uncoupling protein 2 (mitochondrial, proton carrier) |
| 10374356 | Vstm2a | V-set and transmembrane domain containing 2A |
| 10410931 | Vcan | versican |
| 10447006 | Vit | vitrin |
| 10374315 | Vwc2 | von Willebrand factor C domain containing 2 |
| 10389877 | Wfikkn2 | WAP, follistatin/kazal, immunoglobulin, kunitz and netrin domain containing 2 |
| 10505163 | Zkscan16 | zinc finger with KRAB and SCAN domains 16 |
| 10599187 | Zcchc12 | zinc finger, CCHC domain containing 12 |
| 10601903 | Zcchc18 | zinc finger, CCHC domain containing 18 |
| 10606366 | Zcchc5 | zinc finger, CCHC domain containing 5 |
| 10571399 | Zdhhc2 | zinc finger, DHHC domain containing 2 |

Noteworthy, many representative genes of the DA phenotype like Th, Vmat2, Aadc, Ret, Gfra 1, Foxa1, Gdnf and Drd2 were highly enriched (FIG. 2d). Conversely, genes coding for adrenergic and serotonergic biosynthetic enzymes were found not up-regulated in the reprogrammed cells (FIG. 2e). Moreover, the fibroblast markers Twist2, Zeb2, Tgfb1i1 and Chd2¹² were down-regulated in iDAN cells (FIG. 2f). These findings indicate that the genetic reprogramming has erased the majority of the evident expression hallmarks of the cell of origin, while specifically inducing the DA neuronal phenotype and not the one of other closely related neuronal subtypes. It should be noted that iDAN expression profiling was close but distinguishable from that of mDA neurons with 160 genes differently expressed with a ≦5 fold change (Table IV).

TABLE IV
List of all genes differentially expressed (>5 fold change) between 16 DIV
iDAN cells and A9 and A10 mesencephalic DA neurons.

| AFFY ID | SYMBOL | DESCRIPTION |
|---|---|---|
| 10577641 | 1810011O10Rik | RIKEN cDNA 1810011O10 gene |
| 10440522 | Adamts1 | a disintegrin-like and metallopeptidase (reprolysin type) with thrombospondin type 1 motif, 1 |
| 10523451 | Anxa3 | annexin A3 |
| 10414065 | Anxa8 | annexin A8 |
| 10485963 | Arhgap11a | Rho GTPase activating protein 11A |
| 10536324 | Asb4 | ankyrin repeat and SOCS box-containing protein 4 |
| 10405047 | Aspn | asporin |
| 10499358 | Bglap2 | bone gamma-carboxyglutamate protein 2 |
| 10487480 | Bub1 | budding uninhibited by benzimidazoles 1 homolog (S. cerevisiae) |
| 10517517 | C1qa | complement component 1, q subcomponent, alpha polypeptide |
| 10423080 | C1qtnf3 | C1q and tumor necrosis factor related protein 3 |
| 10452316 | C3 | complement component 3 |
| 10547657 | C3ar1 | complement component 3a receptor 1 |
| 10490913 | Car3 | carbonic anhydrase 3 |
| 10474875 | Casc5 | cancer susceptibility candidate 5 |
| 10583008 | Casp12 | caspase 12 |
| 10587383 | Cd109 | CD109 antigen |
| 10406928 | Cd180 | CD180 antigen |
| 10528207 | Cd36 | CD36 antigen |
| 10501063 | Cd53 | CD53 antigen |
| 10387536 | Cd68 | CD68 antigen |
| 10435704 | Cd80 | CD80 antigen |
| 10488382 | Cd93 | CD93 antigen |
| 10575034 | Cdh3 | cadherin 3 |
| 10496204 | Cenpe | centromere protein E |
| 10450325 | Cfb | complement factor B |
| 10503161 | Chd7 | chromodomain helicase DNA binding protein 7 |
| 10502552 | Clca1 | chloride channel calcium activated 1 |
| 10502575 | Clca4 | chloride channel calcium activated 4 |
| 10541614 | Clec4d | C-type lectin domain family 4, member d |
| 10498337 | Clrn1 | clarin 1 |
| 10528864 | Cnpy1 | canopy 1 homolog (zebrafish) |
| 10595211 | Col12a1 | collagen, type XII, alpha 1 |
| 10569008 | Cox8b | cytochrome c oxidase, subunit VIIIb |
| 10554789 | Ctsc | cathepsin C |
| 10533401 | Cux2 | cut-like homeobox 2 |
| 10523145 | Cxcl15 | chemokine (C—X—C motif) ligand 15 |
| 10603551 | Cybb | cytochrome b-245, beta polypeptide |
| 10496125 | Dkk2 | dickkopf homolog 2 (Xenopus laevis) |
| 10351111 | Dnm3os | dynamin 3, opposite strand |
| 10500204 | Ecm1 | extracellular matrix protein 1 |
| 10542355 | Emp1 | epithelial membrane protein 1 |
| 10446282 | Emr1 | EGF-like module containing, mucin-like, hormone receptor-like sequence 1 |
| 10377938 | Eno3 | enolase 3, beta muscle |
| 10368289 | Enpp1 | ectonucleotide pyrophosphatase/phosphodiesterase 1 |
| 10523175 | Ereg | epiregulin |
| 10495675 | F3 | coagulation factor III |
| 10540085 | Fbln2 | fibulin 2 |
| 10409999 | Fbp2 | fructose bisphosphatase 2 |
| 10360070 | Fcer1g | Fc receptor, IgE, high affinity I, gamma polypeptide |
| 10360040 | Fcgr3 | Fc receptor, IgG, low affinity III |
| 10475643 | Fgf7 | fibroblast growth factor 7 |
| 10422760 | Fyb | FYN binding protein |
| 10464471 | Gal | galanin |
| 10472136 | Galnt13 | UDP-N-acetyl-alpha-D-galactosamine:polypeptide N-acetylgalactosaminyltransferase 13 |
| 10489179 | Ghrh | growth hormone releasing hormone |
| 10380571 | Gngt2 | guanine nucleotide binding protein (G protein), gamma transducing activity polypeptide 2 |
| 10602896 | Gpr64 | G protein-coupled receptor 64 |
| 10569335 | H19 | H19 fetal liver mRNA |
| 10385248 | Hmmr | hyaluronan mediated motility receptor (RHAMM) |
| 10417326 | Hn1l | hematological and neurological expressed 1-like |
| 10581605 | Hp | haptoglobin |
| 10571840 | Hpgd | hydroxyprostaglandin dehydrogenase 15 (NAD) |
| 10531737 | Hpse | heparanase |
| 10593233 | Htr3a | 5-hydroxytryptamine (serotonin) receptor 3A |
| 10462623 | Ifit1 | interferon-induced protein with tetratricopeptide repeats 1 |
| 10345791 | Il1rl1 | interleukin 1 receptor-like 1 |
| 10469816 | Il1rn | interleukin 1 receptor antagonist |
| 10403743 | Inhba | inhibin beta-A |
| 10586079 | Itga11 | integrin, alpha 11 |
| 10480090 | Itga8 | integrin alpha 8 |
| 10480003 | Itih2 | inter-alpha trypsin inhibitor, heavy chain 2 |
| 10462632 | Kif20b | kinesin family member 20B |
| 10508663 | Laptm5 | lysosomal-associated protein transmembrane 5 |
| 10481627 | Lcn2 | lipocalin 2 |
| 10416437 | Lcp1 | lymphocyte cytosolic protein 1 |
| 10358272 | Lhx9 | LIM homeobox protein 9 |
| 10363082 | Lilrb4 | leukocyte immunoglobulin-like receptor, subfamily B, member 4 |
| 10351443 | Lmx1a | LIM homeobox transcription factor 1 alpha |
| 10601412 | Lpar4 | lysophosphatidic acid receptor 4 |
| 10401527 | Ltbp2 | latent transforming growth factor beta binding protein 2 |
| 10444674 | Ly6g6c | lymphocyte antigen 6 complex, locus G6C |
| 10372648 | Lyz2 | lysozyme 2 |
| 10561187 | Mia1 | melanoma inhibitory activity 1 |
| 10492355 | Mme | membrane metallo endopeptidase |
| 10461614 | Ms4a6c | membrane-spanning 4-domains, subfamily A, member 6C |
| 10479698 | Myt1 | myelin transcription factor 1 |
| 10565067 | Nmb | neuromedin B |
| 10510265 | Nppa | natriuretic peptide precursor type A |
| 10510260 | Nppb | natriuretic peptide precursor type B |
| 10544704 | Npvf | neuropeptide VF precursor |
| 10601942 | Nrk | Nik related kinase |
| 10405063 | Ogn | osteoglycin |
| 10427471 | Osmr | oncostatin M receptor |
| 10476628 | Otor | otoraplin |
| 10555323 | P4ha3 | procollagen-proline, 2-oxoglutarate 4-dioxygenase (proline 4-hydroxylase), alpha polypeptide III |
| 10498018 | Pcdh18 | protocadherin 18 |
| 10544941 | Pde1c | phosphodiesterase 1C |
| 10443786 | Pde9a | phosphodiesterase 9A |
| 10384015 | Pgam2 | phosphoglycerate mutase 2 |
| 10358434 | Pla2g4a | phospholipase A2, group IVA (cytosolic, calcium-dependent) |
| 10413047 | Plau | plasminogen activator, urokinase |
| 10360463 | Pld5 | phospholipase D family, member 5 |
| 10384458 | Plek | pleckstrin |
| 10394240 | Pomc | pro-opiomelanocortin-alpha |
| 10361023 | Prox1 | prospero-related homeobox 1 |
| 10565456 | Prss23 | protease, serine, 23 |
| 10358224 | Ptprc | protein tyrosine phosphatase, receptor type, C |
| 10427744 | Rai14 | retinoic acid induced 14 |
| 10531610 | Rasgef1b | RasGEF domain family, member 1B |
| 10539200 | Reg1 | regenerating islet-derived 1 |
| 10428388 | Rspo2 | R-spondin 2 homolog (Xenopus laevis) |
| 10368495 | Rspo3 | R-spondin 3 homolog (Xenopus laevis) |
| 10607705 | S100g | S100 calcium binding protein G |
| 10563597 | Saa3 | serum amyloid A 3 |
| 10569129 | Sct | secretin |
| 10519717 | Sema3a | sema domain, immunoglobulin domain (Ig), short basic domain, secreted, (semaphorin) 3A |
| 10519693 | Sema3d | sema domain, immunoglobulin domain (Ig), short basic domain, secreted, (semaphorin) 3D |
| 10408557 | Serpinb1a | serine (or cysteine) peptidase inhibitor, clade B, member 1a |
| 10349174 | Serpinb8 | serine (or cysteine) peptdiase inhibitor, clade B, member 8 |
| 10534667 | Serpine1 | serine (or cysteine) peptidase inhibitor, clade E, member 1 |
| 10484463 | Serping1 | serine (or cysteine) peptidase inhibitor, clade G, member 1 |
| 10385466 | Sgcd | sarcoglycan, delta (dystrophin-associated glycoprotein) |
| 10598507 | Slc38a5 | solute carrier family 38, member 5 |
| 10451838 | Slc5a7 | solute carrier family 5 (choline transporter), member 7 |
| 10553430 | Slc6a5 | solute carrier family 6 (neurotransmitter transporter, glycine), member 5 |
| 10419854 | Slc7a8 | solute carrier family 7 (cationic amino acid transporter, y+ system), member 8 |
| 10489463 | Slpi | secretory leukocyte peptidase inhibitor |
| 10492558 | Smc4 | structural maintenance of chromosomes 4 |
| 10396936 | Smoc1 | SPARC related modular calcium binding 1 |
| 10395389 | Sostdc1 | sclerostin domain containing 1 |
| 10519497 | Steap4 | STEAP family member 4 |
| 10474700 | Thbs1 | thrombospondin 1 |
| 10598976 | Timp1 | tissue inhibitor of metalloproteinase 1 |
| 10416230 | Tnfrsf10b | tumor necrosis factor receptor superfamily, member 10b |
| 10489545 | Tnnc2 | troponin C2, fast |
| 10498620 | Trim59 | tripartite motif-containing 59 |
| 10505614 | Tyrp1 | tyrosinase-related protein 1 |
| 10555389 | Ucp2 | uncoupling protein 2 (mitochondrial, proton carrier) |
| 10577996 | Unc5d | unc-5 homolog D (C. elegans) |
| 10410931 | Vcan | versican |
| 10447006 | Vit | vitrin |
| 10389877 | Wfikkn2 | WAP, follistatin/kazal, immunoglobulin, kunitz and netrin domain containing 2 |
| 10606366 | Zcchc5 | zinc finger, CCHC domain containing 5 |
| 10603066 | Ace2 | angiotensin I converting enzyme (peptidyl-dipeptidase A) 2 |
| 10429029 | Adcy8 | adenylate cyclase 8 |
| 10366528 | Best3 | bestrophin 3 |
| 10540333 | Cntn6 | contactin 6 |
| 10573054 | Gypa | glycophorin A |
| 10385096 | Kcnip1 | Kv channel-interacting protein 1 |
| 10390860 | Krt23 | keratin 23 |
| 10470175 | Lcn13 | lipocalin 13 |
| 10513514 | Mup5 | major urinary protein 5 |
| 10553450 | Nell1 | NEL-like 1 (chicken) |
| 10600707 | Nr0b1 | nuclear receptor subfamily 0, group B, member 1 |
| 10455084 | Pcdhb10 | protocadherin beta 10 |
| 10469255 | Prkcq | protein kinase C, theta |
| 10545569 | Reg3g | regenerating islet-derived 3 gamma |
| 10496975 | Slc44a5 | solute carrier family 44, member 5 |
| 10490663 | Stmn3 | stathmin-like 3 |

Interestingly, Th and Vmat2 promoter regions were highly demethylated in DA neuronal cells while fully methylated in parental fibroblasts indicating their epigenetic reactivation during DA neuronal conversion (FIG. 8).

GFP+ cells induced by the three factors showed an elaborate neuronal morphology with multiple and long processes (FIG. 1d-l). Hence, the authors asked whether induced neuronal cells establish synaptic contacts in culture. Notably, synaptic resident proteins as synaptotagmin I (SYT1), and synapsin (SYN) were localized in discrete puncta and co-localized with TH immunolabeling suggesting the establishment of DA synaptic terminals (FIG. 9). Moreover, the successful FM4-64 dye uptake at the TH+ synaptic boutons indicated active synaptic processes (FIG. 9).

Then, the authors performed patch-clamp recordings of GFP+ iDAN cells (n=16) as well as primary mDA neurons (n=12) to compare their respective physiological properties^{13,14}. iDAN cells had higher cell resistance and lower capacitance than primary DA neurons, but showed normal resting membrane potential, normal Na⁺ currents (FIG. 3a), overshooting action potentials (FIG. 3c), and even more prominent K⁺ currents (FIG. 3b) and afterspike hyperpolarization (Table V). More than 80% of iDAN cells showed rhythmic discharges (FIG. 3d, e) at an average frequency of 2.6 Hz. The identity of voltage-gated inward Na⁺ and outward K⁺ currents in iDAN cells has been verified pharmacologically (FIG. 10). Next, the authors noted that iDAN cells, as mDA neurons, express high levels of the D2 receptor (FIG. 3f). To verify whether DA receptors are functional, the authors applied the specific D2/D3 receptor agonist quinpirole (1 μM), which drastically suppressed neuronal firing in 6 of 10 recorded cells in a reversible manner (FIG. 3g, h). Next, the authors employed amperometry for real-time electrochemical detection of monoamine secretion from iDAN cells^{15,16}. When carbone fiber electrodes were placed adjacent to GFP+ cells (FIG. 3i), depolarization of 4 cells with 25 mM K⁺ resulted in numerous amperometric events (FIG. 3i), reflecting quantal secretion of monoamines. Furthermore, direct HPLC measurements revealed that iDAN cells contain high level of intracellular dopamine detectable in pellets preparations that is released in the extracellular medium upon stimulation with 50 mM KCl (FIG. 3j). Thus, reprogrammed cells exhibit several major properties of DA neurons in terms of spontaneous spiking activity, temporal parameters of action potentials, inhibition of cell firing through D2 autoreceptors and controlled dopamine release.

Next, to determine the temporal requirement for the three exogenous factors to induce a stable reprogrammed cell state, infected MEFs were treated with dox for different time windows and then withdrew. Only when fibroblasts were treated with dox for 6 or more days numerous neuronal cells, mostly TH+, were observed (FIG. 11). Thus, reprogramming is a relative rapid process that requires the expression of the three factors for only 6 days. At the same time, iDAN cells achieved a stable neuronal state over time independently from viral transgene expression and even at 18 days of dox withdrawal, iDAN cells were found at the same number, and exhibited similar spontaneous firing as iDAN cells constantly cultivated in the presence of dox (FIG. 12; Table V).

TABLE V
Comparison between primary mesencephalic DA neurons and iDAN cells 16 days after viral
infection in the presence or absence of doxycycline (+dox and −dox, respectively).

| | | Primary | iDA | iDA |
|---|---|---|---|---|
| | | DA neurons | cells + dox | cells − dox |
| | | (n = 12) | (n = 16) | (n = 7) |
| The Parameters | Unit | Mean ± SEM | Mean ± SEM | Mean ± SEM |
| Cell resistance | (GOhm) | 0.8 ± 0.1 | 1.1 ± 0.1* | 0.9 ± 0.2 |
| Cell capacitance | (pF) | 19.5 ± 1.5 | 10.6 ± 13*** | 9.6 ± 2.1 |
| Resting membrane potential | (mV) | −43.5 ± 2.6 | −41.8 ± 1.8 | −43.0 ± 1.5 |
| Maximal amplitude of Na⁺ current | (nA) | −4.0 ± 0.5 | −4.4 ± 0.5 | −3.9 ± 0.8 |
| Amplitude of delayed rectifier K⁺ currents at +20 mV | (nA) | 1.2 ± 0.1 | 2.0 ± 0.2** | 2.5 ± 0.5 |
| Presence of rapidly inactivating A-type K⁺ currents | yes/no | 91.7% | 81.3% | 85.7% |
| | | (11 in 12) | (13 in 16) | (6 in 7) |
| Amplitude of action potential | mV | 81.7 ± 1.6 | 89.4 ± 4.1 | 90.3 ± 3.6 |
| Half-width of action potential | (ms) | 1.6 ± 0.1 | 1.2 ± 0.2 | 1.1 ± 0.1 |
| Amplitude of afterspike hyperpolarization | mV | 9.3 ± 1.2 | 14.8 ± 0.6*** | 14.1 ± 1.0 |
| Duration of afterspike hyperpolarization | ms | 50.1 ± 4.9 | 38.4 ± 4.1 | 25.9 ± 4.5 |
| Rhythmic spontaneous dischange | yes/no | 66.7% | 81.3% | 85.7% |
| | | (8 in 12) | (13 in 16) | (6 in 7) |
| Mean frequency of spikes | (Hz) | 1.4 ± 0.3 | 2.6 ± 0.4* | 4.5 ± 1.2 |

n, provides the number of recorded cells from at least two independent experiments.
*P < 0.05,
**P < 0.01 and
***P < 0.001, significant differences between primary neurons and iDAN + dox cells, unpaired t-test. No difference between +dox and −dox iDAN cells was detected.

Reprogramming of fibroblasts into differentiated neuronal cells might occur directly or passing first through neural progenitors. When the DNA-base analog BrdU was added from day 2 onwards to label the proliferating cells, virtually all neuronal cells were already post-mitotic after this time (FIG. 13b, c, h). Despite during the first 2 days infected cells were actively proliferating in serum-containing medium, none showed expression of the neural progenitor molecular markers Sox2, Ngn2, Otx2, Lmx1b and En1¹⁷ (FIG. 13i). Furthermore, the authors employed a genetic tracing system based on activation of the Sox2^{β-geo} LacZ reporter¹⁸ showing that, as a proof-of-principle, LacZ activity was easily visualized upon reprogramming of Sox2^{+/β-geo} fibroblasts into iPS cells. By contrast, the reporter was never activated from the same cells when engaged into direct iDAN reprogramming (FIG. 13j). Altogether, these findings are inconsistent with the occurrence of detectable cell intermediates during the reprogramming of fibroblasts into iDAN cells.

Next, in vivo differentiation potential of iDAN cells was assessed by orthotopic transplantations into neonatal mouse brains. Four days after viral transgene induction, infected cells were grafted into the ventricle of mouse newborn brains. Two and 6 weeks after transplantations, GFP+ cells were found integrated in the host tissue displaying an extremely elaborated morphology (FIG. 14 and FIG. 15). Most of the GFP+ grafted neuronal cells were positive to TH, AADC, VMAT2 and DAT indicating the acquirement of a full neuronal DA cell fate (FIG. 15b-g, i-l). Injection of brief supra-threshold current pulses evoked overshooting action potentials, and large Na⁺ and K⁺ currents were activated by depolarizing voltage steps (FIG. 15o, p). Therefore, iDAN cells maintain excitability and major currents in vivo even after extensive period of time from grafting.

The authors then translated the same procedure to the human system by initially infecting IMR90 fetal fibroblasts. After 18 days from infection, the authors scored numerous TuJ1+ and TH+ neuronal cells accounting, respectively, for 10±4% and 6±2% of the infected cells (FIG. 16a-c). The authors then reprogrammed adult human fibroblasts from 2 healthy donors (age 42, 55) and from 2 patients with genetic forms of PD (Table VI).

TABLE VI
Clinical assessment of the PD patients whose primary fibroblasts were utilized in the present study.

| | | | Age of | Year of | | Disease | | |
|---|---|---|---|---|---|---|---|---|
| # | Disease | Sex | Onset | Onset | Gene | duration | Clinical assesment | References |
| A-0433 | Parkinson's | F | 41 | 2004 | Synuclein | 4 | PD with good response to | 22 |
| | Disease | | | | duplication | | dopaminergic therapy, quick | |
| | | | | | | | progression and early development | |
| | | | | | | | of motor complications | |
| B-0097 | Parkinson's | F | 30 | 1982 | Parkin: | 27 | Early onset PD with good response | 23 |
| | Disease | | | | c.C815G: | | to dopaminergic therapy and slow | |
| | | | | | del ex6-7. | | progression. | |

Both healthy and diseased adult cells showed a comparable propensity in converting into neuronal cells accounting for an estimated efficiency for TuJ1+ and TH+ cells of 5±1% and 3±1%, respectively (FIG. 4a-g). iDAN cells were positive to ALDH1A1, TH, AADC, VMAT2 and DAT by immunocytochemistry (FIG. 4a-f) and gene expression analysis (FIG. 17). Cell conversion was stable over time since number and morphology of human iDAN cells was not evidently affected up to day 24 after reprogramming even when dox was withdrawn from day 6 onwards (FIG. 4h, i).

Recordings in 5 infected fetal human iDAN cells showed that the electrophysiological properties of these cells resemble mouse iDAN cells (FIG. 16). Recordings in 8 infected adult human iDAN cells revealed less mature phenotype (FIG. 4j-l), with mean amplitudes: 0.7±0.1 nA for Na⁺ currents, 1.2±0.1 nA for delayed rectifier K+ currents, 78±3 mV for action potentials, and 7.5±2 mV for afterspike hyperpolarization. The identity of Na⁺ and K⁺ currents was confirmed pharmacologically (FIG. 4m,n). Most importantly, depolarization of 3 cells with 25 mM K⁺ elicited numerous release events detected by amperometric measurements, as described above for mouse iDAN cells (FIG. 4o). In summary, these experiments suggest that actively spiking dopamine-secreting cells can be induced by forced expression of the three factors in adult human cells from both healthy donors and PD patients.

Herein, the authors demonstrated that the combination of the transcription factors Mash1, Nurr1, preferably of the three transcription factors Mash1, Nurr1 and Lmx1a or Lmx1b can rapidly and efficiently induce DA neuronal cells from mouse and human fibroblasts. Reprogrammed cells are similar to brain DA neurons in gene expression, and show dopamine release and peacemaking activity that can be modulated via D2 receptors. Importantly, this cell conversion diverges with respect to developmental neuronal lineage commitment since it is not progressing through detectable intermediate neuronal stages.

Thus, the same viral cocktail (ANL) was used to convert human adult fibroblasts. In this case the efficiency of TH+ cells is 3±0.8% (FIG. 4). In order to increase the conversion of human adult cells the authors improved the culture protocol by keeping the cells in 5% O₂ (hypoxia) instead of ˜20% (normoxia), obtaining an efficiency of 5.3±0.5% of TH+ cells (FIG. 19).

In order to assess the impact of reprogramming on the overall functional aspects in vivo, the authors tested, using transplantation studies, the capacity of iDAN cells to rescue the rotational phenotype of 6 hydroxydopamine (6OHDA) lesioned rats. These experiments revealed that since 8 weeks after iDAN cells transplantation in the lesioned striatum, there is a significative reduction of amphetamine-induced rotations (FIG. 20).

Generation of functional DA neuronal cells by direct reprogramming opens new possibilities in the regenerative therapies of neurological disorders, i.e. dopaminergic related disorders, as PD. Pfisterer et al. identified a different gene cocktail (Mash1, Brn2, Myt1l, Lmx1a, Foxa2) capable of inducing DA-like neurons from human fibroblasts²⁰. The dopaminergic-like neurons obtained with such viral cocktail show a less differentiated morphology. In addition, the presence of dopamine was not shown in the reprogrammed neurons obtained with the method of Pfisterer et al. This opens the intriguing possibility that different molecular fate determinants reach a similar endpoint even though starting from different transcriptional cascades.

In all, the present methods do not rely on pluripotent stem cells that are prone to tumors in their undifferentiated state. Moreover, the process described herein does not pass through proliferative progenitors that also might result tumorigenic²¹. Thus, the method of the invention avoids a dangerous drawback of stem cell therapies while providing enough number of functional DA neurons amenable for autologous cell replacement therapies.

### BIBLIOGRAPHIC REFERENCES

1. Lindvall, O. & Björklund, A. NeuroRx 1, 382-393 (2004).
2. Politis, M. et al. Sci. Transl. Med. 2:38ra46 (2010).
3. Kim, J. H. et al. Nature 418, 50-56 (2002).
4. Barberi, T. et al. Nature Biotechnol. 21, 1200-1207 (2003).
5. Wernig, M. et al. Proc. Natl. Acad. Sci. USA. 105, 5856-5861 (2008).
6. Heins, N. et al. Nat. Neurosci. 5, 308-315 (2002).
7. Vierbuchen, T. et al. Nature 463, 1035-1041 (2010).
8. Ang, S.-L. Development 133, 3499-3506 (2006).
9. Smidt, M. P. & Burbach J. P. Nat. Rev. Neurosci. 8, 21-32 (2007).
10. Sawamoto, K. et al. Proc. Natl. Acad. Sci. USA. 98, 6423-6438 (2001).
11. Perlmann, T. & Wallén-Mackenzie, A. Cell Tissue Res. 318, 45-52 (2004).
12. Stadtfeld, M., et al., Cell Stem Cell 2, 230-240 (2008).
13. Grace, A. A. & Bunney, B. S. J. Neurosci. 4, 2866-2876 (1984).
14. Grace, A. A & Onn, S. P. J. Neurosci. 9, 3463-3481 (1989).
15. Pothos, E. N., Davila, V. & Sulzer, D. J Neurosci. 18, 4106-4118 (1998).
16. Staal, R. G. W., Mosharov, E. V. & Sulzer, D. Nat. Neurosci 7, 341-346 (2004).
17. Simeone, A. Trends Neurosci. 28, 62-65 (2005).
18. Zappone M. V. et al. Development 127, 2367-2382 (2000).
19. Pang, Z. P. et al. Nature 476, 220-223 (2011)
20. Pfisterer, U. et al. Proc. Natl. Acad. Sci. U.S.A. 108, 10343-10348 (2011).
21. Amariglio, N. et al. PLoS Med. 6:e1000029 (2009).
22. Sironi, F. et al. Parkinsonism Relat. Disord. 16, 228-231 (2010).
23. Sironi, F. et al. Parkinsonism Relat Disord. 14, 326-333 (2008).
24. Pruszak, J. et al. Curr Prot Stem Cell Biol 2D.5.1-2D.5.21 (2009).
25. Broccoli, V. et al. Nature 401, 164-168 (1999).
26. Irizarry, R. A. et al. Nucleic Acids Res 31:e15 (2003).
27. Smyth, G. K. Stat. Appl. Genet. Mol. Biol. 3: Article 3 (2004).
28. Hochberg, Y. & Benjamin, Y. Stat. Med. 9, 811-818. (1990).
29. Huang, D. W et al. Nucleic. Acids Res. 37, 1-13 (2009).
30. Biagioli, M et al. Proc. Natl. Acad. USA 106, 15454-15459 (2009).
31. Pothos, E. et al. J Neurochem 66, 629-636 (1996).
32. Mundroff, M. L. & Wightman, R. M. Curr Protoc Neurosci Chapter 6, Unit 6.14 (2002).
33. Menegon, A. et al. J Neurosci. 26, 11670-11681.
34. Colasante G. et al. J Neurosci 28, 10674-10686 (2008).
35. West M. J. et al. The Anatomical record 231, 482-497 (1991).
36. Bacigaluppi M. et al. Brain 132, 2239-2251 (2009).

1. A method for reprogramming a differentiated non neuronal cell into a dopaminergic neuron comprising the step of inducing the expression in the differentiated non neuronal cell of at least the protein encoded by the Mash1 human gene or orthologues thereof and the protein encoded by the Nurr1 human gene or orthologues thereof.

2. The method according to claim 1 further comprising the step of inducing the expression in the differentiated non neuronal cell of the protein encoded by the Lmx1a human gene and/or by the Lmx1b human gene or orthologues thereof.

3. The method according to claim 1 further comprising the step of inducing the expression in the differentiated non neuronal cell of at least a protein encoded by a gene selected from the group of: Brn2, Myth1l, En-1, En-2, Pitx3, Foxa1, Foxa2, Otx2, Msx1 or Neurog2 human genes or orthologues thereof.

4. The method according to claim 3 comprising the step of inducing the expression in the differentiated non neuronal cell of proteins encoded by each of the following human genes or orthologues thereof: Mash1, Nurr1, Lmx1a, Lmx1b, Brn2, Myth1l, En-1, En-2, Pitx3, Foxa1, Foxa2, Otx2, Msx1 and Neurog2.

5. The method according to claim 1 wherein the differentiated non neuronal cell is a mouse or a human cell.

6. The method according to claim 1 wherein the differentiated non neuronal cell is selected from the group consisting of: a cell of mesoderm origin, a cell of ectoderm origin, a fibroblast, an astroglial cell, a skin keratinocyte and an hematopoietic cell.

7. The method according to claim 5 wherein the differentiated non neuronal cell is an adult cell.

8. The method according to claim 7 wherein the differentiated non neuronal cell is an adult cell of a healthy subject or of a subject affected by a neurological disorder.

9. The method according to claim 8 wherein the neurological disorder is characterized by dopaminergic system dysfunction.

10. The method according to claim 9 wherein the neurological disorder characterized by dopaminergic system dysfunction is Parkinson's disease.

11. The method according to claim 1 wherein the step of inducing the expression is obtained by genetically transforming the differentiated non neuronal cell with at least one vector containing and expressing the coding sequences of the protein encoded by the Mash1 human gene or orthologues thereof or the protein encoded by the Nurr1 human gene or orthologues thereof.

12. The method according to claim 11 wherein the genetic transformation is performed by transfecting or infecting the differentiated non neuronal cell.

13. The method according to claim 12 wherein the differentiated non neuronal cell is infected by a recombinant lentivirus.

14. The method according to claim 1 wherein the step of inducing the expression is performed in hypoxia conditions.

15. The method according to claim 1 wherein the step of inducing the expression is performed in the presence of 2 to 6% O₂.

16. An eukaryotic vector comprising and expressing under appropriated promoter and regulatory sequences the coding sequences of the proteins as defined in claim 1.

17. The eukaryotic vector according to claim 16 comprising and expressing under appropriated promoter and regulatory sequences the coding sequences of the proteins Mash1, Nurr1 and either Lmx1a or Lmx1b.

18. The eukaryotic vector according to claim 17 wherein the coding sequences of the proteins Mash1, Nurr1 and either Lmx1a or Lmx1b are in the following order: 5′ Mash1-Nurr1 and Lmx1a or Lmx1b 3′.

19. A method for the treatment of a neurological disorder comprising administering the vector of claim 16 to a patient in need thereof.

20. The vector according to claim 19 wherein the neurological disorder is characterized by dopaminergic system dysfunction.

21. The vector according to claim 20 wherein the neurological disorder is Parkinson's disease.

22. A reprogrammed dopaminergic neuron prepared according to the method of claim 1.

23-26. (canceled)

27. A pharmaceutical composition comprising the reprogrammed dopaminergic neuron according to claim 22.

28. A method for the screening of putative therapeutic agents comprising the step of:
incubating the reprogrammed dopaminergic neuron according to claim 22 with the putative therapeutic agents;
measuring and/or observing an appropritate phenotype in said reprogrammed dopaminergic neuron; and
comparing said measured and/or observed phenotype with an appropriated control phenotype.

29. A pharmaceutical composition comprising the vector according to claim 16.
